(19) United States
(12) Patent Application Publication
(10) Pub. No.: US 2024/0401031 A1
(43) Pub. Date: Dec. 5, 2024

(54) Platform Using iPSC-Derived Cardiomyocytes Carrying Gene Variants as Models of Cardiac Disease and Drug-Response

(71) Applicant: SRI INTERNATIONAL, Menlo Park, CA (US)

(72) Inventors: Paul OMAILLE, San Diego, CA (US); Danuta SASTRE, West Sacramento, CA (US); Wei ZHOU, Fremont, CA (US); Leena SHEWADE, Mountain View, CA (US); Joy CALAOAGAN, San Jose, CA (US)

(21) Appl. No.: 18/708,428

(22) PCT Filed: Nov. 9, 2022
(86) PCT No.: PCT/US22/79521

§ 371 Date: May 8, 2024

Related U.S. Application Data
(60) Provisional application No. 63/277,272, filed on Nov. 9, 2021.

(51) Int. Cl. C12N 15/10; C12M 1/32; G01N 33/50

(52) U.S. Cl. CPC C12N 15/1082; C12M 23/12; G01N 33/5044

(57) ABSTRACT

This disclosure is in the field of cardiac diseases. For example, the disclosure provides a new platform comprising iPSC and cardiomyocytes carrying one or more gene mutations, and models to study the effect of those gene mutations on cardiomyocytes, and on drug toxicity. This platform is identified herein as PREDICT PLATFORM.

### FIELD

This disclosure is in the field of cardiac diseases. For example, the disclosure provides a new platform comprising iPSC and cardiomyocytes carrying one or more gene mutations, and models to study the effect of those gene mutations on cardiomyocytes, and on drug toxicity. This platform is identified herein as PREDICT PLATFORM.

### CROSS-REFERENCE TO RELATED APPLICATIONS

This application claims the benefit of priority of U.S. Provisional Application No. 63/277,272, filed on Nov. 9, 2021, the contents of which are incorporated herein by reference in their entirety.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, Nov. 8, 2022, is SRI 210040 202210-20_SL.xls and is 5,495 kilobytes in size.

### BACKGROUND

Recent advances in sequencing technologies and genome wide association studies (GWAS) have identified a spectrum of genetic variants as determinants for an individual's response to drug treatments. However, translating these computational findings into a clinical application remains a major hurdle for personalizing medicine. Testing for cardiotoxicity is one of the key components of the drug development process.

Cardiotoxicity is the lead cause for approved drug withdrawals from the market and FDA mandates cardiotoxicity assessment in preclinical testing adding significantly to drug development costs. For example, several therapeutic drugs have been withdrawn from the market due to unwanted side-effects on the hERG channel that leads to potentially lethal cardiac arrhythmias. Several hERG channel mutations have been identified in the clinical setting but the lack of suitable model systems has prevented the in-depth studies of their role in adverse drug response. KCNH2 gene (hERG) encodes the potassium voltage-gated channel subfamily H member 2, the rapid delayed rectifier potassium channel implicated in cardiotoxicity events (i.e., Long QT 2 syndrome). Human variation in KCNH2 has been directly linked to Long-QT syndrome and might account for some interindividual differences in cardiotoxicity responses to drugs. Current hERG assays use monogenic (i.e., from one individual) cell lines-a one size fits all approach that ignores the contribution of interindividual responses to drugs. Thus, there is a need for hERG testing that introduces genetic diversity for better prediction of a drug's effects on a diverse human population.

Comprehensive testing of the impact of population-wide genetic diversity on adverse drug effects has not been reported to date. There is a need for genotype-phenotype maps that link pharmacologically relevant gene variants to adverse drug reaction phenotypes. These maps could provide an essential framework for prospective preclinical assessment of drug toxicity and, when coupled with companion diagnostics, empower physicians with actionable intelligence to prescribe the most effective medicines while averting adverse drug reactions.

### SUMMARY

In one embodiment, the disclosure provides a platform that includes a panel of cell lines containing genetic diversity of established clinical relevance (and variants of unknown significance) for studying these cell lines' functionalities with different drugs. Specifically, the cardiotoxicity assays using the disclosed cell lines and platform can detect adverse drug effects that arise from interindividual genetic differences.

The cell lines, related methods, and assays provided by the disclosure include cardiomyocytes derived from genome-edited human induced pluripotent stem cells (iPSC) that carry specific genetic variation of KCNH2 (hERG) allele for testing potential cardiotoxicity (and other functional effects) for various compounds. Specifically, the methods test whether the cardiomyocytes with specific KCNH2 alleles exhibit different or disease mimicking functionality compared to common (wildtype) alleles, and whether any of the compounds tested have the effect of cardiotoxicity or other functionality modification.

In addition to KCNH2, multiple genes have been shown to be related to drug induced cardiotoxicity, such as KCNQ1, SCNA5, KCNE1, and KCNE2. The disclosed cell lines, methods, and assays here can also be applied to these genes. The disclosure provides a method for characterizing all polymorphisms in a gene of interest and their potential relationship with cardiac diseases and drug-induced cardiotoxicity. The disclosed experimental data establishes proof of concept here with the KCNH2 alleles because of its high clinical relevance and presence of polymorphisms.

The following embodiments of the disclosure are just exemplary:
1. A prime editing guide RNA (pegRNA) designed for prime editing of a target gene that is hypothesized as having an association with arrhythmia and/or drug-induced toxicity to cardiomyocytes, preferably wherein the cardiomyocytes are derived from induced pluripotent stem cells (iPSC), and preferably wherein the pegRNA is barcoded.
2. The pegRNA of embodiment 1, comprising a spacer sequence and a DNA synthesis template, wherein the spacer sequence comprises a region of complementarity to a target strand of a double stranded target gene DNA sequence to be edited and the DNA synthesis template comprises a region of complementarity to the non-target strand of the double-stranded target gene DNA sequence and one or more nucleotide edits compared to the target strand double-stranded target gene DNA sequence.
3. The pegRNA of any one of embodiments 1 and 2, comprising a guide RNA (gRNA) core, and an extension arm comprising a DNA synthesis template and a primer binding site (PBS),
wherein the gRNA core associates with a nucleic acid programmable DNA binding protein (napDNAbp), which is fused to a domain comprising polymerase (preferably, RNA-dependent DNA polymerase) activity,
wherein the primer binding site comprises a region of complementarity to a non-target strand of the double-stranded target gene DNA sequence.
4. The pegRNA of any one of embodiments 1 to 3, wherein the DNA synthesis template is designed to edit the target gene in at least one site.
5. The pegRNA of embodiment 4, wherein the DNA synthesis template introduces random edits into the target gene.
6. The pegRNA of embodiment 4, wherein the DNA synthesis template introduces pre-selected edits into the target gene.
7. The pegRNA of any one of embodiments 1 to 7, wherein the target gene is selected from genes that encode potassium channels or potassium-channel related genes, sodium channels or sodium channels related genes, calcium channels and calcium channel related genes, and cardiomyocyte structural genes.
8. The pegRNA of embodiment 7, wherein the target gene is selected from potassium channel/related genes selected from human ether-a-go-go related gene (hERG)/KCNH2, Hyperpolarization-activated cyclic nucleotide-gated (HCN) channels; transient outward potassium current channel; Slowly activating delayed rectifier potassium current channels; Rapidly activating delayed rectifier potassium current channels; Inwardly rectifying potassium (Kir) channels; Inwardly rectifying potassium channels; G protein-coupled, inwardly rectifying potassium channels; ATP-sensitive potassium channels; sodium channel/related genes selected from SCN5A, ACN1B, SCN2B, SCN3B, SCN4B, GPD1L, RANGRF, SCN10A; calcium channel/related genes selected from CACNA1C, CACNB2, CACNA2D1, RYR2, CASQ2, TRDN, CALM1-3; and other genes selected from KCNQ1, SCNA5, KCNE1, KCNE2, KvLQT1, Nav1.5, ankyrin-B, MinK, MiRP1, Kir2.1, Cav1.2, caveolin-3, Nav.beta.4 genes, SNTA1, SLMAP, PKP2, ANK2, CAV3, SLC4A3, TRPM4, DPP6, IRX3, GNAI2, ADORA1, GNAS, KCNQ1, SCNA5, KCNE1, KCNE2, KvLQT1, Nav1.5, ankyrin-B, MinK, MiRP1, Kir2.1, Cav1.2, caveolin-3, Nav.beta.4 ATP-binding cassette (ABC) transporters involved in drug transport (e.g., ABCB1, ABCB4, ABCCI, ABCC2, SLC10A2, SLC28A3, SLC22A7, SLC22A17), Carbonyl reductases in drug metabolism (e.g., CBR3), Hyaluronan synthase 3 involved in oxidative stress response (e.g., HAS3), Hereditary hemochromatosis protein in iron metabolism (e.g., HFE), Retinoic acid receptor gamma and DNA topoisomerases in topoisomerase-induced DNA damage (e.g., RARG, TOP2B), CUGBP Elav-like family member 4 in splicing of sarcomere genes (e.g., CELF4), DNA polymerase gamma in mitochondrial replication (e.g., DPOG2), and chaperones involved in ion channel trafficking (e.g., Hsp70 and Hsp90).
9. The pegRNA of any one of embodiments 1 to 8, wherein the prime editing introduces one or more genetic variations into the gene, where the genetic variations have been previously identified as benign, likely benign, pathogenic, or likely pathogenic to cardiac function.
10. The pegRNA of any one of embodiments 1 to 6, wherein the drug has not been previously identified as a drug capable of inducing cardiomyocyte toxicity.
11. The pegRNA of any one of embodiments 1 to 6, wherein the drug is selected from a list of blackbox labeled drugs (currently marketed drugs that contain a label to indicate that Long QT patients should avoid using this drug; including Table 2), ii) drugs withdrawn from the US market due to cardiotoxicity events, iii) drugs that failed clinical trials due to cardiotoxicity events-(some may be marketed in other countries; iv) drugs associated with cardiotoxicity for particular SNPs as documented in the literature (e.g., ClinVar; antiarrhythmics, class i and iii; cisapride, amiodarone, dofetilide, clarithromycin, hydroxyzine, quinidine and disopyramide), and v) TdP associated drugs (including Table 3).
12. The pegRNA of any one of embodiments 1 to 11, wherein the pegRNA is designed to introduce a mutation into the human KCNH2 gene, wherein the mutation is selected from a SNP, preferably wherein the SNP is selected from the SNPs identified in Table 1 and/or SEQ ID Nos. 2-1054.
13. The pegRNA of embodiment 12, wherein the SNP causes a V476I mutation in the KCNH2 protein as in SEQ ID NO. 105.
14. The pegRNA of any one of embodiments 1 through 13, wherein the pegRNA is encoded by the DNA sequence of any one of SEQ ID Nos. 1100 to 1113.
15. A gRNA designed for the regulation of expression of a target gene by CRISPR interference, wherein the target gene is being tested for their role in cardiomyocyte function, proliferation, viability, survival, morphology, the expression of markers and receptors, and “heart beats” in vitro (which can model arrhythmias), preferably wherein the target gene is selected from: potassium channel/related genes selected from human ether-a-go-go related gene (hERG), Hyperpolarization-activated cyclic nucleotide-gated (HCN) channels; transient outward potassium current channel; Slowly activating delayed rectifier potassium current channels; Rapidly activating delayed rectifier potassium current channels; Inwardly rectifying potassium (Kir) channels; Inwardly rectifying potassium channels; G protein-coupled, inwardly rectifying potassium channels; ATP-sensitive potassium channels; sodium channel/related genes selected from SCN5A, ACN1B, SCN2B, SCN3B, SCN4B, GPD1L, RANGRF, SCN10A; calcium channel/related genes selected from CACNA1C, CACNB2, CACNA2D1, RYR2, CASQ2, TRDN, CALM1-3; and other genes selected from KCNQ1, SCNA5, KCNE1, KCNE2, KvLQT1, Nav1.5, ankyrin-B, MinK, MiRP1, Kir2.1, Cav1.2, caveolin-3, Nav.beta.4 genes, SNTAI, SLMAP, PKP2, ANK2, CAV3, SLC4A3, TRPM4, DPP6, IRX3, GNAI2, ADORAI, GNAS, KCNQ1, SCNA5, KCNE1, KCNE2, KvLQT1, Nav1.5, ankyrin-B, MinK, MiRP1, Kir2.1, Cav1.2, caveolin-3, Nav.beta.4 ATP-binding cassette (ABC) transporters involved in drug transport (e.g., ABCB1, ABCB4, ABCCI, ABCC2, SLC10A2, SLC28A3, SLC22A7, SLC22A17), Carbonyl reductases in drug metabolism (e.g., CBR3), Hyaluronan synthase 3 involved in oxidative stress response (e.g., HAS3), Hereditary hemochromatosis protein in iron metabolism (e.g., HFE), Retinoic acid receptor gamma and DNA topoisomerases in topoisomerase-induced DNA damage (e.g., RARG, TOP2B), CUGBP Elav-like family member 4 in splicing of sarcomere genes (e.g., CELF4), DNA polymerase gamma in mitochondrial replication (e.g., DPOG2), and chaperones involved in ion channel trafficking (e.g., Hsp70 and Hsp90).
16. The gRNA of embodiment 15, wherein the gRNA target sequence is selected from those of SEQ ID NO. 1116 to 1155 and described in Table 4.
17. A composition comprising a pegRNA nucleic acid according to any one of embodiments 1 to 14, and, optionally, a prime editor comprising nucleic acid programmable DNA binding protein (napDNAbp), which is fused to or bound to a domain comprising polymerase (preferably, RNA-dependent DNA polymerase) activity, a sgRNA, an iPSC, and a cardiomyocyte.
18. A composition comprising a library of two or more pegRNA nucleic acids according to any one of embodiments 1 to 11, wherein all of the pegRNA nucleic acids are designed for prime editing of one or more (cardiomyocyte) genes at one or more multiple sites, and, optionally, a prime editor comprising nucleic acid programmable DNA binding protein (napDNAbp), which is fused to or bound to a domain comprising polymerase (preferably, RNA-dependent DNA polymerase) activity, a sgRNA, an iPSC, and a cardiomyocyte.
19. The composition of embodiment 12, wherein each of the pegRNA nucleic acids are designed for prime editing of a single (cardiomyocyte) gene.
20. The composition of any one of embodiments 12 and 13, further comprising one or more negative controls each comprising a pegRNA that does not introduce any edits into one or more genes.
21. A composition comprising the gRNA of embodiment 15 and, optionally, a nuclease.
22. A cell modified by CRISPR interference with a DNA polynucleotide coding for the gRNA of embodiment 15.
23. A cell genetically engineered to carry one or more specific mutations in one or more target genes, wherein the target genes are being tested for their role in cardiomyocyte function, proliferation, viability, survival, morphology, the expression of markers and receptors, and “heart beats” in vitro (which can model arrhythmias), preferably wherein the cell is a iPSC or a cardiomyocyte derived from a iPSC.
24. The cell of embodiment 23, wherein the cell is genetically engineered using CRISPR, base editing, or prime editing, preferably prime editing, more preferably with a pegRNA of any one of embodiments 1 through 14.
25. The cell of any one of embodiments 23 and 24, wherein the target gene is selected from KCNH2/hERG, KCNQ1, SCNA5, KCNE1, KCNE2, KvLQT1, Nav1.5, ankyrin-B, MinK, MiRP1, Kir2.1, Cav1.2, caveolin-3, and Nav.beta.4 genes; genes encoding other channels, a sodium channel/related gene selected from SCN5A, ACN1B, SCN2B, SCN3B, SCN4B, GPD1L, RANGRF, SCN10A; a potassium channel/related gene selected from human ether-a-go-go related gene (hERG), Hyperpolarization-activated cyclic nucleotide-gated (HCN) channels; transient outward potassium current channel; Slowly activating delayed rectifier potassium current channels; Rapidly activating delayed rectifier potassium current channels; Inwardly rectifying potassium (Kir) channels; Inwardly rectifying potassium channels; G protein-coupled, inwardly rectifying potassium channels; ATP-sensitive potassium channels; a calcium channel/related gene selected from CACNA1C, CACNB2, CACNA2D1, RYR2, CASQ2, TRDN, CALM1-3; other genes selected from SNTA1, SLMAP, PKP2, ANK2, CAV3, SLC4A3, TRPM4, DPP6, IRX3, GNAI2, ADORAI, GNAS, KCNQ1, SCNA5, KCNE1, KCNE2, KvLQT1, Nav1.5, ankyrin-B, MinK, MiRP1, Kir2.1, Cav1.2, caveolin-3, Nav.beta.4 ATP-binding cassette (ABC) transporters involved in drug transport (e.g., ABCB1, ABCB4, ABCCI, ABCC2, SLC10A2, SLC28A3, SLC22A7, SLC22A17), Carbonyl reductases in drug metabolism (e.g., CBR3), Hyaluronan synthase 3 involved in oxidative stress response (e.g., HAS3), Hereditary hemochromatosis protein in iron metabolism (e.g., HFE), Retinoic acid receptor gamma and DNA topoisomerases in topoisomerase-induced DNA damage (e.g., RARG, TOP2B), CUGBP Elav-like family member 4 in splicing of sarcomere genes (e.g., CELF4), DNA polymerase gamma in mitochondrial replication (e.g., DPOG2), and chaperones involved in ion channel trafficking (e.g., Hsp70 and Hsp90)
26. The cell of embodiment 25, wherein the mutation replicates one or more SNPs listed in Table 1 of SEQ ID NO.2 to 1054, preferably SEQ ID NO.105.
27. The cell of any one of embodiments 23 to 26, wherein the cell carries a V476I mutation in the KCNH2 gene.
28. The cell of any one of embodiments 23 to 27, wherein the mutation was known to be or is being tested as associated with heart disease.
29. The cell of embodiment 28, wherein the heart disease is an arrhythmia, hypertrophic cardiomyopathy, sudden unexplained death, obesity-related cardiac disease, primary dilated cardiomyopathy, primary familial hypertrophic cardiomyopathy, or seizure, preferably an arrhythmia selected from Long QT syndrome (including Congenital long QT syndrome, Long QT syndrome 2, and bradycardia-induced Long QT syndrome), Brugada syndrome, Short QT syndrome 1, Sudden Infant Death Syndrome, Acquired long QT syndrome, ventricular tachycardia, Wolff-Parkinson-White pattern arrhythmia, Arrhythmogenic right ventricular cardiomyopathy, Atrial fibrillation, Catecholaminergic polymorphic ventricular tachycardia type 1, Prolonged QT interval, Paroxysmal familial ventricular fibrillation 1, Sudden cardiac arrest/death, and Torsades de pointes.
30. The cell of any one of embodiments 23 to 29, wherein the cell expresses an mRNA having a sequence comprising SEQ ID NO.2-1054 from Table 1, preferably SEQ ID NO. 105.
31. A composition comprising a library of two or more iPS cells and/or cardiomyocytes wherein each iPS cell/cardiomyocyte has been modified to comprise one or more pegRNA molecules according to any one of embodiments 1 to 11 or is a cell of any one of embodiments 23 to 30.
32. The composition of embodiment 31 further comprising iPS cells and/or cardiomyocytes that have not been exposed to a pegRNA and/or iPS cells that have been exposed to one or more pegRNAs that do not edit one or more of the genes.
33. A method of identifying a target gene or gene mutation as associated with or cause for cardiotoxicity, comprising:
   (i) obtaining one or more iPSC-derived cardiomyocytes carrying one or more (e.g., library) of wild-type and target gene editing mutations in one or more (cardiomyocyte) genes; or obtaining one or more iPSC-derived cardiomyocytes wherein expression of one or more target genes has been altered; and
   (ii) identifying the target gene or gene mutation and/or altered gene expression level as associated with or a cause for cardiotoxicity if
   the mutation and/or gene expression level has a negative effect on cardiomyocytes' function, proliferation, viability, survival, morphology, the expression of certain markers and receptors, or “heart beats” in vitro (which can model arrhythmias) relative to wild-type (e.g., isogenic) cells.
34. A method of identifying a target gene mutation as a mutation associated with drug-induced cardiotoxicity, comprising:
   (i) obtaining one or more iPSC-derived cardiomyocytes carrying one or more (e.g., library) wild-type and target gene editing mutations in one or more (cardiomyocyte) genes; or obtaining one or more iPSC-derived cardiomyocytes wherein expression of one or more genes has been altered; and
   (ii) exposing the cardiomyocytes to a drug;
   (ii) identifying the target gene or gene mutation as associated with drug-induced cardiotoxicity if exposure of the cardiomyocytes carrying the mutation and/or the altered gene expression level to the drug has a negative effect on cardiomyocytes' function, proliferation, viability, survival, morphology, the expression of certain markers and receptors, or “heart beats” in vitro (which can model arrhythmias) relative to wild-type (e.g., isogenic) cells exposed and to cells not exposed to the drug.
35. A method of assessing whether a drug is cardiotoxic to a wild-type subject comprising:
   (i) obtaining one or more iPSC-derived wild-type cardiomyocytes;
   (ii) exposing the cardiomyocytes to a drug; and
   (ii) identifying the drug as cardiotoxic to a wild-type subject if exposure of the wild-type cardiomyocytes to the drug has a negative effect on cardiomyocytes' function, proliferation, viability, survival, morphology, the expression of certain markers and receptors, or “heart beats” in vitro (which can model arrhythmias) relative to wild-type (e.g., isogenic) cardiomyocytes not exposed to the drug.
36. A method for identifying a drug as causing cardiotoxicity in a subject due to the presence of a target gene mutation and/or due to the altered expression of a gene comprising:
   (i) obtaining one or more iPSC-derived cardiomyocytes carrying one or more (e.g. library) of wild-type and target gene editing mutations in a target gene; or obtaining one or more iPSC-derived cardiomyocytes wherein expression of one or more target genes has been altered; and
   (ii) exposing the cardiomyocytes to the drug;
   (iii) identifying the drug as causing cardiotoxicity due to the presence of the target gene mutation and/or due to the altered target gene expression when the cardiomyocytes carrying the mutation or altered target gene expression exhibit signs of cardiotoxicity in response to exposure to the drug in a cardiotoxicity assay but the wild-type cardiomyocytes do not; wherein signs of cardiotoxicity comprise a negative effect on cardiomyocytes' function, proliferation, viability, survival, morphology, the expression of certain markers and receptors, or “heart beats” in vitro (which can model arrhythmias) relative to wild-type (e.g., isogenic) cells not exposed to the drug; wherein sequencing of the gene or portion of the gene to determine the identity of the mutation takes place before or after the cardiotoxicity assay.
37. The method of any one of embodiments 33 through 36, wherein the mutation is introduced in the target gene by gene editing (including CRISPR, base editing, prime editing).
38. The method of embodiment 37, wherein the mutation is introduced in the target gene by prime editing, preferably prime editing with any one of the pegRNAs of any one of embodiments 1 through 14 and/or the target gene expression is altered by CRISPR interference, preferably using a gRNA of any one of embodiments 15 and 16.
39. The method of any one of embodiments 33 to 38, wherein at least one of the target genes that is edited or has its expression altered is selected from the KONH2 hERG, KCNQ1, SCNA5, KONE1, KONE2, KvLQT1, Nav1.5, ankyrin-B, MinK, MiRP1, Kir 2.1, Cav1.2, caveolin-3, and Nav.beta.4 genes; genes encoding other channels, a sodium channel/related gene selected from SCN5A, ACN1B, SCN2B, SCN3B, SCN4B, GPD1L, RANGRF, SCN10A; a potassium channel/related gene selected from human ether-a-go-go related gene (hERG), Hyperpolarization-activated cyclic nucleotide-gated (HCN) channels; transient outward potassium current channel; Slowly activating delayed rectifier potassium current channels; Rapidly activating delayed rectifier potassium current channels; Inwardly rectifying potassium (Kir) channels; Inwardly rectifying potassium channels; G protein-coupled, inwardly rectifying potassium channels; ATP-sensitive potassium channels; a calcium channel/related gene selected from CACNA1C, CACNB2, CACNA2D1, RYR2, CASQ2, TRDN, CALM1-3; other genes selected from SNTAI, SLMAP, PKP2, ANK2, CAV3, SLC4A3, TRPM4, DPP6, IRX3, GNAI2, ADORAI, GNAS, KCNQ1, SCNA5, KCNE1, KCNE2, KvLQT1, Nav1.5, ankyrin-B, MinK, MiRP1, Kir2.1, Cav1.2, caveolin-3, Nav.beta.4 ATP-binding cassette (ABC) transporters involved in drug transport (e.g., ABCB1, ABCB4, ABCCI, ABCC2, SLC10A2, SLC28A3, SLC22A7, SLC22A17), Carbonyl reductases in drug metabolism (e.g., CBR3), Hyaluronan synthase 3 involved in oxidative stress response (e.g., HAS3), Hereditary hemochromatosis protein in iron metabolism (e.g., HFE), Retinoic acid receptor gamma and DNA topoisomerases in topoisomerase-induced DNA damage (e.g., RARG, TOP2B), CUGBP Elav-like family member 4 in splicing of sarcomere genes (e.g., CELF4), DNA polymerase gamma in mitochondrial replication (e.g., DPOG2), and chaperones involved in ion channel trafficking (e.g., Hsp70 and Hsp90).
40. The method of embodiment 39, wherein the target gene is the KONH2 hERG gene.
41. The method of embodiment 40, wherein the mutation is selected from those that may result from the SNPs of Table 1 and SEQ ID NO.: 2-1054, preferably V476I, preferably SEQ ID NO: 105.
42. A method for identifying a subject in need of treatment with a drug as being sensitive to said drug-induced cardiotoxicity comprising testing a DNA sample from the subject (e.g., collected from a tissue comprising cardiomyocytes and/or from cardiomyocytes derived from iPSC isolated from the subject) for the presence of one or more target gene mutations or altered expression levels that have been identified as associated with said drug-induced cardiotoxicity by the method of any one of embodiments 33 to 41 and, optionally, choosing a different drug for treating the subject than the drug that induces cardiotoxicity.
43. The method of embodiment 42, wherein the subject is identified as suffering from, or being susceptible to, an arrhythmia, hypertrophic cardiomyopathy, sudden unexplained death, obesity-related cardiac disease, primary dilated cardiomyopathy, primary familial hypertrophic cardiomyopathy, or seizure, preferably an arrhythmia selected from Long QT syndrome (including Congenital long QT syndrome, Long QT syndrome 2, and bradycardia-induced Long QT syndrome), Brugada syndrome, Short QT syndrome 1, Sudden Infant Death Syndrome, Acquired long QT syndrome, ventricular tachycardia, Wolff-Parkinson-White pattern arrhythmia, Arrhythmogenic right ventricular cardiomyopathy, Atrial fibrillation, Catecholaminergic polymorphic ventricular tachycardia type 1, Prolonged QT interval, Paroxysmal familial ventricular fibrillation 1, Sudden cardiac arrest/death, and Torsades de pointes, when the subject carries one or more of the gene mutations.
44. The cells of any one of embodiments 22 to 30, or of the method of any one of embodiments 33 to 41, wherein the iPSC are prepared from any somatic cell including skin-derived fibroblasts, and peripheral blood mononuclear cells, and reprogrammed by any integrative (i.e. lentiviral) or episomal (i.e. plasmid, Sendai virus) vectors containing any combination of transcriptional factors, including OCT4, SOX2, KLF4 and MYC, or transcriptional factors delivered via RNA (mRNA or miRNA) or combinations of small molecules and growth factors, or direct modulation of gene expression via RNAi or CRISPRi/a.
45. The method of any one of embodiments 33 to 41, wherein the cardiomyocytes are derived from iPSC by in vitro differentiation using combinations of small molecules, and/or growth factors, including modulation of Wnt pathway followed by glucose starvation for selection of highly purified cardiomyocyte populations, and combinations with nucleic acids (including RNA, miRNA, siRNA).
46. The method of any one of embodiments 33 to 41, wherein signs of cardiotoxicity comprise changes in cardiomyocyte function, cell viability, survival, morphology, the expression of certain markers and receptors, heart beats in vitro and, and where these signs are measured, preferably, with models arrhythmias, a patch clamp technique, an external recording method, a voltage-sensitive dye, or an intracellular ion-sensitive dye.
47. A method of prime editing an iPS cell or an iPSC-derived cardiomyocyte cell comprising contacting the cell's double stranded target DNA with a pegRNA designed to edit a target gene (e.g., cardiomyocyte), preferably a pegRNA according to any one of embodiments 1 through 14, and with a prime editor, and optionally a sgRNA, preferably wherein the prime editor comprises a nucleic acid programmable DNA binding protein (napDNAbp), which is fused to a domain comprising polymerase (preferably, RNA-dependent DNA polymerase) activity, wherein the contacting prime edits the cells by installing one or more nucleotide edits in the double stranded target DNA, thereby editing the double stranded target DNA.
48. The method of embodiment 47, wherein the pegRNA and/or a nucleic acid encoding the napDNAbp fused to a domain comprising RNA-dependent DNA polymerase activity, are introduced into the iPSC or IPSC-derived cardiomyocytes by transfection, viral transduction (lentiviral, AAV, etc.), nanoparticles, or nucleofection.
49. The method of any one of embodiments 47 and 48, wherein the napDNAbp is selected from the group consisting of Cas9, Cas12e, Cas12d, Cas12a, Cas12b1, Cas13a, Cas12c, Cas12b2, Cas13a, Cas12c, Cas12d, Cas12e, Cas12h, Cas12i, Cas12g, Cas12f (Cas14), Cas12f1, Cas12j (Casǐ), and Argonaute and, optionally, has a nickase activity.
50. The method of any one of embodiments 47 to 49, wherein the napDNAbp is a nuclease active Cas9 domain, a nuclease inactive Cas9 domain, or a Cas9 nickase domain or variant thereof.
51. The method of any one of embodiments 47 to 50, wherein the RNA-dependent DNA polymerase is a reverse transcriptase, preferable selected from Moloney Murine Leukemia virus reverse transcriptase (MMLV-RT), optionally wherein the MMLV-RT comprises one or more amino acid substitutions selected from D200N, T306K, W313F, T330P, and L603W compared to a wild type MMLV-RT.
52. The method of any one of embodiments 29 to 46, wherein the pegRNA and/or the gRNA are designed by PrimeDesign or another commercially or publicly available method known to one of ordinary skill in the art.
53. A polynucleotide comprising a DNA sequence coding for the pegRNA of any one of embodiments 1 through 14 or coding the gRNA of any one of embodiments 15 and 16, preferably the sequence of SEQ Nos: 1116 to 1134, or a pegRNA coded by the same.
54. A vector comprising the polynucleotide of embodiment 53, optionally wherein expression of the pegRNA or gRNA is under the control of a promoter.
55. The method of any one of embodiments 33 to 52, wherein the iPSCs or iPSC-derived cardiomyocytes are cultured and/or tested in multi-well (e.g. 96 well) plates.
56. The method of embodiment 55, wherein each well comprises 1 cell or its progeny.
57. The method of any one of embodiments 55 or 56, wherein each cell comprises only one type of pegRNA, or nucleic acid encoding the pegRNA, preferably wherein the cells are sorted through the barcodes in each pegRNA and/or wherein the pegRNAs are sorted into each well prior to introduction into the cells.
58. The method of any one of embodiments 33 to 41, 45, and 46, wherein the drug is a drug that is hypothesized to cause arrhythmia.
59. The method of any one of embodiments 33 to 41, 45, 46, 55 to 58, wherein the drug is selected from a list of blackbox labeled drugs (currently marketed drugs that contain a label to indicate that Long QT patients should avoid using this drug; including Table 2), ii) drugs withdrawn from the US market due to cardiotoxicity events, iii) drugs that failed clinical trials due to cardiotoxicity events—(some may be marketed in other countries; iv) drugs associated with cardiotoxicity for particular SNPs as documented in the literature (e.g., ClinVar; antiarrhythmics, class i and iii; cisapride, amiodarone, dofetilide, clarithromycin, hydroxyzine, quinidine and disopyramide), and v) TdP associated drugs (including Table 3).
60. A method of treating a subject for arrhythmia and/or preventing arrhythmia in the subject, wherein the subject is identified by the method of embodiment 42 or 43 as carrying a variant mutation, wherein the treatment comprises genome editing of the genome of at least some of the subject's cardiomyocytes to edit the variant mutation and/or alter the expression of the target gene.
61. An array of cell culture wells or vessels each comprising at least one iPSC or iPSC-derived cardiomyocyte and at least one component of a gene editing system and/or gene expression altering system, wherein the gene editing system is designed to introduce one or more gene edits per iPSC/cardiomyocyte into at least one target gene and the gene expression altering system is designed to alter the expression of at least one target gene, wherein the target gene edit and/or altered expression level is hypothesized to cause cardiotoxicity or drug-induced cardiotoxicity when introduced into or present in the genome of a cardiomyocyte.
62. The array of embodiment 61, wherein the array comprises a multi-well tissue culture plate.
63. The array of embodiment 61, wherein each well or vessel carries only cells comprising a single gene edit or a single level of altered gene expression.
64. The array of any one of embodiments 62 and 63, wherein the array further comprises one or more elements of an in vitro cardiotoxicity assay.
65. The array of any one of embodiments 61 to 64, wherein the gene editing or gene expression altering system comprises one or more pegRNA according to any one of embodiments 1 to 14, one or more gRNA according to any one of embodiments 15 and 16, and/or any composition according to any one of embodiments 17 to 21.
66. The method, pegRNA. gRNA, polynucleotide, vector, and cell of any one of embodiments 1-66, wherein the coding DNA sequence coding for the pegRNA comprises any one of SEQ ID NO. 1100 to 1113 and/or the coding sequence coding for the gRNA comprises any one of SEQ ID Nos 1116-1134 (or where the target is that of anyone one of SEQ ID Nos. 1055 to 1073), and the DNA coding sequence for the mRNA/SNP comprises any one of SEQ ID NO. 2 to 1054.

### BRIEF DESCRIPTION OF THE DRAWINGS

The patent or application file contains at least one drawing executed in color. Copies of this patent or patent application publication with color drawings will be provided by the U.S. Patent and Trademark Office upon request and payment of the necessary fee.

FIG. 1A: SNPs selected for prime editing into hiPSC. Structure of hERG channel and how V476I SNP changes its structure. FIG. 1B: Structure of hERG channel and modeling of V476I mutant.

FIG. 2: Sequencing of plasmids containing gRNA sequences for prime editing.

FIG. 3: Efficiency of plasmid DNA electroporation into hiPSC using GenePulser.

FIG. 4: Efficiency of cardiomyocyte differentiation using different Wnt activator concentrations and several starting cell densities in 6 well plates, and the effect of enrichment protocol on cardiomyocyte population purity.

FIG. 5: Expression of cardiac troponin T (cTnT) in iPSC-CM. Nuclei and cytoplasm are stained with whole cell stain CellMask Blue.

FIG. 6: Basal beating of iPSC-derived cardiomyocytes.

FIG. 7: Characterization of drug responses in wild-type and V476I mutant cardiomyocytes. hiPSC-CM were treated with a fluorescent calcium-sensing probe for 2h before addition of drugs. Cells were then incubated with drugs for 1.5-2h. A plate reader (SpectraMax) was used to measure fluctuations in fluorescence for 30 sec. Images were analyzed using PeakPro to generate peak frequency which is a proxy for beats per minute. Both wild-type and V476I mutant show expected responses to pro-arrhythmic drugs, demonstrating functional responsiveness of this cellular model.

FIG. 8: Optimization of CRISPR interference system for regulation of gene expression. SHSY5Y cells, which express high levels of KCNH2, were transduced with a lentivirus carrying Sp dCas9-KRAB-rTTA and selected with antibiotics. Selected populations were transduced with lentivirus carrying sgRNAs for different (positive control) genes, POLR2D or SF3B1, and subjected to another round of antibiotic selection to generate cell lines carrying the complete CRISPRi system. After selection, cells were treated with doxycycline for 72h to activate the expression of the Sp dCas9-KRAB construct, leading to gene repression. Relative gene expression was measured via qPCR using TaqMan probes. Grey bars indicate qPCR for SF3B1 and blue bars qPCR for POLR2D. Marked repression of relative gene expression was observed in wells treated with doxycycline, indicating an active CRISPRi system for the positive control genes

FIG. 9: Replicate arrhythmias with cardiotoxic drugs in the in vitro system.

FIG. 10: B3 clone shows disruption of normal rhythm.

FIG. 11: B3 clone hypersensitive to pro-arrhythmic compounds.

FIG. 12: B3 clone non-responsive to anti-arrhythmic compounds.

FIG. 13: Exemplary pegRNA library design.

### DETAILED DESCRIPTION

### Definitions

In order for the present disclosure to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.,” as used herein, are used merely by way of example, without limitation intended, and should not be construed as referring only those items explicitly enumerated in the specification.

The terms “or more,” “at least,” “more than,” and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000, or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality,” “at least two,” “two or more,” “at least second,” and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000, or more. Also included is any greater number or fraction in between.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided. The term “consisting of” excludes any element, step, or ingredient not specified in the claim. In re Gray, 53 F.2d 520, 11 USPQ 255 (CCPA 1931); Ex parte Davis, 80 USPQ 448, 450 (Bd. App. 1948) (“consisting of” defined as “closing the claim to the inclusion of materials other than those recited except for impurities ordinarily associated therewith”). The term “consisting essentially of” limits the scope of a claim to the specified materials or steps “and those that do not materially affect the basic and novel characteristic(s)” of the claimed invention.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” may mean within one or more than one standard deviation per the practice in the art. “About” or “approximately” may mean a range of up to 10% (i.e., +10%). Thus, “about” may be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg may include any amount between 4.5 mg and 5.5 mg. Furthermore, particularly with respect to biological systems or processes, the terms may mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Système International de Unites (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2nd ed., (2001), CRC Press; “The Dictionary of Cell & Molecular Biology”, 5th ed., (2013), Academic Press; and “The Oxford Dictionary Of Biochemistry And Molecular Biology”, Cammack et al. eds., 2nd ed, (2006), Oxford University Press, provide those of skill in the art with a general dictionary for many of the terms used in this disclosure.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

A “therapeutically effective amount,” “effective dose,” “effective amount,” or “therapeutically effective dosage” of a therapeutic agent, e.g., engineered CAR T cells, small molecules, “agents” described in the specification, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. Such terms may be used interchangeably. The ability of a therapeutic agent to promote disease regression may be evaluated using a variety of methods known to the skilled practitioner, such as in human subjects during clinical trials, in animal model systems predictive of efficacy in humans, or by assaying the activity of the agent in in vitro assays. Therapeutically effective amounts and dosage regimens can be determined empirically by testing in known in vitro or in vivo (e.g., animal model) systems.

The term “combination” refers to either a fixed combination in one dosage unit form, or a combined administration where a compound of the present invention and a combination partner (e.g., another drug as explained below, also referred to as “therapeutic agent” or “agent”) may be administered independently at the same time or separately within time intervals, especially where these time intervals allow that the combination partners show a cooperative, e.g., synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time.

The term “genetically engineered” or “engineered” refers to a method of modifying the genome of a cell, including, but not limited to, deleting a coding or non-coding region or a portion thereof or inserting a coding region or a portion thereof.

he terms “homologous,” “homology,” or “percent homology” as used herein refer to the degree of sequence identity between an amino acid or polynucleotide sequence and a corresponding reference sequence. “Homology” can refer to polymeric sequences, e.g., polypeptide or DNA sequences that are similar. Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity. In other embodiments, a “homologous sequence” of nucleic acid sequences may exhibit 93%, 95%, or 98% sequence identity to the reference nucleic acid sequence. For example, a “region of homology to a genomic region” can be a region of DNA that has a similar sequence to a given genomic region in the genome. A region of homology can be of any length that is sufficient to promote binding of a spacer or protospacer sequence to the genomic region. For example, the region of homology can comprise at least 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, or more bases in length such that the region of homology has sufficient homology to undergo binding with the corresponding genomic region. When a percentage of sequence homology or identity is specified, in the context of two nucleic acid sequences or two polypeptide sequences, the percentage of homology or identity generally refers to the alignment of two or more sequences across a portion of their length when compared and aligned for maximum correspondence. When a position in the compared sequence can be occupied by the same base or amino acid, then the molecules can be homologous at that position. Unless stated otherwise, sequence homology or identity is assessed over the specified length of the nucleic acid, polypeptide, or portion thereof. In some embodiments, the homology or identity is assessed over a functional portion or a specified portion of the length. Alignment of sequences for assessment of sequence homology can be conducted by algorithms known in the art, such as the Basic Local Alignment Search Tool (BLAST) algorithm, which is described in Altschul et al, J. Mol. Biol. 215:403-410, 1990. A publicly available, internet interface, for performing BLAST analyses is accessible through the National Center for Biotechnology Information. Additional known algorithms include those published in: Smith & Waterman, “Comparison of Biosequences”, Adv. Appl. Math. 2:482, 1981; Needleman & Wunsch, “A general method applicable to the search for similarities in the amino acid sequence of two proteins” J. Mol. Biol. 48:443, 1970; Pearson & Lipman “Improved tools for biological sequence comparison”, Proc. Natl. Acad. Sci. USA 85:2444, 1988; or by automated implementation of these or similar algorithms. Global alignment programs may also be used to align similar sequences of roughly equal size. Examples of global alignment programs include NEEDLE (available at www.ebi.ac.uk/Tools/psa/emboss_needle/) which is part of the EMBOSS package (Rice P et al., Trends Genet., 2000; 16:276-277), and the GGSEARCH program fasta.bioch.virginia.edu/fasta_www2/, which is part of the FASTA package (Pearson W and Lipman D, 1988, Proc. Natl. Acad. Sci. USA, 85:2444-2448). Both of these programs are based on the Needleman-Wunsch algorithm, which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998). A skilled person understands that amino acid (or nucleotide) positions may be determined in homologous sequences based on alignment.

A “patient” or a “subject” as used herein includes any human who is afflicted with a heart disease or disorder. The terms “subject” and “patient” are used interchangeably herein.

As used herein, the term “in vitro cell” refers to any cell which is cultured ex vivo. In particular, an in vitro cell may include a T cell. The term “in vivo” means within the patient.

The terms “peptide,” “polypeptide,” and “protein” are used interchangeably, and refer to a compound comprised of amino acid residues covalently linked by peptide bonds. A protein or peptide contains at least two amino acids, and no limitation is placed on the maximum number of amino acids that may comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “prime editor (PE)” or “prime editor” refers the compositions involved in the method of genome editing using target-primed reverse transcription (TPRT) describe herein, including, but not limited to the napDNAbps, reverse transcriptases, fusion proteins (e.g., comprising napDNAbps and reverse transcriptases), prime editor guide RNAs, and complexes comprising fusion proteins and prime editor guide RNAs, as well as accessory elements, such as second strand nicking components and 5′ endogenous DNA flap removal endonucleases for helping to drive the prime editing process towards the edited product formation.

Prime editing may also be described as “target-primed reverse transcription” (TPRT) because the target DNA molecule is used to prime the synthesis of a strand of DNA by a polymerase (e.g., reverse transcriptase). The use of the term “reverse transcription” in the name “target-primed reverse transcription” is not intended to limit prime editing to the use of reverse transcriptases, but rather TPRT or prime editors may comprise any polymerase (e.g., DNA-dependent DNA polymerase or RNA-dependent DNA polymerase). In various embodiments, prime editing operates by contacting a target DNA molecule (for which a change in the nucleotide sequence is desired to be introduced) with a nucleic acid programmable DNA binding protein (napDNAbp) complexed with a prime editor guide RNA

As used herein, the term “DNA synthesis template” refers to the region or portion of the extension arm of a PEgRNA that is utilized as a template strand by a polymerase of a prime editor to encode a 3′ single-strand DNA flap that contains the desired edit and which then, through the mechanism of prime editing, replaces the corresponding endogenous strand of DNA at the target site. The extension arm, including the DNA synthesis template, may be comprised of DNA or RNA. In the case of RNA, the polymerase of the prime editor can be an RNA-dependent DNA polymerase (e.g., a reverse transcriptase). In the case of DNA, the polymerase of the prime editor can be a DNA-dependent DNA polymerase. In some embodiments, the DNA synthesis template is a single-stranded portion of the PEgRNA that is 5′ of the PBS and comprises a region of complementarity to the PAM strand (i.e., the non-target strand or the edit strand), and comprises one or more nucleotide edits compared to the endogenous sequence of the double stranded target DNA. In some embodiments, the DNA synthesis template is complementary or substantially complementary to a sequence on the non-target strand that is downstream of a nick site, except for one or more non-complementary nucleotides at the intended nucleotide edit positions. In some embodiments, the DNA synthesis template is complementary or substantially complementary to a sequence on the non-target strand that is immediately downstream (i.e., directly downstream) of a nick site, except for one or more non-complementary nucleotides at the intended nucleotide edit positions. In some embodiments, one or more of the non-complementary nucleotides at the intended nucleotide edit positions are immediately downstream of a nick site. In some embodiments, the DNA synthesis template comprises one or more nucleotide edits relative to the double-stranded target DNA sequence. In some embodiments, the DNA synthesis template comprises one or more nucleotide edits relative to the non-target strand of the double-stranded target DNA sequence. For each PEgRNA described herein, a nick site is characteristic of the particular napDNAbp to which the gRNA core of the PEgRNA associates and is characteristic of the particular PAM required for recognition and function of the napDNAbp. For example, for a PERNA that comprises a gRNA core that associates with a SpCas9, the nick site in the phosphodiester bond between bases three (“−3” position relative to the position 1 of the PAM sequence) and four (“−4” position relative to position 1 of the PAM sequence). In some embodiments, the DNA synthesis template and the primer binding site are immediately adjacent to each other. The terms “nucleotide edit,” “nucleotide change,” “desired nucleotide change,” and “desired nucleotide edit” are used interchangeably to refer to a specific nucleotide edit, e.g., a specific deletion of one or more nucleotides, a specific insertion of one or more nucleotides, a specific substitution (or multiple substitutions) of one or more nucleotides, or a combination thereof, at a specific position in a DNA synthesis template of a PEgRNA to be incorporated in a target DNA sequence. In some embodiments, the DNA synthesis template comprises more than one nucleotide edit relative to the double-stranded target DNA sequence. In such embodiments, each nucleotide edit is a specific nucleotide edit at a specific position in the DNA synthesis template, each nucleotide edit is at a different specific position relative to any of the other nucleotide edits in the DNA synthesis template, and each nucleotide edit is independently selected from a specific deletion of one or more nucleotides, a specific insertion of one or more nucleotides, a specific substitution (or multiple substitutions) of one or more nucleotides, or a combination thereof. A nucleotide edit may refer to the edit on the DNA synthesis template as compared to the sequence on the target strand of the target gene, or a nucleotide edit may refer to the edit encoded by the DNA synthesis template on the newly synthesized single stranded DNA that replaces the endogenous target DNA sequence on the non-target strand.

In one embodiment, the “spacer” sequence is the sequence in the guide RNA or PEgRNA (having about 20 nts in length) which binds to the protospacer in the target DNA. In one embodiment, the “gRNA core” (or gRNA scaffold or backbone sequence) refers to the sequence within the gRNA that is responsible for napDNAbp binding (e.g., Cas9) and does not include the 20 bp spacer/targeting sequence that is used to guide Cas9 to target DNA. In some embodiments, the gRNA core or scaffold comprises a sequence that comprises one or more nucleotide alterations compared to a naturally occurring CRISPR-Cas guide RNA scaffold, for example, a Cas9 guide RNA scaffold. In some embodiments, the sequence of the gRNA core is designed to comprise minimal or no sequence homology to the endogenous sequence of the target nucleic acid at the target site, thereby reducing unintended edits. In some embodiments, the gRNA core comprises minimal sequence homology to the sequence of the target site, optionally wherein the gRNA core comprises no more than 1%, 5%, 10%, 15%, 20%, 25%, or 30% sequence homology to the sequence of the double stranded target DNA that flanks 5, 10, 15, 20, 25, 30, 35, 40, 45, or 50 nucleotides upstream or downstream of the position of the one or more nucleotide edits.

A DNA “barcode” is a unique sequence of nucleotides that may be incorporated into the pegRNA expression vector for identification of pegRNAs in complex mixtures, such as in the context of a pooled library of pegRNAs. Barcodes may be inserted into the pegRNA expression vector by restriction fragment cloning of small oligo nucleotides containing a unique 8-bp DNA sequence that differs from other sequences by at least 2 bases flanked by restriction sites compatible with the acceptor expression vector. Sequencing of expression cassette allows for the identification of pegRNA. Another method is to insert the U6-pegRNA expression cassette downstream from a Polymerase II promoter (such as CMV, EF1a, UBC) and upstream of a poly-A sequence. Flanking the U6-pegRNA expression cassette in this manner in lentiviral expression vectors leads to expression of the pegRNA from a Pol II promoter which allows identification of the respective pegRNA via RNA sequencing using standard poly A capture. In some embodiments, the pegRNA is its own barcode.

### Gene Editing Methods

The genome of the cells of the disclosure may be edited by any method known to one of skill in the art. In one embodiment, the cells are edited with Zinc finger nucleases. In one embodiment, the cells are edited with Transcription activator-like effector nucleases (TALENs). In one embodiment, the cells are edited with Homing Nucleases. In one embodiment, the cells are edited with clustered regularly interspersed short palindromic repeats (CRISPR)-Cas system. In one embodiment, the cells are edited with CRISPR-Cas9 system. In one embodiment, the cells are edited with base editing. In one embodiment, the cells are edited with Prime Editing. The components of each of these systems are well known in the art.

In some embodiments, the expression of one or more target genes is altered in the cells and the effect of the alteration in gene expression on the resulting cells (e.g., cardiotoxicity) is assessed. In some embodiments, the expression level is changed by CRISPR interference. CRISPR/Cas9-mediated transcriptional interference (CRISPRi) enables programmable gene knock-down, yielding loss-of-function phenotypes for nearly any gene. Genes can be analyzed with CRISPRi, and knock-down can be activated and relieved by conditional expression of the dCas9 fusion protein or the guide RNA. In some embodiments, the expression of one or more genes is completely blocked. In other embodiments, the expression of one or more genes is changed in various percentages. In some embodiments, the gene expression may be changed by a Cas9: gRNA complex, by a dCas9-SAM system, or a dCas9-KRAB system.

### Genetic Engineering of Progenitor Stem Cells

In some embodiments, the disclosure provides that certain gene mutations (e.g., SNPs) engineered into iPSC by gene editing may generate iPSC-derived cardiomyocytes that may be used as models of cardiac disease. In some embodiments, the mutations represent known SNPs of known and unknow clinical significance. In some embodiments, the mutations are not SNPs. In some embodiments, the mutations are introduced via Prime Editing (PE), which is described herein in more detail.

### KCNH2 Mutations

The disclosure provides that KONH2 encodes a voltage-gated transmembrane potassium channel called hERG (ether-a-go-go). hERG is expressed primarily on the surface of cardiac muscle cells, where it controls the efflux ok K+ ions during the rectifying phase of the action potential. Blockage of hERG causes a delay on the return of the membrane potential to its normal value, which leads to proarrhythmic events. V476I (rs199472908) is a mutation in the transmembrane domain of the hERG channel. Accordingly, in one embodiment, the disclosure provides genetically engineered iPSC, and cardiomyocytes derived therefrom, carrying the mutation V476I. In some embodiments, the mutation is introduced via genetic engineering with CRISPR or CRISPR-like systems. In some embodiments, the mutation is introduced via any other means of recombinantly changing one or more nucleic acid bases in a gene. In some embodiments, the genetic modification is introduced via prime-editing. In some embodiments, the disclosure provides prime-editing (pegRNAs) designed to introduce such mutations into the cells.

In some embodiments, the mutations are selected from a collection of more than 2,347 kcnh-2 SNPs from ClinVar. In some embodiments, the mutations are the SNPs described in SEQ ID Nos: 1-1054 and Table 1. Note that, due to the large number of resulting mRNA sequences, the exact sequences are only included in the Sequence Listing, which is part of the invention and disclosure. In the database, SNPs are categorized according to sequence location (e.g., promoter, exon, intron, UTRs, etc.) and type (e.g., missense mutation, nonsense mutation, insertion, deletion, etc.). ClinVar provides a list of attributes for each kcnh-2 SNP including the clinical severity (e.g., pathogenic, uncertain significance, not provided, etc.) and a ranking based on review status. Review status provides a reliability score, viz. how well supported a clinical attribution is based on number of submissions, review by an expert panel, etc. In one embodiment, the inventors curated a subset of 700 nonsense mutations (SNPs that encode changes to the protein coding sequence) for use according to the methods of the invention. In some embodiments, the results may subsequently be confirmed in biophysical models for machine learning prediction. In some embodiments, the results are confirmed according to the methods of the disclosure.

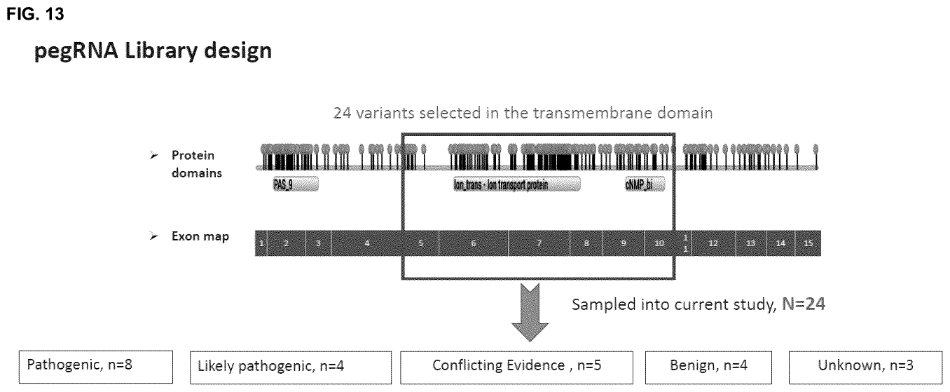

In some embodiments, the mutations are predicted to be clinically pathogenic. In some embodiments, the mutations are predicted to be likely clinically pathogenic. In some embodiments, there is conflicting clinical evidence about the specific mutation. In some embodiments, the mutation is clinically benign. In some embodiments, the mutations have not been clinically classified. See, for example, Table 1 and FIG. 13.

### Other Mutations

The disclosure also provides for genetic modifications of iPSCs and/or cardiomyocytes in other target genes. Accordingly, in some embodiments, the disclosure provides genetically engineered iPSC, and cardiomyocytes derived therefrom, as well as pegRNAs, for introducing and carrying mutations in any target gene. In some embodiments, the target gene is known to modify the activity, proliferation and growth of cardiomyocytes. In some embodiments, the target gene is not known to modify the activity, proliferation and growth of cardiomyocytes but may be identified as such with the methods of the disclosure. In some embodiments, the target genes may be chosen from KCNQ1, SCNA5, KCNE1, KCNE2, and genes encoding other channels, ATP-binding cassette (ABC) transporters involved in drug transport (e.g., ABCB1, ABCB4, ABCCI, ABCC2, SLC10A2, SLC28A3, SLC22A7, SLC22A17), Carbonyl reductases in drug metabolism (e.g., CBR3), Hyaluronan synthase 3 involved in oxidative stress response (e.g., HAS3), Hereditary hemochromatosis protein in iron metabolism (e.g., HFE), Retinoic acid receptor gamma and DNA topoisomerases in topoisomerase-induced DNA damage (e.g., RARG, TOP2B), CUGBP Elav-like family member 4 in splicing of sarcomere genes (e.g., CELF4), DNA polymerase gamma in mitochondrial replication (e.g., DPOG2), and Chaperones involved in ion channel trafficking (e.g., Hsp70 and Hsp90). In other embodiments, the target gene is selected from potassium channel/related genes selected from human ether-a-go-go related gene (hERG), Hyperpolarization-activated cyclic nucleotide-gated (HCN) channels; transient outward potassium current channel; Slowly activating delayed rectifier potassium current channels; Rapidly activating delayed rectifier potassium current channels; Inwardly rectifying potassium (Kir) channels; Inwardly rectifying potassium channels; G protein-coupled, inwardly rectifying potassium channels; ATP-sensitive potassium channels; sodium channel/related genes selected from SCN5A, ACN1B, SCN2B, SCN3B, SCN4B, GPD1L, RANGRF, SCN10A; calcium channel/related genes selected from CACNA1C, CACNB2, CACNA2D1, RYR2, CASQ2, TRDN, CALM1-3; and other genes selected from KCNQ1, SCNA5, KCNE1, KCNE2, KvLQT1, Nav1.5, ankyrin-B, MinK, MiRP1, Kir2.1, Cav1.2, caveolin-3, Nav.beta.4 genes, SNTA1, SLMAP, PKP2, ANK2, CAV3, SLC4A3, TRPM4, DPP6, IRX3, GNAI2, ADORAI, GNAS, KCNQ1, SCNA5, KCNE1, KCNE2, KvLQT1, Nav1.5, ankyrin-B, MinK, MiRP1, Kir2.1, Cav1.2, caveolin-3, Nav.beta.4 ATP-binding cassette (ABC) transporters involved in drug transport (e.g., ABCB1, ABCB4, ABCCI, ABCC2, SLC10A2, SLC28A3, SLC22A7, SLC22A17), Carbonyl reductases in drug metabolism (e.g., CBR3), Hyaluronan synthase 3 involved in oxidative stress response (e.g., HAS3), Hereditary hemochromatosis protein in iron metabolism (e.g., HFE), Retinoic acid receptor gamma and DNA topoisomerases in topoisomerase-induced DNA damage (e.g., RARG, TOP2B), CUGBP Elav-like family member 4 in splicing of sarcomere genes (e.g., CELF4), DNA polymerase gamma in mitochondrial replication (e.g., DPOG2), and chaperones involved in ion channel trafficking (e.g., Hsp70 and Hsp90).

### Computational Approaches to Predicting the Pathogenicity of SNPS.

In one embodiment, the mutations that may be analyzed by the methods of the disclosure may be identified in public databases. In one embodiment, the database is selected from ClinVar, dbSNP, gnomAD, SNPnexus, EMBI-EBI, SNPedia, and European Variant Archive. These and other databases provide a resource for identifying SNPs of interest to engineer into iPSC cell lines for drug testing on differentiated cardiomyocytes. The disclosure provides that interindividual differences in LQTS-associated genes (e.g., hERG, etc.) affect the control of the QT interval and hence form the biochemical basis for adverse cardiotoxicity responses to drugs.

Clinical presentation of cardiotoxicity for numerous gene alleles (e.g., hERG (AKA kcnh-2)) have been reported in the literature and organized into databases (e.g., ClinVar, dbSNP, etc.) The frequency of alleles in the human population, with a breakdown according to ethnicity, have been tabulated from large scale genomics studies and made available as an online resource (e.g., gnomAD). Consequently, in one embodiment, the target genes for the methods of the disclosure are selected based on these features of the databases.

In other embodiments, SNPs have been identified by sequencing studies but are as yet unclassified with regards to LQTS and/or drug induced cardiotoxicity. Accordingly, in one embodiment, these SNPs are referred to as variants of unknown significance (VUSs). In one embodiment, available protein structures in the RSC-PDB database may be used to derive inferences about the consequences of a given VUS, e.g., benign or pathogenic. As noted above, variation in kcnh-2 is extensive, with >1,300 SNPs identified, some of which are listed in Table 1. In some embodiments, these SNPs are tested with the cells and methods of the disclosure.

In one embodiment, the inventors have developed computational methods that use i) SNP data from online resources (i.e., ClinVar, dbSNP, gnomAD) in combination with ii) protein structures (pdbid 5val) and derived biophysical features (e.g., solvent exposed surface area, conservation, subunit interface, etc.) from physical and biochemical analysis to identify potentially clinically relevant SNPs. Using the above combination of data as a feature set for machine learning (ML) with inputs from domain expertise in biochemistry, the inventors have accurately predicted the effects of SNPs on QT interval and cardiotoxic response to drugs (data not shown).

Accordingly, in one embodiment, computational methods provide a means to down select the number of SNPs that may be experimentally tested using the cells and methods of the disclosure. In one embodiment, one may draw inferences from experimental data to accurately predict (>80% accuracy) cardiotoxic effects. These embodiments, and derivatives thereof, are generalizable to other LQTS-associated genes to guide selection of SNPs for genome editing of iPSCs for drug response studies.

In some embodiments, one may prioritize the most frequent variants as down selection criteria for identification of target SNIPs that may be tested with the methods of the disclosure, e.g., allele frequencies equal to or greater than 3.4×10⁻⁵ encompasses 99 nonsynonymous SNPs. In some embodiments, the variants are KCNH-2/hERG variants.

In one embodiment, allele frequency in the human population and ethnic groups therein may be used as a criterion for prioritizing SNPs for experimental characterization using the methods of the disclosure. In some embodiments, assessing allele frequency may be done with ALFA: Allele Frequency Aggregator. NCBI developed the Allele Frequency Aggregator (ALFA) pipeline to compute allele frequency for variants in dbGaP across approved unrestricted studies and to provide the data as open-access to the public through dbSNP. The goal of the ALFA project is to make frequency data from over IM subjects from the database of Genotypes and Phenotypes (dbGaP) available via open-access in future releases to facilitate discoveries and interpretations of common and rare variants with biological impacts or causing diseases.

In some embodiments, the target gene mutations are selected using PharmGKB. PharmGKB is a comprehensive resource that curates knowledge about the impact of genetic variation on drug responses for clinicians and researchers. In some embodiments, data entries in PharmGKB for kenh-2 may be downloaded and cross-checked with Clin Var entries for drug-gene interactions.

In some embodiments, the target gene mutations are selected from the Genome Aggregation Database (gnomAD). The gnomAD is a resource developed by an international coalition of investigators, with the goal of aggregating and harmonizing both exome and genome sequencing data from a wide variety of large-scale sequencing projects and making summary data available for the wider scientific community. The v2.1.1 data set (GRCh37/hg19) provided on this website spans 125,748 exome sequences and 15,708 whole-genome sequences from unrelated individuals sequenced as part of various disease-specific and population genetic studies. The v3.1.2 data set (GRCh38) spans 76,156 genomes, selected as in v2.

Genetic Modification of iPSC and Cardiomyocytes via Prime Editing

The iPSC and cardiomyocytes of the disclosure may be genetically modified through any gene editing method, including CRISPR, base editing, and prime editing. They can also be modified by CRISPR interference as a means to regulate the expression level of any gene. In one embodiment, the gene editing method is prime editing. Prime editing enables the insertion, deletion, and/or replacement of genomic DNA sequences without requiring error-prone double-strand DNA breaks. It was first described by Anzalone et al., “Search-and-replace genome editing without double-strand breaks or donor DNA,” Nature, 2019, Vol. 576, pp. 149-157, the contents of which are incorporated herein by reference. Since then, multiple variations of the original prime editing technique, some of which are known as PE1, PE2, PE3, PE4, PE5, continue to be developed. Prime editing generally involves a prime editor (PE) and a prime editing guide RNA (pegRNA). In general, prime editing uses an engineered Cas9 nickase-reverse transcriptase fusion protein (PE1, PE2) paired with an engineered prime editing guide RNA (pegRNA) that not only directs Cas9 to a target genomic site, but also which encodes the information for installing the desired edit in the target gene. The first version of prime editing is believed to proceed through multi-step editing process: 1) the Cas9 domain binds and nicks the target genomic DNA site, which is specified by the pegRNA's spacer sequence; 2) the reverse transcriptase domain uses the nicked genomic DNA as a primer to initiate the synthesis of an edited DNA strand using an engineered extension on the pegRNA as a template for reverse transcription—this generates a single-stranded 3′ flap containing the edited DNA sequence; 3) cellular DNA repair resolves the 3′ flap intermediate by the displacement of a 5′ flap species that occurs via invasion by the edited 3′ flap, excision of the 5′ flap containing the original DNA sequence, and ligation of the new 3′ flap to incorporate the edited DNA strand, forming a heteroduplex of one edited and one unedited strand; and 4) cellular DNA repair replaces the unedited strand within the heteroduplex using the edited strand as a template for repair, completing the editing process.

In some embodiments, the target genes are mutated by different base editing and prime editing methods. Newer prime editing versions include approaches that rely on the addition of other elements to the system to reduce errors. In PE2 a PE complex comprising a fusion protein comprising Cas9(H840A) and a variant MMLV RT is used. This enhances DNA-RNA affinity, enzyme processivity, and thermostability. In addition, PE3 is a modification of PE2, in which an additional nick on the opposite DNA strand is created. Despite the increased efficacy of PE2, the edit inserted by PE2 might still be removed due to DNA mismatch repair of the edited strand. To avoid this problem during DNA heteroduplex resolution, an additional single guide RNA (sgRNA) is introduced. This sgRNA is designed to match the edited sequence introduced by the pegRNA, but not the original allele. It directs the Cas9 nickase portion of the fusion protein to nick the unedited strand at a nearby site, opposite to the original nick. Nicking the non-edited strand causes the cell's natural repair system to copy the information in the edited strand to the complementary strand, permanently installing the edit. PE4 includes PE2 plus an MLH1 dominant negative protein (e.g., wild-type MLH1 with amino acids 754-756 truncated). Dominant negative MLH1 is able to essentially knock out endogenous MLH1 by inhibition, thereby reducing cellular DNA mismatch repair response and increasing prime editing efficiency.

In one embodiment, the prime editor comprises a fusion protein comprising (i) a nucleic acid programmable DNA binding protein (napDNAbp) and (ii) a DNA polymerase, wherein the napDNAbp is a Cas9 nickase (nCas9) and/or the DNA polymerase is a Reverse Transcriptase (RT). In some embodiments, Cas9 is replaced by another napDNAbp. In some embodiments, the napDNAbp is a nuclease active Cas9 domain, a nuclease inactive Cas9 domain, or a Cas9 nickase domain or variant thereof. In some embodiments, the napDNAbp is selected from Cas9, Cas12e, Cas12d, Cas12a, Cas12b1, Cas13a, Cas12c, ArgonauteCas12b2, Cas13a, Cas12c, Cas12d, Cas12e, Cas12h, Cas12i, Cas12g, Cas12f (Cas14), Cas12f1, Cas12j (Casǐ), and Argonaute and optionally has a nickase activity.

In some embodiments, the RT is replaced by another DNA polymerase. In some embodiments, the polymerase is a DNA-dependent DNA polymerase or an RNA-dependent DNA polymerase. In some embodiments, the polymerase is a reverse transcriptase. In some embodiments, the reverse transcriptase is a retroviral reverse transcriptase, optionally wherein the reverse transcriptase is a Moloney Murine Leukemia virus reverse transcriptase (MMLV-RT), optionally wherein the MMLV-RT comprises one or more amino acid substitutions compared to a wild type MMLV-RT. In some embodiment, the napDNAbp and the polymerase of the prime editor are joined to form a fusion protein, optionally wherein the napDNAbp and the polymerase are joined by a linker. In some embodiments, the DNA polymerase is provided in trans. In some embodiments, the prime editor and the pegRNA are encoded by one or more DNA vectors. In some embodiments, the one or more DNA vectors comprise AAV or lentivirus DNA vectors.

In addition to the prime editor, the prime editing requires a prime editing guide RNA (pegRNA) or a nucleic acid sequence encoding the pegRNA, wherein the pegRNA comprises a spacer sequence, a gRNA backbone, and an extension arm comprising a DNA synthesis template (RT template) and a primer binding site (PBS), wherein the spacer sequence comprises a region of complementarity to a target strand of a double stranded target DNA sequence, wherein the gRNA core associates with the napDNAbp, wherein the DNA synthesis template comprises a region of complementarity to the non-target strand of the double-stranded target DNA sequence and one or more nucleotide edits compared to the target strand double-stranded target DNA sequence, and wherein the primer binding site comprises a region of complementarity to a non-target strand of the double-stranded target DNA sequence. In one embodiment, a different pegRNA is designed specifically for each of the target gene mutations. In one embodiment, the pegRNA is the pegRNA coded by a DNA sequence comprising the sequence of any one of SEQ ID Nos: 1100-1113. In one embodiment, the polynucleotide is an RNA that comprises the RNA coded by the DNA of SEQ ID Nos: 1100-1113. Once a specific desired mutation has been identified, there are multiple publicly available computational methods that may be used to design a pegRNA sequence for a specific desired mutation in a specific gene. See, e.g., Hsu, J. Y., Grünewald, J., Szalay, R. et al. PrimeDesign software for rapid and simplified design of prime editing guide RNAs (e.g., Hsu, J.Y., Grünewald, J., Szalay, R. et al. PrimeDesign software for rapid and simplified design of prime editing guide RNAs. Nat Commun 12, 1034 (2021).) accessible providers of pegRNA sequences for desired mutations.

In some embodiments, the pegRNA are G-quadruplexes modified pegRNAs. In some embodiments, the pegRNA is a xrRNA motif-joined pegRNA. In some embodiments, the pegRNA is a tethered or split pegRNA.

In some embodiments, once a pegRNA has been designed, the first step of prime editing comprises contacting a target nucleotide molecule with a prime editor, which may include (i) delivering directly to a cell an effective amount of a prime editor fusion protein (e.g., PEI or PE2) complexed with a lipid delivery system; (ii) delivering to a cell a mRNA or delivery complex comprising an mRNA that encodes a prime editor fusion protein and/or a suitable pegRNA; and/or (iii) delivering to the cell a DNA vector that encodes a prime editor fusion protein and/or a suitable pegRNA on one or more DNA vectors. In some embodiments, the RT is provided in trans. In some embodiments, the nucleic acid delivery may occur through a virus vector, plasmid, or other nucleic acid delivery vector. In some embodiments, the vector is an adeno-associated (AAV) vector or a lentivirus vector.

In one embodiment, prime editing reproduces a known single nucleotide polymorphism, or SNP. If more than 1% of a population does not carry the same nucleotide at a specific position in the DNA sequence, then this variation may be classified as a SNP. If a SNP occurs within a gene, then the gene is described as having more than one allele. In these cases, SNPs may lead to variations in the amino acid sequence. SNPs, however, are not just associated with genes; they can also occur in noncoding regions of DNA.

In some embodiments, the one or more modifications to the nucleic acid molecule installed at the target site comprises one or more transitions, one or more transversions, one or more insertions, one or more deletions, one more inversions, or any combination thereof, and optionally are less than 15 bp. In one embodiment, the one or more transitions are selected from the group consisting of: (a) T to C; (b) A to G; (c) C to T; and (d) G to A. In one embodiment, the one or more transversions are selected from the group consisting of: (a) T to A; (b) T to G; (c) C to G; (d) C to A; (e) A to T; (f) A to C; (g) G to C; and (h) G to T. In one embodiment, the one or more modifications comprises changing (1) a G: C basepair to a T: A basepair, (2) a G: C basepair to an A: T basepair, (3) a G: C basepair to a C: G basepair, (4) a T: A basepair to a G: C basepair, (5) a T: A basepair to an A: T basepair, (6) a T: A basepair to a C: G basepair, (7) a C: G basepair to a G: C basepair, (8) a C: G basepair to a T: A basepair, (9) a C: G basepair to an A: T basepair, (10) an A: T basepair to a T: A basepair, (11) an A: T basepair to a G: C basepair, or (12) an A: T basepair to a C: G basepair. In one embodiment, the one or more modifications comprises an insertion or deletion of 1, 2, 3, 4, 5, 6, 7, 8, 9, 10, 11, 12, 13, 14, 15, 16, 17, 18, 19, 20, 21, 22, 23, 24, or 25 nucleotides, optionally wherein the one or more edits comprises an insertion or deletion of 1-15 nucleotides.

In still other embodiments, the method introduces a desired nucleotide change that is an insertion. In certain cases, the insertion is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 nucleotides in length. In other embodiments, the method introduces a desired nucleotide change that is a deletion. In certain other cases, the deletion is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 nucleotides in length.

In some embodiments, the mutation is the V476I SNP rs199472908 C>T, which results from the SNP GRCh38.p13 (version) chr 7: NC_000007.14: g.150952556C>T (KCNH2 RefSeqGene (LRG_288): NG_008916.1: g.30371G>A). I (protein alteration). In other embodiments, the mutation may be selected by those identified as described above. In some embodiments, the mutation is selected from the SNPs of Table 1, represented in mRNA SEQ ID Nos: 2-1054.

### Other Components of the Gene Editing System

In some embodiments, the disclosure provides vectors. In some embodiments, the disclosure provides vectors that may encode the components of a gene editing system. In some embodiments, the gene editing system is a prime editing (PE) system. In some embodiments, the vectors encode a component of a PE system (e.g., PE fusion proteins, or any of the components thereof (e.g., napDNAbp, linkers, or polymerases). In other embodiments, the vectors may encode the pegRNAs, and/or the accessory gRNA for second strand nicking. In some embodiments, the vectors are capable of driving expression of one or more coding sequences in a cell. In some embodiment, the cell is a pluripotent cell. In some embodiments, the cell is an iPSC. In some embodiments, the cell is a cardiomyocyte. In some embodiments, the cell is an iPSC-derived cardiomyocyte. In some embodiments, the cell may be a prokaryotic cell, such as, e.g., a bacterial cell when used to prepare the components of the editing system. In some embodiments, the cell may be another eukaryotic cell, such as, e.g., a yeast, plant, insect, or mammalian cell, preferably for expressing the components of the editing system. In some embodiments, the eukaryotic cell may be a mammalian cell. In some embodiments, the eukaryotic cell may be a rodent cell. In some embodiments, the eukaryotic cell may be a human cell.

Suitable promoters to drive expression in different types of cells are known in the art. In some embodiments, the promoter may be wild-type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus. In some embodiments, the promoters that may be used in the prime editor vectors may be constitutive, inducible, or tissue-specific. In some embodiments, the promoters may be a constitutive promoters. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EFla) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EFla promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level.

In some embodiments, the nucleotide encoding the crRNA of the guide RNA and the nucleotide encoding the tracr RNA of the guide RNA may be provided on the same vector. In some embodiments, the nucleotide encoding the crRNA and the nucleotide encoding the tracr RNA may be driven by the same promoter. In some embodiments, the crRNA and tracr RNA may be transcribed into a single transcript. For example, the crRNA and tracr RNA may be processed from the single transcript to form a double-molecule guide RNA. Alternatively, the crRNA and tracr RNA may be transcribed into a single-molecule guide RNA. In some embodiments, the nucleotide sequence encoding the guide RNA may be located on the same vector comprising the nucleotide sequence encoding the PE fusion protein. In some embodiments, expression of the guide RNA and of the PE fusion protein may be driven by their corresponding promoters. In some embodiments, expression of the guide RNA may be driven by the same promoter that drives expression of the PE fusion protein. In some embodiments, the guide RNA and the PE fusion protein transcript may be contained within a single transcript. For example, the guide RNA may be within an untranslated region (UTR) of the Cas9 protein transcript. In some embodiments, the guide RNA may be within the 5′ UTR of the PE fusion protein transcript. In other embodiments, the guide RNA may be within the 3′ UTR of the PE fusion protein transcript.

### Delivery

Exemplary delivery strategies include vector-based strategies, (PE) ribonucleoprotein complex delivery, and delivery of prime editors by mRNA methods. In some embodiments, the method of delivery provided comprises nucleofection, microinjection, biolistics, virosomes, liposomes, immunoliposomes, polycation or lipid: nucleic acid conjugates, naked DNA, artificial virions, and agent-enhanced uptake of DNA. Exemplary methods of delivery of nucleic acids include lipofection, nucleofection, electoporation, stable genome integration (e.g., piggybac), microinjection, biolistics, virosomes, liposomes, immunoliposomes, polycation or lipid: nucleic acid conjugates, naked DNA, artificial virions, and agent-enhanced uptake of DNA. In one embodiment, the lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides are known in the art. In one embodiment, delivery may be to iPSC or cardiomyocyte cells (e.g., in vitro or ex vivo administration). Delivery may be achieved through the use of RNP complexes. The preparation of lipid: nucleic acid complexes, including targeted liposomes such as immunolipid complexes, is well known to one of skill in the art. In other embodiments, the method of delivery and vector provided herein is an RNP complex. RNP delivery of fusion proteins markedly increases the DNA specificity of base editing. RNP delivery of fusion proteins leads to decoupling of on- and off-target DNA editing. Additional methods for the delivery of nucleic acids to cells are known to those skilled in the art. See, for example, WO2022150790, incorporated herein by reference.

In a specific embodiment, the disclosure provides methods of delivering the prime editor constructs into a cell to form a complete and functional prime editor within a cell. For example, in some embodiments, a cell is contacted with a composition described herein (e.g., compositions comprising nucleotide sequences encoding the Cas9-RT protein or the prime editor or AAV particles containing nucleic acid vectors comprising such nucleotide sequences). In some embodiments, the contacting results in the delivery of such nucleotide sequences into a cell, wherein the N-terminal portion of the Cas9-RT protein or the prime editor and the C-terminal portion of the Cas9-RT protein or the prime editor are expressed in the cell and are joined to form a complete Cas9-RT protein or a complete prime editor. Any rAAV particle, nucleic acid molecule or composition provided herein may be introduced into the cell in any suitable way, either stably or transiently. In some embodiments, the disclosed proteins may be transfected into the iPSC cell. In some embodiments, the disclosed proteins may be transfected into the cardiomyocyte cell. In some embodiments, the cell may be transduced or transfected with a nucleic acid molecule. For example, a cell may be transduced (e.g., with a virus encoding a split protein), or transfected (e.g., with a plasmid encoding a split protein) with a nucleic acid molecule that encodes a protein, or an rAAV particle containing a viral genome encoding one or more nucleic acid molecules. Such transduction may be a stable or transient transduction. In some embodiments, cells expressing a split protein or containing a split protein may be transduced or transfected with one or more guide RNA sequences, for example in delivery of Cas9-RT (e.g., nCas9) protein. In some embodiments, a plasmid expressing a Cas9-RT protein may be introduced into cells through electroporation, transient (e.g., lipofection) and stable genome integration (e.g., piggybac) and viral transduction or other methods known to those of skill in the art.

In some embodiments, human iPSC are transfected with two separate plasmids. In some embodiments, one plasmid encodes a Cas9-RT fusion protein and the other plasmid encodes the pegRNA. In some embodiments, the plasmids are transfected via electroporation. In some embodiments, the plasmids are transfected with a nucleogector. In some embodiments, this method is used to introduce the V476I variant into iPSCs.

### Pluripotent Stem Cells

The disclosure provides that cardiomyocytes derived from pluripotent stem cells genetically engineered to carry mutations in specific genes are useful and novel models for studying the cardiotoxicity of those mutations and for testing drugs for their cardiotoxicity. In some other embodiments, the cardiomyoctes may be derived from human embryonic stem cells, mesenchymal stem cells, multipotent cardiac stem cells, or cardiac mesenchymal stem cells. In some embodiments, these cells may be genetically engineered to carry one or more specific mutations in one or more target genes.

Accordingly, in one embodiment, the disclosure provides induced pluripotent stem cells (iPSC) genetically engineered to carry one or more specific mutations in one or more target genes. In some embodiments, the target genes are genes known to or suspected of having a role in cardiac function. In some embodiments, the genes are selected from KCNH2, KCNQ1, SCNA5, KCNE1, KCNE2 and genes encoding other channels, ATP-binding cassette (ABC) transporters involved in drug transport (e.g., ABCB1, ABCB4, ABCCI, ABCC2, SLC10A2, SLC28A3, SLC22A7, SLC22A17), Carbonyl reductases in drug metabolism (e.g., CBR3), Hyaluronan synthase 3 involved in oxidative stress response (e.g., HAS3), Hereditary hemochromatosis protein in iron metabolism (e.g., HFE), Retinoic acid receptor gamma and DNA topoisomerases in topoisomerase-induced DNA damage (e.g., RARG, TOP2B), CUGBP Elav-like family member 4 in splicing of sarcomere genes (e.g., CELF4), DNA polymerase gamma in mitochondrial replication (e.g., DPOG2), and Chaperones involved in ion channel trafficking (e.g., Hsp70 and Hsp90). In some embodiments, the target gene is selected from potassium channel/related genes selected from human ether-a-go-go related gene (hERG), Hyperpolarization-activated cyclic nucleotide-gated (HCN) channels; transient outward potassium current channel; Slowly activating delayed rectifier potassium current channels; Rapidly activating delayed rectifier potassium current channels; Inwardly rectifying potassium (Kir) channels; Inwardly rectifying potassium channels; G protein-coupled, inwardly rectifying potassium channels; ATP-sensitive potassium channels; sodium channel/related genes selected from SCN5A, ACN1B, SCN2B, SCN3B, SCN4B, GPD1L, RANGRF, SCN10A; calcium channel/related genes selected from CACNA1C, CACNB2, CACNA2DI, RYR2, CASQ2, TRDN, CALM1-3; and other genes selected from KCNQ1, SCNA5, KCNE1, KCNE2, KvLQT1, Nav1.5, ankyrin-B, MinK, MiRP1, Kir2.1, Cav1.2, caveolin-3, Nav.beta.4 genes, SNTAI, SLMAP, PKP2, ANK2, CAV3, SLC4A3, TRPM4, DPP6, IRX3, GNAI2, ADORAI, GNAS, KCNQ1, SCNA5, KCNE1, KCNE2, KvLQT1, Nav1.5, ankyrin-B, MinK, MiRP1, Kir2.1, Cav1.2, caveolin-3, Nav.beta.4 ATP-binding cassette (ABC) transporters involved in drug transport (e.g., ABCB1, ABCB4, ABCCI, ABCC2, SLC10A2, SLC28A3, SLC22A7, SLC22A17), Carbonyl reductases in drug metabolism (e.g., CBR3), Hyaluronan synthase 3 involved in oxidative stress response (e.g., HAS3), Hereditary hemochromatosis protein in iron metabolism (e.g., HFE), Retinoic acid receptor gamma and DNA topoisomerases in topoisomerase-induced DNA damage (e.g., RARG, TOP2B), CUGBP Elav-like family member 4 in splicing of sarcomere genes (e.g., CELF4), DNA polymerase gamma in mitochondrial replication (e.g., DPOG2), and chaperones involved in ion channel trafficking (e.g., Hsp70 and Hsp90).

In some embodiments, the target gene is KONH2, which encodes a voltage-gated transmembrane potassium channel called hERG. In some embodiments, the mutation is a SNP. In some embodiments, the SNP is in the KNH2 gene. In some embodiments, the SNP is selected from those in SEQ ID Nos: 2-1054, which are listed in Table 1, obtained from the ClinVar database. In some embodiments, the SNP is V476I in the KONH2 gene. In some embodiments, the SNP is introduced in the iPSC cell by prime editing, CRISPR, base editing, or any other method that may be used to mutate one or more bases in a target gene. In some embodiments, the SNP may be introduced directly into cardiomyocytes from any source.

In some embodiments, the cardiomyocytes are differentiated from stem cells. In some embodiments, the stem cells are induced pluripotent stem cells. In some embodiments, the stem cells are human embryonic stem cells, mesenchymal stem cells, multipotent cardiac stem cells, or cardiac mesenchymal stem cells. In some embodiments, the stem cells are obtained from commercial sources. In some embodiments, the iPSC are developed as described in the literature. See, e.g. Dowey, S., Huang, X., Chou, B K. et al. Generation of integration-free human induced pluripotent stem cells from postnatal blood mononuclear cells by plasmid vector expression. Nat Protoc 7, 2013-2021 (2012). doi.org/10.1038/nprot.2012.121. In some embodiments, the iPSC are generated as described in the Examples.

In some embodiments, the disclosure provides an iPSC cell. In some embodiments, the cell is carrying one or more of the mutations of the disclosure or is carrying a genetically engineering means for altering the expression of one or more genes. In some embodiments, the disclosure provides a composition (e.g., culture) comprising or consisting of said iPSC cells. In some embodiments, the composition comprises or consists of more than one iPSC cell, each cell carrying one or more mutations as described herein. In some embodiments, the composition comprises a library of cells of the disclosure.

### Cardiomyocytes

In some embodiments, the disclosure provides a cardiomyocyte carrying one or more of the mutations of the disclosure, or one or more genes whose gene expression level has been altered. In some embodiments, the cardiomyocyte is a primary cardiomyocyte. In some embodiments, the cardiomyocyte is differentiated from stem cells in vitro. In some embodiments, the cardiomyocyte is differentiated from iPSC, in vitro. In some embodiments, the cardiomyocytes are obtained through a method comprising steps of the method of Example 6. In some embodiments, the disclosure provides a panel of cardiomyocyte cells or cell lines containing genetic diversity of established clinical relevance and variants of unknown significance for studying the cell's functionalities with different drugs. In some embodiments, the cardiomyocytes may be derived from iPSC by in vitro differentiation using combinations of small molecules, and/or growth factors, such as described in Lian et al, Nature Protocols. 8, 162-175, 2013 and Sharma et al, Journal of visualized experiments JoVE. 2015, including modulation of Wnt pathway followed by glucose starvation for selection of highly purified cardiomyocyte populations, as described here Cyganek et al, JCI insight. 3, 2018, and combinations with nucleic acids (i.e., RNA, miRNA, siRNA).

In this application, a mutation, gene expression level, or drug is associated with cardiomyocyte cardiotoxicity (and hence clinical toxicity) if the mutation, gene expression level, or drug has a negative effect on one or more of cardiomyocyte function, proliferation, viability, survival, morphology, the expression of certain markers and receptors, “heart beats” in vitro (which can model arrhythmias) relative to wild-type (e.g., isogenic) cells, wherein a negative effect is a statistically significant effect. In some embodiments, a mutation, gene expression level, or drug is associated with cardiomyocyte cardiotoxicity (and hence clinical toxicity) if the mutation, gene expression level, or drug has a negative effect on one or more of cardiomyocyte function, proliferation, viability, survival, morphology, the expression of certain markers and receptors, “heart beats” in vitro (which can model arrhythmias) relative to wild-type (e.g., isogenic) cells, wherein there is a negative effect if there is a change of at least 5% relative to wild-type cells.

In this application, a “wild-type” subject is a healthy subject, without clinical evidence of cardiac disease. In this application, “iPSC-derived wild-type cardiomyocytes” are myocytes obtained from iPSC derived from a healthy subject, without clinical evidence of cardiac disease. In some embodiments, “standard iPSC-derived wild-type cardiomyocytes” are cardiomyocytes derived from an iPSC cell line that is generally considered a standard in the art. In some embodiments, it may be specified which gene must be wild-type. In those embodiments, the gene is a gene associated with cardiac disease. In some embodiments, the gene is selected from KCNH2/hERG, KCNQ1, SCNA5, KCNE1, KCNE2, KvLQT1, Nav1.5, ankyrin-B, MinK, MiRP1, Kir2.1, Cav1.2, caveolin-3, and Nav.beta.4 genes; genes encoding other channels, a sodium channel/related gene selected from SCN5A, ACN1B, SCN2B, SCN3B, SCN4B, GPD1L, RANGRF, SCN10A; a potassium channel/related gene selected from human ether-a-go-go related gene (hERG), Hyperpolarization-activated cyclic nucleotide-gated (HCN) channels; transient outward potassium current channel; Slowly activating delayed rectifier potassium current channels; Rapidly activating delayed rectifier potassium current channels; Inwardly rectifying potassium (Kir) channels; Inwardly rectifying potassium channels; G protein-coupled, inwardly rectifying potassium channels; ATP-sensitive potassium channels; a calcium channel/related gene selected from CACNA1C, CACNB2, CACNA2D1, RYR2, CASQ2, TRDN, CALM1-3; other genes selected from SNTA1, SLMAP, PKP2, ANK2, CAV3, SLC4A3, TRPM4, DPP6, IRX3, GNAI2, ADORAI, GNAS, KCNQ1, SCNA5, KCNE1, KCNE2, KvLQT1, Nav1.5, ankyrin-B, MinK, MiRP1, Kir2.1, Cav1.2, caveolin-3, Nav.beta.4 ATP-binding cassette (ABC) transporters involved in drug transport (e.g., ABCB1, ABCB4, ABCCI, ABCC2, SLC10A2, SLC28A3, SLC22A7, SLC22A17), Carbonyl reductases in drug metabolism (e.g., CBR3), Hyaluronan synthase 3 involved in oxidative stress response (e.g., HAS3), Hereditary hemochromatosis protein in iron metabolism (e.g., HFE), Retinoic acid receptor gamma and DNA topoisomerases in topoisomerase-induced DNA damage (e.g., RARG, TOP2B), CUGBP Elav-like family member 4 in splicing of sarcomere genes (e.g., CELF4), DNA polymerase gamma in mitochondrial replication (e.g., DPOG2), and chaperones involved in ion channel trafficking (e.g., Hsp70 and Hsp90).

### Exemplary Non-Limiting Methods of Using the Cells of the Disclosure

Method of Identifying a Gene or Gene Mutation as Associated with or Cause for Cardiac Disease

For the purpose of the following methods or models, the cells of the disclosure comprise any kind of stem cell, iPSC, cardiomyocytes derived from stem cells, and cardiomyocytes derived from any other source, wherein the cells have been genetically modified to carry any one of the mutations described in this disclosure, to fix any mutation previously unknown or previously known related to cardiac disease or disorder, or to have altered expression of one or more genes. For example, the following methods may be practiced with cardiomyocytes derived from iPSC or cardiomyocytes from any other source, so long as they have been genetically modified to introduce or correct a mutation in a gene, or to alter the expression of one or more genes, that is related to cardiac function, preferably, cardiomyocytes' function, proliferation, viability, survival, morphology, the expression of certain markers and receptors, “heart beats” in vitro (which can model arrhythmias) relative to wild-type (e.g., isogenic) cells.

In one embodiment, the disclosure provides a method whereby iPSC (or other stem cells) are genetically modified to carry one or more mutations in one or more genes. Cardiomyocytes are derived from these cells. The cardiomyocytes are tested for the effects of the mutation on their function, proliferation, viability, survival, morphology, the expression of certain markers and receptors, and “heart beats” in vitro (which can model arrhythmias) relative to wild-type (e.g., isogenic) cells. Gene mutations that are associated with negative effects on one or more of these cardiomyocyte properties are classified as mutations associated with or cause for cardiac disease.

In one embodiment, the disclosure provides a method whereby iPSC (or other stem cells) are genetically modified to alter the gene expression of one or more genes. Cardiomyocytes are derived from these cells. In some embodiments, the cardiomyocytes are the ones that are genetically modified to alter the gene expression of one or more genes. The cardiomyocytes are tested for the effects of change in gene expression/gene expression level on their function, proliferation, viability, survival, morphology, the expression of certain markers and receptors, and “heart beats” in vitro (which can model arrhythmias) relative to wild-type (e.g., isogenic) cells. Changes in gene expression levels that are associated with negative effects on one or more of these cardiomyocyte properties are classified as genes whose expression level is associated with or cause for cardiac disease.

In all embodiments of this application, the cardiomyocytes may be derived from iPSC or not. The cardiomyocytes may be from any source. The genetic alterations that are described for the iPSC may be also introduced directly into cardiomyocytes instead of the iPSC.

Throughout this application, any gene that is mutated may also or instead have its gene expression altered (e.g., CRISPR interference). Consequently, all embodiments directed to gene mutations can equally be practiced with genes whose expression level is changed by genetic engineering and are included in this application even when not explicitly disclosed. Accordingly, in one embodiment, the mutations and alterations in gene expression levels are as described in the previous sections, including mutations selected from any of the listed databases. In one embodiment, the databases list gene mutations identified in the population in any gene. In one embodiment, the gene is selected from HERG/KCNH2, SCN5A, KCNQ1, SCNA5, KCNE1, KCNE2, KvLQT1, Nav1.5, ankyrin-B, MinK, MiRP1, Kir2.1, Cav1.2, caveolin-3, Nav.beta.4 ATP-binding cassette (ABC) transporters involved in drug transport (e.g., ABCB1, ABCB4, ABCCI, ABCC2, SLC10A2, SLC28A3, SLC22A7, SLC22A17), Carbonyl reductases in drug metabolism (e.g., CBR3), Hyaluronan synthase 3 involved in oxidative stress response (e.g., HAS3), Hereditary hemochromatosis protein in iron metabolism (e.g., HFE), Retinoic acid receptor gamma and DNA topoisomerases in topoisomerase-induced DNA damage (e.g., RARG, TOP2B), CUGBP Elav-like family member 4 in splicing of sarcomere genes (e.g., CELF4), DNA polymerase gamma in mitochondrial replication (e.g., DPOG2), Chaperones involved in ion channel trafficking (e.g., Hsp70 and Hsp90). In some embodiments, a sodium channel/relate gene is selected from SCN5A, ACN1B, SCN2B, SCN3B, SCN4B, GPD1L, RANGRF, SCN10A; a potassim channel/related gene is selected from human ether-a-go-go related gene (hERG), Hyperpolarization-activated; a sodium channel/relate gene is selected from SCN5A, ACN1B, SCN2B, SCN3B, SCN4B, GPD1L, RANGRF, SCN10A; a potassim channel/related gene is selected from human ether-a-go-go related gene (hERG), Hyperpolarization-activated cyclic nucleotide-gated (HCN) channels; transient outward potassium current channel; slowly activating delayed rectifier potassium current channels; Rapidly activating delayed rectifier potassium current channels; Inwardly rectifying potassium (Kir) channels; Inwardly rectifying potassium channels; G protein-coupled, inwardly rectifying potassium channels; ATP-sensitive potassium channels; a calcium channel/related gene is selected from CACNA1C, CACNB2, CACNA2D1, RYR2, CASQ2, TRDN, CALM1-3; other genes are selected from SNTAI, SLMAP, PKP2, ANK2, CAV3, SLC4A3, TRPM4, DPP6, IRX3, GNAI2, ADORA1, GNAS cyclic nucleotide-gated (HCN) channels; transient outward potassium current channel; Slowly activating delayed rectifier potassium current channels; Rapidly activating delayed rectifier potassium current channels; Inwardly rectifying potassium (Kir) channels; Inwardly rectifying potassium channels; G protein-coupled, inwardly rectifying potassium channels; ATP-sensitive potassium channels; the calcium channel/related gene is selected from CACNA1C, CACNB2, CACNA2DI, RYR2, CASQ2, TRDN, CALM1-3; other genes are selected from SNTA1, SLMAP, PKP2, ANK2, CAV3, SLC4A3, TRPM4, DPP6, IRX3, GNAI2, ADORAI, GNAS.

In one embodiment, the mutations are/represent SNPs. In one embodiment, the SNPs are as described in the previous sections and in Table 1 and/or SEQ ID Nos. 2-1054. In one embodiment, the mutation results in a In one embodiment, the SNP is chosen for testing according to the methods of the disclosure based on its “clinical significance.” (See Table 1). In one embodiment, the SNP is chosen for testing according to the methods of the disclosure based on the “Condition(s)” the SNP has been associated with in the ClinVar database. (See Table 1 and updated versions of the database). Though no specific SNP is being at this time selected for testing other than the ones in the Examples, each individual SNP is considered relevant for testing according to the methods of the disclosure and can be independently claimed as an individual embodiment. The clinical significance and the conditions associated with are sufficient “blaze marks” directing one skilled in the art to any of these SNPs and any SNP can be picked from among the remaining SNPs in the table without the need for further guidance.

In one embodiment, a panel of cells carrying a panel of gene mutations prepared by the methods of the disclosure may be tested for the effect of the panel of mutations on cardiomyocytes. This panel may be a library of cells with known and unknown mutations. In one embodiment, the cells are not part of a library. The results may then be compared with databases of mutations in the gene to either confirm the negative effects of the mutation on cardiac function in patients or confirm that the mutations are not pathogenic. In some embodiments, the mutations have been previously identified as influencing cardiomyocytes. In other embodiments, the disclosure provides the first identification of the gene or gene mutation as being important for cardiomyocyte function, proliferation, viability, survival, morphology, the expression of certain markers and receptors, and “heart beats” in vitro (which can model arrhythmias) relative to wild-type (e.g., isogenic) cells. In some embodiments, the disclosure provides mutations that are associated with or the cause of cardiac disease. In some embodiments, the subject can be diagnosed as potentially having one of the diseases listed in Table 1 based on the associations listed in the table.

In some embodiments, the disease is an arrhythmia. In some embodiments, the arrhythmia is selected from Long QT syndrome (including Congenital long QT syndrome, Long QT syndrome 2, and bradycardia-induced Long QT syndrome), Brugada syndrome, Short QT syndrome 1, Sudden Infant Death Syndrome, Acquired long QT syndrome, ventricular tachycardia, Wolff-Parkinson-White pattern arrhythmia, Arrhythmogenic right ventricular cardiomyopathy, Atrial fibrillation, Catecholaminergic polymorphic ventricular tachycardia type 1, Prolonged QT interval, Paroxysmal familial ventricular fibrillation 1, Sudden cardiac arrest/death and Torsades de pointes. In some embodiments, the disease is hypertrophic cardiomyopathy, sudden unexplained death, obesity-related cardiac disease, primary dilated cardiomyopathy, primary familial hypertrophic cardiomyopathy, or Seizures.

In some embodiments, the effect of the gene mutation on cardiomyocytes is assessed by a cardiotoxicity assay, which may measure the effect of the mutation on cardiomyocyte cell viability, survival, morphology, the expression of certain markers and receptors, and/or “heart beats” in vitro (which can model arrhythmias). In some embodiments, the cardiotoxicity assay comprises using a patch clamp technique, an external recording method, a voltage-sensitive dye, or an intracellular ion-sensitive dye. These assays may be used in any of the methods of the disclosure.

Method of Diagnosing a Subject as being Susceptible to Cardiac Disease

In some embodiments, the disclosure provides a means for identifying a subject as someone susceptible to cardiac disease. In some embodiments, the subject is identified as someone susceptible to cardiac disease when the subject carries one or more of the mutations in their genome identified as affecting cardiomyocyte function, proliferation, viability, survival, morphology, the expression of certain markers and receptors, “heart beats” in vitro (which can model arrhythmias) relative to wild-type (e.g., isogenic) cells, preferably through the methods disclosed herein. In one embodiment, negative effects on any one or more of each of these cardiomyocyte properties is representative of cardiotoxicity. In some embodiments, the subject was already aware that they had a cardiac disease. In some embodiments, the subject was not aware that they carried a cardiac disease. In some embodiments, the subject was not previously aware that they carried the gene mutation. In some embodiments, the subject was not previously aware that it carried a gene mutation that was associated with susceptibility to cardiac disease. In some embodiments, the subject may be subsequently diagnosed as potentially having one of the diseases listed in Table 1 based on the associations listed in the table.

In some embodiments, the disease is an arrhythmia. In some embodiments, the arrhythmia is selected from Long QT syndrome (including Congenital long QT syndrome, Long QT syndrome 2, and bradycardia-induced Long QT syndrome), Brugada syndrome, Short QT syndrome 1, Sudden Infant Death Syndrome, Acquired long QT syndrome, ventricular tachycardia, Wolff-Parkinson-White pattern arrhythmia, Arrhythmogenic right ventricular cardiomyopathy, Atrial fibrillation, Catecholaminergic polymorphic ventricular tachycardia type 1, Prolonged QT interval, Paroxysmal familial ventricular fibrillation 1, Sudden cardiac arrest/death and Torsades de pointes. In some embodiments, the disease is hypertrophic cardiomyopathy, sudden unexplained death, obesity-related cardiac disease, primary dilated cardiomyopathy, primary familial hypertrophic cardiomyopathy, or Seizures.

In some embodiments, the subject's family members (e.g., progeny) are tested for the presence of one or more of the mutations identified as associated with cardiotoxicity or cardiac disease by the methods of the disclosure.

In some embodiments, the mutation is incorporated into commercial diagnostic tests. In some embodiments, the disclosure provides a kit for diagnosing an individual as carrying one or more of the mutations originally identified as cardiotoxic or associated with cardiac disease by the methods of the invention. In some embodiments, the kits are as described earlier in this application.

Method of Identifying a Gene Mutation as Associated with Drug-Induced Cardiac Disease

In one embodiment, the disclosure provides a method of identifying gene mutations that confer to cardiomyocytes carrying those mutations sensitivity to one or more drugs. In one embodiment, an iPSC-derived cardiomyocyte carrying one or more gene mutations as described above is exposed to a drug and the effect of the drug exposure on the cardiomyocyte function, proliferation, viability, survival, morphology, the expression of certain markers and receptors, and/or “heart beats” in vitro (which can model arrhythmias) is assessed. In one embodiment, if a drug has a negative effect on any one or more of these cardiomyocyte properties, the drug is said to be “cardiotoxic” or “associated with cardiotoxicity” in subjects carrying that mutation. In some embodiments, the cardiomyocytes are derived from iPSC with the methods of the disclosure.

In one embodiment, the presence of the cardiotoxic gene mutation in a subject is used to identify subjects that will present cardiac disease if exposed to the drug. In some embodiments, this method identifies subjects that should not be exposed to said drug.

In one embodiment, an individual drug is assessed at a time. In another embodiment, a library or panel of drugs may be assessed in multiple cardiomyocytes at the same time. In one embodiment, the cardiomyocytes are cultured in multi-well plates (e.g., 96 well plates) and a different drug is tested in each well or set of wells (e.g., 1 drug per each 3 wells to have triplicate results).

In one embodiment, the drug is a new drug. In another embodiment, the drug is not a new drug. In one embodiment, the assay is used as a model of drug-induced cardiotoxicity for regulatory purposes (e.g., FDA approval). In one embodiment, the drug is selected from those listed in Table 2 and Table 3 and other drugs of the same classes. In one embodiment, the drug is selected from sotalol, propranolol, and isoproterenol.

### Method to Assess Whether a Drug is Cardiotoxic

In one embodiment, the disclosure provides a method of identifying a drug as cardiotoxic to the “wild-type” population in general. In one embodiment, an iPSC-derived cardiomyocyte not carrying one or more gene mutations identified herein (i.e., cardiomyocytes from one or more representatives of wild-type individuals, wherein a nucleotide sequence is dominant or most frequently observed in the population) is exposed to a drug and the effect of the drug exposure on the cardiomyocyte function, proliferation, viability, survival, morphology, the expression of certain markers and receptors, and/or “heart beats” in vitro (which can model arrhythmias) is assessed. In one embodiment, if a drug has a negative effect on any one or more of these cardiomyocyte properties, the drug is said to be cardiotoxic or associated with cardiotoxicity. In some embodiments, the cardiomyocytes are derived from iPSC with the methods of the disclosure.

In one embodiment, an individual drug is assessed at a time. In another embodiment, a library or panel of drugs may be assessed in multiple cardiomyocytes at the same time. In one embodiment, the cardiomyocytes are cultured in multi-well plates (e.g., 96 well plates) and a different drug is tested in each well or set of wells (e.g., 1 drug per each 3 wells to have triplicate results).

In one embodiment, the drug is a new drug. In another embodiment, the drug is not a new drug. In one embodiment, the assay is used as a model of drug-induced cardiotoxicity for regulatory purposes (e.g., FDA approval). In one embodiment, the drug is selected from those listed in Table 2 and Table 3 and other drugs of the same classes.

Method of Treating a Subject with a Cardiovascular Disease

In one embodiment, a subject is identified as having a cardiovascular disease using the methods disclosed above. In one embodiment, the subject is further treated for the disease after experiencing symptoms of the disease. In one embodiment, the subject is treated prophylactically. In one embodiment, the subject is identified as being at risk for any one of the cardiovascular diseases associated with arrhythmia. In one embodiment, a cardiac defibrillator is implanted in the subject prophylactically or after experiencing arrhythmia.

In one embodiment, the subject is treated by correction of the mutation that is associated with or causes the disease, as discovered by the methods of the invention. In one embodiment, the correction of the mutation is done by CRISPR, base editing, or prime editing.

### Reverse Methods

In one embodiment, the methods just described are carried out not by introducing a mutation into a gene but by correcting a mutation in a gene and assessing the effect of the correction on cardiomyocyte function, proliferation, viability, survival, morphology, the expression of certain markers and receptors, and/or “heart beats” in vitro (which can model arrhythmias). In one embodiment, the mutation is known to be cardiotoxic, or suspected to be cardiotoxic and the effect of changing or correcting that mutation to a different base/sequence (e.g., wild-type sequence) on cardiotoxicity is assessed with the methods of the disclosure.

In some embodiments, the discovery that the change or correction of the mutation in the cardiomyocytes genome may improve the cardiomyocytes' function, proliferation, viability, survival, morphology, the expression of certain markers and receptors, and/or “heart beats” in vitro (which can model arrhythmias) may be used to treat cardiac disease or reduce drug cardiotoxicity in a subject, by altering or correcting the subject's cardiomyocytes through gene editing. In some embodiments, the gene editing comprises CRISPR, base editing, or prime editing. In some embodiments, the subject is a human.

### Other Methods

The nucleic acids and cells of the disclosure may be used in any other method whereby the relationship between a specific gene mutation and/or gene expression level and cardiotoxicity (as defined above) play a role. Such methods are all within the scope of the disclosure.

### Compositions

In one embodiment, the disclosure provides compositions comprising or consisting of one or more nucleic acids and/or proteins of the disclosure. In one embodiment, the disclosure provides compositions comprising one or more cells of the disclosure (i.e., iPSC and cardiomyocytes). In some embodiments, the compositions are pharmacological compositions. In some embodiments, the compositions comprise or consist of one or more components of a gene editing system described herein and are capable of being administered to a cell, tissue, or organism by any suitable means, such as by gene therapy, mRNA delivery, virus-like particle delivery, or ribonucleoprotein (RNP) delivery, and combinations thereof, as described above.

In one embodiment, the disclosure provides compositions for delivering the nucleic acids of the disclosure to a cell. In one embodiment, the compositions comprise or consist of a pegRNA of the disclosure. In one embodiment, the compositions comprise or consisting of a prime editor of the disclosure. In one embodiment, the compositions or consisting of comprise both. More compositions are described above in the methods of delivery of the gene editing system.

In one embodiment, the one or more modifications comprises a correction to a mutation associated with a disease in a disease-associated gene. In one embodiment, the one or more modifications comprises the introduction of a mutation associated with a disease in a disease-associated gene. In one embodiment, the mutation is a SNP of the disclosure. In one embodiment, the disease-associated gene is associated with cardiac disease. In one embodiment, the disease is selected from those diseases described elsewhere in the application. In one embodiment, the disease-associated gene is the HERG gene. In one embodiment, the disease-associated gene is selected from those listed elsewhere in the specification.

In some embodiments, the compositions are pharmaceutical compositions. In some embodiments, the pharmaceutical composition comprises any of the compositions disclosed herein. In some embodiments, the pharmaceutical composition comprises any of the compositions disclosed herein and a pharmaceutically acceptable carrier. In some embodiments, the compositions comprise or consists of one or more cells of the disclosure. In one embodiment, the composition comprises iPSC. In one embodiment, the composition comprises cardiomyocytes. In one embodiment, the composition comprises or consists of both iPSC and cardiomyocytes. In some embodiments, the composition comprises or consists of any one of these cells and one or more components of a gene editing system (nucleic acids/polynucleotides; proteins; combinations). In some embodiments, the pharmaceutical composition comprises or consists of any of the polynucleotides disclosed herein. In some embodiments, the pharmaceutical composition comprises or consists of any of the polynucleotides disclosed herein and a pharmaceutically acceptable carrier. Any reference to a composition of the disclosure as “comprising” something, is also a reference to the same composition as “consisting of” that something, and also a reference to the same composition as “consisting essentially of” that something, even if not explicitly disclosed or enumerated herein.

Some examples of materials which may serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

### Kits

The compositions of the present disclosure may be assembled into kits. In some embodiments, the kit comprises the cells (iPSC and/or cardiomyocytes) of the disclosure. In some embodiments, the kits, instead of the cells or in addition to the cells, comprise nucleic acid vectors for the expression of a prime editor. In other embodiments, the kit further comprises appropriate guide nucleotide sequences (e.g., PERNAs and second-site gRNAs) or nucleic acid vectors for the expression of such guide nucleotide sequences, to target the Cas9 protein or prime editor to the desired target sequence. In some embodiments, the kit described herein may include one or more containers housing components for performing the methods described herein and optionally instructions for use. Any of the kit described herein may further comprise components needed for performing the assay methods. In some embodiments, each component of the kits, where applicable, may be provided in liquid form (e.g., in solution) or in solid form, (e.g., a dry powder). In certain embodiments, some of the components may be reconstitutable or otherwise processible (e.g., to an active form), for example, by the addition of a suitable solvent or other species (for example, water), which may or may not be provided with the kit. In some embodiments, the kits may optionally include instructions and/or promotion for use of the components provided. As used herein, “instructions” may define a component of instruction and/or promotion, and typically involve written instructions on or associated with packaging of the disclosure. Instructions also may include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use, or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the disclosure. In some embodiments, the kits may include other components depending on the specific application, as described herein. In some embodiments, the kits may contain any one or more of the components described herein in one or more containers. In some embodiments, the components may be prepared sterilely, packaged in a syringe and shipped refrigerated. Alternatively, it may be housed in a vial or other container for storage. A second container may have other components prepared sterilely. In some embodiments, the kits may include the active agents premixed and shipped in a vial, tube, or other container. The kits may have a variety of forms, such as a blister pouch, a shrink-wrapped pouch, a vacuum sealable pouch, a sealable thermoformed tray, or a similar pouch or tray form, with the accessories loosely packed within the pouch, one or more tubes, containers, a box or a bag. The kits may be sterilized after the accessories are added, thereby allowing the individual accessories in the container to be otherwise unwrapped. In some embodiments, the kits may be sterilized using any appropriate sterilization techniques, such as radiation sterilization, heat sterilization, or other sterilization methods known in the art. In some embodiments, the kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc. In some embodiments, the disclosure provides kits comprising a nucleic acid construct comprising a nucleotide sequence encoding the various components of the prime editing system utilized in the methods and compositions described herein (e.g., including, but not limited to, the napDNAbps, reverse transcriptases, polymerases, fusion proteins (e.g., comprising napDNAbps and reverse transcriptases (or more broadly, polymerases), extended guide RNAs, and complexes comprising fusion proteins and extended guide RNAs, as well as accessory elements, such as second strand nicking components (e.g., second strand nicking gRNA) and 5′ endogenous DNA flap removal endonucleases for helping to drive the prime editing process towards the edited product formation). In some embodiments, the nucleotide sequence(s) comprises a heterologous promoter (or more than a single promoter) that drives expression of the prime editing system components. Other embodiments of this disclosure provide kits comprising one or more nucleic acid constructs encoding the various components of the prime editing systems utilized in the methods and compositions described herein, e.g., the comprising a nucleotide sequence encoding the components of the gene editing (e.g., prime editing) system capable of modifying a target DNA sequence.

### Examples for Characterization of the Predict Platform

The PREDICT PLATFORM comprises a combination of iPSC and iPSC-derived cardiomyocytes genetically modified to carry cardiac disease-related gene mutations, which may be used for any model of applying such cells to study gene mutation effects on cardiomyocytes and drug-related cardiotoxicity. It also comprises a combination of iPSC and iPSC-derived cardiomyocytes modified to carry genetic engineering alterations in the expression of one or more genes, which may be used for any model of applying such cells to study gene expression effects on cardiomyocytes and drug-related cardiotoxicity.

### Example 1: Bioinformatic Selection of SNPS

Public databases were searched for variations in KCHN2 gene. SNPs were ranked based on Clin Var classification into known drug interaction, pathogenic and uncertain. SNPs were then ranked based on frequency. Highest frequency SNPs were selected for experimental validation.

### Predicting the Pathogenicity of KCNH-2 SNPs of Unknown Significance.

Biochemical logic, as used by a domain expert, may predict the pathogenicity of SNPs with reasonable accuracy for clear-cut cases. For example, a nonsynonymous mutation may introduce a helix-breaking substitution (i.e., Pro or Gly) in an alpha helix or insert a sterically bulky side chain (e.g., Trp) into the channel pore that would block the flow of ions—both SNPs would be predicted to disrupt channel function. Conversely, a SNP that occurs in a highly variable (low conservation) position that resides on the surface of the protein would likely be well tolerated and have negligible effects on function. However, many SNPs in which biophysical modeling and analysis suggest subtle effects are the most challenging cases to predict. The inventors developed a new method for discovering and characterizing mutations in various genes that may be associated with cardiac diseases. These methods serve to screen large libraries of mutants for mutations that lead to cardiomyocyte toxicity. They also serve for drug screening.

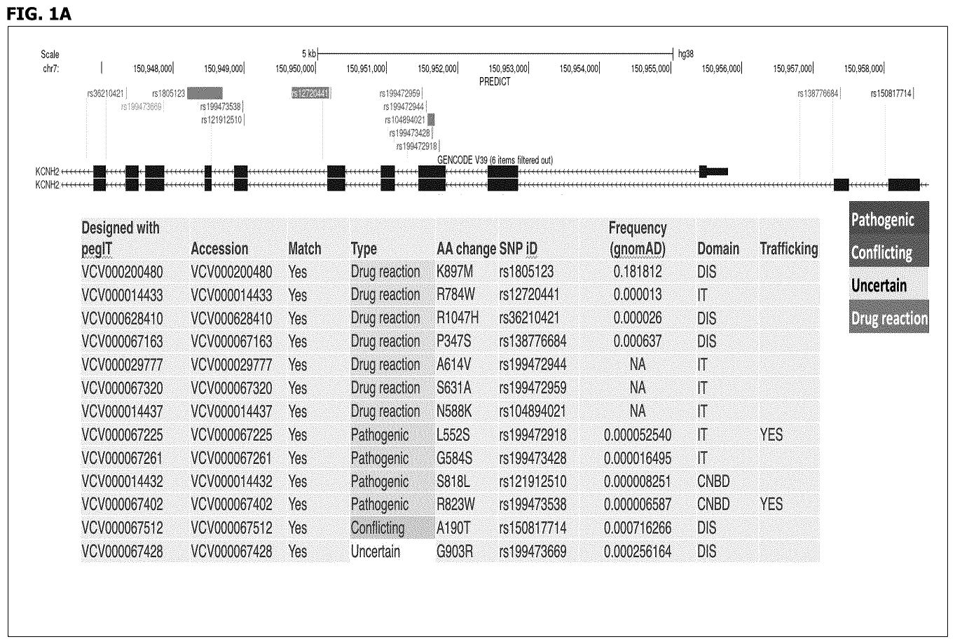

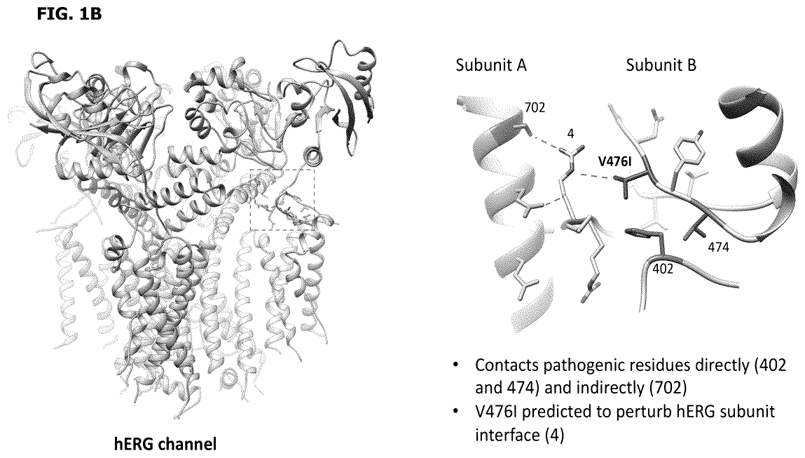

In this example, the inventors introduced a known SNP into iPSC via prime editing and studied its effect in iPSC-derived cardiomyocytes. They selected V476I—a SNP that may be associated with cardiac diseases. The V476I SNP appeared in the FAMILION® long QT syndrome genetic test as described by Kapplinger et al., Heart rhythm. 6:1297-1303, 2009. This Retrospective analysis of the first 2,500 cases (1,515 female patients, average age at testing 23 17 years, range 0 to 90 years) scanned for mutations in 5 of the LQTS-susceptibility genes also referenced in Duzkale et al, Clinical genetics. 84:453-463, 2013 and Ware et al., Human mutation. 33:1188-1191, 2012 and documented in ClinVar https://www.ncbi.nlm.nih.gov/clinvar/RCV000057908/. The inventors applied proprietary machine learning (ML) artificial intelligence to multiple data sets, and the review of the ML training data reveals no notable observations about the significance (disease relatedness) of this mutation. Specifically, V476I was found to have i) moderate centrality (network) scores, ii) low conservation (0.2) so this position should tolerate substitutions, iii) epistatic score (−4.58) of borderline significance, iv) no involvement in subunit interactions, pore, etc. The inventors' structural analysis reveals that V476I participates as a central “packing” residue for helix-loop-helix fold, contacts pathogenic residues (407 and 407) identified in ClinVar (FIG. 1B). Therefore, it is possible that the knock on effect of V476I may impact the kchn-2 subunit interface via residue interaction network effects. From prior computational work, V476I was predicted to be pathogenic mutation by SNP&Go5 and KvSNP6 algorithms.

### Example 2: Design and Cloning of Prime Editing Constructs

A list of SNP IDs including V476I were inputted into an online tool (pegIT https://pegit.giehmlab.dk/) for design of prime editing pegRNAs, consisting of 3 pairs of oligos for each pegRNA: spacer, extension, and scaffold oligos. DNA oligos were ordered from IDT and annealed in house. Only the scaffold oligo was ordered phosphorylated at 5′ end. Prime editing plasmids pCMV-PE2-P2A-GFP and pU6-pegRNA-GG-acceptor were gifts from David Liu (AddGene #132776, #132777, and/or #132775). Cloning of pegRNAs was performed as previously described (Anzalone et al., 2019). Briefly, pU6-pegRNA-GG-acceptor was digested with BsaI to generate an open backbone for cloning. Backbone fragment was purified from a 2% agarose gel. A ligation reaction was performed with the 3 oligos and digested pU6-pegRNA-GG-acceptor backbone, which was subsequently transformed into E. coli. Resulting plasmids sequences were confirmed using Sanger sequencing (primer 5′-GAGGGCCTATTTCCCATGATT-3′). The components of the pegRNA for introduction of the V476I mutation are the mRNAs coded by the following DNA sequences:

CTCCTCGTTGGCATTGAcGTGTTTTAGAGCTAGAAATAGCAAGTTAAAATAAGGCTAGT
CCGTTATCAACTTGAAAAAGTGGCACCGAGTCGGTGCGCACCACCTACaTCAATGCCAA
CGA
Spacer-pegRNA backbone-RT template-PBS
CTCCTCGTTGGCATTGAcGT-Spacer
GTTTTAGAGCTAGAAATAGCAAGTTAAAATAAGGCTAGTCCGTTATCAACTTGAAAAAG
TGGCACCGAGTCGGTGC-pegRNA backbone
GCACCACCTACa-RT Template
TCAATGCCAACGA-PBS

Other pegRNAs are those coded by the DNA sequences referred to in FIG. 1 are as follows (ard correspond to SEQ ID Nos 1100-1113 in order of appearance.:

rs1805123 VCV000200480 132777_K897M
TTCCGCAGGCGCACGGACAaGTTTTAGAGCTAGAAATAGCAAGTTAAAATAAGG
CTAGTCCGTTATCAACTTGAAAAAGTGGCACCGAGTCGGTGCTCCCCCGCCTCACCCaTG
TCCGTGCGCCTGCGGAAGGA
rs12720441 VCV000014433 132777_R784W
GCAGGATCTCGATGGAGCCCGTTTTAGAGCTAGAAATAGCAAGTTAAAATAAGG
CTAGTCCGTTATCAACTTGAAAAAGTGGCACCGAGTCGGTGCACTTCATCTCCtGGGGCT
CCATCGAGATC
rs36210421 VCV000628410 132777_R1047H
CTCACCTGTTGAGCTGGcGCGTTTTAGAGCTAGAAATAGCAAGTTAAAATAAGGC
TAGTCCGTTATCAACTTGAAAAAGTGGCACCGAGTCGGTGCGCCCTCCAGCaCCAGCTC
AACAGGT
rs138776684 VCV000067163 132777_P347S
TGGTGGGCGAAGCCAAGAAGGTTTTAGAGCTAGAAATAGCAAGTTAAAATAAGG
CTAGTCCGTTATCAACTTGAAAAAGTGGCACCGAGTCGGTGCTCAAGGGCGACtCCTTCT
TGGCTTCGCCCACCAGT
rs199472944 VCV000029777 132777_A614V
CATCAAGGACAAGTATGTGAGTTTTAGAGCTAGAAATAGCAAGTTAAAATAAGG
CTAGTCCGTTATCAACTTGAAAAAGTGGCACCGAGTCGGTGCGAAGTAGAGCaCCGTCA
CATACTTGTCCTTG
rs199472959 VCV000067320 132777_S631A
ATCTTCTCTGAGTTGGTGTTGTTTTAGAGCTAGAAATAGCAAGTTAAAATAAGGC
TAGTCCGTTATCAACTTGAAAAAGTGGCACCGAGTCGGTGCTCGGCAACGTCgCTCCCA
ACACCAACTCAGAGA
rs104894021 VCV000014437 132777_N588K
GTTTGCCTATCTGGTCGCCCGTTTTAGAGCTAGAAATAGCAAGTTAAAATAAGGC
TAGTCCGTTATCAACTTGAAAAAGTGGCACCGAGTCGGTGCGGCTGCACAAaCTGGGCG
ACCAGATAGGCA
rs199472918 VCV000067225 132777_L552S
CCAGTGCGCGATGAGCGCAAGTTTTAGAGCTAGAAATAGCAAGTTAAAATAAGG
CTAGTCCGTTATCAACTTGAAAAAGTGGCACCGAGTCGGTGCGTGCTGTTCTcGCTCATG
TGCACCTTTGCGCTCATCGCGCACTGGCTA
rs199473428 VCV000067261 132777_G584S
CCACACATGGACTCACGCATGTTTTAGAGCTAGAAATAGCAAGTTAAAATAAGG
CTAGTCCGTTATCAACTTGAAAAAGTGGCACCGAGTCGGTGCTGCAGCCAGCtGATGCG
TGAGTCCATGT
rs121912510 VCV000014432 132777_S818L
CTGAACCTGTATGCAAGGCCGTTTTAGAGCTAGAAATAGCAAGTTAAAATAAGG
CTAGTCCGTTATCAACTTGAAAAAGTGGCACCGAGTCGGTGCATCCCCGTTCaACTTGCC
AGGCCTTGCATACAGGT
rs199473538 VCV000067402 132777_R823W
CAAGTCGAACGGGGATGTGcGTTTTAGAGCTAGAAATAGCAAGTTAAAATAAGG
CTAGTCCGTTATCAACTTGAAAAAGTGGCACCGAGTCGGTGCGTGAGGGCCCaCACATC
CCCGTTCGACT
rs150817714 VCV000067512 132777_A190T
GTCCACCACCACGGCCCCCGGTTTTAGAGCTAGAAATAGCAAGTTAAAATAAGG
CTAGTCCGTTATCAACTTGAAAAAGTGGCACCGAGTCGGTGCGCGGCGCGGGCaCCCCG
GGGGCCGTGGTGGTGGACGT
rs199473669 VCV000067428 132777_G903R
CCCACAGACACGGAGCAGCCGTTTTAGAGCTAGAAATAGCAAGTTAAAATAAGG
CTAGTCCGTTATCAACTTGAAAAAGTGGCACCGAGTCGGTGCGACACCTCCCtTGGCTGC
TCCGTGTCTGT
rs199472908 VCV000067200 PE2_V476I
CTCCTCGTTGGCATTGAcGTGTTTTAGAGCTAGAAATAGCAAGTTAAAATAAGGC
TAGTCCGTTATCAACTTGAAAAAGTGGCACCGAGTCGGTGCGCACCACCTACaTCAATG
CCAACGA

Barcodes may be inserted into the pegRNA expression vector by restriction fragment cloning of small oligo nucleotides containing a unique 8-bp DNA sequence that differs from other sequences by at least 2 bases flanked by restriction sites compatible with the acceptor expression vector. Sequencing of expression cassette allows for the identification of pegRNA. Another method is to insert the U6-pegRNA expression cassette downstream from a Polymerase II promoter (such as CMV, EF1a, UBC) and upstream of a poly-A sequence. Flanking the U6-pegRNA expression cassette in this manner in lentiviral expression vectors leads to expression of the pegRNA from a Pol II promoter which allows identification of the respective pegRNA via RNA sequencing using standard poly A capture. In this Example, the pegRNA is its own barcode.

### Example 3: hiPSC Culture

Human induced pluripotent stem cells (hiPSC) were obtained from a commercial source and/or generated via reprogramming of PMBCs from a healthy donor using non-integrative methods (Dowey et al., 2012). hiPSC cultures were cultured in matrigel (Corning) coated plates using mTeSR plus (STEMCELL) and maintained in a humidified incubator set at 5% CO2 and ambient O₂ tension. Colonies were passaged using Accutase every 4-5 days. ROCK inhibitor (Y-27632) was added to hiPSC cultures for the first 24h post-passaging.

### Example 4: Transfections of HIPSC

For the V476I mutant, hiPSC transfection was performed on an Amaxa Nucleofector platform, using the P3 solution and program CB150. For the remaining mutants, DNA plasmid transfection was performed via electroporation using GenePulser xCell 0.2 mm cuvettes. Briefly, 100,000 cells were collected in 100 uL Ingenio electroporation solution (Mirus Bio) and 2 ug of total DNA was added (1.5 ug of plasmid #132776 plus 0.5 ug of plasmid #132777). Cell suspension with DNA was added to electroporation cuvettes. Electroporation protocol consisted of a square wave of 160 V, 950 uF, 20 ms. Transfection efficiency was estimated via electroporation of a EGFP expressing plasmid, generating ˜30% transfection efficiency.

### Example 5: Isolation of Prime-Edited HIPSC Clones

For V476I mutant (generated using PE2 plasmid #132775), clones were selected by plating of pooled (unsorted) transfected cells into 10 cm dishes at very low cell density (5,000 cells per dish). Colonies formed after 1-2 weeks were collected and clonally expanded for sequencing. For other mutants, generated using PE2 plasmid #132776, transfected hiPSC will become GFP fluorescent. Cells are FACS sorted ˜5 days post-transfection to generate a pool of transfected cells. Clone isolation is performed by limiting dilution into 96 well plates (˜0.7 cell/well). Clones are expanded and DNA extracted for sequencing.

### Example 6: Differentiation of HIPSC into Cardiomyocytes

The process of hiPSC differentiation into cardiomyocytes that was used is based on previously published protocols from Allen Institute by an (Sop_for_cardiomyocyte_differentiation_methods_v1.2_200211.Pdf, n.d.), followed enrichment step based on another published protocol (Cyganek et al., 2018), with modifications.

Protocol for Differentiation of hiPSC into Cardiomyocytes (CM)

| Day | Differentiation steps |
|---|---|
| D −3 | hiPSC maintained in Matrigel/mTeSR plus are allowed to achieve confluence of ~85% before |
| | passaging. |
| | 125,000 cells are seeded into each well of 6-well Matrigel coated plates using mTeSR supplemented |
| | with 10 uM of ROCK inhibitor (Y27632) |
| D −2 | Media change to mTeSR plus without ROCK inhibitor |
| D −1 | Media change to fresh mTeSR plus |
| D 0 | Start of differentiation protocol |
| | Media is changed to RPMI 1640 (Gibco, 11875093) supplemented with 1X B27 without insulin |
| | (Gibco, #1895601) and 7.5 uM of Wnt inhibitor CHIR99021 |
| D 2 | Media is changed to RPMI 1640 (Gibco, 11875093) supplemented with 1X B27 without insulin |
| | (Gibco, #1895601) and 7.5 uM IWP2 (Tocris, #13122) |
| D 4 | Media is changed to RPMI 1640 (Gibco, 11875093) supplemented with 1X B27 without insulin |
| | (Gibco, #1895601) |
| D 6 | Media is changed to RPMI 1640 (Gibco, 11875093) supplemented with 1X B27 PLUS (Gibco, # |
| | A3582801) |
| D 8-D 11 | Media is changed every 3 days until start of enrichment |
| D 12 | Start of enrichment |
| | Media is changed to RPMI 1640 without glucose (Gibco, 11879020) supplemented with 4 mM |
| | lactate, 0.2 mg/ml L-ascorbic acid 2-phosphate, and 0.5 mg/ml human recombinant albumin. CM |
| | are kept in enrichment media for 3-4 days |
| D 16 | Media is changed back to RPMI 1640 supplemented with B27 PLUS and cells allowed to recover 1- |
| | 2 days |
| D 17-18 | Enriched CM are harvested and seeded onto microscopy-grade flat bottom Matrigel-coated 96-well |
| | plates at 80,000 cells/well. |
| D 25-D 30 | After ~7 days the CM are ready for functional characterization |

### Example 7: Testing of Proarrhythmic Drugs in IPSC-Derived Cardiomyocytes

hiPSC-derived cardiomyocytes are incubated with an intracellular calcium-sensing fluorescent dye (Early Tox Cardiotoxicity kit, Molecular Devices, #R8211) for 2 h at 37° C. before addition of drugs for testing. Fluctuations of dye fluorescence correlate directly with intracellular calcium level fluctuations that happen during cardiomyocyte beating. This assay allows to collect data related to beating rate, and peak amplitude and width, among other parameters, in a high-throughput manner. An initial reading of calcium influx is performed to determine the basal level of cardiomyocyte activity. After basal reading, drugs or vehicle are added and incubated for 1.5-2h before a second reading is performed. Fluorescence intensity reading can be performed using plate readers or high-content microscopy. Using a plate reader, we collect a 30 sec reading with 300 frames per read. Using high-content microscopy, a stream of 600 images is collected from 30 sec. Fluorescence intensity data is analyzed with software PeakPro to determine peak characteristics. Peak frequency is determined and utilized as beating per minute (BPM). Alternatively, electrical activity of iPSC-CM can be measured with multielectrode arrays, although at a generally lower throughput.

### Example 8: Immunocytochemistry

hiPSC-derived cardiomyocytes were fixed with 4% formaldehyde for 10 min and washed 3× with DPBS. They were permeabilized with 0.1% Triton-X in PBS and incubated with primary antibody (Troponin T, Novus Biologicals #NBP27543) overnight. Cells were washed 3X, incubated with secondary antibodies for 1-2h, and washed twice before short incubation with whole cell stain CellMask Blue (ThermoFisher, #H32720). Cells were washed once more and covered in DPBS. ImageXpress Micro was used to acquire images from 12 wells for each genotype iPSC-CM.

### Example 9: Prime Editing

### Design, Cloning and Sanger Sequencing of pegRNA Constructs

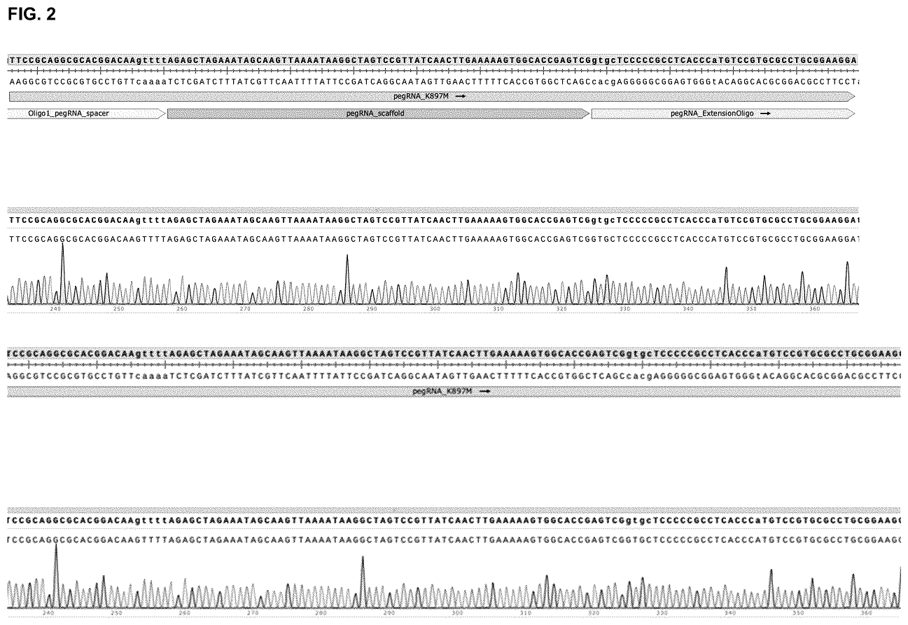

A total of 14 SNPs were selected for CRISPR prime editing into hiPSC. FIG. 1A. gRNA sequences and prime editing template fragments were synthesized, annealed in house, and cloned into a digested backbone containing the gRNA scaffold. Resulting plasmids were Sanger-sequenced. FIG. 2.

### Example 10: Prime Editing

### Transfection Optimization

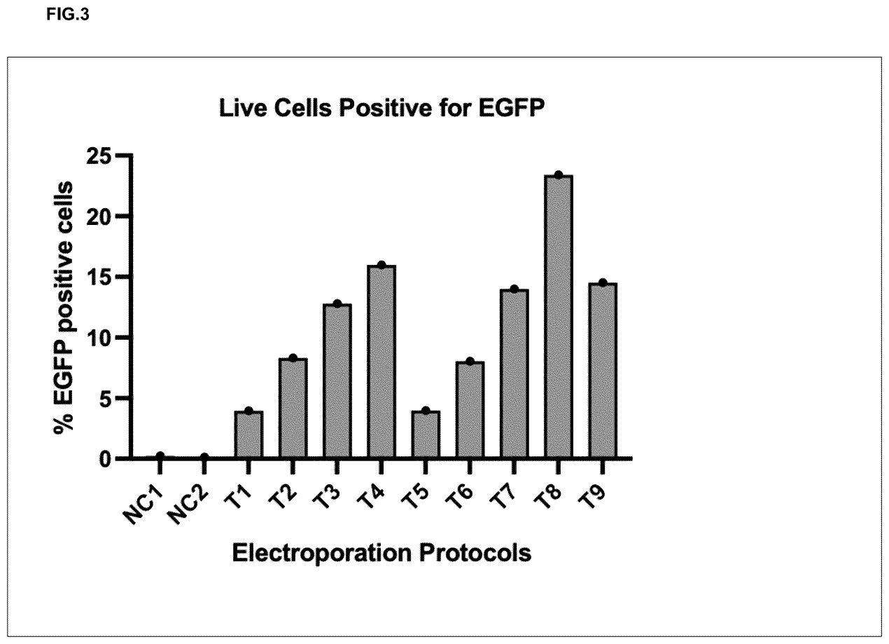

Electroporation parameters for delivery of plasmid DNA was performed in hIPSC cells using a CMV-EGFP plasmid. Briefly, 2 ug total of DNA was added into 100,000 cells resuspended in Ingenio electroporation solution and transferred to 0.2 cm GenePulser (BioRad) cuvettes. Eight distinct electroporation protocols were tested, including both square wave and exponential decay curve protocols. Transfection efficiency (GFP positive cells) and toxicity (propidium iodide positive cells) were measured via microscopy. The protocol with highest percentage of live cells positive for EGFP was used for follow-up experiments and further validation. FIG. 3.

### Example 11: Prime Editing

### Differentiation of hiPSC into Cardiomyocytes

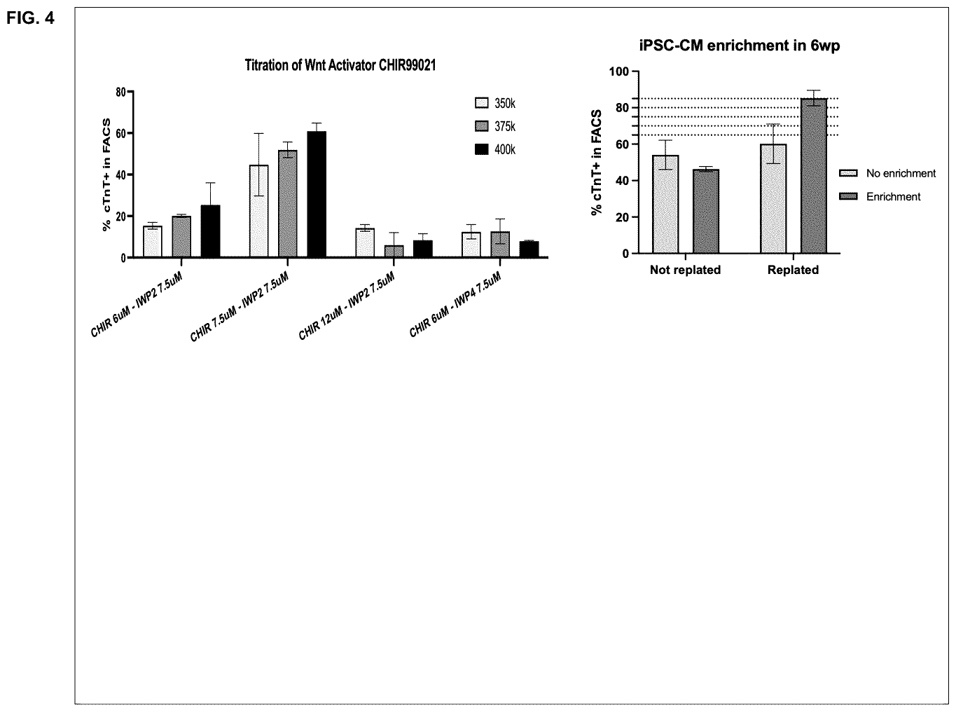

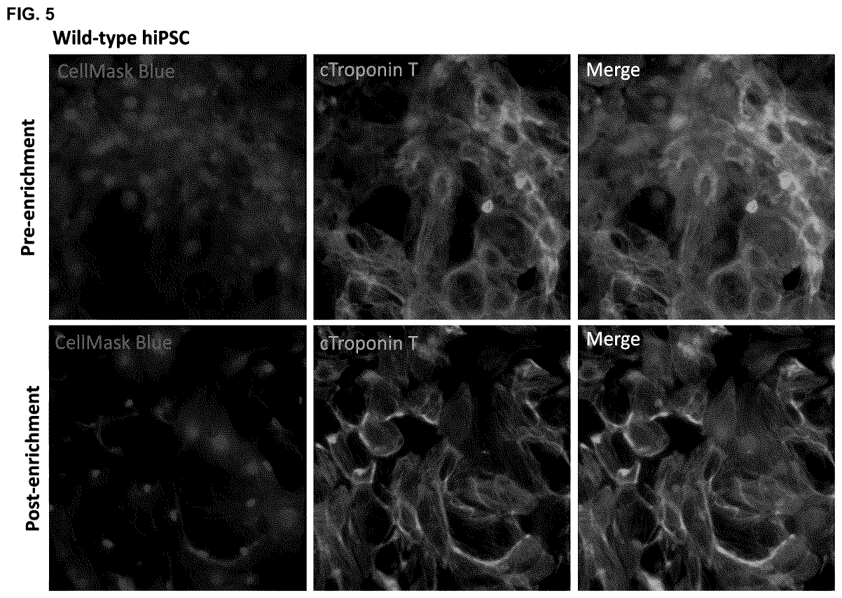

Wild-type and V476I hiPSC clonal lines were successfully differentiated into cardiomyocytes using a protocol that manipulates the Wnt pathway. See Example 6. Differentiation efficiency was measured via flow cytometry, using an FITC-tagged antibody against troponin T (cTnT), a bona-fide cardiomyocyte marker. The protocol for generation of hiPSC-CM was optimized by titrating the starting cell density and the concentration of the Wnt pathway modulators. After optimization, CM populations that are over >80% cTnT positive were routinely obtained. Expression of cTnT was confirmed using fluorescence microscopy. FIG. 4 and FIG. 5

### Example 12: Functional Characterization of HIPSC-Cm

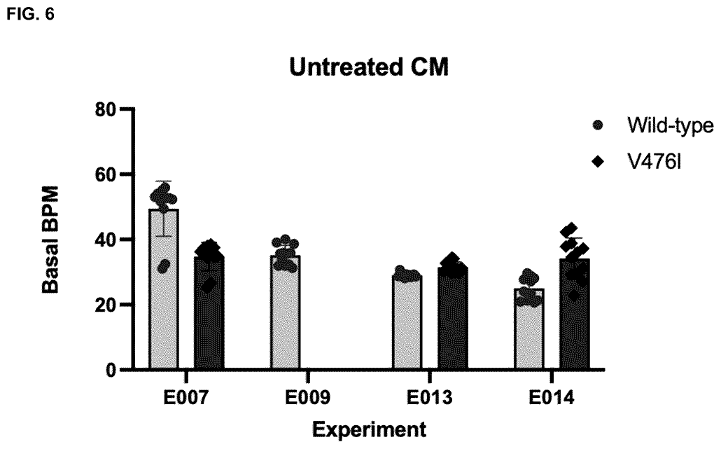

Unedited and V476I hiPSC-CM were treated with pro-(isoproterenol and propranolol) and anti-arrhythmic (sotalol) drugs to observe their ability to replicate cardiac muscle physiology. The observed basal rate of beats per minute (BPM) was similar to previously published data for hiPSC-derived CM (Bedut et al., 2016). FIG. 6.

### Example 13: Characterization of Drug Responses in Wild-Type and V476I Mutant Cardiomyocytes

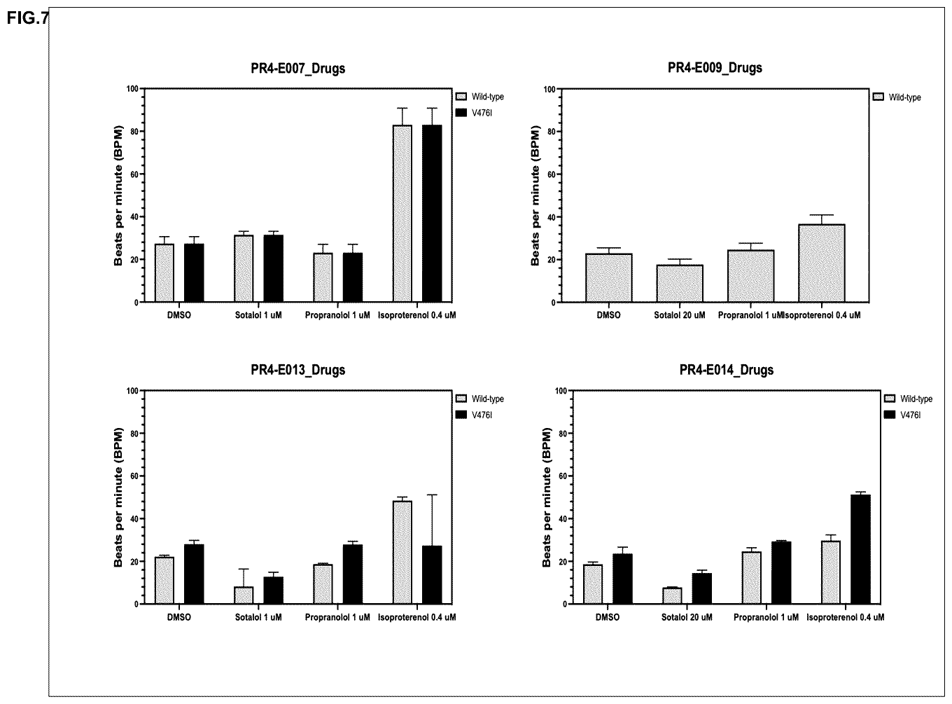

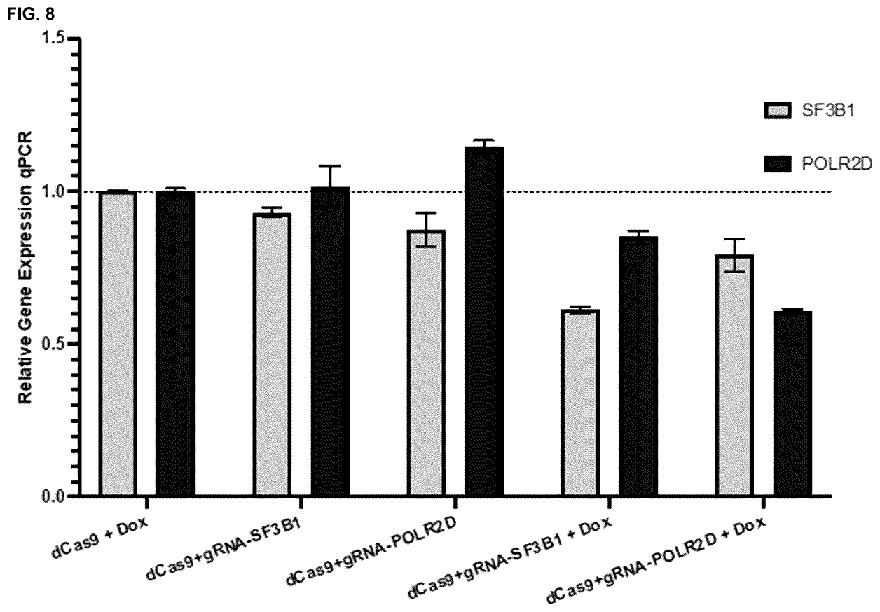

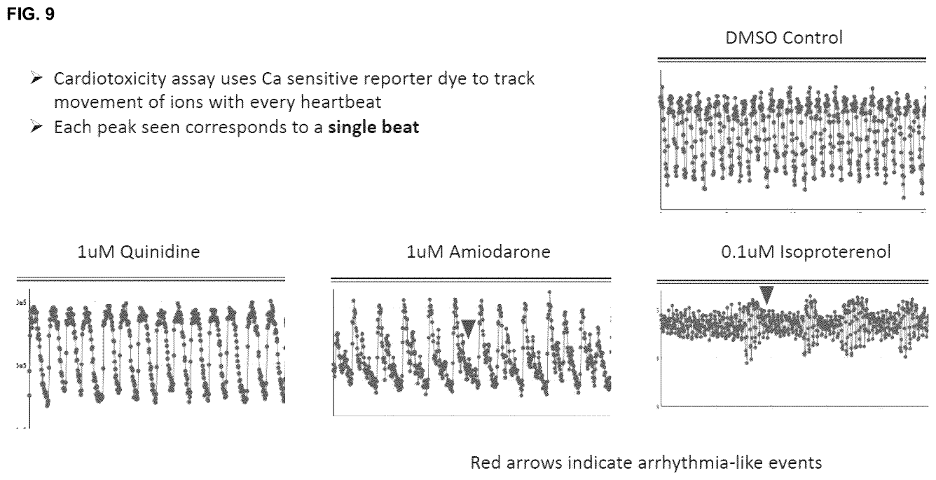

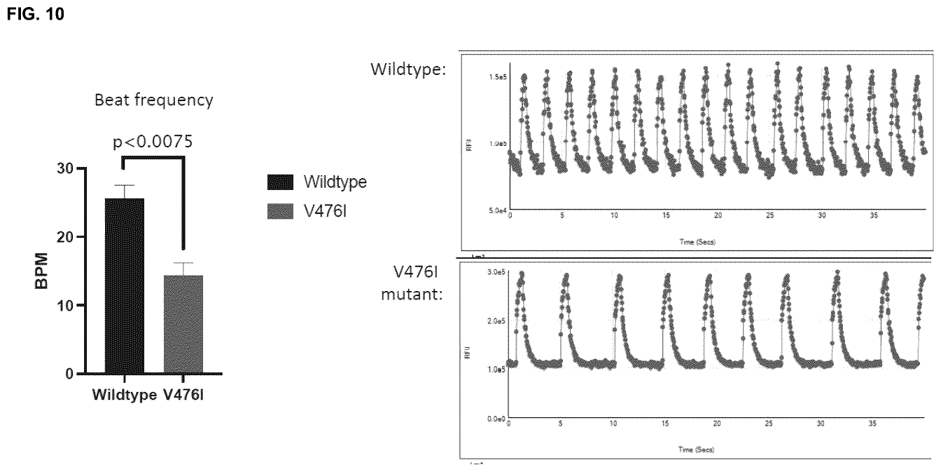

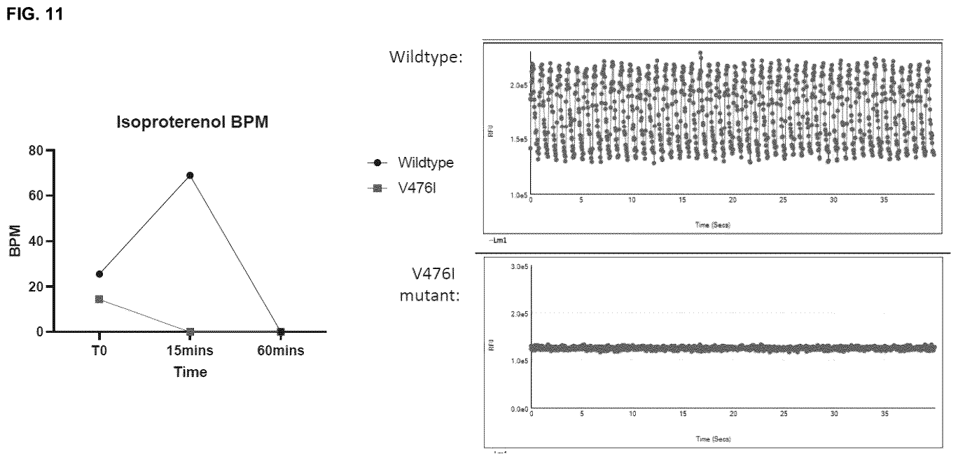

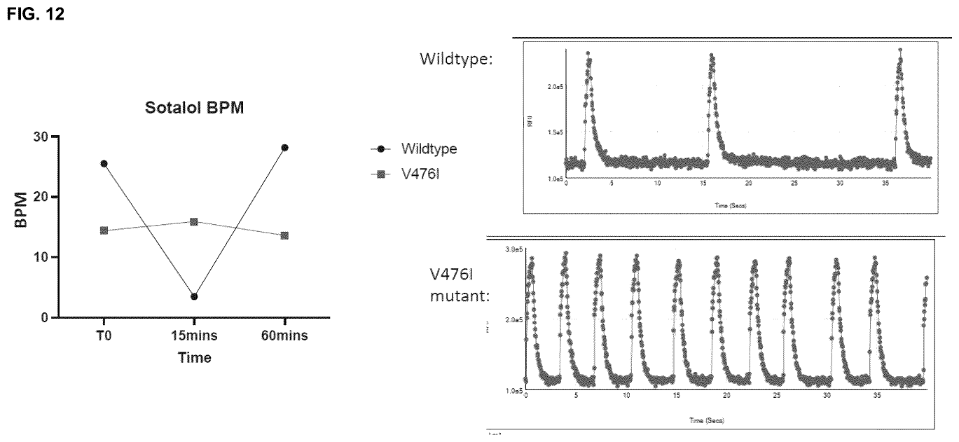

hiPSC-CM were treated with a fluorescent calcium-sensing probe for 2h before addition of drugs. The cells were then incubated with drugs for 1.5-2h. A plate reader (SpectraMax) was used to measure fluctuations in fluorescence for 30 sec. Images were analyzed using PeakPro to generate peak frequency which is a proxy for beats per minute. Both wild-type and V476I mutant show expected responses to pro-arrhythmic drugs, demonstrating functional responsiveness of this cellular model. FIG. 7.

### Example 14: Characterization of Additional Variants

Using the gnomAD browser (Broad Institute) resource, the kcnh-2 variation frequency was examined in the v3.1.2 data set and the Exome Aggregation Consortium (ExAC). For kcnh-2, 555 missense variants with allele frequencies were extracted. Based on this analysis, the most frequent kcnh-2 variants are prioritized as down selection criteria, e.g., allele frequencies equal to or greater than 3.4×10⁻⁵ encompasses 99 nonsynonymous SNPs. The various variants are engineered into the cells of the disclosure. The resulting cells are analyzed for evidence of cardiotoxicity according to any method, including those of the invention. The resulting cells are also used to test the toxicity of any drug. Variants that are pathogenic, of uncertain significance, benign, etc., are identified. Drugs are characterized as causing cardiotoxicity (survival, arrythmias, etc.) and others as not being toxic. The results are used in diagnostic and prognostic methods.

### Example 15: Characterization of Additional Genes

Databases such as ClinVar, dbSNP, and gnomAD provide a resource for identifying SNPs of interest in any other genes to engineer into iPSC cell lines for drug testing on differentiated cardiomyocytes. Some of the genes are selected from ion channels, ATP-binding cassette (ABC) transporters involved in drug transport (e.g., ABCB1, ABCB4, ABCCI, ABCC2, SLC10A2, SLC28A3, SLC22A7, SLC22A17), Carbonyl reductases in drug metabolism (e.g., CBR3), Hyaluronan synthase 3 involved in oxidative stress response (e.g., HAS3), Hereditary hemochromatosis protein in iron metabolism (e.g., HFE), Retinoic acid receptor gamma and DNA topoisomerases in topoisomerase-induced DNA damage (e.g., RARG, TOP2B), CUGBP Elav-like family member 4 in splicing of sarcomere genes (e.g., CELF4), DNA polymerase gamma in mitochondrial replication (e.g., DPOG2), Chaperones involved in ion channel trafficking (e.g., Hsp70 and Hsp90).

### Example 16: Modulation of KCNH2 Gene Expression by CRISPRI GRNAS

CRISPRi is the gRNA-directed targeting of nuclease-dead Cas9 (dCas9) fused to a transcriptional repressor (KRAB) to the promoter region on genes of interest (see, e.g., Larson et al., Nature Protocols. 8:2180-2196, 2013). The anchoring of the dCas9-KRAB to the promoter region interferes with the formation and elongation of the transcriptional machinery and/or recruits additional transcriptional repressors to negatively modulate gene expression. By targeting dCas-KRAB to the promoter region of KCNH2, the expression of the hERG channel is modulated to various levels, simulating the effects of naturally occurring KCNH2 SNPs that result in net reduction of hERG channel units on the surface of cardiomyocytes. To perform this, 42 sgRNAs against the promoter region of KCNH2 gene were designed and are being screened for the effects of these gRNAs on a non-cardiomyocyte cellular system that expresses high levels of KCNH2 (SHSY5Y cells) via lentiviral vectors. Successful gRNAs, defined as able to significantly reduce KCNH2 gene expression, as measured by qPCR, by 25%, 50%, 75% and >90% are transduced into human iPSC. These cells are differentiated into cardiomyocytes and tested against a broad panel of drugs to detect signs of arrhythmia events. This system allows one to mimic the (predicted) effect of dozens of uncharacterized SNPs in a reduced size sample manner.

Table 4 provides examples of gRNA target sequences that are being tested with CRISPRi.

For convenience, given the large number of Sequence IDs, they can be summarized as follows:

| SEQUENCE ID NO. | DESCRIPTION |
|---|---|
| 1 | mRNA for KCNH2 wild-type |
| 2-1054 | mRNA for KCNH2 carrying each of the |
| | identified SNP mutations |
| 1055-1094 | Target sequences from Table 4 |
| 1095 | Sp. Nuclease dead Cas9 |
| 1096 | gRNA expression vector |
| 1097 | Vector primeCas9-GFP |
| 1098 | pegRNA-GG-acceptor vector |
| 1099 | Prime Cas9-addgene plasmid |
| 1100-1113 | Exemplary pegRNAs for gene editing of |
| | KCNH2 SNPs |
| 1114 | Cas9 sequence for prime editing |
| 1115 | Cas9-RT sequence for prime editing |
| 1116-1155 | gRNA for CRISPRinterference Table 4 |

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments/aspects have been illustrated and described, it will be appreciated that various changes can be made without departing from the spirit and scope of the disclosure.

Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art. Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the embodiments explicitly disclosed above and in the claims.

### REFERENCES

Anzalone, A. V., Randolph, P. B., Davis, J. R., Sousa, A. A., Koblan, L. W., Levy, J. M., Chen, P. J., Wilson, C., Newby, G. A., Raguram, A., & Liu, D. R. (2019). Search-and-replace genome editing without double-strand breaks or donor DNA. Nature, 576 (7785), 149-157. https://doi.org/10.1038/s41586-019-1711-4
Bedut, S., Seminatore-Nole, C., Lamamy, V., Caignard, S., Boutin, J. A., Nosjean, O., Stephan, J.-P., & Coge, F. (2016). High-throughput drug profiling with voltage- and calcium-sensitive fluorescent probes in human iPSC-derived cardiomyocytes. American Journal of Physiology-Heart and Circulatory Physiology, 311 (1), H44-H53. https://doi.org/10.1152/ajpheart.00793.2015
Cyganek, L., Tiburcy, M., Sekeres, K., Gerstenberg, K., Bohnenberger, H., Lenz, C., Henze, S., Stauske, M., Salinas, G., Zimmermann, W.-H., Hasenfuss, G., & Guan, K. (2018). Deep phenotyping of human induced pluripotent stem cell-derived atrial and ventricular cardiomyocytes. JCI Insight, 3 (12), e99941. https://doi.org/10.1172/jci.insight.99941.
Dowey, S. N., Huang, X., Chou, B.-K., Ye, Z., & Cheng, L. (2012). Generation of integration-free human induced pluripotent stem cells from postnatal blood mononuclear cells by plasmid vector expression. Nature Protocols, 7 (11), 2013-2021. doi.org/10.1038/nprot.2012.121.
Sop_for_cardiomyocyte_differentiation_methods_v1.2_200211.pdf. (n.d.). Retrieved November www.allencell.org/uploads/8/1/9/9/81996008/sop_for_cardiomyocyte_differention_methods_v1.2_200211.pdf.
Lian, X., Zhang, J., Azarin, S. M., Zhu, K., Hazeltine, L. B., Bao, X., Hsiao, C., Kamp, T. J. & Palecek, S. P. Directed cardiomyocyte differentiation from human pluripotent stem cells by modulating Wnt/B-catenin signaling under fully defined conditions. Nature Protocols 8, 162-175 (2013).
Sharma, A., Li, G., Rajarajan, K., Hamaguchi, R., Burridge, P. W. & Wu, S. M. Derivation of highly purified cardiomyocytes from human induced pluripotent stem cells using small molecule-modulated differentiation and subsequent glucose starvation. Journal of visualized experiments: JoVE 18; (97): 52628. doi: 10.3791/52628 (2015).
Cyganek, L., Tiburcy, M., Sekeres, K., Gerstenberg, K., Bohnenberger, H., Lenz, C., Henze, S., Stauske, M., Salinas, G., Zimmermann, W. H., Hasenfuss, G. & Guan, K. Deep phenotyping of human induced pluripotent stem cell-derived atrial and ventricular cardiomyocytes. JCI insight 3 (12): e99941. doi: 10.1172/jci.insight.99941 (2018).
Kapplinger, J. D., Tester, D. J., Salisbury, B. A., Carr, J. L., Harris-Kerr, C., Pollevick, G. D., Wilde, A. A. & Ackerman, M. J. Spectrum and prevalence of mutations from the first 2,500 consecutive unrelated patients referred for the FAMILION long QT syndrome genetic test. Heart rhythm 6, 1297-1303 (2009).
Kapplinger, J. D., Tester, D. J., Salisbury, B. A., Carr, J. L., Harris-Kerr, C., Pollevick, G. D., Wilde, A. A. M. & Ackerman, M. J. Spectrum and prevalence of mutations from the first 2,500 consecutive unrelated patients referred for the FAMILION (R) long QT syndrome genetic test. Heart Rhythm 6, 1297-1303 (2009).
6. Duzkale, H., Shen, J., Mclaughlin, H., Alfares, A., Kelly, M. A., Pugh, T. J., Funke, B. H., Rehm, H. L. & Lebo, M. S. A systematic approach to assessing the clinical significance of genetic variants. Clinical genetics 84, 453-463 (2013).
7. Ware, J. S., Walsh, R., Cunningham, F., Birney, E. & Cook, S. A. Paralogous annotation of disease-causing variants in long QT syndrome genes. Human mutation 33, 1188-1191 (2012).
8. Larson, M. H., Gilbert, L. A., Wang, X., Lim, W. A., Weissman, J. S. & Qi, L. S. CRISPR interference (CRISPRi) for sequence-specific control of gene expression. Nature Protocols 8, 2180-2196 (2013).

TABLE 1
ClinVar SNPs Identified as Potentially Relevant to Cardiac Disease, which may be examples of SNPs to test with the methods of the disclosure (PREDICT PLATFORM).

| | Name | | | | | |
|---|---|---|---|---|---|---|
| | kenh2_protein_coding Human | | | | | |
| SEQ | putative potassium channel | SEQ | | | | |
| ID | subunit (h-erg) mRNA, | ID | Name | Accession | Condition(s) | Clinical significance (LR) |
| 1 | complete cds; WILD TYPE | 1 | | | | |
| 2 | SNP:c.1001C > T (p.Pro334Leu) | 605 | SNP:c.2690A > C (p.Lys897Thr) | VCV000067427 | Not Prov.\|Long QT Syn.\|Long QT Syn. 2\|Arr [symbol] | Benign(LR: Aug. 19, 2021) |
| 3 | SNP:c.1003C > T (p.Gln335Ter) | 779 | SNP:c.3133C > T (p.Leu1045Phe) | VCV000067473 | Arrhythmia\|Congenital long QT Syn.\|Arrhyth [symbol] | Benign(LR: Jul. 27, 2020) |
| 4 | SNP:c.1009A > G (p.Thr337Ala) | 152 | SNP:c.1598T > G (p.Val533Gly) | VCV000191574 | not spec. | Benign(LR: Jun. 24, 2013) |
| 5 | SNP:c.1010C > G (p.Thr337Ser) | 816 | SNP:c.326T > G (p.Leu109Arg) | VCV000067484 | Not Prov.\|Long QT Syn. 2 | Benign(LR: May 28, 2019) |
| 6 | SNP:c.1017C > G (p.Asn339Lys) | 576 | SNP:c.2617G > A (p.Gly873Ser) | VCV000067419 | Brugada Syn.\|Not Prov.\|Long QT Syn.\|not sp [symbol] | Benign/Likely benign(LR: Dec. 2, 2020) |
| 7 | SNP:c.1025A > T (p.Asp342Val) | 918 | SNP:c.542G > A (p.Arg181Gln) | VCV000067510 | Long QT Syn.\|Not Prov.\|not spec.\|Cardiovas. [symbol] | Benign/Likely benign(LR: Nov. 21, 2020) |
| 8 | SNP:c.1036G > A (p.Asp346Asn) | 700 | SNP:c.2900C > T (p.Pro967Leu) | VCV000067451 | Arrhythmia\|Long QT Syn.\|Not Prov.\|Cardiov. [symbol] | Benign/Likely benign(LR: Nov. 4, 2020) |
| 9 | SNP:c.1039C > T (p.Pro347Ser) | 361 | SNP:c.1918T > C (p.Phe640Leu) | VCV000200751 | Long QT Syn. 2\|Not Prov. | Conf. Interp. of Patho.(LR: Apr. 25, 2018) |
| 10 | SNP:c.1043T > A (p.Phe348Tyr) | 1053 | SNP:c.98A > C (p.Asn33Thr) | VCV000067553 | Long QT Syn.\|Not Prov. | Conf. Interp. of Patho.(LR: Apr. 29, 2021) |
| 11 | SNP:c.1052C > T (p.Ser351Leu) | 52 | SNP:c.1262C > T (p.Thr421Met) | VCV000067173 | Not Prov.\|Long QT Syn.\|Congenital long QT [symbol] | Conf. Interp. of Patho.(LR: Apr. 30, 2021) |
| 12 | SNP:c.1054C > T (p.Pro352Ser) | 188 | SNP:c.1684C > T (p.His562Tyr) | VCV000200733 | Not Prov.\|Long QT Syn. | Conf. Interp. of Patho.(LR: Aug. 1, 2019) |
| 13 | SNP:c.1055C > G (p.Pro352Arg) | 220 | SNP:c.1711A > G (p.Ile571 Val) | VCV000067247 | Congenital long QT Syn.\|Not Prov.\|Long QT [symbol] | Conf. Interp. of Patho.(LR: Aug. 11, 2017) |
| 14 | SNP:c.1058C > T (p.Thr353Ile) | 777 | SNP:c.3125T > C (p.Leu1042Pro) | VCV000264155 | Cardiovas. pheno.\|Not Prov. | Conf. Interp. of Patho.(LR: Aug. 19, 2016) |
| 15 | SNP:c.1066C > T (p.Arg356Cys) | 114 | SNP:c.1469C > T (p.Ala490Val) | VCV000862292 | Inborn genetic diseases\|Long QT Syn.\|Long ( [symbol] | Conf. Interp. of Patho.(LR: Aug. 20, 2020) |
| 16 | SNP:c.1067G > A (p.Arg356His) | 741 | SNP:c.301A > G (p.Lys101Glu) | VCV000067462 | Not Prov.\|Congenital long QT Syn.\|Long QT [symbol] | Conf. Interp. of Patho.(LR: Aug. 22, 2020) |
| 17 | SNP:c.1071G > T (p.Glu357Asp) | 149 | SNP:c.1591C > T (p.Arg531Trp) | VCV000067218 | Congenital long QT Syn.\|Not Prov.\|not spec.\| [symbol] | Conf. Interp. of Patho.(LR: Aug. 24, 2021) |
| 18 | SNP:c.1073T > A (p.Ile358Asn) | 395 | SNP:c.1979C > T (p.Ser660Leu) | VCV000067352 | Not Prov.\|Long QT Syn.\|Congenital long QT [symbol] | Conf. Interp. of Patho.(LR: Aug. 30, 2020) |
| 19 | SNP:c.1076T > A (p.Ile359Lys) | 184 | SNP:c.167G > T (p.Arg56Leu) | VCV000200557 | Not Prov.\|Long QT Syn. | Conf. Interp. of Patho.(LR: Dec. 10, 2020) |
| 20 | SNP:c.1081C > T (p.Pro361Ser) | 592 | SNP:c.2660G > A (p.Arg887His) | VCV000067423 | Arrhythmia\|Congenital long QT Syn.\|not spec [symbol] | Conf. Interp. of Patho.(LR: Dec. 11, 2020) |
| 21 | SNP:c.1096C > T (p.Arg366Ter) | 459 | SNP:c.221C > G (p.Thr74Arg) | VCV000067376 | Long QT Syn.\|Congenital long QT Syn.\|Not I [symbol] | Conf. Interp. of Patho.(LR: Dec. 13, 2019) |
| 22 | SNP:c.1097G > A (p.Arg366Gln) | 892 | SNP:c.442C > T (p.Arg148Trp) | VCV000067505 | Cardiovas. pheno.\|not spec.\|Arrhythmia\|Long [symbol] | Conf. Interp. of Patho.(LR: Dec. 2, 2020) |
| 23 | SNP:c.1099A > G (p.Thr367Ala) | 394 | SNP:c.1973A > G (p.Asn658Ser) | VCV000389141 | Not Prov.\|Long QT Syn. | Conf. Interp. of Patho.(LR: Dec. 20, 2016) |
| 24 | SNP:c.1099A > T (p.Thr367Ser) | 578 | SNP:c.2624C > T (p.Thr875Met) | VCV000067420 | Arrhythmia\|not spec.\|Not Prov.\|Long QT Syn [symbol] | Conf. Interp. of Patho.(LR: Dec. 23, 2020) |
| 25 | SNP:c.1126C > G (p.Gln376Glu) | 11 | SNP:c.1052C > T (p.Ser351Leu) | VCV000864050 | Long QT Syn.\|Long QT Syn. 2 | Conf. Interp. of Patho.(LR: Dec. 30, 2019) |
| 26 | SNP:c.1149C > A (p.Asp383Glu) | 855 | SNP:c.3436A > T (p.Thr1146Ser) | VCV000359301 | Short QT Syn. 1\|Long QT Syn. 2\|Arrhythmia [symbol] | Conf. Interp. of Patho.(LR: Dec. 6, 2020) |
| 27 | SNP:c.115T > C (p.Cys39Arg) | 9 | SNP:c.1039C > T (p.Pro347Ser) | VCV000067163 | not spec.\|Cardiovas. pheno.\|Arrhythmia\|Long [symbol] | Conf. Interp. of Patho.(LR: Dec. 7, 2020) |
| 28 | SNP:c.1178C > T (p.Pro393Leu) | 627 | SNP:c.2738C > T (p.Ala913Val) | VCV000014443 | Cardiovas. pheno.\|Congenital long QT Syn.\| [symbol] | Conf. Interp. of Patho.(LR: Dec. 8, 2020) |
| 29 | SNP:c.1180C > T (p.Arg394Cys) | 785 | SNP:c.3140G > T (p.Arg1047Leu) | VCV000036429 | Not Prov.\|Arrhythmia\|none provided\|Torsade [symbol] | Conf. Interp. of Patho.(LR: Dec. 8, 2020) |
| 30 | SNP:c.1181G > A (p.Arg394His) | 716 | SNP:c.2941A > G (p.Ser981Gly) | VCV000067454 | Not Prov.\|Arrhythmia\|not spec.\|Cardiovas. ph [symbol] | Conf. Interp. of Patho.(LR: Feb. 19, 2021) |
| 31 | SNP:c.1189C > T (p.Arg397Cys) | 495 | SNP:c.2350C > T (p.Arg784Trp) | VCV000014433 | Congenital long QT Syn.\|Long QT Syn. 2\|L [symbol] | Conf. Interp. of Patho.(LR: Feb. 22, 2021) |
| 32 | SNP:c.1190G > A (p.Arg397His) | 887 | SNP:c.422C > T (p.Pro141Leu) | VCV000067504 | Hypertrophic cardiomyopathy\|Not Prov.\|Lo [symbol] | Conf. Interp. of Patho.(LR: Feb. 25, 2021) |
| 33 | SNP:c.1193G > A (p.Trp398Ter) | 2 | SNP:c.1001C > T (p.Pro334Leu) | VCV000067161 | Not Prov.\|Congenital long QT Syn.\|Long QT [symbol] | Conf. Interp. of Patho.(LR: Jan. 29, 2019) |
| 34 | SNP:c.1199T > A (p.Ile400Asn) | 283 | SNP:c.1814C > T (p.Pro605Leu) | VCV000067284 | Long QT Syn.\|Congenital long QT Syn.\|Long [symbol] | Conf. Interp. of Patho.(LR: Jul. 11, 2018) |
| 35 | SNP:c.119C > T (p.Ala40Val) | 460 | SNP:c.221C > T (p.Thr74Met) | VCV000067377 | Long QT Syn.\|Not Prov. | Conf. Interp. of Patho.(LR: Jul. 11, 2018) |
| 36 | SNP:c.1205A > G (p.His402Arg) | 153 | SNP:c.1600C > A (p.Arg534Ser) | VCV000200728 | Long QT Syn.\|Not Prov. | Conf. Interp. of Patho.(LR: Jul. 23, 2018) |
| 37 | SNP:c.121G > C (p.Val41Leu) | 594 | SNP:c.2665T > G (p.Leu889Val) | VCV000200475 | Not Prov.\|Long QT Syn.\|Arrhythmia | Conf. Interp. of Patho.(LR: Jul. 29, 2020) |
| 38 | SNP:c.121G > T (p.Val41Phe) | 136 | SNP:c.1525G > A (p.Asp509Asn) | VCV000200354 | Cardiovas. pheno.\|Long QT Syn. 2\|Long QT [symbol] | Conf. Interp. of Patho.(LR: Jul. 3, 2018) |
| 39 | SNP:c.1225G > A (p.Val409Met) | 988 | SNP:c.80G > A (p.Arg27His) | VCV000200540 | Not Prov.\|Long QT Syn. | Conf. Interp. of Patho.(LR: Jun. 29, 2019) |
| 40 | SNP:c.1226T > A (p.Val409Glu) | 730 | SNP:c.299G > A (p.Arg100Gln) | VCV000067460 | Not Prov.\|Long QT Syn.\|Congenital long QT [symbol] | Conf. Interp. of Patho.(LR: Jun. 30, 2020) |
| 41 | SNP:c.1229G > A (p.Trp410Ter) | 276 | SNP:c.1805T > C (p.Leu602Pro) | VCV000234996 | Cardiovas. pheno.\|not spec.\|Not Prov.\|Long [symbol] | Conf. Interp. of Patho.(LR: Jun. 8, 2020) |
| 42 | SNP:c.1229G > C (p.Trp410Ser) | 524 | SNP:c.2417G > A (p.Gly806Glu) | VCV000067398 | Not Prov.\|Congenital long QT Syn.\|Long QT [symbol] | Conf. Interp. of Patho.(LR: Mar. 12, 2021) |
| 43 | SNP:c.122T > C (p.Val41Ala) | 423 | SNP:c.2081G > A (p.Arg694His) | VCV000200410 | Long QT Syn.\|Not Prov. | Conf. Interp. of Patho.(LR: Mar. 14, 2020) |
| 44 | SNP:c.1231G > A (p.Asp411Asn [symbol] | 804 | SNP:c.3203A > G (p.Gln1068Arg) | VCV000067480 | Arrhythmia\|Not Prov.\|Cardiovas. pheno.\|Lon [symbol] | Conf. Interp. of Patho.(LR: Mar. 16, 2021) |
| 45 | SNP:c.1238T > C (p.Leu413Pro) | 310 | SNP:c.1862G > A (p.Ser621Asn) | VCV000067299 | Long QT Syn.\|Primary familial hypertrophic [symbol] | Conf. Interp. of Patho.(LR: Mar. 18, 2020) |
| 46 | SNP:c.1238T > G (p.Leu413Arg) | 677 | SNP:c.2843G > A (p.Arg948His) | VCV000067443 | Long QT Syn.\|Long QT Syn. 2\|not spec.\|Not [symbol] | Conf. Interp. of Patho.(LR: Mar. 18, 2021) |
| 47 | SNP:c.1240A > G (p.Ile414Val) | 469 | SNP:c.2246G > T (p.Gly749Val) | VCV000067378 | Congenital long QT Syn.\|Not Prov.\|not spec [symbol] | Conf. Interp. of Patho.(LR: Mar. 31, 2016) |
| 48 | SNP:c.1259A > G (p.Tyr420Cys) | 604 | SNP:c.2684C > T (p.Thr895Met) | VCV000067426 | Long QT Syn.\|SUDDEN INFANT DEATH & [symbol] | Conf. Interp. of Patho.(LR: May 17, 2020) |
| 49 | SNP:c.125T > A (p.Ile42Asn) | 552 | SNP:c.2536C > T (p.Pro846Ser) | VCV000067409 | Congenital long QT Syn.\|Not Prov.\|Long QT [symbol] | Conf. Interp. of Patho.(LR: May 20, 2019) |
| 50 | SNP:c.125T > C (p.Ile42Thr) | 772 | SNP:c.3112G > A (p.Val1038Met) | VCV000067471 | Not Prov.\|Congenital long QT Syn.\|Arrhythm [symbol] | Conf. Interp. of Patho.(LR: May 28, 2021) |
| 51 | SNP:c.1262C > A (p.Thr421Lys) | 596 | SNP:c.2674C > T (p.Arg892Cys) | VCV000191470 | Long QT Syn.\|Not Prov.\|not spec.\|Arrhythmi [symbol] | Conf. Interp. of Patho.(LR: Nov. 11, 2020) |
| 52 | SNP:c.1262C > T (p.Thr421Met) | 1050 | SNP:c.982C > T (p.Arg328Cys) | VCV000067552 | Arrhythmia\| Long QT Syn.\|Not Prov.\|Long Q [symbol] | Conf. Interp. of Patho.(LR: Nov. 21, 2020) |
| 53 | SNP:c.1264G > A (p.Ala422Thr) | 415 | SNP:c.2053C > T (p.Arg685Cys) | VCV000448974 | none provided\| Long QT Syn.\|Long QT Syn. 2 [symbol] | Conf. Interp. of Patho.(LR: Nov. 25, 2019) |
| 54 | SNP:c.1277C > A (p.Pro426His) | 727 | SNP:c.298C > G (p.Arg100Gly) | VCV000014442 | Congenital long QT Syn.\|Cardiovas. pheno.\| [symbol] | Conf. Interp. of Patho.(LR: Nov. 28, 2017) |
| 55 | SNP:c.1279T > C (p.Tyr427His) | 441 | SNP:c.2131A > G (p.Ile711Val) | VCV000067367 | Long QT Syn.\|Arrhythmia\|Congenital long Q [symbol] | Conf. Interp. of Patho.(LR: Nov. 8, 2019) |
| 56 | SNP:c.127T > G (p.Tyr43Asp) | 926 | SNP:c.568G > A (p.Ala190Thr) | VCV000067512 | Long QT Syn.\|not spec.\|Not Prov.\|Long QT S [symbol] | Conf. Interp. of Patho.(LR: Oct. 14, 2020) |
| 57 | SNP:c.1280A > C (p.Tyr427Ser) | 1031 | SNP:c.934C > T (p.Arg312Cys) | VCV000067547 | Long QT Syn. 2\|Short QT Syn. 1\|Congenital [symbol] | Conf. Interp. of Patho.(LR: Oct. 14, 2020) |
| 58 | SNP:c.1280A > G (p.Tyr427Cys) | 535 | SNP:c.2468G > A (p.Arg823Gln) | VCV000418246 | Arrhythmia Long QT Syn.\|Not Prov. [symbol] | Conf. Interp. of Patho.(LR: Oct. 15, 2020) |
| 59 | SNP:c.1282T > C (p.Ser428Pro) | 718 | SNP:c.2948C > T (p.Thr983Ile) | VCV000067455 | Long QT Syn.\|Long QT Syn. 2\|not spec.\|Not [symbol] | Conf. Interp. of Patho.(LR: Oct. 15, 2020) |
| 60 | SNP:c.1283C > T (p.Ser428Leu) | 247 | SNP:c.1750G > T (p.Gly584Cys) | VCV000067263 | Congenital long QT Syn.\|Not Prov.\|Long QT [symbol] | Conf. Interp. of Patho.(LR: Oct. 17, 2018) |
| 61 | SNP:c.1285G > C (p.Ala429Pro) | 506 | SNP:c.2371C > T (p.Arg791Trp) | VCV000067391 | Long QT Syn.\|Not Prov.\|Brugada Syn. 1\|Carc [symbol] | Conf. Interp. of Patho.(LR: Oct. 23, 2020) |
| 62 | SNP:c.128A > G (p.Tyr43Cys) | 80 | SNP:c.1352C > T (p.Pro451Leu) | VCV000067190 | Congenital long QT Syn.\|Long QT Syn.\|Long [symbol] | Conf. Interp. of Patho.(LR: Oct. 25, 2020) |
| 63 | SNP:c.1291T > C (p.Phe431Leu) | 77 | SNP:c.1342G > T (p.Ala448Ser) | VCV000448973 | Long QT Syn. 2\|Long QT Syn.\|Arrhythmia [symbol] | Conf. Interp. of Patho.(LR: Oct. 26, 2020) |
| 64 | SNP:c.1293C > A (p.Phe431Leu) | 501 | SNP:c.2360T > A (p.Ile787Asn) | VCV000200428 | Long QT Syn.\|Not Prov. [symbol] | Conf. Interp. of Patho.(LR: Oct. 3, 2019) |
| 65 | SNP:c.1307C > T (p.Thr436Met) | 684 | SNP:c.2860C > T (p.Arg954Cys) | VCV000067444 | SUDDEN INFANT DEATH Syn.\|Cardiovas [symbol] | Conf. Interp. of Patho.(LR: Oct. 4, 2019) |
| 66 | SNP:c.1319C > T (p.Pro440Leu) | 421 | SNP:c.2078T > C (p.Leu693Pro) | VCV000067359 | Long QT Syn.\|Inborn genetic diseases\|Conge [symbol] | Conf. Interp. of Patho.(LR: Oct. 6, 2017) |
| 67 | SNP:c.131G > A (p.Cys44Tyr) | 335 | SNP:c.1888G > A (p.Val630Ile) | VCV000222669 | Long QT Syn.\|Long QT Syn. 2 [symbol] | Conf. Interp. of Patho.(LR: Oct. 6, 2020) |
| 68 | SNP:c.131G > T (p.Cys44Phe) | 686 | SNP:c.2863C > G (p.Leu955Val) | VCV000067445 | Arrhythmia\|Not Prov.\|Long QT Syn.\|Congeni [symbol] | Conf. Interp. of Patho.(LR: Oct. 6, 2020) |
| 69 | SNP:c.1325C > T (p.Ala442Val) | 831 | SNP:c.3322C > G (p.Leu1108Val) | VCV000067488 | Arrhythmia\| Long QT Syn.\|Long QT Syn. 2\|N [symbol] | Conf. Interp. of Patho.(LR: Oct. 6, 2020) |
| 70 | SNP:c.132C > G (p.Cys44Trp) | 758 | SNP:c.3079C > A (p.Leu1027Ile) | VCV000200254 | Cardiac arrhythmia\| Arrhythmia [symbol] | Conf. Interp. of Patho.(LR: Oct. 8, 2020) |
| 71 | SNP:c.1330G > A (p.Glu444Lys) | 171 | SNP:c.164C > T (p.Ser55Leu) | VCV000067224 | Congenital long QT Syn.\|Not Prov.\|Long QT [symbol] | Conf. Interp. of Patho.(LR: Oct. 9, 2020) |
| 72 | SNP:c.1330G > T (p.Glu444Ter) | 889 | SNP:c.431A > T (p.Asp144Val) | VCV000526910 | Long QT Syn.\|Arrhythmia [symbol] | Conf. Interp. of Patho.(LR: Sep. 10, 2020) |
| 73 | SNP:c.1332G > C (p.Glu444Asp) | 622 | SNP:c.2729C > T (p.Pro910Leu) | VCV000067430 | Not Prov.\|Cardiovas. pheno.\|Arrhythmia\|Long [symbol] | Conf. Interp. of Patho.(LR: Sep. 13, 2021) |
| 74 | SNP:c.1332G > T (p.Glu444Asp) | 646 | SNP:c.2771G > A (p.Gly924Glu) | VCV000067437 | Long QT Syn. 2\|Cardiovas. pheno.\|Congenita [symbol] | Conf. Interp. of Patho.(LR: Sep. 15, 2020) |
| 75 | SNP:c.133A > T (p.Asn45Tyr) | 790 | SNP:c.3164G > A (p.Arg1055Gln) | VCV000067475 | Not Prov.\|Cardiovas. pheno.\|not spec.\|Arrhyth [symbol] | Conf. Interp. of Patho.(LR: Sep. 17, 2020) |
| 76 | SNP:c.1342G > A (p.Ala448Thr) | 338 | SNP:c.188C > A (p.Pro63His) | VCV000200559 | Long QT Syn.\|Not Prov. [symbol] | Conf. Interp. of Patho.(LR: Sep. 24, 2021) |
| 77 | SNP:c.1342G > T (p.Ala448Ser) | 914 | SNP:c.526C > T (p.Arg176Trp) | VCV000067509 | Not Prov.\|Long QT Syn. 2\|Short QT Syn. 1\| [symbol] | Conf. Interp. of Patho., risk factor(LR: Jun. 11, 2021) |
| 78 | SNP:c.1348C > T (p.Gln450Ter) | 117 | SNP:c.1471G > A (p.Val491Ile) | VCV000200344 | Long QT Syn.\|not spec.\|Arrhythmia\|Not Prov [symbol] | Likely benign(LR: Dec. 4, 2020) |
| 79 | SNP:c.134A > C (p.Asn45Thr) | 913 | SNP:c.524C > A (p.Ala175Asp) | VCV000200211 | Long QT Syn.\|Long QT Syn. 2\|not spec. [symbol] | Likely benign(LR: Jul. 31, 2019) |
| 80 | SNP:c.1352C > T (p.Pro451Leu) | 968 | SNP:c.736G > T (p.Gly246Cys) | VCV000379687 | not spec. [symbol] | Likely benign(LR: May 11, 2015) |
| 81 | SNP:c.1357G > A (p.Ala453Thr) | 955 | SNP:c.697G > T (p.Ala233Ser) | VCV000527096 | Long QT Syn.\|Not Prov. [symbol] | Likely benign(LR: May 13, 2020) |
| 82 | SNP:c.135C > A (p.Asn45Lys) | 660 | SNP:c.278A > G (p.Lys93Arg) | VCV000413325 | Long QT Syn.\|Cardiovas. pheno.\|Not Prov.\| [symbol] | Likely benign(LR: Nov. 15, 2020) |
| 83 | SNP:c.1366G > T (p.Asp456Tyr) | 712 | SNP:c.2932G > A (p.Glu978Lys) | VCV000067453 | Not Prov.\|Arrhythmia\|not spec.\|Long QT Syn [symbol] | Likely benign(LR: Nov. 15, 2020) |
| 84 | SNP:c.136G > A (p.Asp46Asn) | 217 | SNP:c.1708G > T (p.Ala570Ser) | VCV001284270 | Not Prov. | Likely pathogenic |
| 85 | SNP:c.1372A > T (p.Ile458Phe) | 234 | SNP:c.1738G > C (p.Asp580His) | VCV000191223 | Not Prov. | Likely pathogenic |
| 86 | SNP:c.1378G > T (p.Asp460Tyr) | 293 | SNP:c.1831T > G (p.Tyr611Asp) | VCV000067290 | Congenital long QT Syn.\|Long QT Syn [symbol] | Likely pathogenic |
| 87 | SNP:c.137A > G (p.Asp46Gly) | 573 | SNP:c.260T > C (p.Leu87Pro) | VCV000067418 | Congenital long QT Syn.\|Not Prov [symbol] | Likely pathogenic |
| 88 | SNP:c.1384A > G (p.Met462Val [symbol] | 165 | SNP:c.162C > A (p.Tyr54Ter) | VCV000488895 | Not Prov. | Likely pathogenic(LR: Apr. 19, 2016) |
| 89 | SNP:c.1387T > C (p.Phe463Leu) | 474 | SNP:c.2263G > A (p.Ala755Thr) | VCV000200423 | Not Prov. | Likely pathogenic(LR: Apr. 28, 2014) |
| 90 | SNP:c.138C > G (p.Asp46Glu) | 37 | SNP:c.121G > C (p.Val41Leu) | VCV000372589 | Not Prov. | Likely pathogenic(LR: Apr. 9, 2015) |
| 91 | SNP:c.1391T > C (p.Ile464Thr) | 517 | SNP:c.2396T > C (p.Leu799Pro) | VCV000200442 | Not Prov. | Likely pathogenic(LR: Aug. 13, 2019) |
| 92 | SNP:c.1394T > C (p.Val465Ala) | 46 | SNP:c.1238T > G (p.Leu413Arg) | VCV000279588 | Long QT Syn. 2 | Likely pathogenic(LR: Aug. 30, 2016) |
| 93 | SNP:c.1396G > T (p.Asp466Tyr) | 230 | SNP:c.172G > A (p.Glu58Lys) | VCV000067254 | Not Prov.\|Long QT Syn.\|Congenital long QT [symbol] | Likely pathogenic(LR: Aug. 30, 2021) |
| 94 | SNP:c.1397A > G (p.Asp466Gly [symbol] | 300 | SNP:c.1843C > T (p.Leu615Phe) | VCV000067294 | Not Prov.\|Congenital long QT Syn [symbol] | Likely pathogenic(LR: Aug. 6, 2020) |
| 95 | SNP:c.1397A > T (p.Asp466Val) | 212 | SNP:c.1704G > C (p.Trp568Cys) | VCV000067243 | Congenital long QT Syn.\|Long QT Syn [symbol] | Likely pathogenic(LR: Aug. 8, 2018) |
| 96 | SNP:c.1408A > G (p.Asn470Asp [symbol] | 183 | SNP:c.167G > A (p.Arg56Gln) | VCV000067228 | Long QT Syn.\|Congenital long QT Syn [symbol] | Likely pathogenic(LR: Aug. 9, 2018) |
| 97 | SNP:c.140G > T (p.Gly47Val) | 475 | SNP:c.2266A > G (p.Met756Val) | VCV000067381 | Long QT Syn. 2\| Acquired long QT Syn [symbol] | Likely pathogenic(LR: Dec. 11, 2019) |
| 98 | SNP:c.1415G > C (p.Arg472Pro) | 225 | SNP:c.1715G > C (p.Gly572Ala) | VCV000391858 | Not Prov. | Likely pathogenic(LR: Dec. 19, 2016) |
| 99 | SNP:c.1417A > C (p.Thr473Pro) | 1023 | SNP:c.916G > T (p.Gly306Trp) | VCV000067544 | Congenital long QT Syn.\|Long QT Syn.\|Not [symbol] | Likely pathogenic(LR: Dec. 22, 2017) |
| 100 | SNP:c.1418C > A (p.Thr473Asn) | 161 | SNP:c.1610G > A (p.Arg537Gln) | VCV000200360 | Not Prov. | Likely pathogenic(LR: Dec. 30, 2016) |
| 101 | SNP:c.1418C > T (p.Thr473Ile) | 502 | SNP:c.2362G > A (p.Glu788Lys) | VCV000067389 | Long QT Syn.\|Congenital long QT Syn [symbol] | Likely pathogenic(LR: Feb. 11, 2016) |
| 102 | SNP:c.1421C > T (p.Thr474Ile) | 1028 | SNP:c.92T > A (p.Ile31Asn) | VCV000520423 | Long QT Syn. 2 | Likely pathogenic(LR: Feb. 14, 2018) |
| 103 | SNP:c.1424A > G (p.Tyr475Cys) | 200 | SNP:c.1693G > C (p.Ala565Pro) | VCV000200372 | Not Prov. | Likely pathogenic(LR: Feb. 2, 2015) |
| 104 | SNP:c.1425C > A (p.Tyr475Ter) | 612 | SNP:c.2701C > T (p.Gln901Ter) | VCV000927383 | Arrhythmia | Likely pathogenic(LR: Feb. 21, 2020) |
| 105 | SNP:c.1426G > A (p.Val476Ile) | 249 | SNP:c.1754G > T (p.Trp585Leu) | VCV000200739 | Not Prov. | Likely pathogenic(LR: Feb. 27, 2012) |
| 106 | SNP:c.1447G > A (p.Val483Ile) | 876 | SNP:c.379C > T (p.Leu127Phe) | VCV000200278 | Not Prov. | Likely pathogenic(LR: Feb. 4, 2014) |
| 107 | SNP:c.1447G > T (p.Val483Phe) | 447 | SNP:c.2162C > T (p.Pro721Leu) | VCV000067371 | Long QT Syn.\|Congenital long QT Syn. | Likely pathogenic(LR: Feb. 6, 2019) |
| 108 | SNP:c.1459G > A (p.Gly487Ser) | 384 | SNP:c.1952T > G (p.Met651Arg) | VCV000427945 | Long QT Syn. 2 | Likely pathogenic(LR: Feb. 9, 2017) |
| 109 | SNP:c.1460G > C (p.Gly487Ala) | 1029 | SNP:c.92T > C (p.Ile31Thr) | VCV000067545 | Congenital long QT Syn.\|Not Prov. | Likely pathogenic(LR: Jan. 12, 2021) |
| 110 | SNP:c.1462C > T (p.Arg488Cys) | 236 | SNP:c.173A > G (p.Glu58Gly) | VCV000067257 | Congenital long QT Syn.\|Not Prov. | Likely pathogenic(LR: Jan. 14, 2021) |
| 111 | SNP:c.1465A > T (p.Ile489Phe) | 122 | SNP:c.1478A > G (p.Tyr493Cys) | VCV000067206 | Congenital long QT Syn.\|Long QT Syn.\|Not [symbol] | Likely pathogenic(LR: Jan. 15, 2021) |
| 112 | SNP:c.1468G > A (p.Ala490Thr) | 285 | SNP:c.1819A > T (p.Ile607Phe) | VCV000418245 | Not Prov. | Likely pathogenic(LR: Jan. 2, 2015) |
| 113 | SNP:c.1468G > C (p.Ala490Pro) | 725 | SNP:c.296A > G (p.Tyr99Cys) | VCV000200582 | Not Prov. | Likely pathogenic(LR: Jan. 21, 2020) |
| 114 | SNP:c.1469C > T (p.Ala490Val) | 178 | SNP:c.1673C > A (p.Ala558Glu) | VCV000067226 | Not Prov.\|Congenital long QT Syn. | Likely pathogenic(LR: Jan. 22, 2021) |
| 115 | SNP:c.146G > A (p.Cys49Tyr) | 280 | SNP:c.1811G > T (p.Gly604Val) | VCV000200741 | Not Prov. | Likely pathogenic(LR: Jan. 3, 2018) |
| 116 | SNP:c.146G > T (p.Cys49Phe) | 453 | SNP:c.2192A > C (p.His731Pro) | VCV000200417 | Not Prov. | Likely pathogenic(LR: Jan. 31, 2014) |
| 117 | SNP:c.1471G > A (p.Val491Ile) | 151 | SNP:c.1595T > C (p.Leu532Pro) | VCV000978356 | Long QT Syn. 1 | Likely pathogenic(LR: Jan. 8, 2019) |
| 118 | SNP:c.1474C > T (p.His492Tyr) | 275 | SNP:c.1802G > A (p.Gly601Asp) | VCV000200730 | Not Prov. | Likely pathogenic(LR: Jul. 14, 2021) |
| 119 | SNP:c.1475A > T (p.His492Leu) | 737 | SNP:c.3007G > T (p.Asp1003Tyr) | VCV000200516 | Not Prov. | Likely pathogenic(LR: Jul. 16, 2014) |
| 120 | SNP:c.1477T > C (p.Tyr493His) | 67 | SNP:c.131G > A (p.Cys44Tyr) | VCV001214199 | Not Prov. | Likely pathogenic(LR: Jul. 23, 2021) |
| 121 | SNP:c.1478A > C (p.Tyr493Ser) | 101 | SNP:c.1418C > T (p.Thr473Ile) | VCV000811073 | not spec. | Likely pathogenic(LR: Jul. 24, 2018) |
| 122 | SNP:c.1478A > G (p.Tyr493Cys) | 471 | SNP:c.2255G > A (p.Arg752Gln) | VCV000014435 | Long QT Syn.\|Not Prov.\|Congenital long QT [symbol] | Likely pathogenic(LR: Jul. 29, 2021) |
| 123 | SNP:c.1478A > T (p.Tyr493Phe) | 195 | SNP:c.1689G > C (p.Trp563Cys) | VCV000200368 | Not Prov. | Likely pathogenic(LR: Jul. 31, 2018) |
| 124 | SNP:c.1479C > A (p.Tyr493Ter) | 263 | SNP:c.1781G > A (p.Gly594Asp) | VCV000067271 | Congenital long QT Syn.\|Long QT Syn. | Likely pathogenic(LR: Jul. 31, 2019) |
| 125 | SNP:c.147C > G (p.Cys49Trp) | 197 | SNP:c.1691T > A (p.Leu564Gln) | VCV000572344 | Long QT Syn. | Likely pathogenic(LR: Jul. 9, 2019) |
| 126 | SNP:c.148G > C (p.Glu50Gln) | 516 | SNP:c.2390C > A (p.Ala797Asp) | VCV000200440 | not spec.\|Not Prov. | Likely pathogenic(LR: Jun. 12, 2013) |
| 127 | SNP:c.1496T > G (p.Leu499Arg) | 533 | SNP:c.2464G > C (p.Val822Leu) | VCV000200456 | Not Prov. | Likely pathogenic(LR: Jun. 15, 2017) |
| 128 | SNP:c.1501G > A (p.Asp501Asn [symbol] | 489 | SNP:c.232G > A (p.Ala78Thr) | VCV001197726 | Not Prov. | Likely pathogenic(LR: Jun. 22, 2021) |
| 129 | SNP:c.1501G > C (p.Asp501His) | 393 | SNP:c.1970G > T (p.Gly657Val) | VCV000427076 | Not Prov. | Likely pathogenic(LR: Jun. 26, 2015) |
| 130 | SNP:c.1502A > G (p.Asp501Gly [symbol] | 315 | SNP:c.1873G > C (p.Val625Leu) | VCV000618184 | Not Prov. | Likely pathogenic(LR: Jun. 26, 2017) |
| 131 | SNP:c.150G > T (p.Glu50Asp) | 531 | SNP:c.245T > C (p.Ile82Thr) | VCV000585220 | Long QT Syn. 2 | Likely pathogenic(LR: Jun. 5, 2017) |
| 132 | SNP:c.1516A > G (p.Ile506Val) | 98 | SNP:c.1415G > C (p.Arg472Pro) | VCV000200340 | Not Prov. | Likely pathogenic(LR: Jun. 8, 2012) |
| 133 | SNP:c.1519C > G (p.Pro507Ala) | 58 | SNP:c.1280A > G (p.Tyr427Cys) | VCV000067179 | Long QT Syn. 1\|Congenital long QT Syn. | Likely pathogenic(LR: Mar. 16, 2017) |
| 134 | SNP:c.1519C > T (p.Pro507Ser) | 250 | SNP:c.1755G > C (p.Trp585Cys) | VCV000581669 | Long QT Syn. | Likely pathogenic(LR: Mar. 16, 2018) |
| 135 | SNP:c.1520C > T (p.Pro507Leu) | 118 | SNP:c.1474C > T (p.His492Tyr) | VCV000067204 | Long QT Syn. 2\|Congenital long QT Syn.\|A [symbol] | Likely pathogenic(LR: Mar. 18, 2020) |
| 136 | SNP:c.1525G > A (p.Asp509Asn | 241 | SNP:c.1745G > T (p.Arg582Leu) | VCV000067258 | Congenital long QT Syn.\|Long QT Syn. | Likely pathogenic(LR: Mar. 25, 2020) |
| 137 | SNP:c.1537T > C (p.Phe513Leu) | 59 | SNP:c.1282T > C (p.Ser428Pro) | VCV000200335 | Not Prov. | Likely pathogenic(LR: Mar. 26, 2014) |
| 138 | SNP:c.1564G > A (p.Gly522Arg [symbol] | 304 | SNP:c.1851C > A (p.Phe617Leu) | VCV000392690 | Not Prov. | Likely pathogenic(LR: Mar. 30, 2017) |
| 139 | SNP:c.1567C > A (p.Leu523Met [symbol] | 84 | SNP:c.136G > A (p.Asp46Asn) | VCV000200551 | Not Prov. | Likely pathogenic(LR: Mar. 9, 2015) |
| 140 | SNP:c.156C > A (p.Cys52Ter) | 407 | SNP:c.202T > C (p.Phe68Leu) | VCV000067355 | Congenital long QT Syn.\|Not Prov. | Likely pathogenic(LR: May 10, 2014) |
| 141 | SNP:c.156C > G (p.Cys52Trp) | 306 | SNP:c.1853C > T (p.Thr618Ile) | VCV000067297 | short QT Syn.\|Long QT Syn. | Likely pathogenic(LR: May 10, 2017) |
| 142 | SNP:c.1575G > T (p.Lys525Asn) | 64 | SNP:c.1293C > A (p.Phe431Leu) | VCV000067183 | Not Prov.\|Congenital long Q Syn. | Likely pathogenic(LR: May 10, 2018) |
| 143 | SNP:c.1576A > G (p.Thr526Ala) | 1038 | SNP:c.959C > A (p.Ser320Ter) | VCV001215035 | Not Prov. | Likely pathogenic(LR: May 14, 2021) |
| 144 | SNP:c.157G > A (p.Gly53Ser) | 291 | SNP:c.1828A > G (p.Lys610Glu) | VCV001066309 | Long QT Syn. | Likely pathogenic(LR: May 16, 2020) |
| 145 | SNP:c.157G > C (p.Gly53Arg) | 176 | SNP:c.1664G > A (p.Cys555Tyr) | VCV000372574 | Not Prov. | Likely pathogenic(LR: May 18, 2016) |
| 146 | SNP:c.1582C > T (p.Arg528Trp) | 396 | SNP:c.197G > A (p.Cys66Tyr) | VCV000545688 | Long QT Syn. 2 | Likely pathogenic(LR: May 18, 2018) |
| 147 | SNP:c.1583G > C (p.Arg528Pro) | 327 | SNP:c.1882G > C (p.Gly628Arg) | VCV000200749 | Not Prov. | Likely pathogenic(LR: May 18, 2020) |
| 148 | SNP:c.158G > A (p.Gly53Asp) | 1022 | SNP:c.916G > C (p.Gly306Arg) | VCV000067543 | Long QT Syn. 2\|Long QT Syn.\|Congenital [symbol] | Likely pathogenic(LR: Nov. 10, 2017) |
| 149 | SNP:c.1591C > T (p.Arg531Trp) | 270 | SNP:c.1970A > G (p.Tyr597Cys) | VCV000067277 | Congenital long QT Syn.\|Not Prov.\|Long QT [symbol] | Likely pathogenic(LR: Nov. 18, 2015) |
| 150 | SNP:c.1592G > A (p.Arg531Gln) | 317 | SNP:c.1874T > C (p.Val625Ala) | VCV000222668 | Long QT Syn. 2\|Long QT Syn. | Likely pathogenic(LR: Nov. 26, 2015) |
| 151 | SNP:c.1595T > C (p.Leu532Pro) | 116 | SNP:c.146G > T (p.Cys49Phe) | VCV000972694 | Long QT Syn. 2 | Likely pathogenic(LR: Nov. 26, 2019) |
| 152 | SNP:c.1598T > G (p.Val533Gly) | 57 | SNP:c.1280A > C (p.Tyr427Ser) | VCV000067178 | Cardiovas. pheno.\|Congenital long QT Syn. | Likely pathogenic(LR: Nov. 7, 2016) |
| 153 | SNP:c.1600C > A (p.Arg534Ser) | 878 | SNP:c.387C > G (p.Phe129Leu) | VCV000200280 | Not Prov. | Likely pathogenic(LR: Nov. 7, 2019) |
| 154 | SNP:c.1600C > G (p.Arg534Gly) | 202 | SNP:c.1694C > T (p.Ala565Val) | VCV000200735 | Not Prov. | Likely pathogenic(LR: Oct. 1, 2019) |
| 155 | SNP:c.1600C > T (p.Arg534Cys) | 191 | SNP:c.1686C > G (p.His562Gln) | VCV000379052 | Not Prov. | Likely pathogenic(LR: Oct. 21, 2016) |
| 156 | SNP:c.1601G > T (p.Arg534Leu) | 440 | SNP:c.212G > A (p.Gly71Glu) | VCV000264521 | Cardiovas. pheno. | Likely pathogenic(LR: Oct. 27, 2015) |
| 157 | SNP:c.1603G > A (p.Val535Met) | 488 | SNP:c.2326C > T (p.Leu776Phe) | VCV000200425 | Not Prov. | Likely pathogenic(LR: Sep. 17, 2012) |
| 158 | SNP:c.1603G > T (p.Val535Leu) | 218 | SNP:c.170C > T (p.Ala57Val) | VCV000200775 | Not Prov. | Likely pathogenic(LR: Sep. 3, 2014) |
| 159 | SNP:c.1609C > T (p.Arg537Trp) | 35 | SNP:c.119C > T (p.Ala40Val) | VCV000200544 | Not Prov. | Likely pathogenic(LR: Sep. 30, 2013) |
| 160 | SNP:c.160T > C (p.Tyr54His) | 7 | SNP:c.1025A > T (p.Asp342Val) | VCV000067162 | Acquired long QT Syn. | Not Prov. |
| 161 | SNP:c.1610G > A (p.Arg537Gln) | 34 | SNP:c.1199T > A (p.Ile400Asn) | VCV000067165 | Congenital long QT Syn. | Not Prov. |
| 162 | SNP:c.1612A > G (p.Lys538Glu) | 38 | SNP:c.121G > T (p.Val41Phe) | VCV000067167 | Congenital long QT Syn. | Not Prov. |
| 163 | SNP:c.1621C > T (p.Arg541Cys) | 42 | SNP:c.1229G > C (p.Trp410Ser) | VCV000067168 | Congenital long QT Syn. | Not Prov. |
| 164 | SNP:c.1622G > A (p.Arg541His) | 43 | SNP:c.122T > C (p.Val41Ala) | VCV000067169 | Congenital long QT Syn. | Not Prov. |
| 165 | SNP:c.162C > A (p.Tyr54Ter) | 45 | SNP:c.1238T > C (p.Leu413Pro) | VCV000067170 | Congenital long QT Syn. | Not Prov. |
| 166 | SNP:c.162C > G (p.Tyr54Ter) | 48 | SNP:c.1259A > G (p.Tyr420Cys) | VCV000067171 | Congenital long QT Syn. | Not Prov. |
| 167 | SNP:c.1639G > A (p.Ala547Thr) | 49 | SNP:c.125T > A (p.Ile42Asn) | VCV000067172 | Congenital long QT Syn. | Not Prov. |
| 168 | SNP:c.1640C > T (p.Ala547Val) | 54 | SNP:c.1277C > A (p.Pro426His) | VCV000067175 | Congenital long QT Syn. | Not Prov. |
| 169 | SNP:c.1645G > A (p.Val549Met [symbol] | 55 | SNP:c.1279T > C (p.Tyr427His) | VCV000067176 | Congenital long QT Syn. | Not Prov. |
| 170 | SNP:c.164C > A (p.Ser55Ter) | 56 | SNP:c.127T > G (p.Tyr43Asp) | VCV000067177 | Congenital long QT Syn. | Not Prov. |
| 171 | SNP:c.164C > T (p.Ser55Leu) | 61 | SNP:c.1285G > C (p.Ala429Pro) | VCV000067181 | Congenital long QT Syn. | Not Prov. |
| 172 | SNP:c.1652T > C (p.Phe551Ser) | 68 | SNP:c.131G > T (p.Cys44Phe) | VCV000067186 | Congenital long QT Syn. | Not Prov. |
| 173 | SNP:c.1655T > A (p.Leu552Ter) | 70 | SNP:c.132C > G (p.Cys44Trp) | VCV000067187 | Congenital long QT Syn. | Not Prov. |
| 174 | SNP:c.1655T > C (p.Leu552Ser) | 74 | SNP:c.1332G > T (p.Glu444Asp) | VCV000067188 | Not Prov. | Not Prov. |
| 175 | SNP:c.1661T > C (p.Met554Thr) | 75 | SNP:c.133A > T (p.Asn45Tyr) | VCV000067189 | Congenital long QT Syn. | Not Prov. |
| 176 | SNP:c.1664G > A (p.Cys555Tyr) | 83 | SNP:c.1366G > T (p.Asp456Tyr) | VCV000067191 | Congenital long QT Syn. | Not Prov. |
| 177 | SNP:c.1672G > C (p.Ala558Pro) | 86 | SNP:c.1378G > T (p.asp460Tyr) | VCV000067192 | Congenital long QT Syn. | Not Prov. |
| 178 | SNP:c.1673C > A (p.Ala558Glu) | 89 | SNP:c.1387T > C (p.Phe463Leu) | VCV000067193 | Congenital long QT Syn. | Not Prov. |
| 179 | SNP:c.1675C > T (p.Leu559Phe) | 93 | SNP:c.1396G > T (p.Asp466Tyr) | VCV000067194 | Congenital long QT Syn. | Not Prov. |
| 180 | SNP:c.1676T > A (p.Leu559His) | 97 | SNP:c.140G > T (p.Gly47Val) | VCV000067195 | Congenital long QT Syn. | Not Prov. |
| 181 | SNP:c.1676T > C (p.Leu559Pro) | 99 | SNP:c.1417A > C (p.Thr473Pro) | VCV000067196 | Congenital long QT Syn. | Not Prov. |
| 182 | SNP:c.1678A > G (p.Ile560Val) | 100 | SNP:c.1418C > A (p.Thr473Asn) | VCV000067197 | Congenital long QT Syn. | Not Prov. |
| 183 | SNP:c.167G > A (p.Arg56Gln) | 103 | SNP:c.1424A > G (p.Tyr475Cys) | VCV000067199 | Congenital long QT Syn. | Not Prov. |
| 184 | SNP:c.167G > T (p.Arg56Leu) | 111 | SNP:c.1465A > T (p.Ile489Phe) | VCV000067201 | Congenital long QT Syn. | Not Prov. |
| 185 | SNP:c.1681G > A (p.Ala561Thr) | 113 | SNP:c.1468G > C (p.Ala490Pro) | VCV000067202 | Congenital long QT Syn. | Not Prov. |
| 186 | SNP:c.1681G > C (p.Ala561Pro) | 115 | SNP:c.146G > A (p.Cys49Tyr) | VCV000067203 | Congenital long QT Syn. | Not Prov. |
| 187 | SNP:c.1682C > T (p.Ala561Val) | 121 | SNP:c.1478A > C (p.Tyr493Ser) | VCV000067205 | Congenital long QT Syn. | Not Prov. |
| 188 | SNP:c.1684C > T (p.His562Tyr) | 123 | SNP:c.1478A > T (p.Tyr493Phe) | VCV000067207 | Congenital long QT Syn. | Not Prov. |
| 189 | SNP:c.1685A > C (p.His562Pro) | 129 | SNP:c.1501G > C (p.Asp501His) | VCV000067209 | Congenital long QT Syn. | Not Prov. |
| 190 | SNP:c.1685A > G (p.His562Arg) | 131 | SNP:c.150G > T (p.Glu50Asp) | VCV000067211 | short QT Syn. | Not Prov. |
| 191 | SNP:c.1686C > G (p.His562Gln) | 142 | SNP:c.1575G > T (p.Lys525Asn) | VCV000067213 | Congenital long QT Syn. | Not Prov. |
| 192 | SNP:c.1687T > G (p.Trp563Gly) | 144 | SNP:c.157G > A (p.Gly53Ser) | VCV000067214 | Congenital long QT Syn. | Not Prov. |
| 193 | SNP:c.1688G > A (p.Trp563Ter) | 147 | SNP:c.1583G > C (p.Arg528Pro) | VCV000067216 | Congenital long QT Syn. | Not Prov. |
| 194 | SNP:c.1689G > A (p.Trp563Ter) | 148 | SNP:c.158G > A (p.Gly53Asp) | VCV000067217 | Congenital long QT Syn. | Not Prov. |
| 195 | SNP:c.1689G > C (p.Trp563Cys) | 150 | SNP:c.1592G > A (p.Arg531Gln) | VCV000067219 | Congenital long QT Syn. | Not Prov. |
| 196 | SNP:c.1689G > T (p.Trp563Cys) | 180 | SNP:c.1676T > A (p.Leu559His) | VCV000067227 | Congenital long QT Syn. | Not Prov. |
| 197 | SNP:c.1691T > A (p.Leu564Gln) | 189 | SNP:c.1685A > C (p.His562Pro) | VCV000067231 | Congenital long QT Syn. | Not Prov. |
| 198 | SNP:c.1691T > C (p.Leu564Pro) | 192 | SNP:c.1687T > G (p.Trp563Gly) | VCV000067233 | Congenital long QT Syn. | Not Prov. |
| 199 | SNP:c.1693G > A (p.Ala565Thr) | 196 | SNP:c.1689G > T (p.Trp563Cys) | VCV000067234 | Congenital long QT Syn. | Not Prov. |
| 200 | SNP:c.1693G > C (p.Ala565Pro) | 198 | SNP:c.1691T > C (p.Leu564Pro) | VCV000067235 | Congenital long QT Syn. | Not Prov. |
| 201 | SNP:c.1693G > T (p.Ala565Ser) | 199 | SNP:c.1693G > A (p.Ala565Thr) | VCV000067236 | Congenital long QT Syn. | Not Prov. |
| 202 | SNP:c.1694C > T (p.Ala565Val) | 203 | SNP:c.1696T > G (p.Cys566Gly) | VCV000067237 | Congenital long QT Syn. | Not Prov. |
| 203 | SNP:c.1696T > G (p.Cys566Gly) | 204 | SNP:c.1697G > C (p.Cys566Ser) | VCV000067238 | Congenital long QT Syn. | Not Prov. |
| 204 | SNP:c.1697G > C (p.Cys566Ser) | 205 | SNP:c.1697G > T (p.Cys566Phe) | VCV000067239 | Congenital long QT Syn. | Not Prov. |
| 205 | SNP:c.1697G > T (p.Cys566Phe) | 209 | SNP:c.1702T > C (p.Trp568Arg) | VCV000067242 | Congenital long QT Syn. | Not Prov. |
| 206 | SNP:c.169G > A (p.Ala57Thr) | 213 | SNP:c.1704G > T (p.Trp568Cys) | VCV000067244 | Congenital long QT Syn. | Not Prov. |
| 207 | SNP:c.169G > C (p.Ala57Pro) | 214 | SNP:c.1705T > C (p.Tyr569His) | VCV000067245 | Congenital long QT Syn. | Not Prov. |
| 208 | SNP:c.1700T > C (p.Ile567Thr) | 219 | SNP:c.1711A > C (p.Ile571Leu) | VCV000067246 | Congenital long QT Syn. | Not Prov. |
| 209 | SNP:c.1702T > C (p.Trp568Arg) | 223 | SNP:c.1714G > T (p.Gly572Cys) | VCV000067249 | Congenital long QT Syn. | Not Prov. |
| 210 | SNP:c.1703G > C (p.Trp568Ser) | 224 | SNP:c.1715G > A (p.Gly572Asp) | VCV000067250 | Congenital long QT Syn. | Not Prov. |
| 211 | SNP:c.1704G > A (p.Trp568Ter) | 227 | SNP:c.1720A > G (p.Met574Val) | VCV000067252 | Congenital long QT Syn. | Not Prov. |
| 212 | SNP:c.1704G > C (p.Trp568Cys) | 228 | SNP:c.1724A > G (p.Glu575Gly) | VCV000067253 | Congenital long QT Syn. | Not Prov. |
| 213 | SNP:c.1704G > T (p.Trp568Cys) | 235 | SNP:c.173A > C (p.Glu58Ala) | VCV000067256 | Congenital long QT Syn. | Not Prov. |
| 214 | SNP:c.1705T > C (p.Tyr569His) | 242 | SNP:c.1747A > G (p.Ile583Val) | VCV000067259 | Congenital long QT Syn. | Not Prov. |
| 215 | SNP:c.1707C > G (p.Tyr569Ter) | 244 | SNP:c.174G > C (p.Glu58Asp) | VCV000067260 | Congenital long QT Syn. | Not Prov. |
| 216 | SNP:c.1708G > A (p.Ala570Thr) | 246 | SNP:c.1750G > C (p.Gly584Arg) | VCV000067262 | Congenital long QT Syn. | Not Prov. |
| 217 | SNP:c.1708G > T (p.Ala570Ser) | 248 | SNP:c.1751G > T (p.Gly584Val) | VCV000067264 | Congenital long QT Syn. | Not Prov. |
| 218 | SNP:c.170C > T (p.Ala57Val) | 251 | SNP:c.1755G > T (p.Trp585Cys) | VCV000067265 | Congenital long QT Syn. | Not Prov. |
| 219 | SNP:c.1711A > C (p.Ile571Leu) | 253 | SNP:c.1762A > G (p.Asn588Asp) | VCV000067266 | Congenital long QT Syn. | Not Prov. |
| 220 | SNP:c.1711A > G (p.Ile571Val) | 256 | SNP:c.1769G > T (p.Gly590Val) | VCV000067267 | Congenital long QT Syn. | Not Prov. |
| 221 | SNP:c.1714G > A (p.Gly572Ser) | 258 | SNP:c.1777A > G (p.Ile593Val) | VCV000067268 | Congenital long QT Syn. | Not Prov. |
| 222 | SNP:c.1714G > C (p.Gly572Arg) | 264 | SNP:c.1783A > G (p.Lys595Glu) | VCV000067272 | Congenital long QT Syn. | Not Prov. |
| 223 | SNP:c.1714G > T (p.Gly572Cys) | 265 | SNP:c.1785A > T (p.Lys595Asn) | VCV000067273 | Congenital long QT Syn. | Not Prov. |
| 224 | SNP:c.1715G > A (p.Gly572Asp | 268 | SNP:c.1787C > G (p.Pro596Arg) | VCV000067275 | Congenital long QT Syn. | Not Prov. |
| 225 | SNP:c.1715G > C (p.Gly572Ala) | 269 | SNP:c.1787C > T (p.Pro596Leu) | VCV000067276 | Congenital long QT Syn. | Not Prov. |
| 226 | SNP:c.1715G > T (p.Gly572Val) | 272 | SNP:c.1797C > A (p.Ser599Arg) | VCV000067278 | Congenital long QT Syn. | Not Prov. |
| 227 | SNP:c.1720A > G (p.Met574Val) | 274 | SNP:c.1801G > T (p.Gly601Cys) | VCV000067280 | Congenital long QT Syn. | Not Prov. |
| 228 | SNP:c.1724A > G (p.Glu575Gly) | 279 | SNP:c.1811G > A (p.Gly604Asp) | VCV000067282 | Congenital long QT Syn. | Not Prov. |
| 229 | SNP:c.1726C > T (p.Gln576Ter) | 281 | SNP:c.1813C > T (p.Pro605Ser) | VCV000067283 | Congenital long QT Syn. | Not Prov. |
| 230 | SNP:c.172G > A (p.Glu58Lys) | 284 | SNP:c.1816T > C (p.Ser606Pro) | VCV000067285 | Congenital long QT Syn. | Not Prov. |
| 231 | SNP:c.1730C > A (p.Pro577Gln) | 287 | SNP:c.1825G > C (p.Asp609His) | VCV000067287 | Congenital long QT Syn. | Not Prov. |
| 232 | SNP:c.1733A > C (p.His578Pro) | 288 | SNP:c.1825G > T (p.Asp609Tyr) | VCV000067288 | Congenital long QT Syn. | Not Prov. |
| 233 | SNP:c.1736T > C (p.Met579Thr) | 295 | SNP:c.1834G > T (p.Val612Leu) | VCV000067291 | Congenital long QT Syn. | Not Prov. |
| 234 | SNP:c.1738G > C (p.Asp580His) | 299 | SNP:c.1843C > G (p.Leu615Val) | VCV000067293 | Congenital long QT Syn. | Not Prov. |
| 235 | SNP:c.173A > C (p.Glu58Ala) | 305 | SNP:c.1853C > G (p.Thr618Ser) | VCV000067296 | Congenital long QT Syn. | Not Prov. |
| 236 | SNP:c.173A > G (p.Glu58Gly) | 311 | SNP:c.1863C > G (p.Ser621Arg) | VCV000067300 | Congenital long QT Syn. | Not Prov. |
| 237 | SNP:c.1742C > A (p.Ser581Ter) | 313 | SNP:c.1868C > T (p.Thr623Ile) | VCV000067302 | Congenital long QT Syn. | Not Prov. |
| 238 | SNP:c.1742C > T (p.Ser581Leu) | 316 | SNP:c.1874T > A (p.Val625Glu) | VCV000067303 | Congenital long QT Syn. | Not Prov. |
| 239 | SNP:c.1744C > T (p.Arg582Cys) | 319 | SNP:c.1877G > A (p.Gly626Asp) | VCV000067305 | Congenital long QT Syn. | Not Prov. |
| 240 | SNP:c.1745G > A (p.Arg582His) | 320 | SNP:c.1877G > C (p.Gly626Ala) | VCV000067306 | Congenital long QT Syn. | Not Prov. |
| 241 | SNP:c.1745G > T (p.Arg582Leu) | 321 | SNP:c.1877G > T (p.Gly626Val) | VCV000067307 | Congenital long QT Syn. | Not Prov. |
| 242 | SNP:c.1747A > G (p.Ile583Val) | 322 | SNP:c.1879T > A (p.Phe627Ile) | VCV000067308 | Congenital long QT Syn. | Not Prov. |
| 243 | SNP:c.1749C > G (p.Ile583Met) | 323 | SNP:c.1879T > C (p.Phe627Leu) | VCV000067309 | Congenital long QT Syn. | Not Prov. |
| 244 | SNP:c.174G > C (p.Glu58Asp) | 325 | SNP:c.1881C > G (p.Phe627Leu) | VCV000067310 | Congenital long QT Syn. | Not Prov. |
| 245 | SNP:c.1750G > A (p.Gly584Ser) | 328 | SNP:c.1883G > C (p.Gly628Ala) | VCV000067311 | Congenital long QT Syn. | Not Prov. |
| 246 | SNP:c.1750G > C (p.Gly584Arg) | 329 | SNP:c.1883G > T (p.Gly628Val) | VCV000067312 | Congenital long QT Syn. | Not Prov. |
| 247 | SNP:c.1750G > T (p.Gly584Cys) | 333 | SNP:c.1886A > T (p.Asn629Ile) | VCV000067316 | Congenital long QT Syn. | Not Prov. |
| 248 | SNP:c.1751G > T (p.Gly584Val) | 334 | SNP:c.1887C > A (p.Asn629Lys) | VCV000067317 | Congenital long QT Syn. | Not Prov. |
| 249 | SNP:c.1754G > T (p.Trp585Leu) | 336 | SNP:c.1888G > C (p.Val630Leu) | VCV000067318 | Congenital long QT Syn. | Not Prov. |
| 250 | SNP:c.1755G > C (p.Trp585Cys) | 337 | SNP:c.1889T > C (p.Val630Ala) | VCV000067319 | Congenital long QT Syn. | Not Prov. |
| 251 | SNP:c.1755G > T (p.Trp585Cys) | 339 | SNP:c.1891T > G (p.Ser631Ala) | VCV000067320 | Congenital long QT Syn. | Not Prov. |
| 252 | SNP:c.1756C > G (p.Leu586Val) | 344 | SNP:c.1897A > G (p.Asn633Asp) | VCV000067322 | Congenital long QT Syn. | Not Prov. |
| 253 | SNP:c.1762A > G (p.Asn588Asp | 346 | SNP:c.1898A > T (p.Asn633Ile) | VCV000067324 | Congenital long QT Syn. | Not Prov. |
| 254 | SNP:c.1764C > A (p.Asn588Lys [symbol] | 349 | SNP:c.1903A > G (p.Asn635Asp) | VCV000067326 | Congenital long QT Syn. | Not Prov. |
| 255 | SNP:c.1764C > G (p.Asn588Lys [symbol] | 352 | SNP:c.1905C > G (p.Asn635Lys) | VCV000067328 | Congenital long QT Syn. | Not Prov. |
| 256 | SNP:c.1769G > T (p.Gly590Val) | 356 | SNP:c.1910A > G (p.Glu637Gly) | VCV000067330 | Congenital long QT Syn. | Not Prov. |
| 257 | SNP:c.1771G > A (p.Asp591Asn [symbol] | 357 | SNP:c.1911G > C (p.Glu637Asp) | VCV000067331 | Congenital long QT Syn. | Not Prov. |
| 258 | SNP:c.1777A > G (p.Ile593Val) | 358 | SNP:c.1912A > G (p.Lys638Glu) | VCV000067332 | Congenital long QT Syn. | Not Prov. |
| 259 | SNP:c.1778T > A (p.Ile593Lys) | 360 | SNP:c.1914G > T (p.Lys638Asn) | VCV000067333 | Congenital long QT Syn. | Not Prov. |
| 260 | SNP:c.1778T > C (p.Ile593Thr) | 362 | SNP:c.1918T > G (p.Phe640Val) | VCV000067334 | Congenital long QT Syn. | Not Prov. |
| 261 | SNP:c.1778T > G (p.Ile593 Arg) | 364 | SNP:c.191G > A (p.Cys64Tyr) | VCV000067335 | Congenital long QT Syn. | Not Prov. |
| 262 | SNP:c.1780G > C (p.Gly594Arg) | 368 | SNP:c.1926C > G (p.Ile642Met) | VCV000973054 | Long QT Syn. 2 | Not Prov. |
| 263 | SNP:c.1781G > A (p.Gly594Asp) | 370 | SNP:c.192C > G (p.Cys64Trp) | VCV000067338 | Congenital long QT Syn. | Not Prov. |
| 264 | SNP:c.1783A > G (p.Lys595Glu) | 372 | SNP:c.1930G > C (p.Val644Leu) | VCV000067339 | Congenital long QT Syn. | Not Prov. |
| 265 | SNP:c.1785A > T (p.Lys595Asn) | 376 | SNP:c.1933A > T (p.Met645Leu) | VCV000067342 | Congenital long QT Syn. | Not Prov. |
| 266 | SNP:c.1786C > G (p.Pro596Ala) | 379 | SNP:c.1942G > A (p.Gly648Ser) | VCV000067344 | Congenital long QT Syn. | Not Prov. |
| 267 | SNP:c.1787C > A (p.Pro596His) | 381 | SNP:c.1945T > C (p.Ser649Pro) | VCV000067345 | Congenital long QT Syn. | Not Prov. |
| 268 | SNP:c.1787C > G (p.Pro596Arg) | 387 | SNP:c.1967T > G (p.Phe656Cys) | VCV000067347 | Congenital long QT Syn. | Not Prov. |
| 269 | SNP:c.1787C > T (p.Pro596Leu) | 389 | SNP:c.1969G > C (p.Gly657Arg) | VCV000067349 | Congenital long QT Syn. | Not Prov. |
| 270 | SNP:c.1790A > G (p.Tyr597Cys) | 390 | SNP:c.1969G > T (p.Gly657Cys) | VCV000067350 | Congenital long QT Syn. | Not Prov. |
| 271 | SNP:c.1796G > A (p.Ser599Asn) | 392 | SNP:c.196T > G (p.Cys66Gly) | VCV000067351 | Congenital long QT Syn. | Not Prov. |
| 272 | SNP:c.1797C > A (p.Ser599Arg) | 398 | SNP:c.1985T > C (p.Ile662Thr) | VCV000067353 | Congenital long QT Syn. | Not Prov. |
| 273 | SNP:c.1801G > A (p.Gly601Ser) | 400 | SNP:c.1A > T (p.Met1Leu) | VCV000067354 | Congenital long QT Syn. | Not Prov. |
| 274 | SNP:c.1801G > T (p.Gly601Cys) | 417 | SNP:c.2059C > T (p.His687Tyr) | VCV000067357 | Congenital long QT Syn. | Not Prov. |
| 275 | SNP:c.1802G > A (p.Gly601Asp [symbol] | 420 | SNP:c.206T > C (p.Leu69Pro) | VCV000067358 | Congenital long QT Syn. | Not Prov. |
| 276 | SNP:c.1805T > C (p.Leu602Pro) | 426 | SNP:c.2087G > C (p.Arg696Pro) | VCV000067361 | Congenital long QT Syn. | Not Prov. |
| 277 | SNP:c.1808G > A (p.Gly603Asp) | 427 | SNP:c.208C > A (p.His70Asn) | VCV000067362 | Congenital long QT Syn. | Not Prov. |
| 278 | SNP:c.1810G > A (p.Gly604Ser) | 433 | SNP:c.2117C > G (p.Ser706Cys) | VCV000067364 | Congenital long QT Syn. | Not Prov. |
| 279 | SNP:c.1811G > A (p.Gly604Asp [symbol] | 434 | SNP:c.2117C > T (p.Ser706Phe) | VCV000067365 | Acquired long QT Syn. | Not Prov. |
| 280 | SNP:c.1811G > T (p.Gly604Val) | 448 | SNP:c.2167T > C (p.Cys723Arg) | VCV000067372 | Not Prov. | Not Prov. |
| 281 | SNP:c.1813C > T (p.Pro605Ser) | 452 | SNP:c.2182A > T (p.Ile728Phe) | VCV000067373 | Congenital long QT Syn. | Not Prov. |
| 282 | SNP:c.1814C > A (p.Pro605His) | 456 | SNP:c.220A > C (p.Thr74Pro) | VCV000067375 | Congenital long QT Syn. | Not Prov. |
| 283 | SNP:c.1814C > T (p.Pro605Leu) | 476 | SNP:c.2271G > C (p.Lys757Asn) | VCV000067382 | Congenital long QT Syn. | Not Prov. |
| 284 | SNP:c.1816T > C (p.Ser606Pro) | 483 | SNP:c.2299G > T (p.Asp767Tyr) | VCV000067383 | Congenital long QT Syn. | Not Prov. |
| 285 | SNP:c.1819A > T (p.Ile607Phe) | 484 | SNP:c.2309T > C (p.Val770Ala) | VCV000067384 | Congenital long QT Syn. | Not Prov. |
| 286 | SNP:c.1825G > A (p.Asp609Asn [symbol] | 490 | SNP:c.232G > C (p.Ala78Pro) | VCV000067386 | Congenital long QT Syn. | Not Prov. |
| 287 | SNP:c.1825G > C (p.Asp609His) | 496 | SNP:c.2354G > C (p.Gly785Ala) | VCV000067387 | Congenital long QT Syn. | Not Prov. |
| 288 | SNP:c.1825G > T (p.Asp609Tyr) | 497 | SNP:c.2354G > T (p.Gly785Val) | VCV000067388 | Congenital long QT Syn. | Not Prov. |
| 289 | SNP:c.1826A > C (p.Asp609Ala) | 503 | SNP:c.2364G > C (p.Glu788Asp) | VCV000067390 | Congenital long QT Syn. | Not Prov. |
| 290 | SNP:c.1826A > G (p.Asp609Gly [symbol] | 515 | SNP:c.238G > C (p.Ala80Pro) | VCV000067392 | Congenital long QT Syn. | Not Prov. |
| 291 | SNP:c.1828A > G (p.Lys610Glu) | 518 | SNP:c.2398G > T (p.Gly800Trp) | VCV000067393 | Congenital long QT Syn. | Not Prov. |
| 292 | SNP:c.1831T > C (p.Tyr611His) | 519 | SNP:c.2399G > A (p.Gly800Glu) | VCV000067394 | Congenital long QT Syn. | Not Prov. |
| 293 | SNP:c.1831T > G (p.Tyr611Asp) | 520 | SNP:c.239C > T (p.Ala80Val) | VCV000067395 | Sudden cardiac death | Not Prov. |
| 294 | SNP:c.1832A > G (p.Tyr611Cys) | 528 | SNP:c.2452T > C (p.Ser818Pro) | VCV000067400 | Congenital long QT Syn. | Not Prov. |
| 295 | SNP:c.1834G > T (p.Val612Leu) | 541 | SNP:c.2503C > T (p.Arg835Trp) | VCV000067404 | Congenital long QT Syn. | Not Prov. |
| 296 | SNP:c.1837A > T (p.Thr613Ser) | 544 | SNP:c.2509G > T (p.Asp837Tyr) | VCV000067406 | Congenital long QT Syn. | Not Prov. |
| 297 | SNP:c.1838C > T (p.Thr613Met) | 550 | SNP:c.2536C > A (p.Pro846Thr) | VCV000067408 | Congenital long QT Syn. | Not Prov. |
| 298 | SNP:c.1841C > T (p.Ala614Val) | 555 | SNP:c.253G > C (p.Ala85Pro) | VCV000067410 | Congenital long QT Syn. | Not Prov. |
| 299 | SNP:c.1843C > G (p.Leu615Val) | 557 | SNP:c.254C > T (p.Ala85Val) | VCV000067411 | Congenital long QT Syn. | Not Prov. |
| 300 | SNP:c.1843C > T (p.Leu615Phe) | 559 | SNP:c.2573T > C (p.Ile858Thr) | VCV000067412 | Congenital long QT Syn. | Not Prov. |
| 301 | SNP:c.1847A > G (p.Tyr616Cys) | 562 | SNP:c.257T > G (p.Leu86Arg) | VCV000067414 | Congenital long QT Syn. | Not Prov. |
| 302 | SNP:c.1848C > G (p.Tyr616Ter) | 563 | SNP:c.2581A > C (p.Asn861His) | VCV000067415 | Congenital long QT Syn. | Not Prov. |
| 303 | SNP:c.1849T > C (p.Phe617Leu) | 564 | SNP:c.2582A > C (p.Asn861Thr) | VCV000067416 | Congenital long QT Syn. | Not Prov. |
| 304 | SNP:c.1851C > A (p.Phe617Leu) | 583 | SNP:c.2635G > C (p.Gly879Arg) | VCV000067421 | Congenital long QT Syn. | Not Prov. |
| 305 | SNP:c.1853C > G (p.Thr618Ser) | 601 | SNP:c.2681G > T (p.Arg894Leu) | VCV000067425 | Congenital long QT Syn. | Not Prov. |
| 306 | SNP:c.1853C > T (p.Thr618Ile) | 629 | SNP:c.2744C > T (p.Ala915Val) | VCV000067431 | Not Prov. | Not Prov. |
| 307 | SNP:c.1858A > G (p.Ser620Gly) | 648 | SNP:c.2773G > A (p.Gly925Arg) | VCV000067439 | Not Prov. | Not Prov. |
| 308 | SNP:c.1859G > A (p.Ser620Asn) | 675 | SNP:c.2842C > A (p.Arg948Ser) | VCV000067442 | Congenital long QT Syn. | Not Prov. |
| 309 | SNP:c.185G > A (p.Arg62Gln) | 687 | SNP:c.286A > G (p.Ile96Val) | VCV000067446 | Congenital long QT Syn. | Not Prov. |
| 310 | SNP:c.1862G > A (p.Ser621Asn) | 692 | SNP:c.2879G > A (p.Ser960Asn) | VCV000067447 | Congenital long QT Syn. | Not Prov. |
| 311 | SNP:c.1863C > G (p.Ser621Arg) | 693 | SNP:c.287T > C (p.Ile96Thr) | VCV000067448 | Congenital long QT Syn. | Not Prov. |
| 312 | SNP:c.1864C > T (p.Leu622Phe) | 724 | SNP:c.296A > C (p.Tyr99Ser) | VCV000067457 | Congenital long QT Syn. | Not Prov. |
| 313 | SNP:c.1868C > T (p.Thr623Ile) | 754 | SNP:c.305A > C (p.Asp102Ala) | VCV000067464 | Congenital long QT Syn. | Not Prov. |
| 314 | SNP:c.1870A > T (p.Ser624Cys) | 755 | SNP:c.305A > T (p.Asp102Val) | VCV000067465 | Congenital long QT Syn. | Not Prov. |
| 315 | SNP:c.1873G > C (p.Val625Leu) | 786 | SNP:c.3146T > C (p.Leu1049Pro) | VCV000067474 | Congenital long QT Syn. | Not Prov. |
| 316 | SNP:c.1874T > A (p.Val625Glu) | 793 | SNP:c.3173C > A (p.Ala1058Glu) | VCV000067476 | Not Prov. | Not Prov. |
| 317 | SNP:c.1874T > C (p.Val625Ala) | 795 | SNP:c.317T > A (p.Phe106Tyr) | VCV000067477 | Congenital long QT Syn. | Not Prov. |
| 318 | SNP:c.1876G > A (p.Gly626Ser) | 798 | SNP:c.318C > A (p.Phe106Leu) | VCV000067478 | Congenital long QT Syn. | Not Prov. |
| 319 | SNP:c.1877G > A (p.Gly626Asp) | 801 | SNP:c.3196C > G (p.Leu1066Val) | VCV000067479 | Congenital long QT Syn. | Not Prov. |
| 320 | SNP:c.1877G > C (p.Gly626Ala) | 826 | SNP:c.3302C > T (p.Pro1101Leu) | VCV000067487 | Congenital long QT Syn. | Not Prov. |
| 321 | SNP:c.1877G > T (p.Gly626Val) | 832 | SNP:c.332A > T (p.Asp111Val) | VCV000067489 | Congenital long QT Syn. | Not Prov. |
| 322 | SNP:c.1879T > A (p.Phe627Ile) | 849 | SNP:c.3404G > A (p.Arg1135His) | VCV000067493 | short QT Syn. | Not Prov. |
| 323 | SNP:c.1879T > C (p.Phe627Leu) | 851 | SNP:c.340C > T (p.Pro114Ser) | VCV000067494 | Congenital long QT Syn. | Not Prov. |
| 324 | SNP:c.1881C > A (p.Phe627Leu) | 854 | SNP:c.3430G > A (p.Ala1144Thr) | VCV000067495 | Congenital long QT Syn. | Not Prov. |
| 325 | SNP:c.1881C > G (p.Phe627Leu) | 871 | SNP:c.371T > C (p.Met124Thr) | VCV000067500 | Congenital long QT Syn. | Not Prov. |
| 326 | SNP:c.1882G > A (p.Gly628Ser) | 872 | SNP:c.371T > G (p.Met124Arg) | VCV000067501 | Congenital long QT Syn. | Not Prov. |
| 327 | SNP:c.1882G > C (p.Gly628Arg) | 907 | SNP:c.47A > C (p.Asp 16Ala) | VCV000067507 | Congenital long QT Syn. | Not Prov. |
| 328 | SNP:c.1883G > C (p.Gly628Ala) | 921 | SNP:c.559G > A (p.Gly187Ser) | VCV000067511 | Not Prov. | Not Prov. |
| 329 | SNP:c.1883G > T (p.Gly628Val) | 939 | SNP:c.644T > G (p.Val215Gly) | VCV000067515 | Not Prov. | Not Prov. |
| 330 | SNP:c.1885A > G (p.Asn629Asp [symbol] | 944 | SNP:c.65T > C (p.Phe22Ser) | VCV000067517 | Congenital long QT Syn. | Not Prov. |
| 331 | SNP:c.1886A > C (p.Asn629Thr) | 960 | SNP:c.712G > A (p.Gly238Ser) | VCV000067519 | Congenital long QT Syn. | Not Prov. |
| 332 | SNP:c.1886A > G (p.Asn629Ser) | 962 | SNP:c.722C > T (p.Pro241Leu) | VCV000067520 | Congenital long QT Syn. | Not Prov. |
| 333 | SNP:c.1886A > T (p.Asn629Ile) | 963 | SNP:c.724C > G (p.Arg242Gly) | VCV000067521 | Congenital long QT Syn. | Not Prov. |
| 334 | SNP:c.1887C > A (p.Asn629Lys) | 971 | SNP:c.751C > G (p.Pro251Ala) | VCV000067522 | Not Prov. | Not Prov. |
| 335 | SNP:c.1888G > A (p.Val630Ile) | 973 | SNP:c.754C > G (p.Arg252Gly) | VCV000067524 | Congenital long QT Syn. | Not Prov. |
| 336 | SNP:c.1888G > C (p.Val630Leu) | 976 | SNP:c.762C > G (p.His254Gln) | VCV000067525 | Not Prov. | Not Prov. |
| 337 | SNP:c.1889T > C (p.Val630Ala) | 984 | SNP:c.77G > T (p.Ser26Ile) | VCV000067528 | Congenital long QT Syn. | Not Prov. |
| 338 | SNP:c.188C > A (p.Pro63His) | 987 | SNP:c.805C > T (p.Arg269Trp) | VCV000067529 | Congenital long QT Syn. | Not Prov. |
| 339 | SNP:c.1891T > G (p.Ser631Ala) | 989 | SNP:c.80G > C (p.Arg27Pro) | VCV000067530 | Congenital long QT Syn. | Not Prov. |
| 340 | SNP:c.1892C > T (p.Ser631Phe) | 993 | SNP:c.818G > A (p.Arg273Gln) | VCV000067531 | SUDDEN INFANT DEATH Syn. | Not Prov. |
| 341 | SNP:c.1894C > G (p.Pro632Ala) | 997 | SNP:c.82A > G (p.Lys28Glu) | VCV000067532 | Congenital long QT Syn. | Not Prov. |
| 342 | SNP:c.1894C > T (p.Pro632Ser) | 1000 | SNP:c.835G > A (p.Val279Met) | VCV000067534 | SUDDEN INFANT DEATH Syn. | Not Prov. |
| 343 | SNP:c.1897A > C (p.Asn633His) | 1008 | SNP:c.86T > C (p.Phe29Ser) | VCV000067536 | Congenital long QT Syn. | Not Prov. |
| 344 | SNP:c.1897A > G (p.Asn633Asp) | 1020 | SNP:c.902G > T (p.Arg301Leu) | VCV000067542 | Congenital long QT Syn. | Not Prov. |
| 345 | SNP:c.1898A > G (p.Asn633Ser) | 1030 | SNP:c.92T > G (p.Ile31Ser) | VCV000067546 | Congenital long QT Syn. | Not Prov. |
| 346 | SNP:c.1898A > T (p.Asn633Ile) | 1034 | SNP:c.940G > A (p.Gly314Ser) | VCV000067548 | Congenital long QT Syn. | Not Prov. |
| 347 | SNP:c.1900A > G (p.Thr634Ala) | 1040 | SNP:c.959C > T (p.Ser320Leu) | VCV000067550 | Congenital long QT Syn. | Not Prov. |
| 348 | SNP:c.1901C > T (p.Thr634Ile) | 104 | SNP:c.1425C > A (p.Tyr475Ter) | VCV001178326 | Long QT Syn. 2 | Pathogenic |
| 349 | SNP:c.1903A > G (p.Asn635Asp [symbol] | 255 | SNP:c.1764C > G (p.Asn588Lys) | VCV000014436 | short QT Syn.\|Short QT Syn. 1 | Pathogenic(LR: Apr. 1, 2005) |
| 350 | SNP:c.1904A > G (p.Asn635Ser) | 858 | SNP:c.344T > G (p.Val115Gly) | VCV000200276 | Not Prov. | Pathogenic(LR: Apr. 10, 2014) |
| 351 | SNP:c.1904A > T (p.Asn635Ile) | 443 | SNP:c.214C > T (p.Pro72Ser) | VCV000200564 | Not Prov. | Pathogenic(LR: Apr. 12, 2014) |
| 352 | SNP:c.1905C > G (p.Asn635Lys) | 174 | SNP:c.1655T > C (p.Leu552Ser) | VCV000067225 | Long QT Syn.\|Long QT Syn. 2\|Congenital [symbol] | Pathogenic(LR: Apr. 15, 2021) |
| 353 | SNP:c.1907C > T (p.Ser636Leu) | 355 | SNP:c.1909G > T (p.Glu637Ter) | VCV000977166 | Long QT Syn. | Pathogenic(LR: Apr. 2, 2019) |
| 354 | SNP:c.1909G > A (p.Glu637Lys) | 566 | SNP:c.2582A > T (p.Asn861Ile) | VCV000014440 | Congenital long QT Syn.\|Long QT Syn. 2\| [symbol] | Pathogenic(LR: Apr. 20, 2004) |
| 355 | SNP:c.1909G > T (p.Glu637Ter) | 324 | SNP:c.1881C > A (p.Phe627Leu) | VCV000200747 | Long QT Syn.\|Not Prov. | Pathogenic(LR: Apr. 22, 2020) |
| 356 | SNP:c.1910A > G (p.Glu637Gly) | 354 | SNP:c.1909G > A (p.Glu637Lys) | VCV000067329 | Long QT Syn.\|Congenital long QT Syn. | Pathogenic(LR: Apr. 26, 2018) |
| 357 | SNP:c.1911G > C (p.Glu637Asp) | 747 | SNP:c.303A > T (p.Lys101Asn) | VCV000200590 | Not Prov. | Pathogenic(LR: Apr. 28, 2014) |
| 358 | SNP:c.1912A > G (p.Lys638Glu) | 367 | SNP:c.1922C > T (p.Ser641Phe) | VCV000067337 | Congenital long QT Syn.\|Long QT Syn. | Pathogenic(LR: Apr. 30, 2019) |
| 359 | SNP:c.1913A > G (p.Lys638Arg) | 170 | SNP:c.164C > A (p.Ser55Ter) | VCV000418597 | Long QT Syn.\|Not Prov. | Pathogenic(LR: Apr. 7, 2021) |
| 360 | SNP:c.1914G > T (p.Lys638Asn) | 330 | SNP:c.1885A > G (p.Asn629Asp) | VCV000067313 | Congenital long QT Syn.\|Not Prov. | Pathogenic(LR: Apr. 9, 2015) |
| 361 | SNP:c.1918T > C (p.Phe640Leu) | 399 | SNP:c.198C > A (p.Cys66Ter) | VCV000863419 | Long QT Syn. | Pathogenic(LR: Apr. 9, 2019) |
| 362 | SNP:c.1918T > G (p.Phe640Val) | 580 | SNP:c.2626G > T (p.Glu876Ter) | VCV000488692 | Long QT Syn.\|Not Prov. | Pathogenic(LR: Aug. 14, 2020) |
| 363 | SNP:c.1919T > G (p.Phe640Cys) | 525 | SNP:c.2417G > C (p.Gly806Ala) | VCV000200453 | Not Prov. | Pathogenic(LR: Aug. 16, 2012) |
| 364 | SNP:c.191G > A (p.Cys64Tyr) | 734 | SNP:c.3003G > A (p.Trp1001Ter) | VCV000014431 | Long QT Syn. 2\|Not Prov. Long QT Syn. | Pathogenic(LR: Aug. 16, 2019) |
| 365 | SNP:c.1920C > A (p.Phe640Leu) | 286 | SNP:c.1825G > A (p.Asp609Asn) | VCV000067286 | Long QT Syn.\|Congenital long QT Syn.\|Not [symbol] | Pathogenic(LR: Aug. 22, 2019) |
| 366 | SNP:c.1920C > G (p.Phe640Leu) | 130 | SNP:c.1502A > G (p.Asp501Gly) | VCV000067210 | Not Prov.\|Congenital long QT Syn. | Pathogenic(LR: Aug. 24, 2012) |
| 367 | SNP:c.1922C > T (p.Ser641Phe) | 733 | SNP:c.3002G > A (p.Trp1001Ter) | VCV000200513 | Not Prov.\|Arrhythmia\|Long QT Syn. | Pathogenic(LR: Aug. 27, 2020) |
| 368 | SNP:c.1926C > G (p.Ile642Met) | 744 | SNP:c.3027C > G (p.Tyr1009Ter) | VCV000379293 | Long QT Syn.\|Not Prov. | Pathogenic(LR: Aug. 29, 2018) |
| 369 | SNP:c.9129C > A (p.Cys643Ter) | 16 | SNP:c.162C > G (p.Tyr54Ter) | VCV000519501 | Cardiovas. pheno. | Pathogenic(LR: Aug. 31, 2017) |
| 370 | SNP:c.192C > G (p.Cys64Trp) | 378 | SNP:c.193A > C (p.Thr65Ter) | VCV000014434 | Congenital long QT Syn.\|Long QT Syn. 2 | Pathogenic(LR: Dec. 13, 2002) |
| 371 | SNP:c.1930G > A (p.Val644Ile) | 385 | SNP:c.1659T > A (p.Tyr652Ter) | VCV000200402 | Not Prov. | Pathogenic(LR: Dec. 14, 2017) |
| 372 | SNP:c.1930G > C (p.Val644Leu) | 719 | SNP:c.2952C > A (p.Cys984Ter) | VCV000200510 | Not Prov. | Pathogenic(LR: Dec. 3, 2013) |
| 373 | SNP:c.1930G > T (p.Val644Phe) | 369 | SNP:c.1929C > A (p.Cys643Ter) | VCV000392341 | Not Prov. | Pathogenic(LR: Dec. 30, 2016) |
| 374 | SNP:c.1933A > C (p.Met645Leu) | 72 | SNP:c.1330G > T (p.Glu444Ter) | VCV000200337 | Not Prov. | Pathogenic(LR: Dec. 7, 2012) |
| 375 | SNP:c.1933A > G (p.Met645Val) | 124 | SNP:c.1479C > A (p.Tyr493Ter) | VCV000237291 | Long QT Syn. | Pathogenic(LR: Dec. 9, 2015) |
| 376 | SNP:c.1933A > T (p.Met645Leu) | 222 | SNP:c.1714G > C (p.Gly572Arg) | VCV000014429 | Congenital long QT Syn.\|Long QT Syn. 2 | Pathogenic(LR: Feb. 1, 2000) |
| 377 | SNP:c.1935G > A (p.Met645Ile) | 436 | SNP:c.2111G > C (p.Gly71Arg) | VCV000067366 | Long QT Syn.\|Congenital long QT Syn.\|Not [symbol] | Pathogenic(LR: Feb. 11, 2019) |
| 378 | SNP:c.193A > C (p.Thr65Pro) | 802 | SNP:c.31C > T (p.Gln11Ter) | VCV000200588 | Not Prov. | Pathogenic(LR: Feb. 15, 2012) |
| 379 | SNP:c.1942G > A (p.Gly648Ser) | 96 | SNP:c.1408A > G (p.Asn470Asp) | VCV000014421 | Congenital long QT Syn.\|Long QT Syn. 2 | Pathogenic(LR: Feb. 17, 2006) |
| 380 | SNP:c.1943G > A (p.Gly648Asp) | 302 | SNP:c.1848C > G (p.Tyr616Ter) | VCV000560696 | Long QT Syn. 2 | Pathogenic(LR: Feb. 17, 2018) |
| 381 | SNP:c.1945T > C (p.Ser649Pro) | 522 | SNP:c.2414T > C (p.Phe805Ser) | VCV000067396 | Congenital long QT Syn.\|Not Prov. | Pathogenic(LR: Feb. 2, 2012) |
| 382 | SNP:c.1946C > T (p.Ser649Phe) | 260 | SNP:c.1778T > C (p.Ile93Thr) | VCV000067270 | Not Prov.\|Congenital long QT Syn. | Pathogenic(LR: Feb. 2, 2014) |
| 383 | SNP:c.194C > G (p.Thr65Ser) | 406 | SNP:c.2026C > T (p.Gln676Ter) | VCV000200405 | Not Prov. | Pathogenic(LR: Feb. 2, 2014) |
| 384 | SNP:c.1952T > G (p.Met651Arg) | 726 | SNP:c.2987A > T (p.Asn996Ile) | VCV000067458 | Not Prov.\|Long QT Syn. 2\|Congenital long Q [symbol] | Pathogenic(LR: Feb. 24, 2020) |
| 385 | SNP:c.1956T > A (p.Tyr652Ter) | 905 | SNP:c.461G > A (p.Trp154Ter) | VCV000456935 | Long QT Syn. | Pathogenic(LR: Feb. 3, 2017) |
| 386 | SNP:c.1963A > G (p.Ile655Val) | 179 | SNP:c.1675C > T (p.Leu559Phe) | VCV000200363 | Not Prov. | Pathogenic(LR: Feb. 5, 2014) |
| 387 | SNP:c.1967T > G (p.Phe656Cys) | 186 | SNP:c.1681G > C (p.Ala561Pro) | VCV000067230 | Congenital long QT Syn.\|Cardiovas. pheno [symbol] | Pathogenic(LR: Feb. 5, 2018) |
| 388 | SNP:c.1969G > A (p.Gly657Ser) | 365 | SNP:c.1920C > A (p.Phe640Leu) | VCV000067336 | Long QT Syn. 2\|Congenital long QT Syn. | Pathogenic(LR: Jan. 1, 2013) |
| 389 | SNP:c.1969G > C (p.Gly657Arg) | 359 | SNP:c.1913A > G (p.Lys638Arg) | VCV000200395 | Not Prov. | Pathogenic(LR: Jan. 10, 2014) |
| 390 | SNP:c.1969G > T (p.Gly657Cys) | 215 | SNP:c.1707C > G (p.Tyr569Ter) | VCV000489342 | Not Prov. | Pathogenic(LR: Jan. 15, 2018) |
| 391 | SNP:c.196T > C (p.Cys66Arg) | 267 | SNP:c.1787C > A (p.Pro596His) | VCV000067274 | Not Prov.\|Congenital long QT Syn. | Pathogenic(LR: Jan. 31, 2014) |
| 392 | SNP:c.196T > G (p.Cys66Gly) | 254 | SNP:c.1764C > A (p.Asn588Lys) | VCV000014437 | short QT Syn.\|Short QT Syn. 1 | Pathogenic(LR: Jan. 6, 2004) |
| 393 | SNP:c.1970G > T (p.Gly657Val) | 177 | SNP:c.1672G > C (p.Ala558Pro) | VCV000014444 | Congenital long QT Syn. Long QT Syn. 2 | Pathogenic(LR: Jul. 1, 2008) |
| 394 | SNP:c.1973A > G (p.Asn658Ser) | 374 | SNP:c.1933A > C (p.Met645LEu) | VCV000869431 | Long QT Syn. 2 | Pathogenic(LR: Jul. 11, 2019) |
| 395 | SNP:c.1979C > T (p.Ser660Leu) | 714 | SNP:c.2932G > T (p.Glu978Ter) | VCV000200484 | Not Prov. | Pathogenic(LR: Jul. 15, 2014) |
| 396 | SNP:c.197G > A (p.Cys66Tyr) | 532 | SNP:c.2464G > A (p.Val822Met) | VCV000014424 | Not Prov.\|Long QT Syn.\|Long QT Syn. 1\| [symbol] | Pathogenic(LR: Jul. 23, 2020) |
| 397 | SNP:c.1982C > T (p.Ala661Val) | 259 | SNP:c.1778T > A (p.Ile593Lys) | VCV000067269 | Long QT Syn.\|Congenital long QT Syn. | Pathogenic(LR: Jul. 25, 2017) |
| 398 | SNP:c.1985T > C (p.Ile662Thr) | 446 | SNP:c.215C > T (p.Pro72Leu) | VCV000067370 | Congenital long QT Syn.\|Not Prov. | Pathogenic(LR: Jul. 3, 2019) |
| 399 | SNP:c.198C > A (p.Cys66Ter) | 53 | SNP:c.1264G > A (p.Ala422Thr) | VCV000067174 | Not Prov.\|Congenital long QT Syn. | Pathogenic(LR: Jul. 31, 2012) |
| 400 | SNP:c.1A > T (p.Met1Leu) | 803 | SNP:c.3202C > T (p.Gln1068Ter) | VCV000446417 | Long QT Syn. 2 | Pathogenic(LR: Jul. 4, 2017) |
| 401 | SNP:c.2003C > A (p.S668Ter) | 710 | SNP:c.2931C > A (p.Cys977Ter) | VCV000568003 | Long QT Syn. | Pathogenic(LR: Jul. 5, 2018) |
| 402 | SNP:c.2003G > C (p.Gly669Ala) | 536 | SNP:c.2477C > T (p.Thr826Ile) | VCV000067403 | Not Prov.\|Congenital long QT Syn. | Pathogenic(LR: Jun. 12, 2013) |
| 403 | SNP:c.200A > G (p.asp67Gly) | 185 | SNP:c.1681G > A (p.Ala561Thr) | VCV000067229 | Long QT Syn.\|Inborn genetic diseases\|Not P [symbol] | Pathogenic(LR: Jun. 15, 2020) |
| 404 | SNP:c.2012C > G (p.Ala671Gly) | 418 | SNP:c.2062C > T (p.Gln688Ter) | VCV000456901 | Long QT Syn. | Pathogenic(LR: Jun. 16, 2017) |
| 405 | SNP:c.2014C > T (p.Arg672Cys) | 125 | SNP:c.147C > G (p.Cys49Trp) | VCV000200555 | Not Prov. | Pathogenic(LR: Jun. 19, 2013) |
| 406 | SNP:c.2026C > T (p.Gln676Ter) | 229 | SNP:c.1726C > T (p.Gln576Ter) | VCV000419311 | Not Prov. | Pathogenic(LR: Jun. 23, 2015) |
| 407 | SNP:c.202T > C (p.Phe68Leu) | 1014 | SNP:c.87C > A (p.Phe29Leu) | VCV000067538 | Long QT Syn.\|Congenital long QT Syn. | Pathogenic(LR: Jun. 29, 2020) |
| 408 | SNP:c.2030T > C (p.Met677Thr) | 33 | SNP:c.1193G > A (p.Trp398Ter) | VCV000200327 | Not Prov.\|Long QT Syn. | Pathogenic(LR: Jun. 4, 2020) |
| 409 | SNP:c.2033T > C (p.Leu678Pro) | 194 | SNP:c.1689G > A (p.Trp563Ter) | VCV000526968 | Not Prov.\|Long QT Syn. | Pathogenic(LR: Jun. 6, 2018) |
| 410 | SNP:c.2035C > T (p.Arg679Trp) | 540 | SNP:c.2494A > T (p.Lys832Ter) | VCV000200460 | Not Prov. | Pathogenic(LR: Jun. 7, 2018) |
| 411 | SNP:c.2036G > A (p.Arg679Gln) | 653 | SNP:c.2780G > A (p.Trp927Ter) | VCV000200499 | Long QT Syn. | Pathogenic(LR: Mar. 1, 2018) |
| 412 | SNP:c.203T > G (p.Phe68Cys) | 50 | SNP:c.125T > C (p.Ile42Thr) | VCV000200546 | Not Prov. | Pathogenic(LR: Mar. 15, 2013) |
| 413 | SNP:c.2042G > A (p.Arg681Gln) | 366 | SNP:c.1920C > G (p.Phe640Leu) | VCV000423753 | Not Prov. | Pathogenic(LR: Mar. 3, 2017) |
| 414 | SNP:c.2046G > C (p.Glu682Asp) | 51 | SNP:c.1262C > A (p.Thr421Lys) | VCV000200331 | Not Prov. | Pathogenic(LR: Mar. 31, 2013) |
| 415 | SNP:c.2053C > T (p.Arg685Cys) | 307 | SNP:c.1858A > G (p.Ser620Gly) | VCV001189275 | Not Prov. | Pathogenic(LR: Mar. 5, 2021) |
| 416 | SNP:c.2054G > A (p.Arg685His) | 326 | SNP:c.1882G > A (p.Gly628Ser) | VCV000014427 | Long QT Syn.\|Congenital long QT Syn.\|Lon [symbol] | Pathogenic(LR: May 12, 2020) |
| 417 | SNP:c.2059C > T (p.His687Tyr) | 292 | SNP:c.1831T > C (p.Tyr611His) | VCV000029778 | Congenital long QT Syn.\|Long QT Syn. 2 | Pathogenic(LR: May 13, 2005) |
| 418 | SNP:c.2062C > T (p.Gln688Ter) | 261 | SNP:c.1778T > G (p.Ile593Arg) | VCV000014423 | Congenital long QT Syn.\|Long QT Syn. 2\|No [symbol] | Pathogenic(LR: May 24, 2019) |
| 419 | SNP:c.206T > A (p.Leu69Gln) | 347 | SNP:c.1900A > G (p.Thr634Ala) | VCV000200391 | Not Prov. | Pathogenic(LR: May 28, 2014) |
| 420 | SNP:c.206T > C (p.Leu69Pro) | 226 | SNP:c.1715G > T (p.Gly572Val) | VCV000067251 | Congenital long QT Syn.\|Not Prov. | Pathogenic(LR: May 4, 2016) |
| 421 | SNP:c.2078T > C (p.Leu693Pro) | 239 | SNP:c.1744C > T (p.Arg582Cys) | VCV000014428 | Not Prov.\|Long QT Syn.\|Congenital long QT [symbol] | Pathogenic(LR: Nov. 10, 2020) |
| 422 | SNP:c.2080C > T (p.Arg694Cys) | 748 | SNP:c.3040C > T (p.Arg1014Ter) | VCV000200518 | Not Prov.\|Long QT Syn. | Pathogenic(LR: Nov. 13, 2019) |
| 423 | SNP:c.2081G > A (p.Arg694His) | 237 | SNP:c.1742C > A (p.Ser581Ter) | VCV000200737 | Not Prov. | Pathogenic(LR: Nov. 16, 2011) |
| 424 | SNP:c.2083C > T (p.Gln695Ter) | 567 | SNP:c.2587C > T (p.Arg863Ter) | VCV000220208 | Inborn genetic diseases\|Not Prov.\|Long QT S [symbol] | Pathogenic(LR: Nov. 2, 2020) |
| 425 | SNP:c.2086C > T (p.Arg696Cys) | 742 | SNP:c.301A > T (p.Lys101Ter) | VCV000200586 | Not Prov. | Pathogenic(LR: Nov. 20, 2013) |
| 426 | SNP:c.2087G > C (p.Arg696Pro) | 21 | SNP:c.1096C > T (p.Arg366Ter) | VCV000200321 | Not Prov.\|Long QT Syn. | Pathogenic(LR: Nov. 21, 2019) |
| 427 | SNP:c.208C > A (p.His70Asn) | 375 | SNP:c.1933A > G (p.Met645Val) | VCV000067341 | Congenital long QT Syn.\|Not Prov. | Pathogenic(LR: Nov. 22, 2013) |
| 428 | SNP:c.2092G > A (p.Glu698Lys) | 41 | SNP:c.1229G > A (p.Trp410Ter) | VCV000972695 | Long QT Syn. 2 | Pathogenic(LR: Nov. 22, 2019) |
| 429 | SNP:c.2094G > T (p.Glu698Asp) | 140 | SNP:c.156C > A (p.Cys52Ter) | VCV000200772 | Long QT Syn. | Pathogenic(LR: Nov. 3, 2019) |
| 430 | SNP:c.2097G > T (p.Glu699Asp) | 266 | SNP:c.1786C > G (p.Pro596Ala) | VCV000218093 | Long QT Syn. 2 | Pathogenic(LR: Nov. 4, 2015) |
| 431 | SNP:c.209A > G (p.His70Arg) | 221 | SNP:c.1714G > A (p.Gly572Ser) | VCV000067248 | Long QT Syn. 2\|Not Prov.\|Congenital long Q [symbol] | Pathogenic(LR: Nov. 8, 2019) |
| 432 | SNP:c.2104C > T (p.Gln702Ter) | 190 | SNP:c.1685A > G (p.His562Arg) | VCV000067232 | Long QT Syn.\|Congenital long QT Syn. | Pathogenic(LR: Oct. 1, 2019) |
| 433 | SNP:c.2117C > G (p.Ser706Cys) | 102 | SNP:c.1421C > T (p.Thr474Ile) | VCV000067198 | Not Prov.\|Congenital long QT Syn. | Pathogenic(LR: Oct. 10, 2014) |
| 434 | SNP:c.2117C > T (p.Ser706Phe) | 3 | SNP:c.1003C > T (p.Gln335Ter) | VCV000968402 | Long QT Syn. | Pathogenic(LR: Oct. 10, 2019) |
| 435 | SNP:c.211G > A (p.Gly71Arg) | 1041 | SNP:c.95C > T (p.Ala32Val) | VCV000869439 | Long QT Syn. 2 | Pathogenic(LR: Oct. 12, 2019) |
| 436 | SNP:c.211G > C (p.Gly71Arg) | 173 | SNP:c.1655T > A (p.Leu552Ter) | VCV000648088 | Not Prov.\|Long QT Syn. | Pathogenic(LR: Oct. 20, 2020) |
| 437 | SNP:c.211G > T (p.Gly71Trp) | 401 | SNP:c.2003C > A (p.Ser668Ter) | VCV000620172 | Not Prov. | Pathogenic(LR: Oct. 22, 2018) |
| 438 | SNP:c.2122A > G (p.Thr708Ala) | 155 | SNP:c.1600C > T (p.Arg534Cys) | VCV000067220 | Not Prov.\|Long QT Syn.\|Congenital long Q [symbol] | Pathogenic(LR: Oct. 24, 2020) |
| 439 | SNP:c.2128G > A (p.Gly710Ser) | 193 | SNP:c.1688G > A (p.Trp563Ter) | VCV000943351 | Long QT Syn. | Pathogenic(LR: Oct. 26, 2019) |
| 440 | SNP:c.212G > A (p.Gly71Glu) | 992 | SNP:c.817C > T (p.Arg273Ter) | VCV001076382 | Long QT Syn. | Pathogenic(LR: Oct. 26, 2020) |
| 441 | SNP:c.2131A > G (p.Ile711Val) | 800 | SNP:c.3193C > T (p.Gln1065Ter) | VCV000519524 | Cardiovas. pheno.\|Long QT Syn. | Pathogenic(LR: Oct. 28, 2020) |
| 442 | SNP:c.2144C > T (p.Ala715Val) | 278 | SNP:c.1810G > A (p.Gly604Ser) | VCV000067281 | Not Prov.\|Congenital long QT Syn.\|Long Q [symbol] | Pathogenic(LR: Oct. 29, 2020) |
| 443 | SNP:c.214C > T (p.Pro72Ser) | 565 | SNP:c.2582A > G (p.Asn861Ser) | VCV000200471 | Not Prov. | Pathogenic(LR: Oct. 30, 2013) |
| 444 | SNP:c.215C > A (p.Pro72Gln) | 332 | SNP:c.1886A > G (p.Asn629Ser) | VCV000067315 | Long QT Syn. 2\|Long QT Syn.\|Congenital [symbol] | Pathogenic(LR: Oct. 31, 2018) |
| 445 | SNP:c.215C > G (p.Pro72Arg) | 432 | SNP:c.2104C > T (p.Gln702Ter) | VCV000200407 | Not Prov.\|Long QT Syn. 2\|Short QT Syn. 1 | Pathogenic(LR: Oct. 31, 2018) |
| 446 | SNP:c.215C > T (p.Pro72Leu) | 273 | SNP:c.1801G > A (p.Gly601Ser) | VCV000067279 | Long QT Syn. 2\|Congenital long QT Syn.\| [symbol] | Pathogenic(LR: Oct. 5, 2020) |
| 447 | SNP:c.2162C > T (p.Pro721Leu) | 919 | SNP:c.545C > A (p.Ser182Ter) | VCV000372376 | Long QT Syn.\|Not Prov. | Pathogenic(LR: Oct. 9, 2018) |
| 448 | SNP:c.2167T > C (p.Cys723Arg) | 296 | SNP:c.1837A > T (p.Thr613Ser) | VCV000200743 | Not Prov. | Pathogenic(LR: Sep. 12, 2014) |
| 449 | SNP:c.2167T > G (p.Cys723Gly) | 211 | SNP:c.1704G > A (p.Trp568Ter) | VCV000200370 | Not Prov.\|Cardiovas. pheno [symbol] | Pathogenic(LR: Sep. 15, 2017) |
| 450 | SNP:c.2168G > A (p.Cys723Tyr) | 112 | SNP:c.1468G > A (p.Ala490Thr) | VCV000014430 | Long QT Syn.\|Congenital long QT Syn.\|Long [symbol] | Pathogenic(LR: Sep. 21, 2019) |
| 451 | SNP:c.2171T > C (p.Leu724Pro) | 463 | SNP:c.2230C > T (p.Arg744Ter) | VCV000180383 | Long QT Syn. 2\|Cardiovas. pheno.\|Long QT [symbol] | Pathogenic(LR: Sep. 23, 2021) |
| 452 | SNP:c.2182A > T (p.Ile728Phe) | 950 | SNP:c.685G > T (p.Glu229Ter) | VCV000180379 | Long QT Syn. 2\|Not Prov.\|Long QT Syn. | Pathogenic(LR: Sep. 26, 2017) |
| 453 | SNP:c.2192A > C (p.His731Pro) | 298 | SNP:c.1841C > T (p.Ala614Val) | VCV000029777 | Long QT Syn.\|Congenital long QT Syn.\|Long [symbol] | Pathogenic(LR: Sep. 27, 2018) |
| 454 | SNP:c.2200C > T (p.Arg734Cys) | 970 | SNP:c.749C > A (p.Ser250Ter) | VCV000200301 | Not Prov. | Pathogenic(LR: Sep. 30, 2019) |
| 455 | SNP:c.2204C > T (p.Ser735Leu) | 380 | SNP:c.1943G > A (p.Gly648Asp) | VCV000200753 | Not Prov. | Pathogenic(LR: Sep. 9, 2011) |
| 456 | SNP:c.220A > C (p.Thr74Pro) | 301 | SNP:c.1847A > G (p.Tyr616Cys) | VCV000067295 | Not Prov.\|Long QT Syn.\|Congenital long QT [symbol] | Pathogenic/Likely pathogenic(LR: Apr. 12, 2019) |
| 457 | SNP:c.220A > G (p.Thr74Ala) | 187 | SNP:c.1682C > T (p.Ala561Val) | VCV000014420 | Cardiovas. pheno.\|Congenital long QT Syn.\| [symbol] | Pathogenic/Likely pathogenic(LR: Apr. 2, 2020) |
| 458 | SNP:c.2216A > G (p.His739Arg) | 534 | SNP:c.2467C > T (p.Arg823Trp) | VCV000067402 | Long QT Syn.\|Congenital long QT Syn.\|Card [symbol] | Pathogenic/Likely pathogenic(LR: Aug. 3, 2020) |
| 459 | SNP:c.221C > G (p.Thr74Arg) | 424 | SNP:c.2083C > T (p.Gln695Ter) | VCV000420923 | Not Prov.\|Long QT Syn. | Pathogenic/Likely pathogenic(LR: Dec. 23, 2017) |
| 460 | SNP:c.221C > T (p.Thr74Met) | 318 | SNP:c.1876G > A (p.Gly626Ser) | VCV000067304 | Long QT Syn. 2\|Congenital long QT Syn.\| [symbol] | Pathogenic/Likely pathogenic(LR: Dec. 27, 2019) |
| 461 | SNP:c.2221A > C (p.Lys741Gln) | 543 | SNP:c.2509G > A (p.Asp837Asn) | VCV000067405 | Long QT Syn. 2\| Long QT Syn.\|Congenital [symbol] | Pathogenic/Likely pathogenic(LR: Feb. 7, 2020) |
| 462 | SNP:c.2222A > G (p.Lys741Arg) | 345 | SNP:c.1898A > G (p.Asn633Ser) | VCV000067323 | Long QT Syn. 2\|Long QT Syn.\|Cardiovas. ph [symbol] | Pathogenic/Likely pathogenic(LR: Jan. 24, 2020) |
| 463 | SNP:c.2230C > T (p.Arg744Ter) | 431 | SNP:c.209A > G (p.His70Arg) | VCV000067363 | Long QT Syn.\|Congenital long QT Syn.\|Not [symbol] | Pathogenic/Likely pathogenic(LR: Jan. 26, 2021) |
| 464 | SNP:c.2231G > A (p.Arg744Gln) | 545 | SNP:c.2510A > G (p.Asp837Gly) | VCV000067407 | Long QT Syn. 2\|Long QT Syn.\|Congenital [symbol] | Pathogenic/Likely pathogenic(LR: Jul. 30, 2020) |
| 465 | SNP:c.2231G > C (p.Arg744Pro) | 331 | SNP:c.1886A > C (p.Asn629Thr) | VCV000067314 | Long QT Syn.\|Congenital long QT Syn.\|Not [symbol] | Pathogenic/Likely pathogenic(LR: Jul. 31, 2016) |
| 466 | SNP:c.2236G > T (p.Ala746Ser) | 523 | SNP:c.2414T > G (p.Phe805Cys) | VCV000067397 | Long QT Syn.\|Cardiovas. pheno.\|Congenital [symbol] | Pathogenic/Likely pathogenic(LR: Jul. 7, 2017) |
| 467 | SNP:c.223C > G (p.Gln75Glu) | 388 | SNP:c.1969G > A (p.Gly657Ser) | VCV000067348 | Congenital long QT Syn. Long QT Syn [symbol] | Pathogenic/Likely pathogenic(LR: Jun. 7, 2021) |
| 468 | SNP:c.2245G > T (p.Gly749Cys) | 470 | SNP:c.2254C > T (p.Arg752Trp) | VCV000067379 | Not Prov.\|Cardiovas. pheno.\|Long QT Syn.\| [symbol] | Pathogenic/Likely pathogenic(LR: Mar. 17, 2021) |
| 469 | SNP:c.2246G > T (p.Gly749Val) | 128 | SNP:c.1501G > A (p.Asp501Asn) | VCV000067208 | Long QT Syn.\|Congenital long QT Syn.\|Card [symbol] | Pathogenic/Likely pathogenic(LR: Mar. 22, 2019) |
| 470 | SNP:c.2254C > T (p.Arg752Trp) | 78 | SNP:c.1348C > T (p.Gln450Ter) | VCV000620154 | Not Prov.\|Long QT Syn. | Pathogenic/Likely pathogenic(LR: Mar. 23, 2021) |
| 471 | SNP:c.2255G > A (p.Arg752Gln) | 487 | SNP:c.2320G > T (p.Asp774Tyr) | VCV000067385 | Congenital long QT Syn.\|Long QT Syn. 2\|No [symbol] | Pathogenic/Likely pathogenic(LR: May 10, 2017) |
| 472 | SNP:c.2257G > T (p.Ala753Ser) | 62 | SNP:c.128A > G (p.Tyr43Cys) | VCV000067182 | Long QT Syn.\|Congenital long QT Syn.\| [symbol] | Pathogenic/Likely pathogenic(LR: May 21, 2020) |
| 473 | SNP:c.2261T > G (p.Leu754Arg) | 245 | SNP:c.1750G > A (p.Gly584Ser) | VCV000067261 | Long QT Syn.\|Long QT Syn. 2\|Short QT Syn [symbol] | Pathogenic/Likely pathogenic(LR: Oct. 11, 2021) |
| 474 | SNP:c.2263G > A (p.Ala755Thr) | 145 | SNP:c.157G > C (p.Gly53Arg) | VCV000067215 | Not Prov.\|Arrhythmia\|Congenital long QT Sy [symbol] | Pathogenic/Likely pathogenic(LR: Oct. 23, 2020) |
| 475 | SNP:c.2266A > G (p.Met756Val) | 444 | SNP:c.215C > A (p.Pro72Gln) | VCV000067368 | Congenital long QT Syn.\|Long QT Syn.\|Long [symbol] | Pathogenic/Likely pathogenic(LR: Oct. 3, 2020) |
| 476 | SNP:c.2271G > C (p.Lys757Asn) | 342 | SNP:c.1894C > T (p.Pro632Ser) | VCV000067321 | Cardiovas. pheno.\|Congenital long QT Syn.\|N [symbol] | Pathogenic/Likely pathogenic(LR: Sep. 17, 2019) |
| 477 | SNP:c.2276A > G (p.Lys759Arg) | 297 | SNP:c.1838C > T (p.Thr613Met) | VCV000067292 | Long QT Syn. 2\|Congenital long QT Syn.\|Sh [symbol] | Pathogenic/Likely pathogenic(LR: Sep. 20, 2020) |
| 478 | SNP:c.227G > T (p.Arg76Leu) | 529 | SNP:c.2453C > T (p.Ser818Leu) | VCV000014432 | Long QT Syn.\|Congenital long QT Syn.\|Long [symbol] | Pathogenic/Likely pathogenic(LR: Sep. 30, 2020) |
| 479 | SNP:c.2291C > G (p.Pro764Arg) | 416 | SNP:c.2054G > A (p.Arg685His) | VCV000979129 | Long QT Syn. 2 | Uncertain significance |
| 480 | SNP:c.2291C > T (p.Pro764Leu) | 491 | SNP:c.2332G > A (p.Ala778Thr) | VCV000979128 | Long QT Syn. 2 | Uncertain significance |
| 481 | SNP:c.2296G > A (p.Gly766Arg) | 688 | SNP:c.2870C > T (p.Pro957Leu) | VCV000979134 | Long QT Syn. 2 | Uncertain significance |
| 482 | SNP:c.2296G > T (p.Gly766Trp) | 956 | SNP:c.704T > C (p.Val235Ala) | VCV001049705 | Not Prov. | Uncertain significance |
| 483 | SNP:c.2299G > T (p.Asp767Tyr) | 1013 | SNP:c.877G > T (p.Ala293Ser) | VCV000200308 | Not Prov. | Uncertain significance(LR: Apr. 1, 2014) |
| 484 | SNP:c.2309T > C (p.Val770Ala) | 539 | SNP:c.2486A > C (p.Asp829Ala) | VCV000851362 | Long QT Syn. | Uncertain significance(LR: Apr. 1, 2020) |
| 485 | SNP:c.2312A > C (p.His771Pro) | 66 | SNP:c.1319C > T (p.Pro440Leu) | VCV000067185 | Arrhythmia\|Congenital long QT Syn.\|Not Pro [symbol] | Uncertain significance(LR: Apr. 1, 2021) |
| 486 | SNP:c.2312A > G (p.His771 Arg) | 1019 | SNP:c.89T > C (p.Ile30Thr) | VCV000067541 | Long QT Syn.\|Congenital long QT Syn. [symbol] | Uncertain significance(LR: Apr. 11, 2018) |
| 487 | SNP:c.2320G > T (p.Asp774Tyr) | 941 | SNP:c.652A > G (p.Met218Val) | VCV000067516 | Long QT Syn.\|Not Prov.\|Congenital long QT [symbol] | Uncertain significance(LR: Apr. 13, 2018) |
| 488 | SNP:c.2326C > T (p.Leu776Phe) | 877 | SNP:c.383A > G (p.Asn128Ser) | VCV000191473 | Long QT Syn.\|Not Prov. | Uncertain significance(LR: Apr. 14, 2017) |
| 489 | SNP:c.232G > A (p.Ala78Thr) | 844 | SNP:c.3379C > T (p.Leu1127Phe) | VCV000923705 | Arrhythmia | Uncertain significance(LR: Apr. 14, 2019) |
| 490 | SNP:c.232G > C (p.Ala78Pro) | 157 | SNP:c.1603G > A (p.Val535Met) | VCV000161256 | Long QT Syn. 2\|Not Prov.\|Long QT Syn.\|Car [symbol] | Uncertain significance(LR: Apr. 15, 2019) |
| 491 | SNP:c.2332G > A (p.Ala778Thr) | 862 | SNP:c.3464C > T (p.Ser1155Leu) | VCV000580441 | Long QT Syn. | Uncertain significance(LR: Apr. 16, 2018) |
| 492 | SNP:c.233C > T (p.Ala78Val) | 13 | SNP:c.1055C > G (p.Pro352Arg) | VCV001038936 | Long QT Syn. | Uncertain significance(LR: Apr. 19, 2018) |
| 493 | SNP:c.2343C > G (p.Phe781Leu) | 405 | SNP:c.2014C > T (p.Arg672Cys) | VCV001020103 | Long QT Syn. | Uncertain significance(LR: Apr. 21, 2020) |
| 494 | SNP:c.2345T > C (p.Ile782Thr) | 932 | SNP:c.58C > G (p.Arg20Gly) | VCV000067513 | Not Prov.\|Congenital long QT Syn. | Uncertain significance(LR: Apr. 22, 2020) |
| 495 | SNP:c.2350C > T (p.Arg784Trp) | 933 | SNP:c.602C > T (p.Thr201Met) | VCV000200295 | Not Prov. | Uncertain significance(LR: Apr. 23, 2013) |
| 496 | SNP:c.2354G > C (p.Gly785Ala) | 494 | SNP:c.2345T > C (p.Ile 782Thr) | VCV000977169 | Long QT Syn. | Uncertain significance(LR: Apr. 23, 2019) |
| 497 | SNP:c.2354G > T (p.Gly785Val) | 159 | SNP:c.1609C > T (p.Arg537Trp) | VCV000067222 | Congenital long QT Syn.\|Arrhythmia\|Long Q [symbol] | Uncertain significance(LR: Apr. 23, 2020) |
| 498 | SNP:c.235G > A (p.Ala79Thr) | 154 | SNP:c.1600C > G (p.Arg534Gly) | VCV000908910 | Long QT Syn. 2 | Uncertain significance(LR: Apr. 27, 2017) |
| 499 | SNP:c.235G > C (p.Ala79Pro) | 32 | SNP:c.1190G > A (p.Arg397His) | VCV000851410 | Long QT Syn.\|Arrhythmia | Uncertain significance(LR: Apr. 27, 2020) |
| 500 | SNP:c.235G > T (p.Ala79Ser) | 343 | SNP:c.1897A > C (p.Asn633His) | VCV001001814 | Long QT Syn. | Uncertain significance(LR: Apr. 27, 2020) |
| 501 | SNP:c.2360T > A (p.Ile787Asn) | 473 | SNP:c.2261T > G (p.Leu754Arg) | VCV000200767 | Not Prov. | Uncertain significance(LR: Apr. 28, 2017) |
| 502 | SNP:c.2362G > A (p.Glu788Lys) | 409 | SNP:c.2033T > C (p.Leu678Pro) | VCV000067356 | Long QT Syn.\|Congenital long QT Syn. | Uncertain significance(LR: Apr. 30, 2016) |
| 503 | SNP:c.2364G > C (p.Glu788Asp) | 776 | SNP:c.3124C > G (p.Leu1042Val) | VCV000427971 | Hypertrophic cardiomyopathy | Uncertain significance(LR: Apr. 30, 2017) |
| 504 | SNP:c.2366T > C (p.Ile789Thr) | 175 | SNP:c.1661T > C (p.Met554Thr) | VCV000642793 | Long QT Syn. | Uncertain significance(LR: Apr. 30, 2019) |
| 505 | SNP:c.2369T > C (p.Leu790Pro) | 1037 | SNP:c.950A > G (p.Asn317Ser) | VCV000405346 | Arrhythmia\|Short QT Syn. 1\|Long QT Syn. 2 [symbol] | Uncertain significance(LR: Apr. 30, 2019) |
| 506 | SNP:c.2371C > T (p.Arg791Trp) | 850 | SNP:c.3406C > T (p.Leu136Phe) | VCV001035669 | Long QT Syn. | Uncertain significance(LR: Apr. 30, 2020) |
| 507 | SNP:c.2372G > A (p.Arg791Gln) | 823 | SNP:c.3296C > T (p.Pro1099Leu) | VCV000526951 | Long QT Syn. | Uncertain significance(LR: Apr. 4, 2018) |
| 508 | SNP:c.2377G > A (p.Asp793Asn) | 97 | SNP:c.1982C > T (p.Ala661Val) | VCV000977179 | Sudden cardiac arrest | Uncertain significance(LR: Apr. 7, 2020) |
| 509 | SNP:c.2380G > A (p.Val794Ile) | 643 | SNP:c.2768C > T (p.Pro926Leu) | VCV000572279 | Long QT Syn.\|Long QT Syn. 2\|Arrhythmia | Uncertain significance(LR: Apr. 7, 2020) |
| 510 | SNP:c.2383G > A (p.Val795Ile) | 771 | SNP:c.3109G > A (p.Asp1037Asn) | VCV000067470 | Long QT Syn.\|Arrhythmia\|Not Prov. | Uncertain significance(LR: Apr. 7, 2020) |
| 511 | SNP:c.2386G > C (p.Val796Leu) | 277 | SNP:c.1808G > A (p.Gly603Asp) | VCV000456894 | Long QT Syn. | Uncertain significance(LR: Apr. 8, 2020) |
| 512 | SNP:c.2386G > T (p.Val796Leu) | 749 | SNP:c.3041G > A (p.Arg1014Gln) | VCV000922413 | Not Prov.\|Long QT Syn.\|Arrhythmia | Uncertain significance(LR: Apr. 8, 2020) |
| 513 | SNP:c.2387T > G (p.Val796Gly) | 994 | SNP:c.823A > C (p.Ser275Arg) | VCV000577151 | Long QT Syn. | Uncertain significance(LR: Apr. 8, 2020) |
| 514 | SNP:c.238G > A (p.Ala80Thr) | 894 | SNP:c.446G > A (p.Gly149Asp) | VCV000563779 | Long QT Syn. | Uncertain significance(LR: Apr. 9, 2019) |
| 515 | SNP:c.238G > C (p.Ala80Pro) | 946 | SNP:c.664G > A (p.Val222Met) | VCV000835460 | Long QT Syn. | Uncertain significance(LR: Apr. 9, 2019) |
| 516 | SNP:c.2390C > A (p.Ala797Asp) | 133 | SNP:c.1519C > G (p.Pro507Ala) | VCV000656162 | Long QT Syn. | Uncertain significance(LR: Aug. 1, 2018) |
| 517 | SNP:c.2396T > C (p.Leu799Pro) | 949 | SNP:c.683C > T (p.Ala228Val) | VCV000659941 | Long QT Syn. | Uncertain significance(LR: Aug. 10, 2018) |
| 518 | SNP:c.2398G > T (p.Gly800Trp) | 1047 | SNP:c.977G > A (p.Arg326His) | VCV000649300 | Long QT Syn. | Uncertain significance(LR: Aug. 10, 2018) |
| 519 | SNP:c.2399G > A (p.Gly800Glu) | 707 | SNP:c.2913G > T (p.Glu971Asp) | VCV000926285 | Arrhythmia | Uncertain significance(LR: Aug. 10, 2020) |
| 520 | SNP:c.239C > T (p.Ala80Val) | 797 | SNP:c.3187G > C (p.Val1063Leu) | VCV000845043 | Long QT Syn. | Uncertain significance(LR: Aug. 10, 2020) |
| 521 | SNP:c.2405A > G (p.Asn802Ser) | 568 | SNP:c.2588G > A (p.Arg863Gln) | VCV001026816 | Long QT Syn. | Uncertain significance(LR: Aug. 11, 2020) |
| 522 | SNP:c.2414T > C (p.Phe805Cys) | 749 | SNP:c.3032A > G (p.Glu1011Gly) | VCV000922211 | Long QT Syn.\|Arrhythmia | Uncertain significance(LR: Aug. 11, 2020) |
| 523 | SNP:c.2414T > G (p.Phe85Glu) | 951 | SNP:c.692G > A (p.Arg231Gln) | VCV000952892 | Long QT Syn. | Uncertain significance(LR: Aug. 11, 2020) |
| 524 | SNP:c.2417G > A (p.Gly806Ala) | 71 | SNP:c.1330G > A (p.Glu444Lys) | VCV000191472 | Arrhythmia\|Not Prov.\|Long QT Syn.\|Long Q [symbol] | Uncertain significance(LR: Aug. 14, 2019) |
| 525 | SNP:c.2417G > C (p.Gly806Thr) | 542 | SNP:c.2504G > A (p.Arg835Gln) | VCV000200464 | Not Prov. | Uncertain significance(LR: Aug. 14, 2019) |
| 526 | SNP:c.2437G > A (p.Ala813His) | 650 | SNP:c.2774G > C (p.Gly925Ala) | VCV000200495 | Not Prov. | Uncertain significance(LR: Aug. 17, 2018) |
| 527 | SNP:c.243G > C (p.Gln81Pro) | 24 | SNP:c.1099A > T (p.Thr367Ser) | VCV000067164 | Not Prov.\|Arrhythmia | Uncertain significance(LR: Aug. 18, 2020) |
| 528 | SNP:c.2452T > C (p.Ser818Leu) | 103 | SNP:c.935G > A (p.Arg312His) | VCV000456942 | Arrhythmia Not Prov.\|Long QT Syn. | Uncertain significance(LR: Aug. 18, 2020) |
| 529 | SNP:c.2453C > T (p.Ser818Arg) | 404 | SNP:c.2012C > G (p.Ala671Gly) | VCV001024776 | Long QT Syn. | Uncertain significance(LR: Aug. 2, 2020) |
| 530 | SNP:c.2458G > A (p.Gly820Thr) | 767 | SNP:c.3098G > T (p.Arg1033Leu) | VCV001059933 | Long QT Syn. | Uncertain significance(LR: Aug. 2, 2020) |
| 531 | SNP:c.245T > C (p.Ile82Met) | 880 | SNP:c.38C > A (p.Thr13Asn) | VCV000923223 | Long QT Syn.\|Arrhythmia | Uncertain significance(LR: Aug. 2, 2020) |
| 532 | SNP:c.2464G > A (p.Val822Leu) | 881 | SNP:c.391G > A (p.Val131Met) | VCV000920295 | Arrhythmia | Uncertain significance(LR: Aug. 20, 2019) |
| 533 | SNP:c.2464G > C (p.Val822Trp) | 373 | SNP:c.1930G > T (p.Val644Phe) | VCV000067340 | Congenital long QT Syn.\|Long QT Syn. | Uncertain significance(LR: Aug. 20, 2020) |
| 534 | SNP:c.2467C > T (p.Arg823Gln) | 1043 | SNP:c.967G > A (p.Asp323Asn) | VCV000067551 | Congenital long QT Syn.\|Long QT Syn. | Uncertain significance(LR: Aug. 20, 2020) |
| 535 | SNP:c.2468G > A (p.Arg823Ile) | 781 | SNP:c.3137A > G (p.Gln1046Arg) | VCV000958659 | Long QT Syn. | Uncertain significance(LR: Aug. 21, 2019) |
| 536 | SNP:c.2477C > T (p.Thr826Ser) | 829 | SNP:c.331G > A (p.Asp111Asn) | VCV000921508 | Arrhythmia | Uncertain significance(LR: Aug. 21, 2019) |
| 537 | SNP:c.247G > T (p.Ala83Arg) | 294 | SNP:c.1832A > G (p.Tyr611Cys) | VCV001019935 | Long QT Syn. | Uncertain significance(LR: Aug. 22, 2020) |
| 538 | SNP:c.2482T > C (p.Cys828Ala) | 942 | SNP:c.655G > A (p.Asp219Asn) | VCV000663570 | Long QT Syn. | Uncertain significance(LR: Aug. 23, 2018) |
| 539 | SNP:c.2486A > C (p.Asp829Ter) | 438 | SNP:c.2122A > G (p.Thr708Ala) | VCV000527003 | Long QT Syn | Uncertain significance(LR: Aug. 24, 2017) |
| 540 | SNP:c.2494A > T (p.Lys832Trp) | 134 | SNP:c.1519C > T (p.Pro507Ser) | VCV000200350 | Not Prov. | Uncertain significance(LR: Aug. 25, 2017) |
| 541 | SNP:c.2503C > T (p.Arg835Gln) | 807 | SNP:c.3215C > T (p.Thr1072Met) | VCV000359303 | Long QT Syn. 2\|Arrhythmia\|Not Prov.\|Long [symbol] | Uncertain significance(LR: Aug. 25, 2020) |
| 542 | SNP:c.2504G > A (p.Arg835Asn) | 864 | SNP:c.3469C > T (p.Pro1157Ser) | VCV000456933 | Arrhythmia\|Long QT Syn. | Uncertain significance(LR: Aug. 26, 2019) |
| 543 | SNP:c.2509G > A (p.Asp837Tyr) | 65 | SNP:c.1307C > T (p.Thr436Met) | VCV000067184 | Arrhythmia\|Long QT Syn.\|Not Prov.\|Congeni [symbol] | Uncertain significance(LR: Aug. 26, 2020) |
| 544 | SNP:c.2509G > T (p.Asp837Gly) | 640 | SNP:c.2765G > A (p.Arg922Gln) | VCV000067436 | Long QT Syn.\|Not Prov.\|Congenital long QT [symbol] | Uncertain significance(LR: Aug. 26, 2020) |
| 545 | SNP:c.2510A > G (p.Asp837Met) | 44 | SNP:c.1231G > A (p.Asp411Asn) | VCV000941613 | Long QT Syn. | Uncertain significance(LR: Aug. 27, 2019) |
| 546 | SNP:c.2515C > A (p.Leu839Arg) | 257 | SNP:c.1771G > A (p.Asp591Asn) | VCV000911854 | Long QT Syn. 2\| Long QT Syn.\|Arrhythmia | Uncertain significance(LR: Aug. 27, 2020) |
| 547 | SNP:c.251A > G (p.Gln84Pro) | 429 | SNP:c.2094G > T (p.Glu698Asp) | VCV001001384 | Long QT Syn. | Uncertain significance(LR: Aug. 27, 2020) |
| 548 | SNP:c.2525T > C (p.Leu842Pro) | 934 | SNP:c.607G > A (p.Ala203Thr) | VCV000067514 | Cardiovas. pheno.\|Not Prov.\|Long QT Syn. | Uncertain significance(LR: Aug. 27, 2020) |
| 549 | SNP:c.2530A > G (p.Met844Val) | 808 | SNP:c.3224C > A (p.Pro1075Gln) | VCV000629130 | Arrhythmia | Uncertain significance(LR: Aug. 28, 2018) |
| 550 | SNP:c.2536C > A (p.Pro846Thr) | 1002 | SNP:c.853G > A (p.Ala285Thr) | VCV000220070 | Long QT Syn. | Uncertain significance(LR: Aug. 29, 2015) |
| 551 | SNP:c.2536C > G (p.Pro846Ala) | 1046 | SNP:c.976C > T (p.Arg326Cys) | VCV001040723 | Long QT Syn. | Uncertain significance(LR: Aug. 29, 2020) |
| 552 | SNP:c.2536C > T (p.Pro846Ser) | 391 | SNP:c.196T > C (p.Cys66Arg) | VCV000456900 | Long QT Syn. | Uncertain significance(LR: Aug. 3, 2017) |
| 553 | SNP:c.2537C > T (p.Pro846Leu) | 105 | SNP:c.1426G > A (p.Val476Ile) | VCV000067200 | Long QT Syn.\|Congenital long QT Syn.\|not [symbol] | Uncertain significance(LR: Aug. 3, 2020) |
| 554 | SNP:c.253G > A (p.Ala85Thr) | 666 | SNP:c.280G > A (p.Val94Met) | VCV001055911 | Long QT Syn. | Uncertain significance(LR: Aug. 3, 2020) |
| 555 | SNP:c.253G > C (p.Ala85Pro) | 210 | SNP:c.1703G > C (p.Trp568Ser) | VCV000520444 | Long QT Syn. | Uncertain significance(LR: Aug. 31, 2016) |
| 556 | SNP:c.2548G > T (p.Asp850Tyr) | 69 | SNP:c.1325C > T (p.Ala442Val) | VCV000456885 | Long QT Syn.\|Long QT Syn. 2\|Short QT Syn [symbol] | Uncertain significance(LR: Aug. 31, 2018) |
| 557 | SNP:c.254C > T (p.Ala85Val) | 208 | SNP:c.1700T > C (p.Ile567Thr) | VCV000067241 | Congenital long QT Syn.\|Long QT Syn. | Uncertain significance(LR: Aug. 31, 2018) |
| 558 | SNP:c.2566C > G (p.Leu856Val) | 967 | SNP:c.731C > T (p.Ala244Val) | VCV000405349 | Long QT Syn. | Uncertain significance(LR: Aug. 31, 2018) |
| 559 | SNP:c.2573T > C (p.Ile858Thr) | 916 | SNP:c.530A > C (p.Glu177Ala) | VCV001045316 | Long QT Syn. | Uncertain significance(LR: Aug. 4, 2020) |
| 560 | SNP:c.2576C > T (p.Thr859Ile) | 958 | SNP:c.707G > T (p.Gly236Val) | VCV000067518 | Long QT Syn.\|Not Prov. | Uncertain significance(LR: Aug. 4, 2021) |
| 561 | SNP:c.257T > C (p.Leu86Pro) | 874 | SNP:c.374T > G (p.Phe125Cys) | VCV000067502 | Cardiovas. pheno.\|Congenital long QT Syn. | Uncertain significance(LR: Aug. 5, 2015) |
| 562 | SNP:c.257T > G (p.Leu86Arg) | 815 | SNP:c.3263C > G (p.Pro1088Arg) | VCV000954908 | Long QT Syn. | Uncertain significance(LR: Aug. 5, 2019) |
| 563 | SNP:c.2581A > C (p.Asn861His) | 351 | SNP:c.1904A > T (p.Asn635Ile) | VCV000067327 | Long QT Syn.\|Congenital long QT Syn. | Uncertain significance(LR: Aug. 6, 2019) |
| 564 | SNP:c.2582A > C (p.Asn861Thr) | 698 | SNP:c.2893G > A (p.Gly965Arg) | VCV000067450 | Long QT Syn.\|Long QT Syn. 2\|Not Prov. | Uncertain significance(LR: Aug. 6, 2020) |
| 565 | SNP:c.2582A > G (p.Asn861Ser) | 1011 | SNP:c.875G > A (p.Arg292His) | VCV001017664 | Long QT Syn. | Uncertain significance(LR: Aug. 7, 2020) |
| 566 | SNP:c.2582A > T (p.Asn861Ile) | 840 | SNP:c.3358C > G (p.Leu1120Val) | VCV000661932 | Long QT Syn. | Uncertain significance(LR: Aug. 8, 2018) |
| 567 | SNP:c.2587C > T (p.Arg863Ter) | 17 | SNP:c.1071G > T (p.Glu357Asp) | VCV000628850 | Arrhythmia | Uncertain significance(LR: Aug. 8, 2019) |
| 568 | SNP:c.2588G > A (p.Arg863Gln [symbol] | 593 | SNP:c.2660G > T (p.Arg887Leu) | VCV000628973 | Arrhythmia | Uncertain significance(LR: Aug. 8, 2019) |
| 569 | SNP:c.2591A > G (p.Asp864Gly [symbol] | 904 | SNP:c.460T > C (p.Trp 154Arg) | VCV000628868 | Arrhythmia | Uncertain significance(LR: Aug. 8, 2019) |
| 570 | SNP:c.2594C > T (p.Thr865Ile) | 308 | SNP:c.1859G > A (p.Ser620Asn) | VCV001017668 | Long QT Syn. | Uncertain significance(LR: Aug. 9, 2020) |
| 571 | SNP:c.2600T > C (p.Met867Thr) | 924 | SNP:c.563C > T (p.Ala188Val) | VCV000200293 | Long QT Syn.\|Not Prov. | Uncertain significance(LR: Aug. 9, 2020) |
| 572 | SNP:c.2606C > T (p.Pro869Leu) | 91 | SNP:c.1391T > C (p.Ile464Thr) | VCV000919164 | Arrhythmia | Uncertain significance(LR: Dec. 10, 2018) |
| 573 | SNP:c.260T > C (p.Leu87Pro) | 1027 | SNP:c.928C > A (p.Pro310Thr) | VCV000925941 | Arrhythmia | Uncertain significance(LR: Dec. 10, 2018) |
| 574 | SNP:c.2612C > G (p.Ser871Cys) | 464 | SNP:c.2231G > A (p.Arg744Gln) | VCV000924279 | Arrhythmia | Uncertain significance(LR: Dec. 10, 2020) |
| 575 | SNP:c.2612C > T (p.Ser871Phe) | 610 | SNP:c.2696C > T (p.Thr899Met) | VCV000862133 | Long QT Syn.\|Arrhythmia | Uncertain significance(LR: Dec. 10, 2020) |
| 576 | SNP:c.2617G > A (p.Gly873Ser) | 891 | SNP:c.440A > G (p.His 147Arg) | VCV000200285 | Not Prov.\|Long QT Syn. 2\|Short QT Syn. 1\| [symbol] | Uncertain significance(LR: Dec. 11, 2018) |
| 577 | SNP:c.2624C > G (p.Thr875 Arg) | 652 | SNP:c.2777C > T (p.Pro926Leu) | VCV000848808 | Long QT Syn. | Uncertain significance(LR: Dec. 11, 2019) |
| 578 | SNP:c.2624C > T (p.Thr875Met) | 863 | SNP:c.3467A > G (p.Asp1156Gly) | VCV000926485 | Arrhythmia | Uncertain significance(LR: Dec. 11, 2019) |
| 579 | SNP:c.2626G > A (p.Glu876Lys) | 419 | SNP:c.206T > A (p.Leu69Gln) | VCV000520445 | Long QT Syn. | Uncertain significance(LR: Dec. 12, 2016) |
| 580 | SNP:c.2626G > T (p.Glu876Ter) | 572 | SNP:c.2606C > T (p.Pro869Leu) | VCV000200473 | Arrhythmia\|Not Prov.\|Brugada Syn. 1 | Uncertain significance(LR: Dec. 13, 2019) |
| 581 | SNP:c.2634G > T (p.Glu878Asp) | 599 | SNP:c.2680C > T (p.Arg894Cys) | VCV000067424 | Long QT Syn.\|Not Prov.\|Congenital long QT [symbol] | Uncertain significance(LR: Dec. 13, 2019) |
| 582 | SNP:c.2635G > A (p.Gly879Ser) | 554 | SNP:c.253G > A (p.Ala85Thr) | VCV000456912 | Long QT Syn. | Uncertain significance(LR: Dec. 14, 2018) |
| 583 | SNP:c.2635G > C (p.Gly879Arg) | 902 | SNP:c.455C > G (p.Thr152Ser) | VCV000200288 | Arrhythmia\|Not Prov. | Uncertain significance(LR: Dec. 14, 2020) |
| 584 | SNP:c.2641T > A (p.Phe881Ile) | 897 | SNP:c.449C > T (p.Pro150Leu) | VCV001171810 | Arrhythmia | Uncertain significance(LR: Dec. 15, 2020) |
| 585 | SNP:c.2647C > T (p.Arg883Trp) | 833 | SNP:c.3343A > G (p.Met1115Val) | VCV000067490 | Not Prov.\|Arrhythmia\|Congenital long QT Sy [symbol] | Uncertain significance(LR: Dec. 16, 2019) |
| 586 | SNP:c.2648G > A (p.Arg883Gln [symbol] | 668 | SNP:c.2810G > A (p.Ser937Asn) | VCV000067440 | Congenital long QT Syn.\|Long QT Syn.\|Arrh [symbol] | Uncertain significance(LR: Dec. 16, 2020) |
| 587 | SNP:c.2651A > G (p.Gln884Arg [symbol] | 980 | SNP:c.773C > T (p.Pro258Leu) | VCV000495747 | Not Prov. | Uncertain significance(LR: Dec. 19, 2016) |
| 588 | SNP:c.2653C > T (p.Arg885Cys) | 683 | SNP:c.2857C > T (p.Leu953Phe) | VCV000924479 | Arrhythmia | Uncertain significance(LR: Dec. 19, 2018) |
| 589 | SNP:c.2654G > A (p.Arg885His) | 702 | SNP:c.2903C > T (p.Pro968Leu) | VCV000067452 | Arrhythmia\|Not Prov.\|Long QT Syn. 2 | Uncertain significance(LR: Dec. 19, 2018) |
| 590 | SNP:c.2659C > G (p.Arg887Gly) | 1015 | SNP:c.880G > A (p.Gly294Arg) | VCV000838034 | Long QT Syn. | Uncertain significance(LR: Dec. 19, 2019) |
| 591 | SNP:c.2659C > T (p.Arg887Cys) | 499 | SNP:c.235G > C (p.Ala79Pro) | VCV000200778 | Not Prov. | Uncertain significance(LR: Dec. 2, 2019) |
| 592 | SNP:c.2660G > A (p.Arg887His) | 271 | SNP:c.1796G > A (p.Ser599Asn) | VCV001172277 | Arrhythmia | Uncertain significance(LR: Dec. 21, 2020) |
| 593 | SNP:c.2660G > T (p.Arg887Leu) | 482 | SNP:c.2296G > T (p.Gly766Trp) | VCV000392164 | Not Prov. | Uncertain significance(LR: Dec. 22, 2016) |
| 594 | SNP:c.2665T > G (p.Leu889Val) | 760 | SNP:c.3089C > T (p.Pro1030Leu) | VCV000847541 | Not Prov.\|Long QT Syn.\|Arrhythmia | Uncertain significance(LR: Dec. 22, 2020) |
| 595 | SNP:c.2668T > C (p.Ser890Pro) | 493 | SNP:c.2343C > G (p.Phe781Leu) | VCV000264633 | Cardiovas. pheno. | Uncertain significance(LR: Dec. 23, 2015) |
| 596 | SNP:c.2674C > T (p.Arg892Cys) | 480 | SNP:c.2291C > T (p.Pro764Leu) | VCV000267296 | Arrhythmia\|Long QT Syn. | Uncertain significance(LR: Dec. 23, 2019) |
| 597 | SNP:c.2675G > A (p.Arg892His) | 635 | SNP:c.2758C > T (p.Arg920Trp) | VCV000067433 | Arrhythmia\|Long QT Syn.\|Congenital long Q [symbol] | Uncertain significance(LR: Dec. 23, 2019) |
| 598 | SNP:c.2678G > A (p.Arg893Lys) | 630 | SNP:c.2684C > A (p.Thr895Lys) | VCV000927873 | Arrhythmia | Uncertain significance(LR: Dec. 23, 2020) |
| 599 | SNP:c.2680C > T (p.Arg894Cys) | 139 | SNP:c.1567C > A (p.Leu523Met) | VCV000405357 | Long QT Syn. | Uncertain significance(LR: Dec. 24, 2016) |
| 600 | SNP:c.2681G > A (p.Arg894His) | 514 | SNP:c.238G > A (p.Ala80Thr) | VCV001311334 | Not Prov. | Uncertain significance(LR: Dec. 27, 2019) |
| 601 | SNP:c.2681G > T (p.Arg894Leu) | 735 | SNP:c.3003G > C (p.Trp1001Cys) | VCV000857986 | Long QT Syn. | Uncertain significance(LR: Dec. 27, 2019) |
| 602 | SNP:c.2684C > A (p.Thr895Lys) | 181 | SNP:c.1676T > C (p.Leu559Pro) | VCV000656408 | Long QT Syn. | Uncertain significance(LR: Dec. 28, 2018) |
| 603 | SNP:c.2684C > G (p.Thr895Arg) | 731 | SNP:c.299G > T (p.Arg100Leu) | VCV000657216 | Long QT Syn. | Uncertain significance(LR: Dec. 28, 2018) |
| 604 | SNP:c.2684C > T (p.Thr895Met) | 636 | SNP:c.2759G > A (p.Arg920Gln) | VCV000067434 | Not Prov.\|Arrhythmia\|Cardiovas. pheno.\|Con [symbol] | Uncertain significance(LR: Dec. 29, 2020) |
| 605 | SNP:c.2690A > C (p.Lys897Thr) | 645 | SNP:c.3392G > T (p.Gly1131Val) | VCV000918364 | Arrhythmia | Uncertain significance(LR: Dec. 3, 2018) |
| 606 | SNP:c.2690A > T (p.Lys897Met) | 168 | SNP:c.1640C > T (p.Ala547Val) | VCV000852580 | Long QT Syn.\|Arrhythmia | Uncertain significance(LR: Dec. 3, 2019) |
| 607 | SNP:c.2695A > T (p.Thr899Ser) | 830 | SNP:c.3320C > T (p.Ser1107Leu) | VCV000843735 | Long QT Syn.\|Short QT Syn. 1 | Uncertain significance(LR: Dec. 3, 2019) |
| 608 | SNP:c.2696C > A (p.Thr899Lys) | 15 | SNP:c.1066C > T (p.Arg356Cys) | VCV000925269 | Arrhythmia | Uncertain significance(LR: Dec. 3, 2020) |
| 609 | SNP:c.2696C > G (p.Thr899Arg) | 386 | SNP:c.1963A > G (p.Ile655Val) | VCV000405359 | Long QT Syn. | Uncertain significance(LR: Dec. 30, 2016) |
| 610 | SNP:c.2696C > T (p.Thr899Met) | 560 | SNP:c.2576C > T (p.Thr859Ile) | VCV000200469 | Not Prov. | Uncertain significance(LR: Dec. 31, 2019) |
| 611 | SNP:c.26C > T (p.Ala9Val) | 846 | SNP:c.3394C > G (p.Pro1132Ala) | VCV000191469 | Arrhythmia\|Long QT Syn.\|Not Prov. | Uncertain significance(LR: Dec. 31, 2019) |
| 612 | SNP:c.2701C > T (p.Gln901Ter) | 561 | SNP:c.257T > C (p.Leu86Pro) | VCV000067413 | Congenital long QT Syn.\|Not Prov. | Uncertain significance(LR: Dec. 31, 2020) |
| 613 | SNP:c.2702A > G (p.Gln901Arg) | 925 | SNP:c.566G > T (p.gly189Val) | VCV000637978 | Long QT Syn. 2 | Uncertain significance(LR: Dec. 4, 2018) |
| 614 | SNP:c.2707G > A (p.Gly903Arg) | 510 | SNP:c.2383G > A (p.val795Ile) | VCV001171063 | Arrhythmia | Uncertain significance(LR: Dec. 4, 2020) |
| 615 | SNP:c.2707G > C (p.Gly903Arg) | 625 | SNP:c.2735G > A (p.Arg912Gln) | VCV000527012 | Long QT Syn.\|Arrhythmia | Uncertain significance(LR: Dec. 4, 2020) |
| 616 | SNP:c.2710G > A (p.Glu904Lys) | 676 | SNP:c.2842C > T (p.Arg948Cys) | VCV000014441 | Congenital long QT Syn.\|Long QT Syn. 1/2, [symbol] | Uncertain significance(LR: Dec. 4, 2020) |
| 617 | SNP:c.2711A > G (p.Glu904Gly) | 739 | SNP:c.3014G > A (p.Arg1005Gln) | VCV000067461 | Arrhythmia\|Congenital long QT Syn. | Uncertain significance(LR: Dec. 6, 2019) |
| 618 | SNP:c.2711A > T (p.Glu904Val) | 95 | SNP:c.1397A > T (p.Asp466Val) | VCV000646851 | Long QT Syn. | Uncertain significance(LR: Dec. 7, 2018) |
| 619 | SNP:c.2717C > T (p.Ser906Leu) | 238 | SNP:c.1742C > T (p.Ser581Leu) | VCV000577995 | Arrhythmia\|Long QT Syn. | Uncertain significance(LR: Dec. 9, 2020) |
| 620 | SNP:c.2719G > A (p.Ala907Thr) | 680 | SNP:c.2854C > T (p.Pro952Ser) | VCV000919402 | Arrhythmia\|Not Prov.\|Long QT Syn. | Uncertain significance(LR: Dec. 9, 2020) |
| 621 | SNP:c.2719G > T (p.Ala907Ser) | 63 | SNP:c.1291T > C (p.Phe431Leu) | VCV000519399 | Cardiovas. pheno.\|Long QT Syn. | Uncertain significance(LR: Feb. 1, 2020) |
| 622 | SNP:c.2729C > T (p.Pro910Leu) | 762 | SNP:c.3094C > T (p.Arg1032Trp) | VCV000200522 | Long QT Syn.\|Arrhythmia\|Not Prov.\|Long Q [symbol] | Uncertain significance(LR: Feb. 1, 2021) |
| 623 | SNP:c.2731G > C (p.Gly911Arg) | 109 | SNP:c.1460G > C (p.Gly487Ala) | VCV000921335 | Arrhythmia | Uncertain significance(LR: Feb. 11, 2019) |
| 624 | SNP:c.2734C > T (p.Arg912Trp) | 607 | SNP:c.2695A > T (p.Thr899Ser) | VCV000919954 | Arrhythmia | Uncertain significance(LR: Feb. 11, 2019) |
| 625 | SNP:c.2735G > A (p.Arg912Gln) | 928 | SNP:c.572C > T (p.Pro191Leu) | VCV001027196 | Long QT Syn. | Uncertain significance(LR: Feb. 11, 2020) |
| 626 | SNP:c.2737G > A (p.Ala913Thr) | 521 | SNP:c.2405A > G (p.Asn802Ser) | VCV000216326 | Long QT Syn. | Uncertain significance(LR: Feb. 12, 2015) |
| 627 | SNP:c.2738C > T (p.Ala913Val) | 608 | SNP:c.2696C > A (p.Thr899Lys) | VCV001006883 | Long QT Syn. | Uncertain significance(LR: Feb. 12, 2020) |
| 628 | SNP:c.273G > T (p.Glu91Asp) | 974 | SNP:c.755G > A (p.Arg252Gln) | VCV000180380 | Long QT Syn. | Uncertain significance(LR: Feb. 12, 2020) |
| 629 | SNP:c.2744C > T (p.Ala915Val) | 614 | SNP:c.2707G > A (p.Gly903Arg) | VCV000067428 | Congenital long QT Syn.\|not spec.\|Long QT [symbol] | Uncertain significance(LR: Feb. 12, 2021) |
| 630 | SNP:c.2746G > A (p.Gly916Arg) | 770 | SNP:c.3107G > A (p.Gly1036Asp) | VCV000067469 | not spec.\|Long QT Syn.\|Not Prov.\|Congenital [symbol] | Uncertain significance(LR: Feb. 12, 2021) |
| 631 | SNP:c.274C > T (p.Arg92Cys) | 613 | SNP:c.2702A > G (p.Gln901Arg) | VCV000851829 | Long QT Syn. | Uncertain significance(LR: Feb. 13, 2019) |
| 632 | SNP:c.2750C > T (p.Pro917Leu) | 1016 | SNP:c.881G > T (p.Gly294Val) | VCV000067539 | Long QT Syn.\|SUDDEN INFANT DEATH S [symbol] | Uncertain significance(LR: Feb. 14, 2019) |
| 633 | SNP:c.2753G > A (p.Ser918Asn) | 1033 | SNP:c.93C > G (p.Ile31Met) | VCV000839172 | Long QT Syn. | Uncertain significance(LR: Feb. 14, 2019) |
| 634 | SNP:c.2758C > G (p.Arg920Gly) | 791 | SNP:c.3170G > A (p.Ser1057Asn) | VCV001022238 | Long QT Syn. | Uncertain significance(LR: Feb. 14, 2020) |
| 635 | SNP:c.2758C > T (p.Arg920Trp) | 745 | SNP:c.302A > G (p.Lys101Arg) | VCV001256427 | Not Prov. | Uncertain significance(LR: Feb. 15, 2021) |
| 636 | SNP:c.2759G > A (p.Arg920Gln) | 383 | SNP:c.194C > G (p.Thr65Ser) | VCV000237295 | Long QT Syn. | Uncertain significance(LR: Feb. 17, 2016) |
| 637 | SNP:c.275G > C (p.Arg92Pro) | 371 | SNP:c.1930G > A (p.Val644Ile) | VCV001009718 | Long QT Syn. | Uncertain significance(LR: Feb. 17, 2020) |
| 638 | SNP:c.275G > T (p.Arg92Leu) | 979 | SNP:c.772C > T (p.Pro258Ser) | VCV001060097 | Long QT Syn. | Uncertain significance(LR: Feb. 17, 2020) |
| 639 | SNP:c.2764C > T (p.Arg922Trp) | 647 | SNP:c.2771G > C (p.Gly924Ala) | VCV000067438 | Long QT Syn.\|Short QT Syn. 1\|not spec.\|Carc [symbol] | Uncertain significance(LR: Feb. 17, 2021) |
| 640 | SNP:c.2765G > A (p.Arg922Gln) | 751 | SNP:c.3052C > G (p.Pro1018Ala) | VCV000200520 | Long QT Syn.\|Arrhythmia\|Not Prov. | Uncertain significance(LR: Feb. 17, 2021) |
| 641 | SNP:c.2765G > T (p.Arg922Leu) | 353 | SNP:c.1907C > T (p.Ser636Leu) | VCV001303526 | Not Prov. | Uncertain significance(LR: Feb. 18, 2020) |
| 642 | SNP:c.2768C > A (p.Pro923Gln) | 477 | SNP:c.2276A > G (p.Lys759Arg) | VCV000999182 | Long QT Syn. | Uncertain significance(LR: Feb. 18, 2020) |
| 643 | SNP:c.2768C > T (p.Pro923Leu) | 697 | SNP:c.2891C > T (p.Pro964Leu) | VCV001046584 | Long QT Syn. | Uncertain significance(LR: Feb. 18, 2020) |
| 644 | SNP:c.2770G > A (p.Gly924Arg) | 999 | SNP:c.832A > T (p.Ser279Cys) | VCV001026541 | Long QT Syn. | Uncertain significance(LR: Feb. 18, 2020) |
| 645 | SNP:c.2770G > T (p.Gly924Trp) | 841 | SNP:c.3361C > T (p.Pro1121Ser) | VCV000920215 | Arrhythmia\|Long QT Syn. | Uncertain significance(LR: Feb. 18, 2021) |
| 646 | SNP:c.2771G > A (p.Gly924Glu) | 911 | SNP:c.523G > A (p.Ala175Thr) | VCV000571170 | Long QT Syn. | Uncertain significance(LR: Feb. 19, 2018) |
| 647 | SNP:c.2771G > C (p.Gly924Ala) | 437 | SNP:c.211G > T (p.Gly71Trp) | VCV000853484 | Long QT Syn. | Uncertain significance(LR: Feb. 19, 2019) |
| 648 | SNP:c.2773G > A (p.Gly925Arg) | 508 | SNP:c.2377G > A (p.Asp793Asn) | VCV000921995 | Arrhythmia | Uncertain significance(LR: Feb. 19, 2020) |
| 649 | SNP:c.2774G > A (p.Gly925Glu) | 240 | SNP:c.1745G > A (p.Arg582His) | VCV000926210 | Arrhythmia\| Long QT Syn. | Uncertain significance(LR: Feb. 19, 2021) |
| 650 | SNP:c.2774G > C (p.Gly925Ala) | 618 | SNP:c.2711A > T (p.Glu904Val) | VCV000193977 | Not Prov. | Uncertain significance(LR: Feb. 2, 2015) |
| 651 | SNP:c.2774G > T (p.Gly925Val) | 998 | SNP:c.830C > A (p.Ala277Asp) | VCV000067533 | Congenital long QT Syn.\|Long QT Syn. | Uncertain significance(LR: Feb. 2, 2020) |
| 652 | SNP:c.2777C > T (p.Pro926Leu) | 882 | SNP:c.391G > T (p.Val131Leu) | VCV000927974 | Not Prov.\|Arrhythmia\| Long QT Syn. | Uncertain significance(LR: Feb. 2, 2021) |
| 653 | SNP:c.2780G > A (p.Trp927Ter) | 201 | SNP:c.1693G > T (p.Ala565Ser) | VCV000519393 | Cardiovas. pheno. | Uncertain significance(LR: Feb. 20, 2017) |
| 654 | SNP:c.2780G > T (p.Trp927Leu) | 138 | SNP:c.1564G > A (p.Gly522Arg) | VCV000067212 | Congenital long QT Syn.\|Arrhythmia | Uncertain significance(LR: Feb. 21, 2020) |
| 655 | SNP:c.2781G > T (p.Trp927Cys) | 156 | SNP:c.1601G > T (p.Arg534Leu) | VCV000067221 | Congenital long QT Syn.\|Long QT Syn. | Uncertain significance(LR: Feb. 22, 2020) |
| 656 | SNP:c.2783G > A (p.Gly928Glu) | 512 | SNP:c.2386G > T (p.Val796Leu) | VCV000940061 | Long QT Syn. | Uncertain significance(LR: Feb. 22, 2020) |
| 657 | SNP:c.2785G > C (p.Glu929Gln) | 759 | SNP:c.3089C > G (p.Pro1030Arg) | VCV000837323 | Long QT Syn. | Uncertain significance(LR: Feb. 22, 2020) |
| 658 | SNP:c.2788A > C (p.Ser930Arg) | 682 | SNP:c.2855C > T (p.Pro952Leu) | VCV000862901 | Long QT Syn. | Uncertain significance(LR: Feb. 24, 2019) |
| 659 | SNP:c.2788A > G (p.Ser930Gly) | 606 | SNP:c.2690A > T (p.Lys897Met) | VCV000200480 | Not Prov.\|Arrhythmia | Uncertain significance(LR: Feb. 25, 2019) |
| 660 | SNP:c.278A > G (p.Lys93Arg) | 30 | SNP:c.1181G > A (p.Arg394His) | VCV000565408 | Long QT Syn.\|Arrhythmia | Uncertain significance(LR: Feb. 25, 2020) |
| 661 | SNP:c.2792C > G (p.Pro931Arg) | 507 | SNP:c.2372G > A (p.Arg791Gln) | VCV000925151 | Long QT Syn.\|Arrhythmia | Uncertain significance(LR: Feb. 25, 2020) |
| 662 | SNP:c.2792C > T (p.Pro931Leu) | 794 | SNP:c.3178A > G (p.Met1060Val) | VCV000924650 | Arrhythmia | Uncertain significance(LR: Feb. 26, 2019) |
| 663 | SNP:c.2794T > C (p.Ser932Pro) | 127 | SNP:c.1496T > G (p.Leu499Arg) | VCV000200346 | not spec.\|Not Prov. | Uncertain significance(LR: Feb. 27, 2020) |
| 664 | SNP:c.2795C > T (p.Ser932Phe) | 885 | SNP:c.416G > C (p.Gly139Ala) | VCV001018296 | Long QT Syn. | Uncertain significance(LR: Feb. 27, 2020) |
| 665 | SNP:c.2803C > G (p.Pro935Ala) | 1012 | SNP:c.877G > A (p.Ala293Thr) | VCV000864669 | Long QT Syn. | Uncertain significance(LR: Feb. 27, 2020) |
| 666 | SNP:c.280G > A (p.Val94Met) | 571 | SNP:c.2600T > C (p.Met867Thr) | VCV000641744 | Arrhythmia\| Long QT Syn. | Uncertain significance(LR: Feb. 3, 2020) |
| 667 | SNP:c.280G > C (p.Val94Leu) | 4 | SNP:c.1009A > G (p.Thr337Ala) | VCV000927557 | Arrhythmia | Uncertain significance(LR: Feb. 6, 2020) |
| 668 | SNP:c.2810G > A (p.Ser937Asn) | 957 | SNP:c.707G > A (p.Gly236Asp) | VCV000952259 | Long QT Syn. | Uncertain significance(LR: Feb. 6, 2020) |
| 669 | SNP:c.2819G > T (p.Ser940Ile) | 350 | SNP:c.1904A > G (p.Asn635Ser) | VCV000200393 | Long QT Syn. | Uncertain significance(LR: Feb. 7, 2018) |
| 670 | SNP:c.281T > G (p.Val94Gly) | 656 | SNP:c.2783G > A (p.Gly928Glu) | VCV000850359 | Long QT Syn. | Uncertain significance(LR: Feb. 7, 2020) |
| 671 | SNP:c.2832G > C (p.Glu944Asp) | 282 | SNP:c.1814C > A (p.Pro605His) | VCV000858913 | Long QT Syn. | Uncertain significance(LR: Feb. 8, 2019) |
| 672 | SNP:c.2832G > T (p.Glu944Asp) | 1037 | SNP:c.959C > G (p.Ser320Trp) | VCV000200312 | Arrhythmia\|Not Prov. | Uncertain significance(LR: Feb. 8, 2021) |
| 673 | SNP:c.2833G > T (p.Gly945Cys) | 411 | SNP:c.2036G > A (p.Arg679Gln) | VCV000922598 | Arrhythmia | Uncertain significance(LR: Jan. 1, 2020) |
| 674 | SNP:c.2837C > T (p.Pro946Leu) | 630 | SNP:c.2746G > A (p.Gly916Arg) | VCV000924836 | Arrhythmia | Uncertain significance(LR: Jan. 1, 2020) |
| 675 | SNP:c.2842C > A (p.Arg948Ser) | 25 | SNP:c.1126C > G (p.Gln376Glu) | VCV000909783 | Long QT Syn. 2 | Uncertain significance(LR: Jan. 13, 2018) |
| 676 | SNP:c.2842C > T (p.Arg948Cys) | 94 | SNP:c.1397A > G (p.Asp466Gly) | VCV000909781 | Long QT Syn. 2 | Uncertain significance(LR: Jan. 13, 2018) |
| 677 | SNP:c.2843G > A (p.Arg948His) | 995 | SNP:c.825C > G (p.Ser275Arg) | VCV000911914 | Long QT Syn. 2 | Uncertain significance(LR: Jan. 13, 2018) |
| 678 | SNP:c.284A > C (p.Glu95Ala) | 694 | SNP:c.2887C > A (p.Pro963Thr) | VCV000067449 | Congenital long QT Syn.\|Not Prov.\|Arrhythm [symbol] | Uncertain significance(LR: Jan. 13, 2020) |
| 679 | SNP:c.2853C > A (p.Ser951Arg) | 860 | SNP:c.3457C > T (p.His1153Tyr) | VCV000067497 | Arrhythmia\|Not Prov.\|Long QT Syn. 2\|Short [symbol] | Uncertain significance(LR: Jan. 13, 2021) |
| 680 | SNP:c.2854C > T (p.Pro952Ser) | 834 | SNP:c.3346G > A (p.Ala1116Thr) | VCV000918904 | Arrhythmia | Uncertain significance(LR: Jan. 14, 2020) |
| 681 | SNP:c.2855C > G (p.Pro952Arg) | 711 | SNP:c.2931C > G (p.Cys977Trp) | VCV000919282 | Arrhythmia | Uncertain significance(LR: Jan. 15, 2019) |
| 682 | SNP:c.2855C > T (p.Pro952Leu) | 569 | SNP:c.2591A > G (p.Asp864Gly) | VCV000067417 | Long QT Syn.\|Congenital long QT Syn. | Uncertain significance(LR: Jan. 15, 2020) |
| 683 | SNP:c.2857C > T (p.Leu953Phe) | 789 | SNP:c.3163C > T (p.Arg1055Trp) | VCV000405340 | Long QT Syn. 2\|Long QT Syn. | Uncertain significance(LR: Jan. 15, 2020) |
| 684 | SNP:c.2860C > T (p.Arg954Cys) | 40 | SNP:c.1226T > A (p.Val409Glu) | VCV000200329 | Not Prov.\|Cardiovas. pheno. | Uncertain significance(LR: Jan. 16, 2020) |
| 685 | SNP:c.2861G > A (p.Arg954His) | 472 | SNP:c.2257G > T (p.Ala753Ser) | VCV000067380 | Congenital long QT Syn.\|Arrhythmia | Uncertain significance(LR: Jan. 17, 2020) |
| 686 | SNP:c.2863C > G (p.Leu955Val) | 705 | SNP:c.2908G > A (p.Gly970Arg) | VCV000926177 | Arrhythmia | Uncertain significance(LR: Jan. 17, 2020) |
| 687 | SNP:c.286A > G (p.Ile96Val) | 875 | SNP:c.377T > A (p.Ile 126Asn) | VCV000859531 | Long QT Syn. | Uncertain significance(LR: Jan. 19, 2019) |
| 688 | SNP:c.2870C > T (p.Pro957Leu) | 60 | SNP:c.1283C > T (p.Ser428Leu) | VCV000067180 | Not Prov.\|Congenital long QT Syn. | Uncertain significance(LR: Jan. 19, 2021) |
| 689 | SNP:c.2875T > A (p.Ser959Thr) | 861 | SNP:c.3460G > A (p.Gly1154Ser) | VCV000067498 | Not Prov.\|Arrhythmia\|Long QT Syn. | Uncertain significance(LR: Jan. 2, 2020) |
| 690 | SNP:c.2876C > G (p.Ser959Cys) | 631 | SNP:c.274C > T (p.Arg92Cys) | VCV000200573 | Arrhythmia\|Not Prov.\|Long QT Syn.\|Long Q [symbol] | Uncertain significance(LR: Jan. 20, 2021) |
| 691 | SNP:c.2876C > T (p.Ser959Phe) | 768 | SNP:c.3103C > T (p.Arg1035Trp) | VCV000067468 | Arrhythmia\|Long QT Syn.\|Not Prov. | Uncertain significance(LR: Jan. 20, 2021) |
| 692 | SNP:c.2879G > A (p.Ser960Asn) | 439 | SNP:c.2128G > A (p.Gly710Ser) | VCV000664978 | Long QT Syn. | Uncertain significance(LR: Jan. 21, 2019) |
| 693 | SNP:c.287T > C (p.Ile96Thr) | 996 | SNP:c.829G > T (p.Ala277Ser) | VCV000834430 | Long QT Syn. | Uncertain significance(LR: Jan. 22, 2019) |
| 694 | SNP:c.2887C > A (p.Pro963Thr) | 915 | SNP:c.527G > A (p.Arg176Gln) | VCV001010337 | Long QT Syn. | Uncertain significance(LR: Jan. 23, 2020) |
| 695 | SNP:c.2890C > G (p.Pro964Ala) | 577 | SNP:c.2624C > G (p.Thr875Arg) | VCV000849676 | Long QT Syn. | Uncertain significance(LR: Jan. 26, 2019) |
| 696 | SNP:c.2891C > G (p.Pro964Arg) | 262 | SNP:c.1780G > C (p.Gly594Arg) | VCV001026973 | Long QT Syn. | Uncertain significance(LR: Jan. 27, 2020) |
| 697 | SNP:c.2891C > T (p.Pro964Leu) | 704 | SNP:c.2906G > T (p.Gly969Val) | VCV000926647 | Arrhythmia | Uncertain significance(LR: Jan. 27, 2020) |
| 698 | SNP:c.2893G > A (p.Gly965Arg) | 782 | SNP:c.3138G > T (p.Gln1046His) | VCV000926330 | Arrhythmia | Uncertain significance(LR: Jan. 27, 2020) |
| 699 | SNP:c.2899C > T (p.Pro967Ser) | 852 | SNP:c.3410C > A (p.Ser1137Tyr) | VCV000966027 | Long QT Syn. | Uncertain significance(LR: Jan. 27, 2020) |
| 700 | SNP:c.2900C > T (p.Pro967Leu) | 556 | SNP:c.2548G > T (p.Asp850Tyr) | VCV000922966 | Arrhythmia | Uncertain significance(LR: Jan. 28, 2019) |
| 701 | SNP:c.2902C > G (p.Pro968Ala) | 14 | SNP:c.1058C > T (p.Thr353Ile) | VCV001047609 | Long QT Syn. | Uncertain significance(LR: Jan. 28, 2020) |
| 702 | SNP:c.2903C > T (p.Pro968Leu) | 1045 | SNP:c.973G > C (p.Val325Leu) | VCV000856033 | Long QT Syn. | Uncertain significance(LR: Jan. 28, 2020) |
| 703 | SNP:c.2906G > A (p.Gly969Asp [symbol] | 972 | SNP:c.751C > T (p.Pro251Ser) | VCV000067523 | Long QT Syn.\|Congenital long QT Syn. | Uncertain significance(LR: Jan. 30, 2018) |
| 704 | SNP:c.2906G > T (p.Gly969Val) | 811 | SNP:c.322T > C (p.Cys108Arg) | VCV000067482 | Congenital long QT Syn.\|Long QT Syn. | Uncertain significance(LR: Jan. 30, 2019) |
| 705 | SNP:c.2908G > A (p.Gly970Arg) | 530 | SNP:c.2458G > A (p.Gly820Arg) | VCV000067401 | Not Prov.\|Congenital long QT Syn.\|Arrhythm [symbol] | Uncertain significance(LR: Jan. 30, 2020) |
| 706 | SNP:c.290C > G (p.Ala97Gly) | 467 | SNP:c.223C > G (p.Gln75Glu) | VCV001023341 | Long QT Syn. | Uncertain significance(LR: Jan. 31, 2020) |
| 707 | SNP:c.2913G > T (p.Glu971Asp) | 164 | SNP:c.1622G > A (p.Arg541His) | VCV000960653 | Long QT Syn.\|Arrhythmia | Uncertain significance(LR: Jan. 5, 2021) |
| 708 | SNP:c.2926G > A (p.Asp976Asn [symbol] | 586 | SNP:c.2648G > A (p.Arg883Gln) | VCV001171663 | Arrhythmia | Uncertain significance(LR: Jan. 5, 2021) |
| 709 | SNP:c.2930G > T (p.Cys977Phe) | 616 | SNP:c.2710G > A (p.Glu904Lys) | VCV001171262 | Arrhythmia | Uncertain significance(LR: Jan. 5, 2021) |
| 710 | SNP:c.2931C > A (p.Cys977Ter) | 662 | SNP:c.2792C > T (p.Pro931Leu) | VCV000654837 | Arrhythmia\|Long QT Syn. | Uncertain significance(LR: Jan. 5, 2021) |
| 711 | SNP:c.2931C > G (p.Cys977Trp) | 92 | SNP:c.1394T > C (p.Val465 Ala) | VCV000374073 | Arrhythmia\|Hypertrophic cardiomyopathy | Uncertain significance(LR: Jan. 8, 2014) |
| 712 | SNP:c.2932G > A (p.Glu978Lys) | 667 | SNP:c.280G > C (p.Val94Leu) | VCV001052852 | Long QT Syn. | Uncertain significance(LR: Jan. 9, 2020) |
| 713 | SNP:c.2932G > C (p.Glu978Gln) | 673 | SNP:c.2833G > T (p.Gly945Cys) | VCV001063652 | Long QT Syn. | Uncertain significance(LR: Jan. 9, 2020) |
| 714 | SNP:c.2932G > T (p.Glu978Ter) | 740 | SNP:c.3019C > T (p.Arg1007Cys) | VCV000918739 | Arrhythmia | Uncertain significance(LR: Jan. 9, 2020) |
| 715 | SNP:c.293T > G (p.Phe98Cys) | 27 | SNP:c.115T > C (p.Cys39Arg) | VCV000456883 | Long QT Syn. | Uncertain significance(LR: Jul. 10, 2017) |
| 716 | SNP:c.2941A > G (p.Ser981Gly) | 796 | SNP:c.3185C > T (p.Thr1062Ile) | VCV000586073 | Long QT Syn.\|Not Prov. | Uncertain significance(LR: Jul. 10, 2018) |
| 717 | SNP:c.2944G > A (p.Asp982Asn | 537 | SNP:c.247G > T (p.Ala83Ser) | VCV000643651 | Long QT Syn. | Uncertain significance(LR: Jul. 11, 2018) |
| 718 | SNP:c.2948C > T (p.Thr983Ile) | 615 | SNP:c.2707G > C (p.Gly903Arg) | VCV000960253 | Long QT Syn. | Uncertain significance(LR: Jul. 11, 2019) |
| 719 | SNP:c.2952C > A (p.Cys984Ter) | 468 | SNP:c.2245G > T (p.Gly749Cys) | VCV000405350 | Long QT Syn. | Uncertain significance(LR: Jul. 12, 2017) |
| 720 | SNP:c.2954A > G (p.Asn985Ser) | 120 | SNP:c.1477T > C (p.Tyr493His) | VCV000658590 | Long QT Syn. | Uncertain significance(LR: Jul. 12, 2018) |
| 721 | SNP:c.2956C > A (p.Pro986Thr) | 964 | SNP:c.724C > T (p.Arg242Cys) | VCV000652662 | Long QT Syn. | Uncertain significance(LR: Jul. 12, 2019) |
| 722 | SNP:c.2962T > G (p.Ser988Ala) | 722 | SNP:c.2962T > G (p.Ser988Ala) | VCV000859417 | Long QT Syn. | Uncertain significance(LR: Jul. 13, 2020) |
| 723 | SNP:c.2968G > A (p.Ala990Thr) | 1021 | SNP:c.908C > G (p.Ala303Gly) | VCV000957218 | Long QT Syn. | Uncertain significance(LR: Jul. 14, 2019) |
| 724 | SNP:c.296A > C (p.Tyr99Ser) | 621 | SNP:c.2719G > T (p.Ala907Ser) | VCV000834753 | Long QT Syn.\|Arrhythmia | Uncertain significance(LR: Jul. 14, 2020) |
| 725 | SNP:c.296A > G (p.Tyr99Cys) | 413 | SNP:c.2042G > A (p.Arg681Gln) | VCV000957302 | Long QT Syn. | Uncertain significance(LR: Jul. 15, 2019) |
| 726 | SNP:c.2987A > T (p.Asn996Ile) | 633 | SNP:c.2753G > A (p.Ser918Asn) | VCV000939122 | Long QT Syn. | Uncertain significance(LR: Jul. 15, 2019) |
| 727 | SNP:c.298C > G (p.Arg100Gly) | 492 | SNP:c.233C > T (p.Ala78Val) | VCV000957483 | Long QT Syn. | Uncertain significance(LR: Jul. 16, 2019) |
| 728 | SNP:c.298C > T (p.Arg100Trp) | 609 | SNP:c.2696C > G (p.Thr899Arg) | VCV000456916 | Long QT Syn.\|Cardiovas. pheno. | Uncertain significance(LR: Jul. 17, 2019) |
| 729 | SNP:c.2996G > A (p.Ser999Asn) | 929 | SNP:c.578C > T (p.Ala193Val) | VCV001304544 | Not Prov. | Uncertain significance(LR: Jul. 17, 2019) |
| 730 | SNP:c.299G > A (p.Arg100Gln) | 1010 | SNP:c.872T > G (p.Met291Arg) | VCV000374955 | Not Prov.\|Long QT Syn.\|Long QT Syn. 2\|Sho [symbol] | Uncertain significance(LR: Jul. 17, 2020) |
| 731 | SNP:c.299G > T (p.Arg100Leu) | 314 | SNP:c.1870A > T (p.Ser624Cys) | VCV000644725 | Long QT Syn. | Uncertain significance(LR: Jul. 19, 2019) |
| 732 | SNP:c.29C > T (p.Pro10Leu) | 703 | SNP:c.2906G > A (p.Gly969Asp) | VCV000918705 | Arrhythmia | Uncertain significance(LR: Jul. 19, 2019) |
| 733 | SNP:c.3002G > A (p.Trp1001Ter [symbol] | 901 | SNP:c.451C > T (p.Pro151Ser) | VCV001026596 | Long QT Syn. | Uncertain significance(LR: Jul. 20, 2020) |
| 734 | SNP:c.3003G > A (p.Trp 1001Ter [symbol] | 85 | SNP:c.1372A > T (p.Ile458Phe) | VCV000456887 | Long QT Syn. | Uncertain significance(LR: Jul. 21, 2017) |
| 735 | SNP:c.3003G > C (p.Trp1001Cy [symbol] | 1051 | SNP:c.983G > A (p.Arg328His) | VCV000200315 | Long QT Syn.\|Not Prov.\|Arrhythmia | Uncertain significance(LR: Jul. 21, 2020) |
| 736 | SNP:c.3007G > A (p.Asp1003As [symbol] | 671 | SNP:c.2832G > C (p.Glu944Asp) | VCV000628923 | Arrhythmia | Uncertain significance(LR: Jul. 22, 2018) |
| 737 | SNP:c.3007G > T (p.Asp1003Ty [symbol] | 207 | SNP:c.169G > C (p.Ala57Pro) | VCV000067240 | Congenital long QT Syn.\|Long QT Syn. | Uncertain significance(LR: Jul. 22, 2019) |
| 738 | SNP:c.3013C > T (p.Arg1005Tr [symbol] | 442 | SNP:c.2144C > T (p.Ala715Val) | VCV000200413 | Arrhythmia\|Long QT Syn.\|not spec.\|Cardiova [symbol] | Uncertain significance(LR: Jul. 22, 2020) |
| 739 | SNP:c.3014G > A (p.Arg1005Gl [symbol] | 340 | SNP:c.1892C > T (p.Ser631Phe) | VCV000456898 | Long QT Syn.\|Not Prov. | Uncertain significance(LR: Jul. 24, 2019) |
| 740 | SNP:c.3019C > T (p.Arg1007Cy [symbol] | 868 | SNP:c.352G > C (p.Glu118Gln) | VCV001000649 | Long QT Syn. | Uncertain significance(LR: Jul. 24, 2020) |
| 741 | SNP:c.301A > G (p.Lys101Glu) | 961 | SNP:c.719C > T (p.Pro240Leu) | VCV000200299 | Not Prov.\|Long QT Syn. | Uncertain significance(LR: Jul. 24, 2020) |
| 742 | SNP:c.301A > T (p.Lys101Ter) | 73 | SNP:c.1332G > C (p.Glu444Asp) | VCV000927767 | Arrhythmia | Uncertain significance(LR: Jul. 25, 2019) |
| 743 | SNP:c.3020G > A (p.Arg1007Hi [symbol] | 649 | SNP:c.2774G > A (p.Gly925Glu) | VCV000921867 | Long QT Syn.\|Arrhythmia | Uncertain significance(LR: Jul. 25, 2020) |
| 744 | SNP:c.3027C > G (p.Tyr1009Te [symbol] | 969 | SNP:c.740A > G (p.Gln247Arg) | VCV001057001 | Long QT Syn. | Uncertain significance(LR: Jul. 25, 2020) |
| 745 | SNP:c.302A > G (p.Lys101Arg) | 581 | SNP:c.2634G > T (p.Glu878Asp) | VCV001304037 | Not Prov. | Uncertain significance(LR: Jul. 26, 2018) |
| 746 | SNP:c.3032A > G (p.Glu1011G [symbol] | 765 | SNP:c.3097C > T (p.Arg1033Trp) | VCV000067467 | Long QT Syn. 1\|Long QT Syn.\|Congenital lo [symbol] | Uncertain significance(LR: Jul. 27, 2021) |
| 747 | SNP:c.303A > T (p.Lys101Asn) | 775 | SNP:c.3119G > A (p.Ser1040Asn) | VCV000200526 | Not Prov. | Uncertain significance(LR: Jul. 29, 2014) |
| 748 | SNP:c.3040C > T (p.Arg1014Ter [symbol] | 857 | SNP:c.3448C > A (p.Leu1150Met) | VCV000456931 | Arrhythmia\|Not Prov.\|Long QT Syn. | Uncertain significance(LR: Jul. 29, 2020) |
| 749 | SNP:c.3041G > A (p.Arg1014Gl [symbol] | 888 | SNP:c.424G > A (p.Ala142Thr) | VCV001025220 | Long QT Syn. | Uncertain significance(LR: Jul. 29, 2020) |
| 750 | SNP:c.3049G > T (p.Ala1017Ser [symbol] | 632 | SNP:c.2750C > T (p.Pro917Leu) | VCV000067432 | Not Prov.\|not spec.\|Arrhythmia\|Long QT Syn [symbol] | Uncertain significance(LR: Jul. 29, 2021) |
| 751 | SNP:c.3052C > G (p.Pro1018Ala [symbol] | 842 | SNP:c.3365C > G (p.Pro1122Arg) | VCV000234998 | not spec. | Uncertain significance(LR: Jul. 3, 2014) |
| 752 | SNP:c.3053C > T (p.Pro1018Leu [symbol] | 947 | SNP:c.670G > A (p.Gly224Arg) | VCV000200297 | Not Prov. | Uncertain significance(LR: Jul. 3, 2019) |
| 753 | SNP:c.3058C > T (p.Pro1020Ser [symbol] | 931 | SNP:c.589G > A (p.Asp197Asn) | VCV000952715 | Long QT Syn. | Uncertain significance(LR: Jul. 31, 2019) |
| 754 | SNP:c.305A > C (p.Asp102Ala) | 943 | SNP:c.656A > T (p.Asp219Val) | VCV000157662 | Long QT Syn.\|Long QT Syn. 2 | Uncertain significance(LR: Jul. 5, 2019) |
| 755 | SNP:c.305A > T (p.Asp102Val) | 1009 | SNP:c.872T > C (p.Met291Thr) | VCV000067537 | Long QT Syn.\|Congenital long QT Syn. | Uncertain significance(LR: Jul. 6, 2018) |
| 756 | SNP:c.3061A > G (p.Ser1021Gly [symbol] | 1052 | SNP:c.985A > G (p.Thr329Ala) | VCV000640482 | Long QT Syn. | Uncertain significance(LR: Jul. 6, 2018) |
| 757 | SNP:c.3070A > T (p.Asn1024Ty [symbol] | 570 | SNP:c.2594C > T (p.Thr865Ile) | VCV000957208 | Long QT Syn. | Uncertain significance(LR: Jul. 6, 2019) |
| 758 | SNP:c.3079C > A (p.Leu1027Ile [symbol] | 787 | SNP:c.3153G > T (p.Arg1051Ser) | VCV000952274 | Long QT Syn. | Uncertain significance(LR: Jul. 6, 2019) |
| 759 | SNP:c.3089C > G (p.Pro1030Arg [symbol] | 990 | SNP:c.812G > A (p.Arg271His) | VCV000200304 | Not Prov. | Uncertain significance(LR: Jul. 7, 2014) |
| 760 | SNP:c.3089C > T (p.Pro 1030Leu [symbol] | 870 | SNP:c.362C > T (p.Ala121Val) | VCV001054947 | Long QT Syn. | Uncertain significance(LR: Jul. 8, 2020) |
| 761 | SNP:c.3092G > A (p.Gly1031As [symbol] | 126 | SNP:c.148G > C (p.Glu50Gln) | VCV000935728 | Long QT Syn. | Uncertain significance(LR: Jul. 9, 2019) |
| 762 | SNP:c.3094C > T (p.Arg1032Trp [symbol] | 551 | SNP:c.2536C > G (p.Pro846Ala) | VCV000161254 | Long QT Syn. 2 | Uncertain significance(LR: Jun. 1, 2014) |
| 763 | SNP:c.3095G > A (p.Arg1032Gl [symbol] | 867 | SNP:c.352G > A (p.Glu118Lys) | VCV000810212 | Not Prov. | Uncertain significance(LR: Jun. 1, 2017) |
| 764 | SNP:c.3095G > C (p.Arg1032Pr [symbol] | 461 | SNP:c.2221A > C (p.Lys741Gln) | VCV000923298 | Arrhythmia | Uncertain significance(LR: Jun. 11, 2019) |
| 765 | SNP:c.3097C > T (p.Arg1033Trp [symbol] | 663 | SNP:c.2794T > C (p.Ser932Pro) | VCV000942612 | Long QT Syn. | Uncertain significance(LR: Jun. 13, 2019) |
| 766 | SNP:c.3098G > A (p.Arg1033Gl [symbol] | 162 | SNP:c.1612A > G (p.Lys538Glu) | VCV000359311 | Long QT Syn. | Uncertain significance(LR: Jun. 14, 2016) |
| 767 | SNP:c.3098G > T (p.Arg1033Le [symbol] | 753 | SNP:c.3058C > T (p.Pro 1020Ser) | VCV000359306 | Long QT Syn. | Uncertain significance(LR: Jun. 14, 2016) |
| 768 | SNP:c.3103C > T (p.Arg1035Trp [symbol] | 853 | SNP:c.3416C > T (p.Pro1139Leu) | VCV000359302 | Long QT Syn. | Uncertain significance(LR: Jun. 14, 2016) |
| 769 | SNP:c.3104G > A (p.Arg1035Gl [symbol] | 1003 | SNP:c.853G > T (p.Ala285Ser) | VCV000359315 | Long QT Syn. | Uncertain significance(LR: Jun. 14, 2016) |
| 770 | SNP:c.3107G > A (p.Gly1036As [symbol] | 1007 | SNP:c.868G > T (p.Ala290Ser) | VCV000359314 | Long QT Syn. | Uncertain significance(LR: Jun. 14, 2016) |
| 771 | SNP:c.3109G > A (p.Asp1037As [symbol] | 119 | SNP:c.1475A > T (p.His492Leu) | VCV000581371 | Long QT Syn. | Uncertain significance(LR: Jun. 14, 2018) |
| 772 | SNP:c.3112G > A (p.Val1038Me [symbol] | 158 | SNP:c.1603G > T (p.Val535Leu) | VCV000570842 | Long QT Syn. | Uncertain significance(LR: Jun. 14, 2018) |
| 773 | SNP:c.3112G > T (p.Val1038Le [symbol] | 462 | SNP:c.2222A > G (p.Lys741Arg) | VCV000566154 | Long QT Syn. | Uncertain significance(LR: Jun. 14, 2018) |
| 774 | SNP:c.3118A > G (p.Ser1040Gly [symbol] | 597 | SNP:c.2675G > A (p.Arg892His) | VCV001054268 | Long QT Syn.\|Not Prov. | Uncertain significance(LR: Jun. 16, 2020) |
| 775 | SNP:c.3119G > A (p.Ser1040As [symbol] | 695 | SNP:c.2890C > G (p.Pro964Ala) | VCV000359307 | Long QT Syn.\|Long QT Syn. 2 | Uncertain significance(LR: Jun. 16, 2020) |
| 776 | SNP:c.3124C > G (p.Leu1042Va [symbol] | 29 | SNP:c.1180C > T (p.Arg394Cys) | VCV000855813 | Long QT Syn. | Uncertain significance(LR: Jun. 18, 2020) |
| 777 | SNP:c.3125T > C (p.Leu1042Pro [symbol] | 22 | SNP:c.1097G > A (p.Arg366Gln) | VCV000918732 | Arrhythmia Not Prov. | Uncertain significance(LR: Jun. 18, 2021) |
| 778 | SNP:c.312C > G (p.Ser104Arg) | 19 | SNP:c.1076T > A (p.Ile359Lys) | VCV000456877 | Long QT Syn. | Uncertain significance(LR: Jun. 19, 2017) |
| 779 | SNP:c.3133C > T (p.Leu1045Phe [symbol] | 410 | SNP:c.2035C > T (p.Arg679Trp) | VCV000581266 | Long QT Syn. | Uncertain significance(LR: Jun. 2, 2018) |
| 780 | SNP:c.3134T > G (p.Leu1045Arg [symbol] | 548 | SNP:c.2525T > C (p.Leu842Pro) | VCV000519212 | Cardiovas. pheno. | Uncertain significance(LR: Jun. 22, 2017) |
| 781 | SNP:c.3137A > G (p.Gln1046Arg [symbol] | 445 | SNP:c.215C > G (p.Pro72Arg) | VCV000067369 | Long QT Syn.\|Congenital long QT Syn. | Uncertain significance(LR: Jun. 22, 2019) |
| 782 | SNP:c.3138G > T (p.Gl1046His [symbol] | 865 | SNP:c.3470C > T (p.Pro1157Leu) | VCV000067499 | Arrhythmia\|SUDDEN INFANT DEATH Syn [symbol] | Uncertain significance(LR: Jun. 22, 2021) |
| 783 | SNP:c.3139C > T (p.Arg1047Cys [symbol] | 107 | SNP:c.1447G > T (p.Val483Phe) | VCV000949825 | Long QT Syn. | Uncertain significance(LR: Jun. 24, 2019) |
| 784 | SNP:c.3140G > A (p.Arg1047His [symbol] | 920 | SNP:c.545C > G (p.Ser182Trp) | VCV000405351 | Not Prov.\|Long QT Syn. | Uncertain significance(LR: Jun. 24, 2019) |
| 785 | SNP:c.3140G > T (p.Arg1047Leu [symbol] | 743 | SNP:c.3020G > A (p.Arg1007His) | VCV000067463 | Arrhythmia\|Congenital long QT Syn.\|Long Q [symbol] | Uncertain significance(LR: Jun. 24, 2020) |
| 786 | SNP:c.3146T > C (p.Leu1049Pro [symbol] | 638 | SNP:c.275G > T (p.Arg92Leu) | VCV000020057 | Not Prov. | Uncertain significance(LR: Jun. 25, 2019) |
| 787 | SNP:c.3153G > T (p.Arg1051Ser [symbol] | 930 | SNP:c.580G > A (p.Val194Met) | VCV000640808 | Long QT Syn.\|Not Prov. | Uncertain significance(LR: Jun. 25, 2019) |
| 788 | SNP:c.3161C > T (p.Thr1054Ile) | 936 | SNP:c.614C > T (p.Pro205Leu) | VCV000829864 | Short QT Syn. 1 | Uncertain significance(LR: Jun. 25, 2019) |
| 789 | SNP:c.3163C > T (p.Arg1055Trp [symbol] | 591 | SNP:c.2659C > T (p.Arg887Cys) | VCV000193802 | Long QT Syn.\|Not Prov.\|Arrhythmia | Uncertain significance(LR: Jun. 25, 2020) |
| 790 | SNP:c.3164G > A (p.Arg1055Gln [symbol] | 637 | SNP:c.275G > C (p.Arg92Pro) | VCV000947667 | Long QT Syn. | Uncertain significance(LR: Jun. 26, 2019) |
| 791 | SNP:c.3170G > A (p.Ser1057Asn [symbol] | 651 | SNP:c.2774G > T (p.Gly925Val) | VCV000200497 | Long QT Syn.\|Sudden unexplained death\|Not [symbol] | Uncertain significance(LR: Jun. 26, 2019) |
| 792 | SNP:c.3172G > A (p.Ala1058Thr [symbol] | 927 | SNP:c.56T > G (p.Ile19Ser) | VCV000949641 | Long QT Syn. | Uncertain significance(LR: Jun. 27, 2019) |
| 793 | SNP:c.3173C > A (p.Ala1058Gln [symbol] | 843 | SNP:c.3365C > T (p.Pro112Leu) | VCV000200534 | Arrhythmia\|Long QT Syn.\|Not Prov. | Uncertain significance(LR: Jun. 30, 2020) |
| 794 | SNP:c.3178A > G (p.Met1060Val [symbol] | 799 | SNP:c.3191T > G (p.Leu1064Arg) | VCV000845095 | Long QT Syn. | Uncertain significance(LR: Jun. 4, 2019) |
| 795 | SNP:c.317T > A (p.Phe106Tyr) | 79 | SNP:c.134A > C (p.Asn45Thr) | VCV000949254 | Long QT Syn. | Uncertain significance(LR: Jun. 5, 2019) |
| 796 | SNP:c.3185C > T (p.Thr1062Ile) | 6 | SNP:c.1017C > G (p.Asn339Lys) | VCV000042831 | Long QT Syn. | Uncertain significance(LR: Jun. 7, 2019) |
| 797 | SNP:c.3187G > C (p.Val1063Leu [symbol] | 290 | SNP:c.1826A > G (p.Asp609Gly) | VCV000067289 | Congenital long QT Syn.\|Long QT Syn. | Uncertain significance(LR: Jun. 7, 2019) |
| 798 | SNP:c.318C > A (p.Phe106Leu) | 838 | SNP:c.3352G > A (p.Glu1118Lys) | VCV000855130 | Long QT Syn. | Uncertain significance(LR: Jun. 7, 2020) |
| 799 | SNP:c.3191T > G (p.Leu1064Arg [symbol] | 18 | SNP:c.1073T > A (p.Ile358Asn) | VCV001172314 | Arrhythmia | Uncertain significance(LR: Jun. 8, 2020) |
| 800 | SNP:c.3193C > T (p.Gln1065Ter [symbol] | 900 | SNP:c.451C > G (p.Pro151Ala) | VCV000405361 | Arrhythmia\|Not Prov.\|Short QT Syn. 1\|Long [symbol] | Uncertain significance(LR: Jun. 8, 2020) |
| 801 | SNP:c.3196C > G (p.Leu1066Val [symbol] | 422 | SNP:c.2080C > T (p.Arg694Cys) | VCV000651990 | Arrhythmia\|Long QT Syn. | Uncertain significance(LR: Jun. 9, 2019) |
| 802 | SNP:c.31C > T (p.Gln11Ter) | 908 | SNP:c.491G > A (p.Arg164His) | VCV000067508 | Long QT Syn.\|Congenital long QT Syn.\|Lon [symbol] | Uncertain significance(LR: Mar. 1, 2020) |
| 803 | SNP:c.3202C > T (p.Gln106Ter8 [symbol] | 546 | SNP:c.2515C > A (p.Leu839Met) | VCV000920490 | Long QT Syn.\|Arrhythmia | Uncertain significance(LR: Mar. 10, 2020) |
| 804 | SNP:c.3205A > G (p.Gln1068Arg [symbol] | 657 | SNP:c.2785G > C (p.Glu929Gln) | VCV000926191 | Arrhythmia | Uncertain significance(LR: Mar. 11, 2020) |
| 805 | SNP:c.3209A > T (p.Gln1070Leu [symbol] | 736 | SNP:c.3007G > A (p.Asp1003Asn) | VCV000373622 | not spec.\|Long QT Syn. | Uncertain significance(LR: Mar. 11, 2020) |
| 806 | SNP:c.3211A > G (p.Met1071Val [symbol] | 773 | SNP:c.3112G > T (p.Val1038Leu) | VCV000405360 | Arrhythmia\|Long QT Syn. | Uncertain significance(LR: Mar. 12, 2019) |
| 807 | SNP:c.3215C > T (p.Thr1072Met [symbol] | 909 | SNP:c.499C > G (p.Leu167Val) | VCV000929250 | not spec. | Uncertain significance(LR: Mar. 12, 2019) |
| 808 | SNP:c.3224C > A (p.Pro1075Gln [symbol] | 312 | SNP:c.1864C > T (p.Leu622Phe) | VCV000037301 | Congenital long QT Syn.\|Long QT Syn. | Uncertain significance(LR: Mar. 15, 2017) |
| 809 | SNP:c.3224C > T (p.Pro1075Leu [symbol] | 538 | SNP:c.2482T > C (p.Cys828Arg) | VCV000842229 | Long QT Syn. | Uncertain significance(LR: Mar. 15, 2019) |
| 810 | SNP:c.3229G > A (p.Ala1077Thr [symbol] | 720 | SNP:c.2954A > G (p.Asn985Ser) | VCV000067456 | Arrhythmia Not Prov.\|Long QT Syn. | Uncertain significance(LR: Mar. 16, 2019) |
| 811 | SNP:c.322T > C (p.Cys108Arg) | 884 | SNP:c.409A > G (p.Met137Val) | VCV000930960 | Short QT Syn. 1 | Uncertain significance(LR: Mar. 16, 2020) |
| 812 | SNP:c.3233A > G (p.Tyr1078Cys [symbol] | 428 | SNP:c.2092G > A (p.Glu698Lys) | VCV000945957 | Not Prov.\|Long QT Syn. | Uncertain significance(LR: Mar. 16, 2021) |
| 813 | SNP:c.3247A > G (p.Thr1083Ala [symbol] | 182 | SNP:c.1678A > G (p.Ile560Val) | VCV001055024 | Long QT Syn. | Uncertain significance(LR: Mar. 17, 2020) |
| 814 | SNP:c.3251C > T (p.Pro1084Leu [symbol] | 642 | SNP:c.2768C > A (p.Pro925Gln) | VCV001008348 | Long QT Syn. | Uncertain significance(LR: Mar. 17, 2020) |
| 815 | SNP:c.3263C > G (p.Pro1088Arg [symbol] | 655 | SNP:c.2781G > T (p.Trp927Cys) | VCV001037100 | Not Prov.\|Long QT Syn. | Uncertain significance(LR: Mar. 17, 2020) |
| 816 | SNP:c.326T > G (p.Leu109Arg) | 639 | SNP:c.2764C > T (p.Arg922Trp) | VCV000067435 | Long QT Syn.\|Congenital long QT Syn. | Uncertain significance(LR: Mar. 18, 2020) |
| 817 | SNP:c.3278C > T (p.Pro1093Leu [symbol] | 598 | SNP:c.2678G > A (p.Arg893Lys) | VCV001025677 | Long QT Syn. | Uncertain significance(LR: Mar. 2, 2020) |
| 818 | SNP:c.3286C > A (p.Pro1096Thr [symbol] | 898 | SNP:c.44T > A (p.Leu15Gln) | VCV000200594 | Not Prov. | Uncertain significance(LR: Mar. 20, 2018) |
| 819 | SNP:c.3286C > T (p.Pro1096Ser [symbol] | 729 | SNP:c.2996G > A (p.Ser999Asn) | VCV000919130 | Arrhythmia | Uncertain significance(LR: Mar. 20, 2020) |
| 820 | SNP:c.3289G > A (p.Val1097Ile [symbol] | 812 | SNP:c.3233A > G (p.Tyr1078Cys) | VCV000067483 | Long QT Syn.\|Congenital long QT Syn.\|not [symbol] | Uncertain significance(LR: Mar. 22, 2019) |
| 821 | SNP:c.3292A > C (p.Ser1098Arg [symbol] | 465 | SNP:c.2231G > C (p.Arg744Pro) | VCV001017667 | Long QT Syn. | Uncertain significance(LR: Mar. 22, 2020) |
| 822 | SNP:c.3294C > G (p.Ser1098Arg [symbol] | 500 | SNP:c.235G > T (p.Ala79Ser) | VCV000456907 | Long QT Syn. | Uncertain significance(LR: Mar. 23, 2017) |
| 823 | SNP:c.3296C > T (p.Pro1099Leu [symbol] | 879 | SNP:c.388G > A (p.Glu130Lys) | VCV000067503 | Arrhythmia\|Long QT Syn.\|Congenital long Q [symbol] | Uncertain significance(LR: Mar. 23, 2019) |
| 824 | SNP:c.32A > G (p.Gln11Arg) | 81 | SNP:c.1357G > A (p.Ala453Thr) | VCV000919895 | Arrhythmia | Uncertain significance(LR: Mar. 24, 2020) |
| 825 | SNP:c.3301C > T (p.Pro1101Ser [symbol] | 160 | SNP:c.160T > C (p.Tyr54His) | VCV000067223 | Sudden cardiac death\|Prolonged QT interval\| [symbol] | Uncertain significance(LR: Mar. 25, 2019) |
| 826 | SNP:c.3302C > T (p.Pro1101Leu [symbol] | 243 | SNP:c.1749C > G (p.Ile583Met) | VCV001023474 | Long QT Syn. | Uncertain significance(LR: Mar. 25, 2020) |
| 827 | SNP:c.3305C > T (p.Thr1102Ile) | 923 | SNP:c.562G > A (p.Ala188Thr) | VCV001035380 | Long QT Syn. | Uncertain significance(LR: Mar. 25, 2020) |
| 828 | SNP:c.3315G > C (p.Leu1105Ph [symbol] | 87 | SNP:c.137A > G (p.Asp46Gly) | VCV001303967 | Not Prov. | Uncertain significance(LR: Mar. 25, 2021) |
| 829 | SNP:c.331G > A (p.Asp111Asn) | 983 | SNP:c.779C > T (p.Ala260Val) | VCV000571094 | Long QT Syn. | Uncertain significance(LR: Mar. 26, 2018) |
| 830 | SNP:c.3320C > T (p.Ser1107Leu [symbol] | 623 | SNP:c.2731G > C (p.Gly911Arg) | VCV000918838 | Arrhythmia | Uncertain significance(LR: Mar. 27, 2019) |
| 831 | SNP:c.3322C > G (p.Leu1108Va [symbol] | 715 | SNP:c.293T > G (p.Phe98Cys) | VCV000946662 | Long QT Syn. | Uncertain significance(LR: Mar. 29, 2019) |
| 832 | SNP:c.332A > T (p.Asp111Val) | 450 | SNP:c.2168G > A (p.Cys723Tyr) | VCV000200763 | Not Prov. | Uncertain significance(LR: Mar. 3, 2016) |
| 833 | SNP:c.3343A > G (p.Met1115Va [symbol] | 36 | SNP:c.1205A > G (p.His402Arg) | VCV000067166 | Long QT Syn.\|Congenital long QT Syn. | Uncertain significance(LR: Mar. 3, 2017) |
| 834 | SNP:c.3346G > A (p.Ala1116Th [symbol] | 603 | SNP:c.2684C > G (p.Thr895Arg) | VCV000919273 | Arrhythmia | Uncertain significance(LR: Mar. 4, 2019) |
| 835 | SNP:c.3347C > T (p.Ala1116Val [symbol] | 1042 | SNP:c.961G > T (p.Asp321Tyr) | VCV000359312 | Long QT Syn. 2\|Not Prov. | Uncertain significance(LR: Mar. 4, 2019) |
| 836 | SNP:c.3350G > A (p.Cys1117Ty [symbol] | 449 | SNP:c.2167T > G (p.Cys723Gly) | VCV000922895 | Arrhythmia | Uncertain significance(LR: Mar. 4, 2020) |
| 837 | SNP:c.3350G > T (p.Cys1117Ph [symbol] | 455 | SNP:c.2204C > T (p.Ser735Leu) | VCV000067374 | Long QT Syn.\|Congenital long QT Syn.\|Not [symbol] | Uncertain significance(LR: Mar. 4, 2020) |
| 838 | SNP:c.3352G > A (p.Glu1118Ly [symbol] | 886 | SNP:c.421C > T (p.Pro141Ser) | VCV001045443 | Long QT Syn. | Uncertain significance(LR: Mar. 4, 2020) |
| 839 | SNP:c.3355G > C (p.Glu1119Gl [symbol] | 810 | SNP:c.3229G > A (p.Ala1077Thr) | VCV000526983 | Long QT Syn.\|Arrhythmia | Uncertain significance(LR: Mar. 5, 2020) |
| 840 | SNP:c.3358C > G (p.Leu1120Va [symbol] | 303 | SNP:c.1849T > C (p.Phe617Leu) | VCV000207940 | Long QT Syn. | Uncertain significance(LR: Mar. 6, 2019) |
| 841 | SNP:c.3361C > T (p.Pro1121Ser [symbol] | 527 | SNP:c.243G > C (p.Gln81His) | VCV000067399 | Congenital long QT Syn.\|Long QT Syn.\|Long [symbol] | Uncertain significance(LR: Mar. 7, 2018) |
| 842 | SNP:c.3365C > G (p.Pro1122Ar [symbol] | 47 | SNP:c.1240A > G (p.Ile414Val) | VCV000924916 | Arrhythmia | Uncertain significance(LR: Mar. 7, 2019) |
| 843 | SNP:c.3365C > T (p.Pro1122Leu [symbol] | 289 | SNP:c.1826A > C (p.Asp609Ala) | VCV000456895 | Long QT Syn. | Uncertain significance(LR: Mar. 9, 2017) |
| 844 | SNP:c.3379C > T (p.Leu1127Phe [symbol] | 818 | SNP:c.3286C > A (p.Pro1096Thr) | VCV000456927 | Long QT Syn. | Uncertain significance(LR: Mar. 9, 2017) |
| 845 | SNP:c.3392G > T (p.Gly1131Val [symbol] | 822 | SNP:c.3294C > G (p.Ser1098Arg) | VCV000928123 | Arrhythmia | Uncertain significance(LR: Mar. 9, 2019) |
| 846 | SNP:c.3394C > G (p.Pro1132Ala [symbol] | 252 | SNP:c.1756C > G (p.Leu586Val) | VCV000993664 | none provided | Uncertain significance(LR: Mar. 9, 2020) |
| 847 | SNP:c.3400C > T (p.Arg1134Ter [symbol] | 1026 | SNP:c.927C > G (p.His309Gln) | VCV000810210 | Not Prov. | Uncertain significance(LR: May 1, 2018) |
| 848 | SNP:c.3403C > T (p.Arg1135Cys [symbol] | 232 | SNP:c.1733A > C (p.His578Pro) | VCV000200378 | Not Prov. | Uncertain significance(LR: May 10, 2014) |
| 849 | SNP:c.3404G > A (p.Arg1135Hi [symbol] | 309 | SNP:c.185G > A (p.Arg62Gln) | VCV000067298 | Congenital long QT Syn.\|Long QT Syn. | Uncertain significance(LR: May 10, 2019) |
| 850 | SNP:c.3406C > T (p.Leu1136Ph [symbol] | 110 | SNP:c.1462C > T (p.Arg488Cys) | VCV001018981 | Long QT Syn. | Uncertain significance(LR: May 12, 2020) |
| 851 | SNP:c.340C > T (p.Pro114Ser) | 658 | SNP:c.2788A > C (p.Ser930Arg) | VCV000200503 | Not Prov. | Uncertain significance(LR: May 14, 2014) |
| 852 | SNP:c.3410C > A (p.Ser1137Tyr [symbol] | 505 | SNP:c.2369T > C (p.Leu790Pro) | VCV001027323 | Long QT Syn. | Uncertain significance(LR: May 15, 2020) |
| 853 | SNP:c.3416C > T (p.Pro1139Leu [symbol] | 847 | SNP:c.3400C > T (p.Arg1134Ter) | VCV000923242 | Long QT Syn.\|Arrhythmia | Uncertain significance(LR: May 15, 2020) |
| 854 | SNP:c.3430G > A (p.Ala1144Th [symbol] | 706 | SNP:c.290C > G (p.Ala97Gly) | VCV000200580 | Not Prov. | Uncertain significance(LR: May 16, 2012) |
| 855 | SNP:c.3436A > T (p.Thr1146Ser [symbol] | 628 | SNP:c.273G > T (p.Glu91Asp) | VCV001305882 | Not Prov. | Uncertain significance(LR: May 16, 2019) |
| 856 | SNP:c.343G > A (p.Val115Met) | 20 | SNP:c.1081C > T (p.Pro361Ser) | VCV000456878 | Long QT Syn. | Uncertain significance(LR: May 18, 2017) |
| 857 | SNP:c.3448C > A (p.Leu1150Me [symbol] | 1001 | SNP:c.838C > T (p.Arg280Cys) | VCV000943502 | Long QT Syn. | Uncertain significance(LR: May 18, 2019) |
| 858 | SNP:c.344T > G (p.Val115Gly) | 28 | SNP:c.1178C > T (p.Pro393Leu) | VCV000933401 | Long QT Syn. | Uncertain significance(LR: May 19, 2019) |
| 859 | SNP:c.3453C > A (p.His1151Gln [symbol] | 756 | SNP:c.3061A > G (p.Ser1021Gly) | VCV000572915 | Long QT Syn. | Uncertain significance(LR: May 2, 2018) |
| 860 | SNP:c.3457C > T (p.His1153Ty [symbol] | 732 | SNP:c.29C > T (p.Pro10Leu) | VCV000946979 | Long QT Syn. | Uncertain significance(LR: May 2, 2019) |
| 861 | SNP:c.3460G > A (p.Gly1154Se [symbol] | 981 | SNP:c.775G > A (p.Asp259Asn) | VCV000067527 | Congenital long QT Syn.\|Long QT Syn.\|Not [symbol] | Uncertain significance(LR: May 20, 2020) |
| 862 | SNP:c.3464C > T (p.Ser1155Leu [symbol] | 835 | SNP:c.3347C > T (p.Ala1116Val) | VCV000067491 | Arrhythmia\|Congenital long QT Syn.\|Long Q [symbol] | Uncertain significance(LR: May 21, 2019) |
| 863 | SNP:c.3467A > G (p.Asp1156G [symbol] | 717 | SNP:c.2944G > A (p.Asp982Asn) | VCV000237299 | Arrhythmia\|Long QT Syn.\|Long QT Syn. 2 | Uncertain significance(LR: May 21, 2020) |
| 864 | SNP:c.3469C > T (p.Pro1157Ser [symbol] | 893 | SNP:c.443G > A (p.Arg148Gln) | VCV000567306 | Long QT Syn.\|Not Prov.\|Arrhythmia | Uncertain significance(LR: May 21, 2020) |
| 865 | SNP:c.3470C > T (p.Pro1157Leu [symbol] | 454 | SNP:c.2200C > T (p.Arg734Cys) | VCV000200765 | Arrhythmia\| Long QT Syn.\|Not Prov. | Uncertain significance(LR: May 21, 2021) |
| 866 | SNP:c.3473G > C (p.Gly1158Al [symbol] | 665 | SNP:c.2803C > G (p.Pro935Ala) | VCV000922520 | Arrhythmia | Uncertain significance(LR: May 23, 2019) |
| 867 | SNP:c.352G > A (p.Glu118Lys) | 408 | SNP:c.2030T > C (p.Met677Thr) | VCV000939911 | Long QT Syn. | Uncertain significance(LR: May 27, 2019) |
| 868 | SNP:c.352G > C (p.Glu118Gln) | 809 | SNP:c.3224C > T (p.Pro1075Leu) | VCV000067481 | Long QT Syn. 2\|Congenital long QT Syn.\|Lo [symbol] | Uncertain significance(LR: May 28, 2019) |
| 869 | SNP:c.355G > C (p.Asp119His) | 954 | SNP:c.697G > A (p.Ala233Thr) | VCV000848897 | Long QT Syn. | Uncertain significance(LR: May 28, 2019) |
| 870 | SNP:c.362C > T (p.Ala121Val) | 579 | SNP:c.2626G > A (p.Glu876Lys) | VCV000456914 | Long QT Syn.\|Not Prov. | Uncertain significance(LR: May 3, 2017) |
| 871 | SNP:c.371T > C (p.Met124Thr) | 90 | SNP:c.138C > G (p.Asp46Glu) | VCV000921751 | Arrhythmia | Uncertain significance(LR: May 30, 2019) |
| 872 | SNP:c.371T > G (p.Met124Arg) | 873 | SNP:c.373T > C (p.Phe125Leu) | VCV000934591 | Long QT Syn. | Uncertain significance(LR: May 30, 2019) |
| 873 | SNP:c.373T > C (p.Phe125Leu) | 792 | SNP:c.3172G > A (p.Ala1058Thr) | VCV000456924 | Long QT Syn. | Uncertain significance(LR: May 31, 2017) |
| 874 | SNP:c.374T > G (p.Phe125Cys) | 382 | SNP:c.1946C > T (p.Ser649Phe) | VCV000067346 | Long QT Syn.\|Congenital long QT Syn. | Uncertain significance(LR: May 31, 2019) |
| 875 | SNP:c.377T > A (p.Ile126Asn) | 233 | SNP:c.1736T > C (p.Met579Thr) | VCV000067255 | Long QT Syn.\|Congenital long QT Syn. | Uncertain significance(LR: May 31, 2020) |
| 876 | SNP:c.379C > T (p.Leu127Phe) | 526 | SNP:c.2437G > A (p.Ala813Thr) | VCV000928527 | not spec. | Uncertain significance(LR: May 6, 2019) |
| 877 | SNP:c.383A > G (p.Asn128Ser) | 836 | SNP:c.3350G > A (p.Cys1117Tyr) | VCV000180385 | Ventricular tachycardia | Uncertain significance(LR: May 7, 2014) |
| 878 | SNP:c.387C > G (p.Phe129Leu) | 1049 | SNP:c.980A > G (p.Tyr327Cys) | VCV000264025 | Cardiovas. pheno. | Uncertain significance(LR: May 8, 2015) |
| 879 | SNP:c.388G > A (p.Glu130Lys) | 620 | SNP:c.2719G > A (p.Ala907Thr) | VCV000200486 | Long QT Syn.\|Not Prov. | Uncertain significance(LR: May 8, 2019) |
| 880 | SNP:c.38C > A (p.Thr13Asn) | 137 | SNP:c.1537T > C (p.Phe513Leu) | VCV000567702 | Long QT Syn. | Uncertain significance(LR: May 9, 2019) |
| 881 | SNP:c.391G > A (p.Val131Met) | 679 | SNP:c.2853C > A (p.Ser951Arg) | VCV000840645 | Long QT Syn. | Uncertain significance(LR: May 9, 2019) |
| 882 | SNP:c.391G > T (p.Val131Leu) | 231 | SNP:c.1730C > A (p.Pro577Gln) | VCV000872354 | Not Prov. | Uncertain significance(LR: Nov. 1, 2019) |
| 883 | SNP:c.398T > C (p.Met133Thr) | 595 | SNP:c.2668T > C (p.Ser890Pro) | VCV000926306 | Arrhythmia | Uncertain significance(LR: Nov. 1, 2019) |
| 884 | SNP:c.409A > G (p.Met137Val) | 763 | SNP:c.3095G > A (p.Arg1032Gln) | VCV000067466 | Long QT Syn.\|Congenital long QT Syn.\|Not [symbol] | Uncertain significance(LR: Nov. 1, 2020) |
| 885 | SNP:c.416G > C (p.Gly139Ala) | 723 | SNP:c.2968G > A (p.Ala990Thr) | VCV000968306 | Arrhythmia\| Long QT Syn. | Uncertain significance(LR: Nov. 10, 2020) |
| 886 | SNP:c.421C > T (p.Pro141Ser) | 906 | SNP:c.478G > T (p.Ala160Ser) | VCV000955775 | Long QT Syn. | Uncertain significance(LR: Nov. 11, 2019) |
| 887 | SNP:c.422C > T (p.Pro141Leu) | 769 | SNP:c.3104G > A (p.Arg1035Gln) | VCV000456922 | Arrhythmia\|Long QT Syn. | Uncertain significance(LR: Nov. 11, 2020) |
| 888 | SNP:c.424G > A (p.Ala142Thr) | 977 | SNP:c.769A > C (p.Asn257His) | VCV000067526 | Not Prov. | Uncertain significance(LR: Nov. 12, 2018) |
| 889 | SNP:c.431A > T (p.Asp144Val) | 821 | SNP:c.3292A > C (p.Ser1098Arg) | VCV000966117 | Long QT Syn. | Uncertain significance(LR: Nov. 12, 2019) |
| 890 | SNP:c.434C > T (p.Thr145Ile) | 1025 | SNP:c.925C > T (p.His309Tyr) | VCV000526915 | Long QT Syn. | Uncertain significance(LR: Nov. 13, 2017) |
| 891 | SNP:c.440A > G (p.His 147Arg) | 721 | SNP:c.2956C > A (p.Pro986Thr) | VCV000640120 | Arrhythmia\| Long QT Syn. | Uncertain significance(LR: Nov. 14, 2019) |
| 892 | SNP:c.442C > T (p.Arg148Trp) | 12 | SNP:c.1054C > T (p.Pro352Ser) | VCV000918184 | Arrhythmia | Uncertain significance(LR: Nov. 16, 2018) |
| 893 | SNP:c.443G > A (p.Arg148Gln) | 617 | SNP:c.2711A > G (p.Glu904Gly) | VCV000642118 | Long QT Syn. | Uncertain significance(LR: Nov. 16, 2018) |
| 894 | SNP:c.446G > A (p.Gly149Asp) | 689 | SNP:c.2875T > A (p.Ser959Thr) | VCV000921152 | Arrhythmia | Uncertain significance(LR: Nov. 16, 2018) |
| 895 | SNP:c.446G > C (p.Gly149Ala) | 953 | SNP:c.695G > T (p.Arg232Leu) | VCV000264446 | Cardiovas. pheno. | Uncertain significance(LR: Nov. 18, 2015) |
| 896 | SNP:c.448C > T (p.Pro150Ser) | 778 | SNP:c.312C > G (p.Ser104Arg) | VCV000527024 | Long QT Syn. | Uncertain significance(LR: Nov. 18, 2017) |
| 897 | SNP:c.449C > T (p.Pro150Leu) | 509 | SNP:c.2380G > A (p.Val794Ile) | VCV000200434 | Long QT Syn.\|Arrhythmia\|Not Prov. | Uncertain significance(LR: Nov. 18, 2020) |
| 898 | SNP:c.44T > A (p.Leu15Gln) | 978 | SNP:c.76A > G (p.Ser26Gly) | VCV000928116 | Arrhythmia | Uncertain significance(LR: Nov. 18, 2020) |
| 899 | SNP:c.44T > G (p.Leu15Arg) | 922 | SNP:c.55A > T (p.Ile 19Phe) | VCV000527023 | Long QT Syn. | Uncertain significance(LR: Nov. 19, 2018) |
| 900 | SNP:c.451C > G (p.Pro151Ala) | 690 | SNP:c.2876C > G (p.Ser959Cys) | VCV000662474 | Long QT Syn. | Uncertain significance(LR: Nov. 2, 2018) |
| 901 | SNP:c.451C > T (p.Pro151Ser) | 764 | SNP:c.3095G > C (p.Arg1032Pro) | VCV001171150 | Arrhythmia | Uncertain significance(LR: Nov. 2, 2020) |
| 902 | SNP:c.455C > G (p.Thr152Ser) | 896 | SNP:c.448C > T (p.Pro150Ser) | VCV000222667 | Ventricular tachycardia | Uncertain significance(LR: Nov. 20, 2015) |
| 903 | SNP:c.455C > T (p.Thr152Ile) | 513 | SNP:c.2387T > G (p.Val796Gly) | VCV000518748 | Cardiovas. pheno. | Uncertain significance(LR: Nov. 20, 2017) |
| 904 | SNP:c.460T > C (p.Trp154Arg) | 574 | SNP:c.2612C > G (p.Ser871Cys) | VCV000921315 | Arrhythmia | Uncertain significance(LR: Nov. 20, 2018) |
| 905 | SNP:c.461G > A (p.Trp154Ter) | 828 | SNP:c.3315G > C (p.Leu1105Phe) | VCV000926697 | Arrhythmia | Uncertain significance(LR: Nov. 20, 2018) |
| 906 | SNP:c.478G > T (p.Ala160Ser) | 8 | SNP:c.1036G > A (p.Asp346Asn) | VCV000841013 | Long QT Syn. | Uncertain significance(LR: Nov. 22, 2019) |
| 907 | SNP:c.47A > C (p.Asp 16Ala) | 169 | SNP:c.1645G > A (p.Val549Met) | VCV000919780 | Arrhythmia | Uncertain significance(LR: Nov. 24, 2019) |
| 908 | SNP:c.491G > A (p.Arg164His) | 685 | SNP:c.2861G > A (p.Arg954His) | VCV000456917 | Arrhythmia\| Long QT Syn. | Uncertain significance(LR: Nov. 24, 2019) |
| 909 | SNP:c.499C > G (p.Leu167Val) | 414 | SNP:c.2046G > C (p.Glu682Asp) | VCV000487626 | Wolff-Parkinson-White pattern\|Not Prov.\|Ar [symbol] | Uncertain significance(LR: Nov. 25, 2020) |
| 910 | SNP:c.515C > T (p.Ala172Val) | 589 | SNP:c.2654G > A (p.Arg885His) | VCV000402995 | not spec.\|Arrhythmia\|Not Prov.\|Long QT Syn [symbol] | Uncertain significance(LR: Nov. 26, 2019) |
| 911 | SNP:c.523G > A (p.Ala175Thr) | 1054 | SNP:c.995A > G (p.Lys332Arg) | VCV000847270 | Long QT Syn. | Uncertain significance(LR: Nov. 26, 2019) |
| 912 | SNP:c.523G > T (p.Ala175Ser) | 788 | SNP:c.3161C > T (p.Thr1054Ile) | VCV000527001 | Long QT Syn. | Uncertain significance(LR: Nov. 27, 2017) |
| 913 | SNP:c.524C > A (p.Ala175Asp) | 132 | SNP:c.1516A > G (p.Ile506Val) | VCV000645833 | Long QT Syn. | Uncertain significance(LR: Nov. 28, 2018) |
| 914 | SNP:c.526C > T (p.Arg176Trp) | 425 | SNP:c.2086C > T (p.Arg696Cys) | VCV000067360 | Long QT Syn.\|Congenital long QT Syn. | Uncertain significance(LR: Nov. 28, 2018) |
| 915 | SNP:c.527G > A (p.Arg176Gln) | 582 | SNP:c.2635G > A (p.Gly879Ser) | VCV000924749 | Arrhythmia | Uncertain significance(LR: Nov. 29, 2018) |
| 916 | SNP:c.530A > C (p.Glu177Ala) | 498 | SNP:c.235G > A (p.Ala79Thr) | VCV000987798 | not spec. | Uncertain significance(LR: Nov. 30, 2020) |
| 917 | SNP:c.535T > G (p.Ser179Ala) | 76 | SNP:c.1342G > A (p.Ala448Thr) | VCV000924051 | Arrhythmia | Uncertain significance(LR: Nov. 4, 2019) |
| 918 | SNP:c.542G > A (p.Arg181Gln) | 641 | SNP:c.2765G > T (p.Arg922Leu) | VCV000924058 | Arrhythmia | Uncertain significance(LR: Nov. 4, 2019) |
| 919 | SNP:c.545C > A (p.Ser182Ter) | 659 | SNP:c.2788A > G (p.Ser930Gly) | VCV000924230 | Arrhythmia | Uncertain significance(LR: Nov. 4, 2019) |
| 920 | SNP:c.545C > G (p.Ser182Trp) | 1024 | SNP:c.923T > G (p.Met308Arg) | VCV000923222 | Arrhythmia\| Long QT Syn. | Uncertain significance(LR: Nov. 4, 2020) |
| 921 | SNP:c.559G > A (p.Gly187Ser) | 910 | SNP:c.515C > T (p.Ala172Val) | VCV000200290 | Not Prov. | Uncertain significance(LR: Nov. 5, 2013) |
| 922 | SNP:c.55A > T (p.Ile 19Phe) | 587 | SNP:c.2651A > G (p.Gln884Arg) | VCV000633279 | not spec. | Uncertain significance(LR: Nov. 6, 2017) |
| 923 | SNP:c.562G > A (p.Ala188Thr) | 82 | SNP:c.135C > A (p.Asn45Lys) | VCV000569898 | Long QT Syn. | Uncertain significance(LR: Nov. 6, 2018) |
| 924 | SNP:c.563C > T (p.Ala188Val) | 966 | SNP:c.731C > A (p.Ala244Glu) | VCV000969507 | Long QT Syn. | Uncertain significance(LR: Nov. 6, 2019) |
| 925 | SNP:c.566G > T (p.Gly189Val) | 402 | SNP:c.2006G > C (p.Gly669Ala) | VCV000647535 | Long QT Syn. | Uncertain significance(LR: Nov. 7, 2018) |
| 926 | SNP:c.568G > A (p.Ala190Thr) | 959 | SNP:c.710C > A (p.Pro237His) | VCV000526973 | Long QT Syn. | Uncertain significance(LR: Nov. 8, 2018) |
| 927 | SNP:c.56T > G (p.Ile 19Ser) | 485 | SNP:c.2312A > C (p.His771Pro) | VCV000964746 | Long QT Syn. | Uncertain significance(LR: Nov. 8, 2019) |
| 928 | SNP:c.572C > T (p.Pro191Leu) | 26 | SNP:c.1149C > A (p.Asp383Glu) | VCV000925167 | Arrhythmia | Uncertain significance(LR: Nov. 9, 2018) |
| 929 | SNP:c.578C > T (p.Ala193Val) | 761 | SNP:c.3092G > A (p.Gly1031Asp) | VCV000919480 | Arrhythmia | Uncertain significance(LR: Nov. 9, 2018) |
| 930 | SNP:c.580G > A (p.Val194Met) | 940 | SNP:c.647C > T (p.Thr216Ile) | VCV000582107 | Long QT Syn. | Uncertain significance(LR: Nov. 9, 2018) |
| 931 | SNP:c.589G > A (p.Asp197Asn) | 458 | SNP:c.2216A > G (p.His739Arg) | VCV000222670 | Long QT Syn. | Uncertain significance(LR: Oct. 1, 2015) |
| 932 | SNP:c.58C > G (p.Arg20Gly) | 135 | SNP:c.1520C > T (p.Pro507Leu) | VCV000200352 | Not Prov. | Uncertain significance(LR: Oct. 1, 2020) |
| 933 | SNP:c.602C > T (p.Thr201Met) | 866 | SNP:c.3473G > C (p.Gly1158Ala) | VCV000939464 | Long QT Syn. | Uncertain significance(LR: Oct. 10, 2019) |
| 934 | SNP:c.607G > A (p.Ala203Thr) | 553 | SNP:c.2537C > T (p.Pro846Leu) | VCV000405353 | Long QT Syn. | Uncertain significance(LR: Oct. 11, 2017) |
| 935 | SNP:c.613C > T (p.Pro205Ser) | 377 | SNP:c.1935G > A (p.Met645Ile) | VCV000067343 | Congenital long QT Syn.\|Not Prov. | Uncertain significance(LR: Oct. 11, 2018) |
| 936 | SNP:c.614C > T (p.Pro205Leu) | 806 | SNP:c.3211A > G (p.Met1071 Val) | VCV000629473 | Arrhythmia | Uncertain significance(LR: Oct. 11, 2018) |
| 937 | SNP:c.629T > C (p.Leu210Pro) | 859 | SNP:c.3453C > A (p.His1151Gln) | VCV000629472 | Arrhythmia | Uncertain significance(LR: Oct. 11, 2018) |
| 938 | SNP:c.638A > G (p.Asp213Gly) | 674 | SNP:c.2837C > T (p.Pro946Leu) | VCV001008413 | Long QT Syn. | Uncertain significance(LR: Oct. 12, 2020) |
| 939 | SNP:c.644T > G (p.Val215Gly) | 634 | SNP:c.2758C > G (p.Arg920Gly) | VCV000200490 | Arrhythmia\| Not Prov.\|Long QT Syn. | Uncertain significance(LR: Oct. 13, 2020) |
| 940 | SNP:c.647C > T (p.Thr216Ile) | 738 | SNP:c.3013C > T (p.Arg1005Trp) | VCV001019746 | Long QT Syn. | Uncertain significance(LR: Oct. 13, 2020) |
| 941 | SNP:c.652A > G (p.Met218Val) | 883 | SNP:c.398T > C (p.Met133Thr) | VCV000200282 | Not Prov.\|Long QT Syn. 2\|Long QT Syn. | Uncertain significance(LR: Oct. 14, 2019) |
| 942 | SNP:c.655G > A (p.Asp219Asn) | 549 | SNP:c.2530A > G (p.Met844Val) | VCV000658202 | Arrhythmia\|Not Prov.\|Long QT Syn. | Uncertain significance(LR: Oct. 14, 2020) |
| 943 | SNP:c.656A > T (p.Asp219Val) | 600 | SNP:c.2681G > A (p.Arg894His) | VCV000448972 | Inborn genetic diseases\|Long QT Syn. 2\|Arrh [symbol] | Uncertain significance(LR: Oct. 14, 2020) |
| 944 | SNP:c.65T > C (p.Phe22Ser) | 654 | SNP:c.2780G > T (p.Trp927Leu) | VCV000200501 | Arrhythmia\|Long QT Syn.\|Long QT Syn. 2\|N [symbol] | Uncertain significance(LR: Oct. 14, 2020) |
| 945 | SNP:c.663C > G (p.His221Gln) | 937 | SNP:c.629T > C (p.Leu210Pro) | VCV000938315 | Long QT Syn. | Uncertain significance(LR: Oct. 15, 2019) |
| 946 | SNP:c.664G > A (p.Val222Met) | 143 | SNP:c.1576A > G (p.Thr526Ala) | VCV001038284 | Long QT Syn. | Uncertain significance(LR: Oct. 15, 2020) |
| 947 | SNP:c.670G > A (p.Gly224Arg) | 624 | SNP:c.2734C > T (p.Arg912Trp) | VCV000180384 | Arrhythmia\|Not Prov.\|Long QT Syn. | Uncertain significance(LR: Oct. 15, 2020) |
| 948 | SNP:c.682G > C (p.Ala228Pro) | 678 | SNP:c.284A > C (p.Glu95Ala) | VCV000200578 | Long QT Syn.\|Not Prov. | Uncertain significance(LR: Oct. 15, 2020) |
| 949 | SNP:c.683C > T (p.Ala228Val) | 982 | SNP:c.778G > C (p.Ala260Pro) | VCV000427970 | Hypertrophic cardiomyopathy\|Long QT Syn. [symbol] | Uncertain significance(LR: Oct. 17, 2018) |
| 950 | SNP:c.685G > T (p.Glu229Ter) | 412 | SNP:c.203T > G (p.Phe68Cys) | VCV000940257 | Long QT Syn. | Uncertain significance(LR: Oct. 18, 2019) |
| 951 | SNP:c.692G > A (p.Arg231Gln) | 669 | SNP:c.2819G > T (p.Ser940Ile) | VCV000923239 | Arrhythmia | Uncertain significance(LR: Oct. 18, 2019) |
| 952 | SNP:c.694C > T (p.Arg232Cys) | 839 | SNP:c.3355G > C (p.Glu1119Gln) | VCV000067492 | Long QT Syn.\|Not Prov.\|Arrhythmia | Uncertain significance(LR: Oct. 18, 2019) |
| 953 | SNP:c.695G > T (p.Arg232Leu) | 848 | SNP:c.3403C > T (p.Arg1135Cys) | VCV000920427 | Not Prov.\|Arrhythmia | Uncertain significance(LR: Oct. 18, 2019) |
| 954 | SNP:c.697G > A (p.Ala233Thr) | 965 | SNP:c.729C > G (p.Ser243Arg) | VCV000648410 | Long QT Syn. | Uncertain significance(LR: Oct. 19, 2018) |
| 955 | SNP:c.697G > T (p.Ala233Ser) | 108 | SNP:c.1459G > A (p.Gly487Ser) | VCV000200342 | not spec.\|Arrhythmia\|Not Prov.\|Long QT Syn [symbol] | Uncertain significance(LR: Oct. 19, 2020) |
| 956 | SNP:c.704T > C (p.Val235Ala) | 172 | SNP:c.1652T > C (p.Phe551Ser) | VCV000392546 | Long QT Syn.\|Not Prov. | Uncertain significance(LR: Oct. 20, 2020) |
| 957 | SNP:c.707G > A (p.Gly236Asp) | 466 | SNP:c.2236G > T (p.Ala746Ser) | VCV000222671 | Not Prov.\|Long QT Syn. | Uncertain significance(LR: Oct. 20, 2020) |
| 958 | SNP:c.707G > T (p.Gly236Val) | 817 | SNP:c.3278C > T (p.Pro1093Leu) | VCV000067485 | Congenital long QT Syn.\|not spec.\|Long QT [symbol] | Uncertain significance(LR: Oct. 20, 2020) |
| 959 | SNP:c.710C > A (p.Pro237His) | 588 | SNP:c.2653C > T (p.Arg885Cys) | VCV000067422 | Congenital long QT Syn.\|not spec.\|Arrhythm [symbol] | Uncertain significance(LR: Oct. 21, 2020) |
| 960 | SNP:c.712G > A (p.Gly238Ser) | 699 | SNP:c.2899C > T (p.Pro967Ser) | VCV000923330 | Arrhythmia\|Long QT Syn. | Uncertain significance(LR: Oct. 22, 2019) |
| 961 | SNP:c.719C > T (p.Pro240Leu) | 486 | SNP:c.2312A > G (p.His771 Arg) | VCV000405338 | Long QT Syn. | Uncertain significance(LR: Oct. 24, 2018) |
| 962 | SNP:c.722C > T (p.Pro241Leu) | 430 | SNP:c.2097G > T (p.Glu699Asp) | VCV001001725 | Long QT Syn. | Uncertain significance(LR: Oct. 24, 2020) |
| 963 | SNP:c.724C > G (p.Arg242Gly) | 348 | SNP:c.1901C > T (p.Thr634Ile) | VCV000067325 | Congenital long QT Syn.\|Long QT Syn. | Uncertain significance(LR: Oct. 25, 2020) |
| 964 | SNP:c.724C > T (p.Arg242Cys) | 813 | SNP:c.3247A > G (p.Thr1083Ala) | VCV000200529 | Long QT Syn.\|Not Prov.\|Arrhythmia | Uncertain significance(LR: Oct. 26, 2020) |
| 965 | SNP:c.729C > G (p.Ser243 Arg) | 975 | SNP:c.758C > G (p.Ala253Gly) | VCV000200723 | Not Prov. | Uncertain significance(LR: Oct. 27, 2014) |
| 966 | SNP:c.731C > A (p.Ala244Glu) | 39 | SNP:c.1225G > A (p.Val409Met) | VCV000456884 | Arrhythmia\|Long QT Syn.\|Wolff-Parkinson- [symbol] | Uncertain significance(LR: Oct. 27, 2020) |
| 967 | SNP:c.731C > T (p.Ala244Val) | 952 | SNP:c.694C > T (p.Arg232Cys) | VCV000855032 | Long QT Syn. | Uncertain significance(LR: Oct. 27, 2020) |
| 968 | SNP:c.736G > T (p.Gly246Cys) | 991 | SNP:c.817C > G (p.Arg273Gly) | VCV001044178 | Long QT Syn. | Uncertain significance(LR: Oct. 27, 2020) |
| 969 | SNP:c.740A > G (p.Gln247Arg) | 31 | SNP:c.1189C > T (p.Arg397Cys) | VCV000405343 | Arrhythmia\|Cardiovas. pheno.\|Long QT Syn. [symbol] | Uncertain significance(LR: Oct. 28, 2018) |
| 970 | SNP:c.749C > A (p.Ser250Ter) | 774 | SNP:c.3118A > G (p.Ser1040Gly) | VCV000067472 | SUDDEN INFANT DEATH Syn.\|Long QT [symbol] | Uncertain significance(LR: Oct. 28, 2019) |
| 971 | SNP:c.751C > G (p.Pro251Ala) | 938 | SNP:c.638A > G (p.Asp213Gly) | VCV000971664 | Long QT Syn. | Uncertain significance(LR: Oct. 28, 2019) |
| 972 | SNP:c.751C > T (p.Pro251Ser) | 88 | SNP:c.1384A > G (p.Met462Val) | VCV001002624 | Long QT Syn. | Uncertain significance(LR: Oct. 28, 2020) |
| 973 | SNP:c.754C > G (p.Arg252Gly) | 824 | SNP:c.32A > G (p.Gln11Arg) | VCV001063296 | Long QT Syn. | Uncertain significance(LR: Oct. 28, 2020) |
| 974 | SNP:c.755G > A (p.Arg252Gln) | 837 | SNP:c.3350G > T (p.Cys1117Phe) | VCV000582288 | Long QT Syn. | Uncertain significance(LR: Oct. 29, 2018) |
| 975 | SNP:c.758C > G (p.Ala253Gly) | 945 | SNP:c.663C > G (p.His221Gln) | VCV000647893 | Long QT Syn. | Uncertain significance(LR: Oct. 29, 2018) |
| 976 | SNP:c.762C > G (p.His254Gln) | 728 | SNP:c.298C > T (p.Arg100Trp) | VCV000067459 | Congenital long QT Syn.\|Long QT Syn. | Uncertain significance(LR: Oct. 3, 2019) |
| 977 | SNP:c.769A > C (p.Asn257His) | 912 | SNP:c.523G > T (p.Ala175Ser) | VCV000937283 | Long QT Syn. | Uncertain significance(LR: Oct. 3, 2019) |
| 978 | SNP:c.76A > G (p.Ser26Gly) | 701 | SNP:c.2902C > G (p.Pro968Ala) | VCV000237298 | Not Prov.\|Long QT Syn.\|Arrhythmia | Uncertain significance(LR: Oct. 3, 2020) |
| 979 | SNP:c.772C > T (p.Pro258Ser) | 146 | SNP:c.1582C > T (p.Arg528Trp) | VCV000220162 | Long QT Syn.\|Arrhythmia | Uncertain significance(LR: Oct. 30, 2018) |
| 980 | SNP:c.773C > T (p.Pro258Leu) | 478 | SNP:c.227G > T (p.Arg76Leu) | VCV000629614 | Arrhythmia | Uncertain significance(LR: Oct. 30, 2018) |
| 981 | SNP:c.775G > A (p.Asp259Asn) | 948 | SNP:c.682G > C (p.Ala228Pro) | VCV000526974 | Long QT Syn. | Uncertain significance(LR: Oct. 30, 2020) |
| 982 | SNP:c.778G > C (p.Ala260Pro) | 1006 | SNP:c.868G > C (p.Ala290Pro) | VCV000200306 | Not Prov. | Uncertain significance(LR: Oct. 31, 2016) |
| 983 | SNP:c.779C > T (p.Ala260Val) | 895 | SNP:c.446G > C (p.Gly149Ala) | VCV000067506 | Long QT Syn. 2\|Short QT Syn. 1\|Long QT S [symbol] | Uncertain significance(LR: Oct. 31, 2018) |
| 984 | SNP:c.77G > T (p.Ser26Ile) | 869 | SNP:c.355G > C (p.Asp119His) | VCV000405352 | Long QT Syn.\|Not Prov. | Uncertain significance(LR: Oct. 31, 2019) |
| 985 | SNP:c.797G > T (p.Ser266Ile) | 1018 | SNP:c.896C > T (p.Pro299Leu) | VCV000641100 | Long QT Syn. | Uncertain significance(LR: Oct. 4, 2018) |
| 986 | SNP:c.803C > A (p.Ala268Asp) | 611 | SNP:c.26C > T (p.Ala9Val) | VCV000200592 | Arrhythmia\|Not Prov.\|Long QT Syn. | Uncertain significance(LR: Oct. 4, 2019) |
| 987 | SNP:c.805C > T (p.Arg269Trp) | 558 | SNP:c.2566C > G (p.Leu856Val) | VCV000629380 | Arrhythmia | Uncertain significance(LR: Oct. 5, 2018) |
| 988 | SNP:c.80G > A (p.Arg27His) | 585 | SNP:c.2647C > T (p.Arg883Trp) | VCV000925591 | Arrhythmia\| Long QT Syn.\|Not Prov. | Uncertain significance(LR: Oct. 5, 2020) |
| 989 | SNP:c.80G > C (p.Arg27Pro) | 766 | SNP:c.3098G > A (p.Arg1033Gln) | VCV000456920 | Long QT Syn. | Uncertain significance(LR: Oct. 5, 2020) |
| 990 | SNP:c.812G > A (p.Arg271His) | 784 | SNP:c.3140G > A (p.Arg1047His) | VCV000628410 | Long QT Syn.\|Arrhythmia | Uncertain significance(LR: Oct. 5, 2020) |
| 991 | SNP:c.817C > G (p.Arg273Gly) | 1036 | SNP:c.94G > A (p.Ala32Thr) | VCV000067549 | Congenital long QT Syn.\|Arrhythmia | Uncertain significance(LR: Oct. 6, 2020) |
| 992 | SNP:c.817C > T (p.Arg273Ter) | 547 | SNP:c.251A > G (p.Gln84Arg) | VCV001299593 | Long QT Syn. 2 | Uncertain significance(LR: Oct. 6, 2021) |
| 993 | SNP:c.818G > A (p.Arg273Gln) | 163 | SNP:c.1621C > T (p.Arg541Cys) | VCV000937094 | Long QT Syn.\|Arrhythmia | Uncertain significance(LR: Oct. 7, 2020) |
| 994 | SNP:c.823A > C (p.Ser275Arg) | 626 | SNP:c.2737G > A (p.Ala913Thr) | VCV001188698 | Not Prov. | Uncertain significance(LR: Oct. 7, 2020) |
| 995 | SNP:c.825C > G (p.Ser275Arg) | 1044 | SNP:c.973G > A (p.Val325Met) | VCV000526931 | Long QT Syn. | Uncertain significance(LR: Oct. 8, 2017) |
| 996 | SNP:c.829G > T (p.Ala277Ser) | 504 | SNP:c.2366T > C (p.Ile789Thr) | VCV000200431 | Cardiovas. pheno.\|Long QT Syn. | Uncertain significance(LR: Oct. 9, 2019) |
| 997 | SNP:c.82A > G (p.Lys28Glu) | 661 | SNP:c.2792C > G (p.Pro931Arg) | VCV000928265 | Arrhythmia | Uncertain significance(LR: Oct. 9, 2019) |
| 998 | SNP:c.830C > A (p.Ala277Asp) | 814 | SNP:c.3251C > T (p.Pro1084Leu) | VCV000456925 | Cardiovas. pheno.\|Long QT Syn.\|Arrhythmia [symbol] | Uncertain significance(LR: Oct. 9, 2019) |
| 999 | SNP:c.832A > T (p.Ser278Cys) | 23 | SNP:c.1099A > G (p.Thr367Ala) | VCV001047434 | Long QT Syn. | Uncertain significance(LR: Oct. 9, 2020) |
| 1000 | SNP:c.835G > A (p.Val279Met) | 167 | SNP:c.1639G > A (p.Ala547Thr) | VCV000663436 | Arrhythmia\| Long QT Syn. | Uncertain significance(LR: Oct. 9, 2020) |
| 1001 | SNP:c.838C > T (p.Arg280Cys) | 403 | SNP:c.200A > G (p.Asp67Gly) | VCV001047548 | Long QT Syn. | Uncertain significance(LR: Oct. 9, 2020) |
| 1002 | SNP:c.853G > A (p.Ala285Thr) | 783 | SNP:c.3139C > T (p.Arg1047Cys) | VCV000161255 | Not Prov.\|Long QT Syn. | Uncertain significance(LR: Oct. 9, 2020) |
| 1003 | SNP:c.853G > T (p.Ala285Ser) | 827 | SNP:c.3305C > T (p.Thr1102Ile) | VCV000200531 | Long QT Syn.\|Arrhythmia\|Not Prov. | Uncertain significance(LR: Oct. 9, 2020) |
| 1004 | SNP:c.863T > C (p.Ile288Thr) | 985 | SNP:c.797G > T (p.Ser266Ile) | VCV001060485 | Long QT Syn. | Uncertain significance(LR: Oct. 9, 2020) |
| 1005 | SNP:c.865G > A (p.Glu289Lys) | 670 | SNP:c.281T > G (p.Val94Gly) | VCV000067441 | Congenital long QT Syn.\|Long QT Syn. | Uncertain significance(LR: Sep. 10, 2018) |
| 1006 | SNP:c.868G > C (p.Ala290Pro) | 825 | SNP:c.3301C > T (p.Pro1101Ser) | VCV000456928 | Long QT Syn.\|Arrhythmia | Uncertain significance(LR: Sep. 10, 2018) |
| 1007 | SNP:c.868G > T (p.Ala290Ser) | 664 | SNP:c.2795C > T (p.Ser932Phe) | VCV000925842 | Arrhythmia | Uncertain significance(LR: Sep. 10, 2019) |
| 1008 | SNP:c.86T > C (p.Phe29Ser) | 1035 | SNP:c.941G > A (p.Gly314Asp) | VCV000925690 | Arrhythmia | Uncertain significance(LR: Sep. 10, 2019) |
| 1009 | SNP:c.872T > C (p.Met291Thr) | 141 | SNP:c.156C > G (p.Cys52Trp) | VCV000972150 | Long QT Syn. | Uncertain significance(LR: Sep. 10, 2020) |
| 1010 | SNP:c.872T > G (p.Met291Arg) | 575 | SNP:c.2612C > T (p.Ser871Phe) | VCV000954476 | Long QT Syn. | Uncertain significance(LR: Sep. 11, 2019) |
| 1011 | SNP:c.875G > A (p.Arg292His) | 708 | SNP:c.2926G > A (p.Asp976Asn) | VCV000928384 | Arrhythmia\|Long QT Syn. | Uncertain significance(LR: Sep. 11, 2019) |
| 1012 | SNP:c.877G > A (p.Ala293Thr) | 16 | SNP:c.1067G > A (p.Arg356His) | VCV000180382 | Not Prov.\|Long QT Syn.\|Cardiovas. pheno.\|A [symbol] | Uncertain significance(LR: Sep. 11, 2020) |
| 1013 | SNP:c.877G > T (p.Ala293Ser) | 1004 | SNP:c.863T > C (p.Ile288Thr) | VCV000598780 | Long QT Syn. 2 | Uncertain significance(LR: Sep. 12, 2018) |
| 1014 | SNP:c.87C > A (p.Phe29Leu) | 584 | SNP:c.2641T > A (p.Phe881Ile) | VCV000855339 | Long QT Syn | Uncertain significance(LR: Sep. 12, 2020) |
| 1015 | SNP:c.880G > A (p.Gly294Arg) | 820 | SNP:c.3289G > A (p.Val1097Ile) | VCV000067486 | Long QT Syn.\|Not Prov.\|Cardiovas. pheno.\|n [symbol] | Uncertain significance(LR: Sep. 13, 2021) |
| 1016 | SNP:c.881G > T (p.Gly294Val) | 691 | SNP:c.2876C > T (p.Ser959Phe) | VCV000657772 | Long QT Syn. | Uncertain significance(LR: Sep. 14, 2018) |
| 1017 | SNP:c.889C > T (p.Pro297Ser) | 619 | SNP:c.2717C > T (p.Ser906Leu) | VCV000067429 | Long QT Syn.\|not spec.\|Long QT Syn. 2\| Sho [symbol] | Uncertain significance(LR: Sep. 15, 2020) |
| 1018 | SNP:c.896C > T (p.Pro299Leu) | 1048 | SNP:c.979T > C (p.Tyr327His) | VCV000629260 | Arrhythmia | Uncertain significance(LR: Sep. 16, 2018) |
| 1019 | SNP:c.89T > C (p.Ile30Thr) | 106 | SNP:c.1447G > A (p.Val483Ile) | VCV000858924 | Long QT Syn.\|Arrhythmia | Uncertain significance(LR: Sep. 16, 2020) |
| 1020 | SNP:c.902G > T (p.Arg301Leu) | 899 | SNP:c.44T > G (p.Leu15Arg) | VCV001206671 | Not Prov. | Uncertain significance(LR: Sep. 16, 2020) |
| 1021 | SNP:c.908C > G (p.Ala303Gly) | 752 | SNP:c.3053C > T (p.Pro1018Leu) | VCV000651422 | Long QT Syn. | Uncertain significance(LR: Sep. 17, 2018) |
| 1022 | SNP:c.916G > C (p.Gly306Arg) | 645 | SNP:c.2770G > T (p.Gly924Trp) | VCV000519287 | Long QT Syn.\|Arrhythmia\|Cardiovas. pheno. [symbol] | Uncertain significance(LR: Sep. 17, 2019) |
| 1023 | SNP:c.916G > T (p.Gly306Trp) | 216 | SNP:c.1708G > A (p.Ala570Thr) | VCV001043586 | Long QT Syn. | Uncertain significance(LR: Sep. 17, 2020) |
| 1024 | SNP:c.923T > G (p.Met308Arg) | 451 | SNP:c.2171T > C (p.Leu724Pro) | VCV000527007 | Long QT Syn.\|Not Prov. | Uncertain significance(LR: Sep. 19, 2019) |
| 1025 | SNP:c.925C > T (p.His309Tyr) | 479 | SNP:c.2291C > G (p.Pro764Arg) | VCV000955346 | Long QT Syn. | Uncertain significance(LR: Sep. 19, 2019) |
| 1026 | SNP:c.927C > G (p.His309Gln) | 481 | SNP:c.2296G > A (p.Gly766Arg) | VCV000519498 | Cardiovas. pheno. | Uncertain significance(LR: Sep. 2, 2017) |
| 1027 | SNP:c.928C > A (p.Pro310Thr) | 1017 | SNP:c.889C > T (p.Pro297Ser) | VCV000067540 | Not Prov.\|Congenital long QT Syn.\|Long QT [symbol] | Uncertain significance(LR: Sep. 2, 2020) |
| 1028 | SNP:c.92T > A (p.Ile31Asn) | 5 | SNP:c.1010C > G (p.Thr337Ser) | VCV001043131 | Long QT Syn. | Uncertain significance(LR: Sep. 20, 2020) |
| 1029 | SNP:c.92T > C (p.Ile31Thr) | 363 | SNP:c.1919T > G (p.Phe640Cys) | VCV001045742 | Long QT Syn. | Uncertain significance(LR: Sep. 20, 2020) |
| 1030 | SNP:c.92T > G (p.Ile31Ser) | 696 | SNP:c.2891C > G (p.Pro964Arg) | VCV001021720 | Long QT Syn. | Uncertain significance(LR: Sep. 20, 2020) |
| 1031 | SNP:c.934C > T (p.Arg312Cys) | 206 | SNP:c.169G > A (p.Ala57Thr) | VCV000654924 | Long QT Syn. | Uncertain significance(LR: Sep. 21, 2018) |
| 1032 | SNP:c.935G > A (p.Arg312His) | 819 | SNP:c.3286C > T (p.Pro1096Ser) | VCV000921691 | Arrhythmia | Uncertain significance(LR: Sep. 23, 2019) |
| 1033 | SNP:c.93C > G (p.Ile31Met) | 1005 | SNP:c.865G > A (p.Glu289Lys) | VCV000067535 | Not Prov.\|Long QT Syn.\|Long QT Syn. 2\|not [symbol] | Uncertain significance(LR: Sep. 23, 2021) |
| 1034 | SNP:c.940G > A (p.Gly314Ser) | 903 | SNP:c.455C > T (p.Thr152Ile) | VCV000940899 | Long QT Syn. | Uncertain significance(LR: Sep. 24, 2019) |
| 1035 | SNP:c.941G > A (p.Gly314Asp) | 341 | SNP:c.1894C > G (p.Pro632Ala) | VCV001054005 | Long QT Syn. | Uncertain significance(LR: Sep. 24, 2020) |
| 1036 | SNP:c.94G > A (p.Ala32Thr) | 457 | SNP:c.220A > G (p.Thr74Ala) | VCV001040732 | Long QT Syn. | Uncertain significance(LR: Sep. 24, 2020) |
| 1037 | SNP:c.950A > G (p.Asn317Ser) | 511 | SNP:c.2386G > C (p.Val796Leu) | VCV000200438 | Long QT Syn. | Uncertain significance(LR: Sep. 24, 2020) |
| 1038 | SNP:c.959C > A (p.Ser320Ter) | 590 | SNP:c.2659C > G (p.Arg887Gly) | VCV000924438 | Arrhythmia Long QT Syn. | Uncertain significance(LR: Sep. 24, 2020) |
| 1039 | SNP:c.959C > G (p.Ser320Trp) | 757 | SNP:c.3070A > T (p.Asn1024Tyr) | VCV001022039 | Long QT Syn. | Uncertain significance(LR: Sep. 24, 2020) |
| 1040 | SNP:c.959C > T (p.Ser320Leu) | 890 | SNP:c.434C > T (p.Thr145Ile) | VCV001047559 | Long QT Syn. | Uncertain significance(LR: Sep. 24, 2020) |
| 1041 | SNP:c.95C > T (p.Ala32Val) | 750 | SNP:c.3049G > T (p.Ala1017Ser) | VCV000565852 | Long QT Syn.\|Arrhythmia | Uncertain significance(LR: Sep. 25, 2020) |
| 1042 | SNP:c.961G > T (p.Asp321Tyr) | 856 | SNP:c.343G > A (p.Val115Met) | VCV000067496 | Long QT Syn.\|Long QT Syn. 2\|Congenital lo [symbol] | Uncertain significance(LR: Sep. 25, 2020) |
| 1043 | SNP:c.967G > A (p.Asp323Asn) | 917 | SNP:c.535T > G (p.Ser179Ala) | VCV001059858 | Long QT Syn. | Uncertain significance(LR: Sep. 25, 2020) |
| 1044 | SNP:c.973G > A (p.Val325Met) | 644 | SNP:c.2770G > A (p.Gly924Arg) | VCV000200492 | Not Prov.\|Long QT Syn. | Uncertain significance(LR: Sep. 26, 2018) |
| 1045 | SNP:c.973G > C (p.Val325Leu) | 672 | SNP:c.2832G > T (p.Glu944Asp) | VCV000526953 | Long QT Syn. | Uncertain significance(LR: Sep. 27, 2017) |
| 1046 | SNP:c.976C > T (p.Arg326Cys) | 435 | SNP:c.211G > A (p.Gly71Arg) | VCV000948713 | Long QT Syn. | Uncertain significance(LR: Sep. 30, 2019) |
| 1047 | SNP:c.977G > A (p.Arg326His) | 713 | SNP:c.2932G > C (p.Glu978Gln) | VCV001042802 | Long QT Syn. | Uncertain significance(LR: Sep. 30, 2020) |
| 1048 | SNP:c.979T > C (p.Tyr327His) | 986 | SNP:c.803C > A (p.Ala268Asp) | VCV000941477 | Long QT Syn. | Uncertain significance(LR: Sep. 4, 2019) |
| 1049 | SNP:c.980A > G (p.Tyr327Cys) | 10 | SNP:c.1043T > A (p.Phe348Tyr) | VCV000200319 | Long QT Syn.\|Not Prov. | Uncertain significance(LR: Sep. 4, 2020) |
| 1050 | SNP:c.982C > T (p.Arg328Cys) | 935 | SNP:c.613C > T (p.Pro205Ser) | VCV000662184 | Long QT Syn. | Uncertain significance(LR: Sep. 6, 2018) |
| 1051 | SNP:c.983G > A (p.Arg328His) | 709 | SNP:c.2930G > T (p.Cys977Phe) | VCV000405354 | Long QT Syn. | Uncertain significance(LR: Sep. 7, 2016) |
| 1052 | SNP:c.985A > G (p.Thr329Ala) | 780 | SNP:c.3134T > G (p.Leu1045Arg) | VCV001025586 | Long QT Syn. | Uncertain significance(LR: Sep. 7, 2020) |
| 1053 | SNP:c.98A > C (p.Asn33Thr) | 681 | SNP:c.2855C > G (p.Pro952Arg) | VCV001036276 | Long QT Syn. | Uncertain significance(LR: Sep. 9, 2020) |
| 1054 | SNP:c.995A > G (p.Lys332Arg) | 805 | SNP:c.3209A > T (p.Gln1070Leu) | VCV001043117 | Long QT Syn. | Uncertain significance(LR: Sep. 9, 2020) |

| | |
|---|---|
| Not. Prov. | not provided |
| Syn. | syndrome |
| Not. Spec. | not specified |
| Cardiovas. | cardiovascular |
| Pheno. | phenotype |
| Conf. Interp. of Patho | conflicting interpretations of pathogenicity |

[symbol]

indicates data missing or illegible when filed

TABLE 2
LQT Black Box Labeled Drugs for Testing by the
Methods of the Disclosure (PREDICT PLATFORM)

| | Molecule | | Revenue | | | |
|---|---|---|---|---|---|---|
| Use | Type | Generic name | 2020* | | Therapy Area | Indication |
| Acute | Small | Clarithromycin | 21 | | Gastrointestinal | Duodenal Ulcer, |
| | Molecule | | | | | Gastric Ulcers; |
| | | | | | | Zollinger-Ellison |
| | | | | | | Syndrome |
| | | | | | | (Gastrinoma) |
| | | Levofloxacin | 119 | | Ear Nose Throat | Acute Bacterial |
| | | | | | Disorders | Sinusitis |
| | | Azithromycin | 276 | | Ear Nose Throat | Acute Bacterial |
| | | | | | Disorders | Sinusitis; Otitis Media; |
| | | | | | | Tonsillitis |
| | | Ciprofloxacin | <1 | | Ear Nose Throat | Otitis Externa; Otitis |
| | | | | | Disorders | Media |
| | | Ondansetron | <1 | | Toxicology | Chemotherapy Induced |
| | | | | | | Nausea and Vomiting; |
| | | | | | | Radiation Induced |
| | | | | | | Nausea and Vomiting |
| | | | | | | (RINV) |
| | | Sevoflurane | 278** | | Central Nervous | General Anesthetic |
| | | | | | System | Effect |
| Chronic | Small | Oxaliplatin | 226 | (1) | Oncology | Endometrial Cancer; |
| | Molecule | | | | | Head And Neck Cancer |
| | | | | | | Squamous Cell |
| | | | | | | Carcinoma; Hodgkin |
| | | | | | | Lymphoma (B-Cell |
| | | | | | | Hodgkin Lymphoma); |
| | | | | | | Metastatic Melanoma; |
| | | | | | | Non-Small Cell Lung |
| | | | | | | Cancer; Primary |
| | | | | | | Mediastinal B-Cell |
| | | | | | | Lymphoma; |
| | | | | | | Transitional Cell |
| | | | | | | Carcinoma (Urothelial |
| | | | | | | Cell Carcinoma) |
| | | Methadone | 38 | | Central Nervous | Cancer Pain; Opium |
| | | | | | System | (Opioid) Addiction |
| | | Cilostazol | 92 | | Cardiovascular | Intermittent |
| | | | | | | Claudication |
| | | Donepezil | 248 | | Central Nervous | Alzheimer's Disease: |
| | | | | | System | Dementia Associated |
| | | | | | | With Alzheimer's |
| | | | | | | Disease |
| | | Meglumine | 1288 | (613) | Metabolic Disorders | Familial Amyloid |
| | | antimoniate | | | | Cardiomyopathy; |
| | | | | | | Familial Amyloid |
| | | | | | | Neuropathies |
| | | Quinidine | 224 | (224) | Central Nervous | Amyotrophic Lateral |
| | | | | | System | Sclerosis |
| | | Citalopram | 364 | (19) | Central Nervous | Agoraphobia; |
| | | | | | System | Generalized Anxiety |
| | | | | | | Disorder (GAD); Major |
| | | | | | | Depressive Disorder; |
| | | | | | | Panic Disorders; Social |
| | | | | | | Anxiety Disorder |
| | | | | | | (SAD/Social Phobia) |
| | | Escitalopram | 364 | (19) | Central Nervous | Agoraphobia; |
| | | | | | System | Generalized Anxiety |
| | | | | | | Disorder (GAD); Major |
| | | | | | | Depressive Disorder; |
| | | | | | | Obsessive-Compulsive |
| | | | | | | Disorder; Social |
| | | | | | | Anxiety Disorder |
| | | | | | | (SAD/Social Phobia) |
| | | Dronedarone | 365 | (312) | Cardiovascular | Atrial Fibrillation |
| | Synthetic | Terlipressin | 39** | | Gastrointestinal | Esophageal |
| | Peptide | | | | | Hemorrhage; |
| | | | | | | Hepatorenal Syndrome |

TABLE 3
Torsade de Pointes (TdP) Drugs to Avoid (source CredibleMeds)

| Brand name | Generic Name | Risk |
|---|---|---|
| Zytiga and others | Abiraterone | (CR) |
| Symmetrel and others | Amantadine | (CR) |
| Barhemsys and others | Amisulpride | (CR) |
| Elavil (Discontinued | Amitriptyline | (CR) |
| 6/13) and others | | |
| Fungilin and others | Amphotericin | (CR) |
| Amsidine | Amsacrine | (CR) |
| Reyataz and others | Atazanavir | (CR) |
| Aprinox and others | Bendroflumethiazide | (CR) |
| Aquachloral and others | Chloral | (CR) |
| Tagamet | Cimetidine | (CR) |
| Anafranil | Clomipramine | (CR) |
| Cardizem | Diltiazem | (CR) |
| Benadryl and others | Diphenhydramine | (CR) |
| Sinequan and others | Doxepin | (CR) |
| Myonal and others | Eperisone | (CR) |
| Nexium and others | Esomeprazole | (CR) |
| Pepcid and others | Famotidine | (CR) |
| Prozac and others | Fluoxetine | (CR) |
| Faverin and others | Fluvoxamine | (CR) |
| Lasix and others | Furosemide | (CR) |
| Reminyl and others | Galantamine | (CR) |
| Geninax | Garenoxacin | (CR) |
| Apo-Hydro and others | Hydrochlorothiazide | (CR) |
| Atarax and others | Hydroxyzine | (CR) |
| Lozol and others | Indapamide | (CR) |
| Sporanox and others | Itraconazole | (CR) |
| Procoralan and others | Ivabradine | (CR) |
| Nizoral and others | Ketoconazole | (CR) |
| Prevacid and others | Lansoprazole | (CR) |
| Imodium | Loperamide | (CR) |
| Reglan and others | Metoclopramide | (CR) |
| Zytanix and others | Metolazone | (CR) |
| Flagyl and others | Metronidazole | (CR) |
| Viracept | Nelfinavir | (CR) |
| Zyprexa and others | Olanzapine | (CR) |
| Losec and others | Omeprazole | (CR) |
| Protonix and others | Pantoprazole | (CR) |
| Paxil and others | Paroxetine | (CR) |
| Tazosyn and others | Piperacillin/Tazobactam | (CR) |
| Noxafil and others | Posaconazole | (CR) |
| Rythmol SR and others | Propafenone | (CR) |
| Seroquel | Quetiapine | (CR) |
| Qualaquin and others | Quinine | (CR) |
| Ranexa and others | Ranolazine | (CR) |
| Risperdal | Risperidone | (CR) |
| Zoloft and others | Sertraline | (CR) |
| Vesicare | Solifenacin | (CR) |
| Incivo and others | Telaprevir | (CR) |
| Demadex and others | Torsemide | (CR) |
| Desyrel and others | Trazodone | (CR) |
| VFend | Voriconazole | (CR) |
| Geodon and others | Ziprasidone | (CR) |
| Aclacin and others | Aclarubicin | (KR) |
| Cordarone and others | Amiodarone | (KR) |
| Agrylin and others | Anagrelide | (KR) |
| Trisenox | Arsenic | (KR) |
| Hismanal | Astemizole | (KR) |
| Zithromax and others | Azithromycin | (KR) |
| Vascor | Bepridil | (KR) |
| Energy Catalyst | Cesium | (KR) |
| Aralen | Chloroquine | (KR) |
| Thorazine and others | Chlorpromazine | (KR) |
| Truxal | Chlorprothixene | (KR) |
| Pletal | Cilostazol | (KR) |
| Cipro and others | Ciprofloxacin | (KR) |
| Propulsid | Cisapride | (KR) |
| Celexa and others | Citalopram | (KR) |
| Biaxin and others | Clarithromycin | (KR) |
| Cocaine | Cocaine | (KR) |
| Norpace | Disopyramide | (KR) |
| Tikosyn | Dofetilide | (KR) |
| Motilium and others | Domperidone | (KR) |
| Aricept | Donepezil | (KR) |
| Multaq | Dronedarone | (KR) |
| Inapsine and others | Droperidol | (KR) |
| E.E.S. and others | Erythromycin | (KR) |
| Cipralex and others | Escitalopram | (KR) |
| Tambocor and others | Flecainide | (KR) |
| Diflucan and others | Fluconazole | (KR) |
| Tequin | Gatifloxacin | (KR) |
| Raxar | Grepafloxacin | (KR) |
| Halfan | Halofantrine | (KR) |
| Haldol and others | Haloperidol | (KR) |
| Serecor | Hydroquinidine | (KR) |
| Plaquenil and others | Hydroxychloroquine | (KR) |
| | Ibogaine | (KR) |
| Corvert | Ibutilide | (KR) |
| Levaquin and others | Levofloxacin | (KR) |
| Nosinan and others | Levomepromazine | (KR) |
| Orlaam | Levomethadyl | (KR) |
| Lesuride and others | Levosulpiride | (KR) |
| Glucantime | Meglumine | (KR) |
| Serentil | Mesoridazine | (KR) |
| Dolophine and others | Methadone | (KR) |
| Avelox and others | Moxifloxacin | (KR) |
| Shinbit | Nifekalant | (KR) |
| Zofran and others | Ondansetron | (KR) |
| Eloxatin | Oxaliplatin | (KR) |
| | Papaverine | (KR) |
| Pentam | Pentamidine | (KR) |
| Orap | Pimozide | (KR) |
| Lorelco | Probucol | (KR) |
| Pronestyl and others | Procainamide | (KR) |
| Diprivan and others | Propofol | (KR) |
| Quinaglute and others | Quinidine | (KR) |
| Rulide and others | Roxithromycin | (KR) |
| Serdolect and others | Sertindole | (KR) |
| Ultane and others | Sevoflurane | (KR) |
| Betapace and others | Sotalol | (KR) |
| Zagam | Sparfloxacin | (KR) |
| Dogmatil and others | Sulpiride | (KR) |
| Barnetil and others | Sultopride | (KR) |
| Seldane | Terfenadine | (KR) |
| Teripress and others | Terlipressin | (KR) |
| Micturin and others | Terodiline | (KR) |
| Mellaril and others | Thioridazine | (KR) |
| Caprelsa | Vandetanib | (KR) |
| Uroxatral | Alfuzosin | (PR) |
| Nedeltran and others | Alimemazine | (PR) |
| Erleada | Apalutamide | (PR) |
| Apokyn and others | Apomorphine | (PR) |
| Abilify and others | Aripiprazole | (PR) |
| Coartem | Artemether/Lumefantrine | (PR) |
| Eurartesim | Artenimol/piperaquine | (PR) |
| Saphris and others | Asenapine | (PR) |
| Strattera | Atomoxetine | (PR) |
| Sirturo | Bedaquiline | (PR) |
| Treanda and others | Bendamustine | (PR) |
| Bevyxxa | Betrixaban | (PR) |
| Casodex | Bicalutamide | (PR) |
| Velcade and others | Bortezomib | (PR) |
| Bosulif | Bosutinib | (PR) |
| Butrans and others | Buprenorphine | (PR) |
| Cometriq | Cabozantinib | (PR) |
| Xeloda | Capecitabine | (PR) |
| Pabal and others | Carbetocin | (PR) |
| Zykadia | Ceritinib | (PR) |
| Lamprene | Clofazimine | (PR) |
| Entumine | Clotiapine | (PR) |
| Clozaril and others | Clozapine | (PR) |
| Cotellic | Cobimetinib | (PR) |
| Xalkori | Crizotinib | (PR) |
| Tercian | Cyamemazine | (PR) |
| Tafinlar | Dabrafenib | (PR) |
| Sprycel | Dasatinib | (PR) |
| Firmagon and others | Degarelix | (PR) |
| Deltyba | Delamanid | (PR) |
| Pertofrane and others | Desipramine | (PR) |
| Austedo | Deutetrabenazine | (PR) |
| Precedex and others | Dexmedetomidine | (PR) |
| Nuedexta | Dextromethorphan/Quinidine | (PR) |
| Anzemet | Dolasetron | (PR) |
| Sustiva | Efavirenz | (PR) |
| Cerdelga | Eliglustat | (PR) |
| Braftovi | Encorafenib | (PR) |
| Rozlytrek | Entrectinib | (PR) |
| Ellence and others | Epirubicin | (PR) |
| Halaven | Eribulin | (PR) |
| Potiga and others | Ezogabine | (PR) |
| Felbatol | Felbamate | (PR) |
| Gilenya | Fingolimod | (PR) |
| Adrucil and others | Fluorouracil | (PR) |
| Depixol and others | Flupentixol | (PR) |
| Factive | Gemifloxacin | (PR) |
| Xospata | Gilteritinib | (PR) |
| Daurismo | Glasdegib | (PR) |
| Kytril and others | Granisetron | (PR) |
| Hysingla ™ ER and others | Hydrocodone | (PR) |
| Fanapt and others | Iloperidone | (PR) |
| Tofranil | Imipramine | (PR) |
| Besponsa | Inotuzumab | (PR) |
| Dynacirc | Isradipine | (PR) |
| Tibsovo | Ivosidenib | (PR) |
| Sufrexal | Ketanserin | (PR) |
| Lacipil and others | Lacidipine | (PR) |
| Tykerb and others | Lapatinib | (PR) |
| Xenleta | Lefamulin | (PR) |
| Lenvima | Lenvatinib | (PR) |
| Lupron and others | Leuprolide | (PR) |
| Keppra | Levetiracetam | (PR) |
| | Levomethadone | (PR) |
| Zyvox and others | Linezolid | (PR) |
| Eskalith and others | Lithium | (PR) |
| Lucemyra | Lofexidine | (PR) |
| Kaletra and others | Lopinavir/Ritonavir | (PR) |
| Latuda | Lurasidone | (PR) |
| Ludiomil | Maprotiline | (PR) |
| Bunil and others | Melperone | (PR) |
| Tolvon | Mianserin | (PR) |
| Rydapt | Midostaurin | (PR) |
| Korlym and others | Mifepristone | (PR) |
| Myrbetriq | Mirabegron | (PR) |
| Remeron | Mirtazapine | (PR) |
| Portrazza | Necitumumab | (PR) |
| Cardene | Nicardipine | (PR) |
| Tasigna | Nilotinib | (PR) |
| Noroxin and others | Norfloxacin | (PR) |
| Pamelor and others | Nortriptyline | (PR) |
| Spinraza | Nusinersen | (PR) |
| Floxin | Ofloxacin | (PR) |
| Olinvyk | Oliceridine | (PR) |
| Isturisa | Osilodrostat | (PR) |
| Tagrisso | Osimertinib | (PR) |
| Pitocin and others | Oxytocin | (PR) |
| Zeposia | Ozanimod | (PR) |
| Invega and others | Paliperidone | (PR) |
| Aloxi | Palonosetron | (PR) |
| Farydak | Panobinostat | (PR) |
| Signifor | Pasireotide | (PR) |
| Votrient | Pazopanib | (PR) |
| Definity and others | Perflutren | (PR) |
| Trilafon and others | Perphenazine | (PR) |
| Sunrythm | Pilsicainide | (PR) |
| Nuplazid | Pimavanserin | (PR) |
| Dipiperon and others | Pipamperone | (PR) |
| Wakix | Pitolisant | (PR) |
| Ponvory | Ponesimod | (PR) |
| | Pretomanid | (PR) |
| | Primaquine | (PR) |
| Phenergan | Promethazine | (PR) |
| Dominal and others | Prothipendyl | (PR) |
| Orgovyx and others | Relugolix | (PR) |
| Byfavo | Remimazolam | (PR) |
| Kisqali | Ribociclib | (PR) |
| Edurant and others | Rilpivirine | (PR) |
| Istodax | Romidepsin | (PR) |
| Rubraca | Rucaparib | (PR) |
| Invirase(combo) | Saquinavir | (PR) |
| Retevmo | Selpercatinib | (PR) |
| Mayzent | Siponimod | (PR) |
| Nexavar | Sorafenib | (PR) |
| Sutent | Sunitinib | (PR) |
| Prograf and others | Tacrolimus | (PR) |
| Nolvadex and others | Tamoxifen | (PR) |
| Tazverik | Tazemetostat | (PR) |
| Vibativ | Telavancin | (PR) |
| Ketek | Telithromycin | (PR) |
| Nitoman and others | Tetrabenazine | (PR) |
| Tiapridal and others | Tiapride | (PR) |
| Lonsurf | Tipiracil/Trifluridine | (PR) |
| Zanaflex and others | Tizanidine | (PR) |
| Detrol and others | Tolterodine | (PR) |
| Fareston | Toremifene | (PR) |
| Crispin and others | Tramadol | (PR) |
| Surmontil and others | Trimipramine | (PR) |
| Navoban and others | Tropisetron | (PR) |
| Ingrezza | Valbenazine | (PR) |
| Levitra | Vardenafil | (PR) |
| Zelboraf | Vemurafenib | (PR) |
| Effexor and others | Venlafaxine | (PR) |
| Lupkynis | Voclosporin | (PR) |
| Zolinza | Vorinostat | (PR) |
| Losizopilon and others | Zotepine | (PR) |
| Cisordinol and others | Zuclopenthixol | (PR) |
| Proventil and others | Albuterol | (SR) |
| Adderal-XR and others | Amphetamine | (SR) |
| Brovana | Arformoterol | (SR) |
| Didrex and others | Benzphetamine | (SR) |
| Focalin and others | Dexmethylphenidate | (SR) |
| Dexedrine and others | Dextroamphetamine | (SR) |
| Tenuate and others | Diethylpropion | (SR) |
| Dobutrex | Dobutamine | (SR) |
| Intropine | Dopamine | (SR) |
| Northera | Droxidopa | (SR) |
| Rynatuss and others | Ephedrine | (SR) |
| Primatene and others | Epinephrine | (SR) |
| Fintepla | Fenfluramine | (SR) |
| Berotec and others | Fenoterol | (SR) |
| Advair and others | Fluticasone | (SR) |
| Foradil and others | Formoterol | (SR) |
| Arcapta Neohaler and others | Indacaterol | (SR) |
| Medihaler-Iso and others | Isoproterenol | (SR) |
| Xopenex and others | Levalbuterol | (SR) |
| Sinu Inhaler and others | Levomethamphetamine | (SR) |
| Vyvanse | Lisdexamfetamine | (SR) |
| Metaprel and others | Metaproterenol | (SR) |
| Desoxyn and others | Methamphetamine | (SR) |
| Ritalin and others | Methylphenidate | (SR) |
| ProAmatine and others | Midodrine | (SR) |
| Levophed | Norepinephrine | (SR) |
| Striverdi Respimat | Olodaterol | (SR) |
| Afrin and others | Oxymetazoline | (SR) |
| Bontril and others | Phendimetrazine | (SR) |
| Adipex P and others | Phentermine | (SR) |
| Neosynephrine | Phenylephrine | (SR) |
| Acutrim and others | Phenylpropanolamine | (SR) |
| PediaCare and others | Pseudoephedrine | (SR) |
| Yutopar | Ritodrine | (SR) |
| Serevent and others | Salmeterol | (SR) |
| Meridia | Sibutramine | (SR) |
| Septra and others | Sulfamethoxazole | (SR) |
| Brethine and others | Terbutaline | (SR) |
| Breo Ellipta | Vilanterol/fluticasone | (SR) |
| Sinosil and others | Xylometazoline | (SR) |

### Abbreviations

(CR) Conditional Risk
(KR) Known Risk
(PR) Possible Risk
(SR) Special Risk

TABLE 4
DNA sequences coding the respective gRNAs and related target sequences in
the KCNH2 gene for CRISPRi

| | | SEQ | | | SEQ |
|---|---|---|---|---|---|
| | | ID | Genomic | | ID |
| Rank | Target sequence 5′-3′ | NO: | location | gRNA Sequence 5′-3′ | NO: |
| 1 | CATCCCGGCCGCCTACAAGC | 1055 | chr7:150978576 | CATCCCGGCCGCCTAC | 1116 |
| | CGG | | | AAGC | |
| 2 | GCATTAACCCTTCCGCGGCCG | 1056 | chr7:150978304 | GCATTAACCCTTCCGC | 1117 |
| | GG | | | GGCC | |
| 3 | CGCGCGCCCGACCTTCCCATT | 1057 | chr7:150978426 | CGCGCGCCCGACCTTC | 1118 |
| | GG | | | CCAT | |
| 9 | GCGCCGCATTAACCCTTCCGC | 1058 | chr7:150978309 | GCGCCGCATTAACCCT | 1119 |
| | GG | | | TCCG | |
| 10 | TCTGGGCGCGCGAGTCCCAG | 1059 | chr7:150978501 | TCTGGGCGCGCGAGTC | 1120 |
| | GGG | | | CCAG | |
| 11 | TTAGTCGCTAATCTGGGGTGG | 1060 | chr7:150978615 | TTAGTCGCTAATCTGG | 1121 |
| | GG | | | GGTG | |
| 15 | ACTCGCGCGCCCAGAAGAGG | 1061 | chr7:150978509 | ACTCGCGCGCCCAGAA | 1122 |
| | GGG | | | GAGG | |
| 17 | ACTCGGCGCGGCTCAGCCAA | 1062 | chr7:150978442 | ACTCGGCGCGGCTCAG | 1123 |
| | TGG | | | CCAA | |
| 18 | GGTTTCTTAGTCGCTAATCTG | 1063 | chr7:150978621 | GGTTTCTTAGTCGCTA | 1124 |
| | GG | | | ATCT | |
| 21 | CGTTGGGGGAGCACTCGGCG | 1064 | chr7:150978454 | CGTTGGGGGAGCACTC | 1125 |
| | CGG | | | GGCG | |
| 22 | TAGTCGCTAATCTGGGGTGG | 1065 | chr7:150978614 | TAGTCGCTAATCTGGG | 1126 |
| | GGG | | | GTGG | |
| 23 | CGGCCGCGGAAGGGTTAATG | 1066 | chr7:150978306 | CGGCCGCGGAAGGGT | 1127 |
| | CGG | | | TAATG | |
| 26 | GTTTCTTAGTCGCTAATCTGG | 1067 | chr7:150978620 | GTTTCTTAGTCGCTAA | 1128 |
| | GG | | | TCTG | |
| 29 | ATGGGAAGGTCGGGCGCGCG | 1068 | chr7:150978423 | ATGGGAAGGTCGGGC | 1129 |
| | CGG | | | GCGCG | |
| 33 | TGCCATGGCCGGCTTGTAGG | 1069 | chr7:150978568 | TGCCATGGCCGGCTTG | 1130 |
| | CGG | | | TAGG | |
| 36 | AAGGTCGGGCGCGCGCGGTG | 1070 | chr7:150978418 | AAGGTCGGGCGCGCG | 1131 |
| | GGG | | | CGGTG | |
| 37 | GCGCGGCTCAGCCAATGGGA | 1071 | chr7:150978437 | GCGCGGCTCAGCCAAT | 1132 |
| | AGG | | | GGGA | |
| 39 | GCAGGGGAAACCGTGAGGAC | 1072 | chr7:150978545 | GCAGGGGAAACCGTG | 1133 |
| | AGG | | | AGGAC | |
| 40 | GACAGCCGCTCCAGCGCCCG | 1073 | chr7:150978258 | GACAGCCGCTCCAGCG | 1134 |
| | CGG | | | CCCG | |
| 42 | CGCTCTGCCAAGGAGCCCCT | 1074 | chr7:150978486 | CGCTCTGCCAAGGAGC | 1135 |
| | GGG | | | CCCT | |
| 44 | AAGAGGGGGAAGGGACCCTT | 1075 | chr7:150978523 | AAGAGGGGGAAGGGA | 1136 |
| | AGG | | | CCCTT | |
| 46 | AACCGTGAGGACAGGTGCCA | 1076 | chr7:150978553 | AACCGTGAGGACAGG | 1137 |
| | TGG | | | TGCCA | |
| 47 | GCTGTCGGCGCGGTGGCAGG | 1077 | chr7:150978241 | GCTGTCGGCGCGGTGG | 1138 |
| | CGG | | | CAGG | |
| 48 | CGCGAGTCCCAGGGGCTCCT | 1078 | chr7:150978493 | CGCGAGTCCCAGGGG | 1139 |
| | TGG | | | CTCCT | |
| 53 | CAGAGCGAGGGGCTGGCGTT | 1079 | chr7:150978470 | CAGAGCGAGGGGCTG | 1140 |
| | GGG | | | GCGTT | |
| 54 | TGGAGCGGCTGTCGGCGCGG | 1080 | chr7:150978248 | TGGAGCGGCTGTCGGC | 1141 |
| | TGG | | | GCGG | |
| 57 | CTTAGGCAGGGGAAACCGTG | 1081 | chr7:150978540 | CTTAGGCAGGGGAAA | 1142 |
| | AGG | | | CCGTG | |
| 60 | GCCAGCCCCTCGCTCTGCCAA | 1082 | chr7:150978476 | GCCAGCCCCTCGCTCT | 1143 |
| | GG | | | GCCA | |
| 66 | CTCGGGCAGCGCCTGCGGCT | 1083 | chr7:150978281 | CTCGGGCAGCGCCTGC | 1144 |
| | CGG | | | GGCT | |
| 69 | TGGAATCCCTTCCCCCATCCC | 1084 | chr7:150978591 | TGGAATCCCTTCCCCC | 1145 |
| | GG | | | ATCC | |
| 70 | GGGAAGGGACCCTTAGGCAG | 1085 | chr7:150978529 | GGGAAGGGACCCTTA | 1146 |
| | GGG | | | GGCAG | |
| 71 | CAGGAGTCCGGGGCTCGGGC | 1086 | chr7:150978214 | CAGGAGTCCGGGGCTC | 1147 |
| | GGG | | | GGGC | |
| 72 | GAGCGAGGGGCTGGCGTTGG | 1087 | chr7:150978468 | GAGCGAGGGGCTGGC | 1148 |
| | GGG | | | GTTGG | |
| 74 | AGGCGGCCGGGATGGGGGAA | 1088 | chr7:150978585 | AGGCGGCCGGGATGG | 1149 |
| | GGG | | | GGGAA | |
| 77 | AGGCGGGAGCCAGGAGTCCG | 1089 | chr7:150978224 | AGGCGGGAGCCAGGA | 1150 |
| | GGG | | | GTCCG | |
| 79 | GGGTCCCTTCCCCCTCTTCTG | 1090 | chr7:150978518 | GGGTCCCTTCCCCCTC | 1151 |
| | GG | | | TTCT | |
| 83 | GAGCCAGGAGTCCGGGGCTC | 1091 | chr7:150978218 | GAGCCAGGAGTCCGG | 1152 |
| | GGG | | | GGCTC | |
| 85 | GGGGGAGAGGGCGGTGCTGC | 1092 | chr7:150978385 | GGGGGAGAGGGCGGT | 1153 |
| | GGG | | | GCTGC | |
| 88 | GGCGGGATGGGGGGGGGAG | 1093 | chr7:150978397 | GGCGGGATGGGGGGG | 1154 |
| | AGGG | | | GGAGA | |
| 89 | GGGATGGGGGGGGGAGAGG | 1094 | chr7:150978394 | GGGATGGGGGGGGGA | 1155 |
| | GCGG | | | GAGGG | |

We claim:

1. A prime editing guide RNA (pegRNA) designed for prime editing of a target gene that is hypothesized as having an association with arrhythmia and/or drug-induced toxicity to cardiomyocytes, preferably wherein the cardiomyocytes are derived from induced pluripotent stem cells (iPSC), and preferably wherein the pegRNA is barcoded.

2. The pegRNA of claim 1, comprising a spacer sequence and a DNA synthesis template, wherein the spacer sequence comprises a region of complementarity to a target strand of a double stranded target gene DNA sequence to be edited and the DNA synthesis template comprises a region of complementarity to the non-target strand of the double-stranded target gene DNA sequence and one or more nucleotide edits compared to the target strand double-stranded target gene DNA sequence.

3. The pegRNA of any one of claims 1 and 2, comprising a guide RNA (gRNA) core, and an extension arm comprising a DNA synthesis template and a primer binding site (PBS),
wherein the gRNA core associates with a nucleic acid programmable DNA binding protein (napDNAbp), which is fused to a domain comprising polymerase (preferably, RNA-dependent DNA polymerase) activity,
wherein the primer binding site comprises a region of complementarity to a non-target strand of the double-stranded target gene DNA sequence.

4. The pegRNA of any one of claims 1 to 3, wherein the DNA synthesis template is designed to edit the target gene in at least one site.

5. The pegRNA of claim 4, wherein the DNA synthesis template introduces random edits into the target gene.

6. The pegRNA of claim 4, wherein the DNA synthesis template introduces pre-selected edits into the target gene.

7. The pegRNA of any one of claims 1 to 7, wherein the target gene is selected from genes that encode potassium channels or potassium-channel related genes, sodium channels or sodium channels related genes, calcium channels and calcium channel related genes, and cardiomyocyte structural genes.

8. The pegRNA of claim 7, wherein the target gene is selected from potassium channel/related genes selected from human ether-a-go-go related gene (hERG)/KCNH2, Hyperpolarization-activated cyclic nucleotide-gated (HCN) channels; transient outward potassium current channel; Slowly activating delayed rectifier potassium current channels; Rapidly activating delayed rectifier potassium current channels; Inwardly rectifying potassium (Kir) channels; Inwardly rectifying potassium channels; G protein-coupled, inwardly rectifying potassium channels; ATP-sensitive potassium channels; sodium channel/related genes selected from SCN5A, ACN1B, SCN2B, SCN3B, SCN4B, GPD1L, RANGRF, SCN10A; calcium channel/related genes selected from CACNA1C, CACNB2, CACNA2D1, RYR2, CASQ2, TRDN, CALM1-3; and other genes selected from KCNQ1, SCNA5, KCNE1, KCNE2, KvLQT1, Nav1.5, ankyrin-B, MinK, MiRP1, Kir2.1, Cav1.2, caveolin-3, Nav.beta.4 genes, SNTAI, SLMAP, PKP2, ANK2, CAV3, SLC4A3, TRPM4, DPP6, IRX3, GNAI2, ADORAI, GNAS, KCNQ1, SCNA5, KCNE1, KCNE2, KvLQT1, Nav1.5, ankyrin-B, MinK, MiRP1, Kir2.1, Cav1.2, caveolin-3, Nav.beta.4 ATP-binding cassette (ABC) transporters involved in drug transport (e.g., ABCB1, ABCB4, ABCCI, ABCC2, SLC10A2, SLC28A3, SLC22A7, SLC22A17), Carbonyl reductases in drug metabolism (e.g., CBR3), Hyaluronan synthase 3 involved in oxidative stress response (e.g., HAS3), Hereditary hemochromatosis protein in iron metabolism (e.g., HFE), Retinoic acid receptor gamma and DNA topoisomerases in topoisomerase-induced DNA damage (e.g., RARG, TOP2B), CUGBP Elav-like family member 4 in splicing of sarcomere genes (e.g., CELF4), DNA polymerase gamma in mitochondrial replication (e.g., DPOG2), and chaperones involved in ion channel trafficking (e.g., Hsp70 and Hsp90).

9. The pegRNA of any one of claims 1 to 8, wherein the prime editing introduces one or more genetic variations into the gene, where the genetic variations have been previously identified as benign, likely benign, pathogenic, or likely pathogenic to cardiac function.

10. The pegRNA of any one of claims 1 to 6, wherein the drug has not been previously identified as a drug capable of inducing cardiomyocyte toxicity.

11. The pegRNA of any one of claims 1 to 6, wherein the drug is selected from a list of blackbox labeled drugs (currently marketed drugs that contain a label to indicate that Long QT patients should avoid using this drug; including Table 2), ii) drugs withdrawn from the US market due to cardiotoxicity events, iii) drugs that failed clinical trials due to cardiotoxicity events-(some may be marketed in other countries; iv) drugs associated with cardiotoxicity for particular SNPs as documented in the literature (e.g., ClinVar; antiarrhythmics, class i and iii; cisapride, amiodarone, dofetilide, clarithromycin, hydroxyzine, quinidine and disopyramide), and v) TdP associated drugs (including Table 3).

12. The pegRNA of any one of claims 1 to 11, wherein the pegRNA is designed to introduce a mutation into the human KCNH2 gene, wherein the mutation is selected from a SNP, preferably wherein the SNP is selected from the SNPs identified in Table 1 and/or SEQ ID Nos. 2-1054.

13. The pegRNA of claim 12, wherein the SNP causes a V476I mutation in the KCNH2 protein as in SEQ ID NO. 105.

14. The pegRNA of any one of claims 1 through 13, wherein the pegRNA is encoded by the DNA sequence of any one of SEQ ID Nos.: 1100 to 1113.

15. A gRNA designed for the regulation of expression of a target gene by CRISPR interference, wherein the target gene is being tested for their role in cardiomyocyte function, proliferation, viability, survival, morphology, the expression of markers and receptors, and “heart beats” in vitro (which can model arrhythmias), preferably wherein the target gene is selected from: potassium channel/related genes selected from human ether-a-go-go related gene (hERG), Hyperpolarization-activated cyclic nucleotide-gated (HCN) channels; transient outward potassium current channel; Slowly activating delayed rectifier potassium current channels; Rapidly activating delayed rectifier potassium current channels; Inwardly rectifying potassium (Kir) channels; Inwardly rectifying potassium channels; G protein-coupled, inwardly rectifying potassium channels; ATP-sensitive potassium channels; sodium channel/related genes selected from SCN5A, ACN1B, SCN2B, SCN3B, SCN4B, GPD1L, RANGRF, SCN10A; calcium channel/related genes selected from CACNA1C, CACNB2, CACNA2D1, RYR2, CASQ2, TRDN, CALM1-3; and other genes selected from KCNQ1, SCNA5, KCNE1, KCNE2, KvLQT1, Nav1.5, ankyrin-B, MinK, MiRP1, Kir2.1, Cav1.2, caveolin-3, Nav.beta.4 genes, SNTAI, SLMAP, PKP2, ANK2, CAV3, SLC4A3, TRPM4, DPP6, IRX3, GNAI2, ADORAI, GNAS, KCNQ1, SCNA5, KCNE1, KCNE2, KvLQT1, Nav1.5, ankyrin-B, MinK, MiRP1, Kir2.1, Cav1.2, caveolin-3, Nav.beta.4 ATP-binding cassette (ABC) transporters involved in drug transport (e.g., ABCB1, ABCB4, ABCCI, ABCC2, SLC10A2, SLC28A3, SLC22A7, SLC22A17), Carbonyl reductases in drug metabolism (e.g., CBR3), Hyaluronan synthase 3 involved in oxidative stress response (e.g., HAS3), Hereditary hemochromatosis protein in iron metabolism (e.g., HFE), Retinoic acid receptor gamma and DNA topoisomerases in topoisomerase-induced DNA damage (e.g., RARG, TOP2B), CUGBP Elav-like family member 4 in splicing of sarcomere genes (e.g., CELF4), DNA polymerase gamma in mitochondrial replication (e.g., DPOG2), and chaperones involved in ion channel trafficking (e.g., Hsp70 and Hsp90).

16. The gRNA of claim 15, wherein the gRNA target sequence is selected from those of SEQ ID NO. 1116 to 1155 and described in Table 4.

17. A composition comprising a pegRNA nucleic acid according to any one of claims 1 to 14, and, optionally, a prime editor comprising nucleic acid programmable DNA binding protein (napDNAbp), which is fused to or bound to a domain comprising polymerase (preferably, RNA-dependent DNA polymerase) activity, a sgRNA, an iPSC, and a cardiomyocyte.

18. A composition comprising a library of two or more pegRNA nucleic acids according to any one of claims 1 to 11, wherein all of the pegRNA nucleic acids are designed for prime editing of one or more (cardiomyocyte) genes at one or more multiple sites, and, optionally, a prime editor comprising nucleic acid programmable DNA binding protein (napDNAbp), which is fused to or bound to a domain comprising polymerase (preferably, RNA-dependent DNA polymerase) activity, a sgRNA, an iPSC, and a cardiomyocyte.

19. The composition of claim 12, wherein each of the pegRNA nucleic acids are designed for prime editing of a single (cardiomyocyte) gene.

20. The composition of any one of claims 12 and 13, further comprising one or more negative controls each comprising a pegRNA that does not introduce any edits into one or more genes.

21. A composition comprising the gRNA of claim 15 and, optionally, a nuclease.

22. A cell modified by CRISPR interference with a DNA polynucleotide coding for the gRNA of claim 15.

23. A cell genetically engineered to carry one or more specific mutations in one or more target genes, wherein the target genes are being tested for their role in cardiomyocyte function, proliferation, viability, survival, morphology, the expression of markers and receptors, and “heart beats” in vitro (which can model arrhythmias), preferably wherein the cell is a iPSC or a cardiomyocyte derived from a iPSC.

24. The cell of claim 23, wherein the cell is genetically engineered using CRISPR, base editing, or prime editing, preferably prime editing, more preferably with a pegRNA of any one of claims 1 through 14.

25. The cell of any one of claims 23 and 24, wherein the target gene is selected from KCNH2/hERG, KCNQ1, SCNA5, KCNE1, KCNE2, KvLQT1, Nav1.5, ankyrin-B, MinK, MiRP1, Kir2.1, Cav1.2, caveolin-3, and Nav.beta.4 genes; genes encoding other channels, a sodium channel/related gene selected from SCN5A, ACN1B, SCN2B, SCN3B, SCN4B, GPD1L, RANGRF, SCN10A; a potassium channel/related gene selected from human ether-a-go-go related gene (hERG), Hyperpolarization-activated cyclic nucleotide-gated (HCN) channels; transient outward potassium current channel; Slowly activating delayed rectifier potassium current channels; Rapidly activating delayed rectifier potassium current channels; Inwardly rectifying potassium (Kir) channels; Inwardly rectifying potassium channels; G protein-coupled, inwardly rectifying potassium channels; ATP-sensitive potassium channels; a calcium channel/related gene selected from CACNA1C, CACNB2, CACNA2D1, RYR2, CASQ2, TRDN, CALM1-3; other genes selected from SNTA1, SLMAP, PKP2, ANK2, CAV3, SLC4A3, TRPM4, DPP6, IRX3, GNAI2, ADORAI, GNAS, KCNQ1, SCNA5, KCNE1, KCNE2, KvLQT1, Nav1.5, ankyrin-B, MinK, MiRP1, Kir2.1, Cav1.2, caveolin-3, Nav.beta.4 ATP-binding cassette (ABC) transporters involved in drug transport (e.g., ABCB1, ABCB4, ABCCI, ABCC2, SLC10A2, SLC28A3, SLC22A7, SLC22A17), Carbonyl reductases in drug metabolism (e.g., CBR3), Hyaluronan synthase 3 involved in oxidative stress response (e.g., HAS3), Hereditary hemochromatosis protein in iron metabolism (e.g., HFE), Retinoic acid receptor gamma and DNA topoisomerases in topoisomerase-induced DNA damage (e.g., RARG, TOP2B), CUGBP Elav-like family member 4 in splicing of sarcomere genes (e.g., CELF4), DNA polymerase gamma in mitochondrial replication (e.g., DPOG2), and chaperones involved in ion channel trafficking (e.g., Hsp70 and Hsp90)

26. The cell of claim 25, wherein the mutation replicates one or more SNPs listed in Table 1 of SEQ ID NO.2 to 1054, preferably SEQ ID NO.105.

27. The cell of any one of claims 23 to 26, wherein the cell carries a V476I mutation in the KCNH2 gene.

28. The cell of any one of claims 23 to 27, wherein the mutation was known to be or is being tested as associated with heart disease.

29. The cell of claim 28, wherein the heart disease is an arrhythmia, hypertrophic cardiomyopathy, sudden unexplained death, obesity-related cardiac disease, primary dilated cardiomyopathy, primary familial hypertrophic cardiomyopathy, or seizure, preferably an arrhythmia selected from Long QT syndrome (including Congenital long QT syndrome, Long QT syndrome 2, and bradycardia-induced Long QT syndrome), Brugada syndrome, Short QT syndrome 1, Sudden Infant Death Syndrome, Acquired long QT syndrome, ventricular tachycardia, Wolff-Parkinson-White pattern arrhythmia, Arrhythmogenic right ventricular cardiomyopathy, Atrial fibrillation, Catecholaminergic polymorphic ventricular tachycardia type 1, Prolonged QT interval, Paroxysmal familial ventricular fibrillation 1, Sudden cardiac arrest/death, and Torsades de pointes.

30. The cell of any one of claims 23 to 29, wherein the cell expresses an mRNA having a sequence comprising SEQ ID NOs.: 2-1054 from Table 1, preferably SEQ ID NO.: 105.

31. A composition comprising a library of two or more iPS cells and/or cardiomyocytes wherein each iPS cell/cardiomyocyte has been modified to comprise one or more pegRNA molecules according to any one of claims 1 to 11 or is a cell of any one of claims 23 to 30.

32. The composition of claim 31 further comprising iPS cells and/or cardiomyocytes that have not been exposed to a pegRNA and/or iPS cells that have been exposed to one or more pegRNAs that do not edit one or more of the genes.

33. A method of identifying a target gene or gene mutation as associated with or cause for cardiotoxicity, comprising:
(i) obtaining one or more iPSC-derived cardiomyocytes carrying one or more (e.g., library) of wild-type and target gene editing mutations in one or more (cardiomyocyte) genes; or
obtaining one or more iPSC-derived cardiomyocytes wherein expression of one or more target genes has been altered; and
(ii) identifying the target gene or gene mutation and/or altered gene expression level as associated with or a cause for cardiotoxicity if the mutation and/or gene expression level has a negative effect on cardiomyocytes' function, proliferation, viability, survival, morphology, the expression of certain markers and receptors, or “heart beats” in vitro (which can model arrhythmias) relative to wild-type (e.g., isogenic) cells.

34. A method of identifying a target gene mutation as a mutation associated with drug-induced cardiotoxicity, comprising:
(i) obtaining one or more iPSC-derived cardiomyocytes carrying one or more (e.g., library) wild-type and target gene editing mutations in one or more (cardiomyocyte) genes; or
obtaining one or more iPSC-derived cardiomyocytes wherein expression of one or more genes has been altered; and
(ii) exposing the cardiomyocytes to a drug;
(ii) identifying the target gene or gene mutation as associated with drug-induced cardiotoxicity if exposure of the cardiomyocytes carrying the mutation and/or the altered gene expression level to the drug has a negative effect on cardiomyocytes' function, proliferation, viability, survival, morphology, the expression of certain markers and receptors, or “heart beats” in vitro (which can model arrhythmias) relative to wild-type (e.g., isogenic) cells exposed and to cells not exposed to the drug.

35. A method of assessing whether a drug is cardiotoxic to a wild-type subject comprising:
(i) obtaining one or more iPSC-derived wild-type cardiomyocytes;
(ii) exposing the cardiomyocytes to a drug; and
(ii) identifying the drug as cardiotoxic to a wild-type subject if exposure of the wild-type cardiomyocytes to the drug has a negative effect on cardiomyocytes' function, proliferation, viability, survival, morphology, the expression of certain markers and receptors, or “heart beats” in vitro (which can model arrhythmias) relative to wild-type (e.g., isogenic) cardiomyocytes not exposed to the drug.

36. A method for identifying a drug as causing cardiotoxicity in a subject due to the presence of a target gene mutation and/or due to the altered expression of a gene comprising:
(i) obtaining one or more iPSC-derived cardiomyocytes carrying one or more (e.g. library) of wild-type and target gene editing mutations in a target gene; or
obtaining one or more iPSC-derived cardiomyocytes wherein expression of one or more target genes has been altered; and
(ii) exposing the cardiomyocytes to the drug;
(iii) identifying the drug as causing cardiotoxicity due to the presence of the target gene mutation and/or due to the altered target gene expression when the cardiomyocytes carrying the mutation or altered target gene expression exhibit signs of cardiotoxicity in response to exposure to the drug in a cardiotoxicity assay but the wild-type cardiomyocytes do not;
wherein signs of cardiotoxicity comprise a negative effect on cardiomyocytes' function, proliferation, viability, survival, morphology, the expression of certain markers and receptors, or “heart beats” in vitro (which can model arrhythmias) relative to wild-type (e.g., isogenic) cells not exposed to the drug; wherein sequencing of the gene or portion of the gene to determine the identity of the mutation takes place before or after the cardiotoxicity assay.

37. The method of any one of claims 33 through 36, wherein the mutation is introduced in the target gene by gene editing (including CRISPR, base editing, prime editing).

38. The method of claim 37, wherein the mutation is introduced in the target gene by prime editing, preferably prime editing with any one of the pegRNAs of any one of claims 1 through 14 and/or the target gene expression is altered by CRISPR interference, preferably using a gRNA of any one of claims 15 and 16.

39. The method of any one of claims 33 to 38, wherein at least one of the target genes that is edited or has its expression altered is selected from the KONH2 hERG, KCNQ1, SCNA5, KONE1, KCNE2, KvLQT1, Nav1.5, ankyrin-B, MinK, MiRP1, Kir2.1, Cav1.2, caveolin-3, and Nav.beta.4 genes; genes encoding other channels, a sodium channel/related gene selected from SCN5A, ACN1B, SCN2B, SCN3B, SCN4B, GPD1L, RANGRF, SCN10A; a potassium channel/related gene selected from human ether-a-go-go related gene (hERG), Hyperpolarization-activated cyclic nucleotide-gated (HCN) channels; transient outward potassium current channel; Slowly activating delayed rectifier potassium current channels; Rapidly activating delayed rectifier potassium current channels; Inwardly rectifying potassium (Kir) channels; Inwardly rectifying potassium channels; G protein-coupled, inwardly rectifying potassium channels; ATP-sensitive potassium channels; a calcium channel/related gene selected from CACNA1C, CACNB2, CACNA2D1, RYR2, CASQ2, TRDN, CALM1-3; other genes selected from SNTA1, SLMAP, PKP2, ANK2, CAV3, SLC4A3, TRPM4, DPP6, IRX3, GNAI2, ADORAI, GNAS, KCNQ1, SCNA5, KCNE1, KCNE2, KvLQT1, Nav1.5, ankyrin-B, MinK, MiRP1, Kir2.1, Cav1.2, caveolin-3, Nav.beta.4 ATP-binding cassette (ABC) transporters involved in drug transport (e.g., ABCB1, ABCB4, ABCCI, ABCC2, SLC10A2, SLC28A3, SLC22A7, SLC22A17), Carbonyl reductases in drug metabolism (e.g., CBR3), Hyaluronan synthase 3 involved in oxidative stress response (e.g., HAS3), Hereditary hemochromatosis protein in iron metabolism (e.g., HFE), Retinoic acid receptor gamma and DNA topoisomerases in topoisomerase-induced DNA damage (e.g., RARG, TOP2B), CUGBP Elav-like family member 4 in splicing of sarcomere genes (e.g., CELF4), DNA polymerase gamma in mitochondrial replication (e.g., DPOG2), and chaperones involved in ion channel trafficking (e.g., Hsp70 and Hsp90).

40. The method of claim 39, wherein the target gene is the KONH2 hERG gene.

41. The method of claim 40, wherein the mutation is selected from those that may result from the SNPs of Table 1 and SEQ ID NOs.: 2-1054, preferably V476I, preferably SEQ ID NO: 105.

42. A method for identifying a subject in need of treatment with a drug as being sensitive to said drug-induced cardiotoxicity comprising testing a DNA sample from the subject (e.g., collected from a tissue comprising cardiomyocytes and/or from cardiomyocytes derived from iPSC isolated from the subject) for the presence of one or more target gene mutations or altered expression levels that have been identified as associated with said drug-induced cardiotoxicity by the method of any one of claims 33 to 41 and, optionally, choosing a different drug for treating the subject than the drug that induces cardiotoxicity.

43. The method of claim 42, wherein the subject is identified as suffering from, or being susceptible to, an arrhythmia, hypertrophic cardiomyopathy, sudden unexplained death, obesity-related cardiac disease, primary dilated cardiomyopathy, primary familial hypertrophic cardiomyopathy, or seizure, preferably an arrhythmia selected from Long QT syndrome (including Congenital long QT syndrome, Long QT syndrome 2, and bradycardia-induced Long QT syndrome), Brugada syndrome, Short QT syndrome 1, Sudden Infant Death Syndrome, Acquired long QT syndrome, ventricular tachycardia, Wolff-Parkinson-White pattern arrhythmia, Arrhythmogenic right ventricular cardiomyopathy, Atrial fibrillation, Catecholaminergic polymorphic ventricular tachycardia type 1, Prolonged QT interval, Paroxysmal familial ventricular fibrillation 1, Sudden cardiac arrest/death, and Torsades de pointes, when the subject carries one or more of the gene mutations.

44. The cells of any one of claims 22 to 30, or of the method of any one of claims 33 to 41, wherein the iPSC are prepared from any somatic cell including skin-derived fibroblasts, and peripheral blood mononuclear cells, and reprogrammed by any integrative (i.e. lentiviral) or episomal (i.e. plasmid, Sendai virus) vectors containing any combination of transcriptional factors, including OCT4, SOX2, KLF4 and MYC, or transcriptional factors delivered via RNA (mRNA or miRNA) or combinations of small molecules and growth factors, or direct modulation of gene expression via RNAi or CRISPRi/a.

45. The method of any one of claims 33 to 41, wherein the cardiomyocytes are derived from iPSC by in vitro differentiation using combinations of small molecules, and/or growth factors, including modulation of Wnt pathway followed by glucose starvation for selection of highly purified cardiomyocyte populations, and combinations with nucleic acids (including RNA, miRNA, siRNA).

46. The method of any one of claims 33 to 41, wherein signs of cardiotoxicity comprise changes in cardiomyocyte function, cell viability, survival, morphology, the expression of certain markers and receptors, heart beats in vitro and, and where these signs are measured, preferably, with models arrhythmias, a patch clamp technique, an external recording method, a voltage-sensitive dye, or an intracellular ion-sensitive dye.

47. A method of prime editing an iPS cell or an iPSC-derived cardiomyocyte cell comprising contacting the cell's double stranded target DNA with a pegRNA designed to edit a target gene (e.g., cardiomyocyte), preferably a pegRNA according to any one of claims 1 through 14, and with a prime editor, and optionally a sgRNA, preferably wherein the prime editor comprises a nucleic acid programmable DNA binding protein (napDNAbp), which is fused to a domain comprising polymerase (preferably, RNA-dependent DNA polymerase) activity, wherein the contacting prime edits the cells by installing one or more nucleotide edits in the double stranded target DNA, thereby editing the double stranded target DNA.

48. The method of claim 47, wherein the pegRNA and/or a nucleic acid encoding the napDNAbp fused to a domain comprising RNA-dependent DNA polymerase activity, are introduced into the iPSC or IPSC-derived cardiomyocytes by transfection, viral transduction (lentiviral, AAV, etc.), nanoparticles, or nucleofection.

49. The method of any one of claims 47 and 48, wherein the napDNAbp is selected from the group consisting of Cas9, Cas12e, Cas12d, Cas12a, Cas12b1, Cas13a, Cas12c, Cas12b2, Cas13a, Cas12c, Cas12d, Cas12e, Cas12h, Cas12i, Cas12g, Cas12f (Cas14), Cas12f1, Cas12j (Casǐ), and Argonaute and, optionally, has a nickase activity.

50. The method of any one of claims 47 to 49, wherein the napDNAbp is a nuclease active Cas9 domain, a nuclease inactive Cas9 domain, or a Cas9 nickase domain or variant thereof.

51. The method of any one of claims 47 to 50, wherein the RNA-dependent DNA polymerase is a reverse transcriptase, preferable selected from Moloney Murine Leukemia virus reverse transcriptase (MMLV-RT), optionally wherein the MMLV-RT comprises one or more amino acid substitutions selected from D200N, T306K, W313F, T330P, and L603W compared to a wild type MMLV-RT.

52. The method of any one of claims 29 to 46, wherein the pegRNA and/or the gRNA are designed by PrimeDesign or another commercially or publicly available method known to one of ordinary skill in the art.

53. A polynucleotide comprising a DNA sequence coding for the pegRNA of any one of claims 1 through 14 or coding the gRNA of any one of claims 15 and 16, preferably the sequence of SEQ Nos: 1116 to 1134, or a pegRNA coded by the same.

54. A vector comprising the polynucleotide of claim 53, optionally wherein expression of the pegRNA or gRNA is under the control of a promoter.

55. The method of any one of claims 33 to 52, wherein the iPS cells or iPS cell-derived cardiomyocytes are cultured and/or tested in multi-well (e.g. 96 well) plates.

56. The method of claim 55, wherein each well comprises 1 cell or its progeny.

57. The method of any one of claim 55 or 56, wherein each cell comprises only one type of pegRNA, or nucleic acid encoding the pegRNA, preferably wherein the cells are sorted through the barcodes in each pegRNA and/or wherein the pegRNAs are sorted into each well prior to introduction into the cells.

58. The method of any one of claims 33 to 41, 45, and 46, wherein the drug is a drug that is hypothesized to cause arrhythmia.

59. The method of any one of claims 33 to 41, 45, 46, 55 to 58, wherein the drug is selected from a list of blackbox labeled drugs (currently marketed drugs that contain a label to indicate that Long QT patients should avoid using this drug; including Table 2), ii) drugs withdrawn from the US market due to cardiotoxicity events, iii) drugs that failed clinical trials due to cardiotoxicity events-(some may be marketed in other countries; iv) drugs associated with cardiotoxicity for particular SNPs as documented in the literature (e.g., ClinVar; antiarrhythmics, class i and iii; cisapride, amiodarone, dofetilide, clarithromycin, hydroxyzine, quinidine and disopyramide), and v) TdP associated drugs (including Table 3).

60. A method of treating a subject for arrhythmia and/or preventing arrhythmia in the subject, wherein the subject is identified by the method of claim 42 or 43 as carrying a variant mutation, wherein the treatment comprises genome editing of the genome of at least some of the subject's cardiomyocytes to edit the variant mutation and/or alter the expression of the target gene.

61. An array of cell culture wells or vessels each comprising at least one iPSC or iPSC-derived cardiomyocyte and at least one component of a gene editing system and/or gene expression altering system, wherein the gene editing system is designed to introduce one or more gene edits per iPSC/cardiomyocyte into at least one target gene and the gene expression altering system is designed to alter the expression of at least one target gene, wherein the target gene edit and/or altered expression level is hypothesized to cause cardiotoxicity or drug-induced cardiotoxicity when introduced into or present in the genome of a cardiomyocyte.

62. The array of claim 61, wherein the array comprises a multi-well tissue culture plate.

63. The array of claim 61, wherein each well or vessel carries only cells comprising a single gene edit or a single level of altered gene expression.

64. The array of any one of claims 62 and 63, wherein the array further comprises one or more elements of an in vitro cardiotoxicity assay.

65. The array of any one of claims 61 to 64, wherein the gene editing or gene expression altering system comprises one or more pegRNA according to any one of claims 1 to 14, one or more gRNA according to any one of claims 15 and 16, and/or any composition according to any one of claims 17 to 21.

66. The method, pegRNA, gRNA, polynucleotide, vector, and cell of any one of claims 1-66, wherein the coding DNA sequence coding for the pegRNA comprises any one of SEQ ID NOs: 1100 to 1113 and/or the coding sequence coding for the gRNA comprises any one of SEQ ID Nos 1116-1134 (or where the target is that of anyone one of SEQ ID NOs: 1055 to 1073), and the DNA coding sequence for the mRNA/SNP comprises any one of SEQ ID NOs.: 2 to 1054.
